(19) United States
(12) Patent Application Publication
(10) Pub. No.: US 2026/0176396 A1
(43) Pub. Date: Jun. 25, 2026

(54) POLYMERIZABLE MONOMER, POLYMER, CHEMICALLY-AMPLIFIED RESIST COMPOSITION, AND PATTERNING PROCESS

(71) Applicant: SHIN-ETSU CHEMICAL CO.,LTD., Tokyo (JP)

(72) Inventors: Masaki OHASHI, Joetsu-shi (JP); Masahiro FUKUSHIMA, Joetsu-shi (JP); Yutaro OTOMO, Joetsu-shi (JP)

(73) Assignee: SIN-ETSU CHEMICAL CO., LTD., Tokyo (JP)

(21) Appl. No.: 19/426,978

(22) Filed: Dec. 19, 2025

(30) Foreign Application Priority Data
Dec. 24, 2024 (JP) 2024-227711

(51) Int. Cl. C08F 220/26; G03F 7/00; G03F 7/004; G03F 7/039

(52) U.S. Cl. CPC C08F 220/26; G03F 7/0045; G03F 7/0392; G03F 7/70033

(57) ABSTRACT

The present invention is a polymerizable monomer having one or more of each of acid-labile groups having different structures. This can provide: a chemically-amplified resist composition that can have sensitivity and resolution that exceed those of conventional positive resist materials, can have low LWR and low size variation, and can give an excellent process window; a polymer contained in the chemically-amplified resist composition; a polymerizable monomer used for the polymer; and a patterning process using the composition.

### TECHNICAL FIELD

The present invention relates to: a polymerizable monomer; a polymer; a chemically-amplified resist composition; and a patterning process.

### BACKGROUND ART

As LSIs advance toward higher integration and higher processing speed, miniaturization of pattern rule is progressing rapidly. This is because the spread of high-speed communication of 5 G and artificial intelligence (AI) has progressed, and high-performance devices for processing these are needed. As a cutting-edge technology for miniaturization, 5-nm node devices have been mass-produced by extreme ultraviolet ray (EUV) lithography at a wavelength of 13.5 nm. Furthermore, studies are also in progress on employing EUV lithography in next-generation 3-nm node and the following-generation 2-nm node devices.

As the miniaturization progresses, image blurs due to acid diffusion become a problem. To ensure resolution for fine patterns with dimensional sizes of 45 nm and smaller, there is a proposal that it is important to not only improve dissolution contrast as previously reported, but also control acid diffusion. Nevertheless, since chemically-amplified resist materials enhance the sensitivity and contrast through acid diffusion, an attempt to minimize acid diffusion by reducing the temperature and/or time of post-exposure baking (PEB) results in significant reductions of sensitivity and contrast.

A triangular tradeoff relationship among sensitivity, resolution, and line width roughness (LWR) has been pointed out. Specifically, resolution improvement requires suppression of acid diffusion, whereas shortening acid diffusion distance results in the reduction of sensitivity.

The addition of an acid generator capable of generating a bulky acid is effective in suppressing acid diffusion. Hence, it has been proposed to incorporate in a polymer a repeating unit derived from an onium salt having a polymerizable unsaturated bond. In this case, the polymer also functions as an acid generator (polymer-bound acid generator). Patent Document 1 proposes a sulfonium and iodonium salt having a polymerizable unsaturated bond that generates a particular sulfonic acid. Patent Document 2 proposes a sulfonium salt having a sulfonic acid moiety directly bonded to the main chain.

As resist patterns advance toward further miniaturization, extreme ultraviolet ray (EUV) and electron beam have come to be used as exposure light, and issues that cannot be resolved by the suppression of acid diffusion alone have come to light. One such issue is the improvement of a process window. Due to the miniaturization of patterns, there is an increase in the effect that a slight deviation in exposure and development conditions has on the shapes of resist patterns and the generation of defects. There are also demands for a radiation-sensitive resin composition that has a wide process window and can absorb such slight deviations in process conditions. For example, in a case where a line-and-space pattern is formed with a positive resist, the greater the exposure dose, the thinner the pattern line width, and it is preferable for the shape of a pattern to be maintained as much as possible without the pattern collapsing or being chipped. Meanwhile, in regions where the exposure dose is small, it is desirable for the line-and-space pattern to be maintained as much as possible without spaces in the pattern being bridged. However, the above-described conventional radiation-sensitive resin composition is not yet capable of satisfying a sufficient process window.

### CITATION LIST

### Patent Literature

Patent Document 1: JP 2006-045311 A
Patent Document 2: JP 2006-178317 A

### SUMMARY OF INVENTION

### Technical Problem

The present invention has been made in view of the above circumstances. An object of the present invention is to provide: a chemically-amplified resist composition that can have sensitivity and resolution that exceed those of conventional positive resist materials in particular, can have low LWR and low size variation, and can give an excellent process window; a polymer contained in the chemically-amplified resist composition; a polymerizable monomer used for the polymer; and a patterning process using the composition.

### Solution to Problem

To achieve the object, the present invention provides a polymerizable monomer having one or more of each of acid-labile groups having different structures.

A chemically-amplified resist composition containing a polymer using the inventive polymerizable monomer can have sensitivity and resolution that exceed those of conventional positive resist materials, can have low LWR and low size variation, and can give an excellent process window.

In the present invention, the polymerizable monomer is preferably represented by the following general formula (A1),

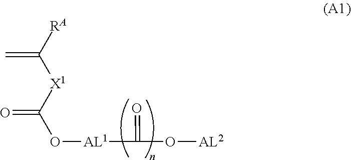

wherein R^{A} represents a hydrogen atom, a fluorine atom, a methyl group, or a trifluoromethyl group; X¹ represents a single bond or an arylene group; “n” represents 0 or 1; and AL¹ and AL² each represent an acid-labile group, provided that AL¹ and AL² each have a different structure.

In the present invention, such a polymerizable monomer is more preferable.

In this case, the polymerizable monomer of the general formula (A1) is preferably represented by the following general formula (A1-1) or (A1-2),

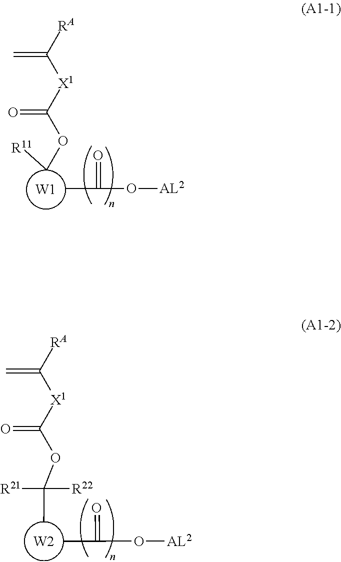

wherein R^{A}, X¹, “n”, and AL² are as defined above; R¹¹, R²¹, and R²² each independently represent a halogen atom, a hydroxy group, a nitro group, or a hydrocarbyl group having 1 to 40 carbon atoms and optionally containing a heteroatom; W1 represents an alicyclic hydrocarbon group; and W2 represents an alicyclic hydrocarbon group or an aromatic hydrocarbon group.

In the present invention, such a polymerizable monomer is further preferable.

In this case, it is preferable that the polymerizable monomer of the general formula (A1-1) is represented by either of the following general formulae (A2-1) and (A2-2), and the polymerizable monomer of the general formula (A1-2) is represented by either of the following general formulae (A2-3) and (A2-4),

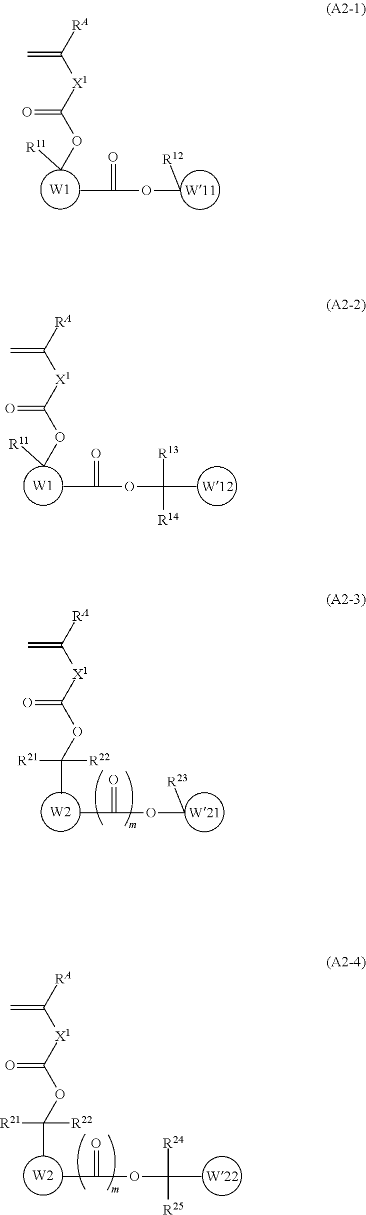

wherein R^{A}, X¹, R¹¹, R²¹, R²², W1, and W2 are as defined above; R¹², R¹³, R¹⁴, R²³, R²⁴, and R²⁵ each independently represent a halogen atom, a hydroxy group, a nitro group, or a hydrocarbyl group having 1 to 40 carbon atoms and optionally containing a heteroatom; W′11 and W′21 each independently represent an alicyclic hydrocarbon group; W′12 and W′22 each independently represent an alicyclic hydrocarbon group or an aromatic hydrocarbon group; and “m” represents 0 or 1, provided that when the W2 is an alicyclic hydrocarbon group, “m” is 1, and when the W2 is an aromatic hydrocarbon group, “m” is 0 or 1.

In the present invention, such a polymerizable monomer is particularly preferable.

The present invention also provides s polymer comprising a repeating unit obtained from the above-described polymerizable monomer.

A chemically-amplified resist composition containing such a polymer can have sensitivity and resolution that exceed those of conventional positive resist materials, can have low LWR and low size variation, and can give an excellent process window.

In this case, the polymer preferably further comprises a repeating unit represented by the following general formula (b1),

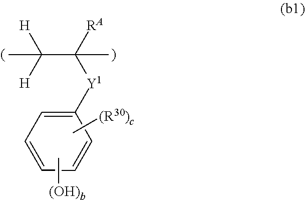

wherein R^{A} represents a hydrogen atom, a fluorine atom, a methyl group, or a trifluoromethyl group; Y¹ represents a single bond or *—C(═O)—O—; “*” represents an attachment point to the carbon atom of the main chain; R³⁰ represents a halogen atom, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and “b” represents an integer of 1 to 4 and “c” represents an integer of 0 to 4, provided that 1≤b+c≤5.

In the present invention, the polymer preferably has such a repeating unit.

In the present invention, the polymer preferably further comprises at least one selected from a repeating unit represented by the following general formula (c1), a repeating unit represented by the following general formula (c2), a repeating unit represented by the following general formula (c3), and a repeating unit represented by the following general formula (c4),

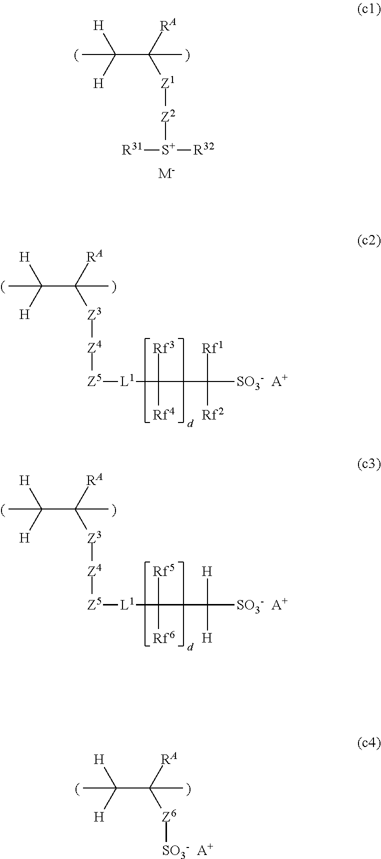

wherein R^{A} represents a hydrogen atom, a fluorine atom, a methyl group, or a trifluoromethyl group; Z¹ represents a single bond or a phenylene group; Z² represents *—C(═O)—O—Z²¹—, *—C(═O)—NH—Z²¹—, or *—O—Z²¹—; Z²¹ represents an aliphatic hydrocarbylene group having 1 to 6 carbon atoms, a phenylene group, or a divalent group derived from a combination of these groups, and optionally contains a carbonyl group, an ester bond, an ether bond, or a hydroxy group; each Z³ independently represents a single bond, a phenylene group, a naphthylene group, or *—C(═O)—O—Z³¹—; Z³¹ represents an aliphatic hydrocarbylene group having 1 to 10 carbon atoms, a phenylene group, or a naphthylene group, the aliphatic hydrocarbylene group optionally containing a hydroxy group, an ether bond, an ester bond, or a lactone ring; each Z⁴ independently represents a single bond, **—Z⁴—C(═O)—O—, **—C(═O)—NH—Z⁴¹—, or **—O—Z⁴—; Z⁴¹ represents a hydrocarbylene group having 1 to 20 carbon atoms and optionally containing a heteroatom; each Z⁵ independently represents a single bond, ***—Z⁵¹—C(═O)—O—, ***—C(═O)—NH—Z⁵¹—, or ***—O—Z⁵¹—; Z⁵¹ represents a hydrocarbylene group having 1 to 20 carbon atoms and optionally containing a heteroatom; Z⁶ represents a single bond, a methylene group, an ethylene group, a phenylene group, a fluorinated phenylene group, a phenylene group having a substituent which is a trifluoromethyl group, *—C(═O)—O—Z⁶¹—, *—C(═O)—NH—Z⁶¹—, or *—O—Z⁶¹—; Z⁶¹ represents an aliphatic hydrocarbylene group having 1 to 6 carbon atoms, a phenylene group, a fluorinated phenylene group, or a phenylene group having a substituent which is a trifluoromethyl group, and optionally contains a carbonyl group, an ester bond, an ether bond, or a hydroxy group; “*” represents an attachment point to the carbon atom of the main chain; “**” represents an attachment point to Z³; “***” represents an attachment point to Z⁴; R³¹ and R³² each independently represent a hydrocarbyl group having 1 to 20 carbon atoms and optionally containing a heteroatom, the R³¹ and the R³² optionally being bonded to each other to form a ring together with the sulfur atom bonded thereto; L¹ represents a single bond, an ether bond, an ester bond, a carbonyl group, a sulfonic acid ester bond, a carbonate bond, or a carbamate bond; Rf¹ and Rf² each independently represent a fluorine atom or a fluorinated saturated hydrocarbyl group having 1 to 6 carbon atoms; Rf³ and Rf⁴ each independently represent a hydrogen atom, a fluorine atom, or a fluorinated saturated hydrocarbyl group having 1 to 6 carbon atoms; Rf⁵ and Rf⁶ each independently represent a hydrogen atom, a fluorine atom, or a fluorinated saturated hydrocarbyl group having 1 to 6 carbon atoms, provided that not all of the Rf⁵ and the Rf⁶ are hydrogen atoms simultaneously; M⁻ represents a non-nucleophilic counter ion; A⁺ represents an onium cation; and “d” represents an integer of 0 to 3.

In the present invention, the polymer preferably has such a repeating unit.

The present invention also provides a chemically-amplified resist composition comprising: a base resin including the above-described polymer; and an organic solvent.

Such a chemically-amplified resist composition can have sensitivity and resolution that exceed those of conventional positive resist materials, can have low LWR and low size variation, and can give an excellent process window.

In this case, the chemically-amplified resist composition preferably further comprises one or more selected from a quencher, a photo-acid generator, and a surfactant.

The inventive chemically-amplified resist composition can contain these additives.

The present invention also provides a patterning process comprising the steps of:
forming a resist film on a substrate by using the above-described chemically-amplified resist composition;
exposing the resist film by using a high-energy beam; and
developing the exposed resist film by using a developer.

Such a patterning process can provide a patterning process that uses a chemically-amplified resist composition that has sensitivity and resolution that exceed those of conventional positive resist materials, has low LWR and low size variation, and gives an excellent process window.

In this case, the high-energy beam is preferably a KrF excimer laser beam, an ArF excimer laser beam, an electron beam, or an extreme ultraviolet ray having a wavelength of 3 to 15 nm.

In the present invention, such high-energy beams can be used.

### Advantageous Effects of Invention

As described above, when patterning is performed by using a chemically-amplified resist composition that contains, as a base resin, a polymer using the inventive polymerizable monomer, it is possible to form a resist pattern having a high contrast, favorable sensitivity, and excellent lithography properties, such as LWR and process window.

### DESCRIPTION OF EMBODIMENTS

As stated above, there have been demands for the development of: a chemically-amplified resist composition that can have sensitivity and resolution that exceed those of conventional positive resist materials in particular, can have low LWR and low size variation, and can give an excellent process window; a polymer contained in the chemically-amplified resist composition; a polymerizable monomer used for the polymer; and a patterning process using the composition.

To achieve the object, the present inventors have earnestly studied, and found out that a chemically-amplified resist composition containing, as a base resin, a polymer obtained from a polymerizable monomer having a particular structure has high sensitivity and high contrast, is excellent in lithography properties, such as LWR and process window, and is extremely effective in fine patterning. Thus, the present invention has been completed.

That is, the present invention is a polymerizable monomer having one or more of each of acid-labile groups having different structures.

Hereinafter, the present invention will be described in detail, but the present invention is not limited thereto.

### [Polymerizable Monomer]

The inventive polymerizable monomer has one or more of each of acid-labile groups having different structures. By one or more of each of acid-labile groups having different structures being contained per molecule, the dissolution contrast between before and after deprotection caused by an acid is increased, and resolution is enhanced. Such a material having multiple acid-labile groups is also disclosed in JP 2017-019911 A, but in this case, only examples of compounds having a plurality of acid-labile groups having identical structures are given. In this case, there is great influence due to undesirable reaction in unexposed portions caused by acid diffusion, and degradation of resolution and LWR occurs. On the other hand, the inventive polymerizable monomer has acid-labile groups having different reactivities. Therefore, even if such an undesirable reaction in a boundary region occurs, the probability that multiple acid-labile groups will undergo deprotection simultaneously is low, and it is conjectured that lithography performance will not be degraded.

It is preferable that the inventive polymerizable monomer is represented by the following general formula (A1).

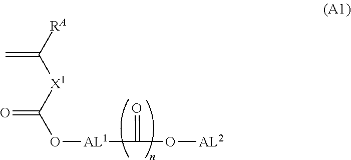

In the formula, R^{A} represents a hydrogen atom, a fluorine atom, a methyl group, or a trifluoromethyl group; X¹ represents a single bond or an arylene group; “n” represents 0 or 1; and AL¹ and AL² each represent an acid-labile group, provided that AL¹ and AL² each have a different structure.

In the general formula (A1), R^{A} represents a hydrogen atom, a fluorine atom, a methyl group, or a trifluoromethyl group. X¹ represents a single bond or an arylene group. Specific examples of the arylene group include a phenylene group, a naphthylene group, and an anthranylene group. “n” represents 0 or 1. AL¹ and AL² each independently represent an acid-labile group, provided that AL¹ and AL² each have a different structure. Specific examples of the acid-labile group include ones disclosed in JP 2013-080033 A and JP 2013-083821 A.

Typically, specific examples of the acid-labile group include ones represented by the following general formulae (AL-1) to (AL-3).

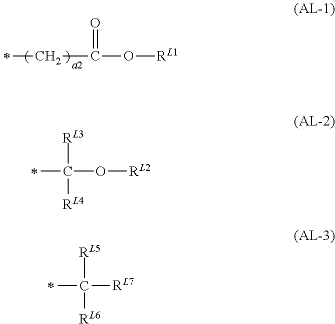

In the formulae, “*” represents an attachment point.

In the general formulae (AL-1) and (AL-2), R^{L1} and R^{L2} each independently represent a hydrocarbyl group having 1 to 40 carbon atoms, and optionally contain a heteroatom, such as an oxygen atom, a sulfur atom, a nitrogen atom, and a fluorine atom. The hydrocarbyl group may be saturated or unsaturated, and may be linear, branched, or cyclic. As the hydrocarbyl group, ones having 1 to 20 carbon atoms are preferable.

In the general formula (AL-1), “a2” represents an integer of 0 to 10, preferably an integer of 1 to 5.

In the general formula (AL-2), R^{L3} and R^{L4} each independently represent a hydrogen atom or a hydrocarbyl group having 1 to 20 carbon atoms, and optionally contain a heteroatom, such as an oxygen atom, a sulfur atom, a nitrogen atom, and a fluorine atom. The hydrocarbyl group may be saturated or unsaturated, and may be linear, branched, or cyclic. As the hydrocarbyl group, ones having 1 to 20 carbon atoms are preferable. Furthermore, any two of R^{L2}, R^{L3}, and R^{L4} may be bonded to each other to form a ring having 3 to 20 carbon atoms together with the carbon atom or the carbon atom and the oxygen atom bonded thereto. As the ring, rings having 4 to 16 carbon atoms are preferable, and alicyclic groups are particularly preferable.

In the general formula (AL-3), R^{L5}, R^{L6}, and R^{L7} each independently represent a hydrocarbyl group having 1 to 20 carbon atoms, and optionally contain a heteroatom, such as an oxygen atom, a sulfur atom, a nitrogen atom, and a fluorine atom. The hydrocarbyl group may be saturated or unsaturated, and may be linear, branched, or cyclic. As the hydrocarbyl group, ones having 1 to 20 carbon atoms are preferable. Furthermore, any two of R^{L5}, R^{L6}, and R^{L7} may be bonded to each other to form a ring having 3 to 20 carbon atoms together with the carbon atom bonded thereto. As the ring, rings having 4 to 16 carbon atoms are preferable, and alicyclic groups are particularly preferable.

Examples of structures of other acid-labile groups include ones disclosed in paragraphs [0064] to [0068] of JP 2023-123222 A and ones disclosed in paragraphs [0013] and [0014] of JP 7492842 B2. These structures use the fact that a conjugated olefin or an acrylic acid ester derivative is generated after an acid removal reaction as a driving force for the progress of the reaction.

In the present invention, it is preferable that the polymerizable monomer of the general formula (A1) is represented by the following general formula (A1-1) or (A1-2).

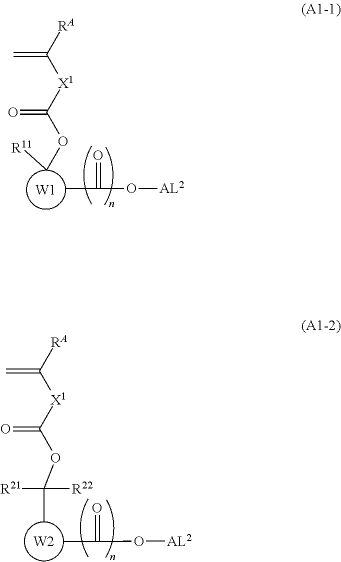

In the formulae, R^{A}, X¹, “n”, and AL² are as defined above; R¹¹, R²¹, and R²² each independently represent a halogen atom, a hydroxy group, a nitro group, or a hydrocarbyl group having 1 to 40 carbon atoms and optionally containing a heteroatom; W1 represents an alicyclic hydrocarbon group; and W2 represents an alicyclic hydrocarbon group or an aromatic hydrocarbon group.

In the general formulae (A1-1) and (A1-2), R^{A}, X¹, AL², and “n” are as defined above. R¹¹, R²¹, and R²² each independently represent a halogen atom, a hydroxy group, a nitro group, or a hydrocarbyl group having 1 to 40 carbon atoms, preferably 1 to 20 carbon atoms and optionally containing a heteroatom. Specific examples of such a hydrocarbyl group include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; aryl groups having 6 to 40 carbon atoms, such as a phenyl group, a naphthyl group, and an anthracenyl group; etc. Moreover, part or all of the hydrogen atoms of the hydrocarbyl groups may be substituted with a group containing a heteroatom, such as an oxygen atom, a sulfur atom, a nitrogen atom, or a halogen atom, and part of the —CH₂— of the hydrocarbyl groups may be substituted with a group containing a heteroatom, such as an oxygen atom, a sulfur atom, or a nitrogen atom. Thus, the resulting hydrocarbyl groups may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, carboxylic anhydride (—C(═O)—O—C(═O)—), a haloalkyl group, etc.

W1 represents an alicyclic hydrocarbon group. W2 represents an alicyclic hydrocarbon group or an aromatic hydrocarbon group. Specific examples of the alicyclic hydrocarbon group of W1 and W2 include cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group. Specific examples of the aromatic hydrocarbon group of W2 include aryl groups having 6 to 40 carbon atoms, such as a phenyl group, a naphthyl group, and an anthracenyl group. Moreover, part or all of the hydrogen atoms of the cyclic saturated hydrocarbyl groups may be substituted with a group containing a heteroatom, such as an oxygen atom, a sulfur atom, a nitrogen atom, or a halogen atom, and part of the —CH₂— of the cyclic saturated hydrocarbyl groups may be substituted with a group containing a heteroatom, such as an oxygen atom, a sulfur atom, or a nitrogen atom. Thus, the resulting cyclic saturated hydrocarbyl groups may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, carboxylic anhydride (—C(═O)—O—C(═O)—), a haloalkyl group, etc.

In the present invention, it is more preferable that the polymerizable monomer of the general formula (A1-1) is represented by either of the following general formulae (A2-1) and (A2-2), and the polymerizable monomer of the general formula (A1-2) is represented by either of the following general formulae (A2-3) and (A2-4).

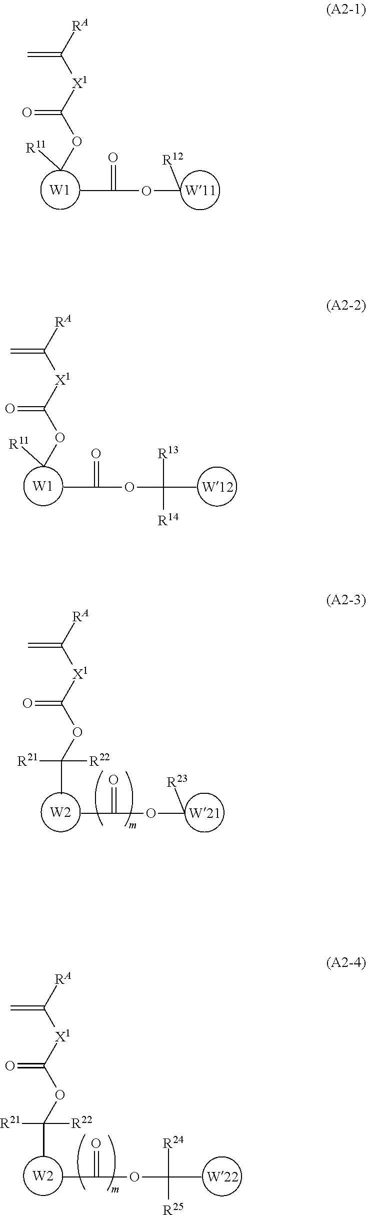

In the formulae, R^{A}, X¹, R¹¹, R²¹, R²², W1, and W2 are as defined above; R¹², R¹³, R¹⁴, R²³, R²⁴, and R²⁵ each independently represent a halogen atom, a hydroxy group, a nitro group, or a hydrocarbyl group having 1 to 40 carbon atoms and optionally containing a heteroatom; W′11 and W′21 each independently represent an alicyclic hydrocarbon group; W′12 and W′22 each independently represent an alicyclic hydrocarbon group or an aromatic hydrocarbon group; and “m” represents 0 or 1, provided that when the W2 is an alicyclic hydrocarbon group, “m” is 1, and when the W2 is an aromatic hydrocarbon group, “m” is 0 or 1.

In the general formulae (A2-1) to (A2-4), R^{A}, X¹, R¹¹, R²¹, R²², W1, and W2 are as defined above. R¹², R¹³, R¹⁴, R²³, R²⁴, and R²⁵ each independently represent a halogen atom, a hydroxy group, a nitro group, or a hydrocarbyl group having 1 to 40 carbon atoms, preferably 1 to 20 carbon atoms, and optionally containing a heteroatom. Specific examples of the hydrocarbyl group include those given for R¹¹.

W′11 and W′21 each independently represent an alicyclic hydrocarbon group, and specific examples thereof are similar to those given for W1. W′12 and W′22 each independently represent an alicyclic hydrocarbon group or an aromatic hydrocarbon group, and specific examples thereof are similar to those given for W2. “m” represents 0 or 1, provided that when the W2 is an alicyclic hydrocarbon group, “m” is 1, and when the W2 is an aromatic hydrocarbon group, “m” is 0 or 1.

Examples of the inventive polymerizable monomer include the following, but are not limited thereto.

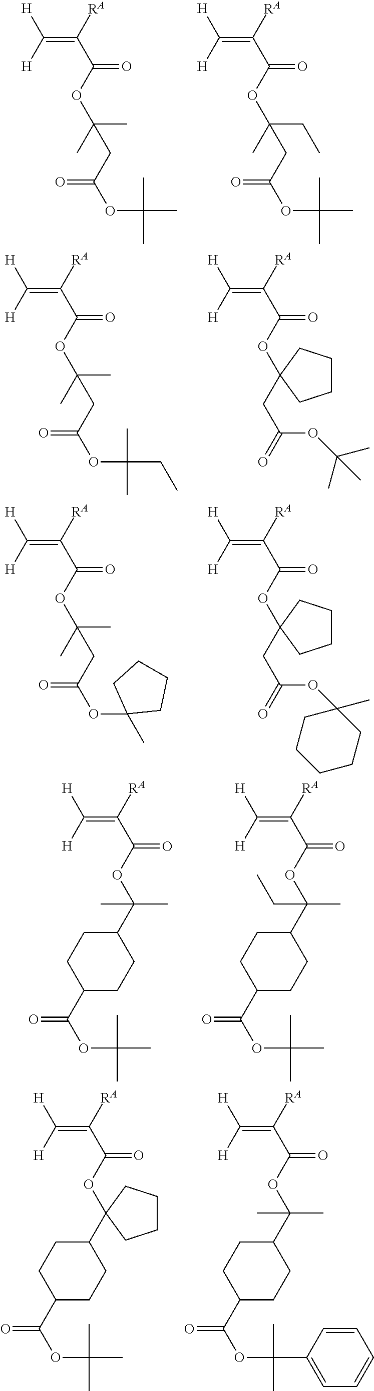

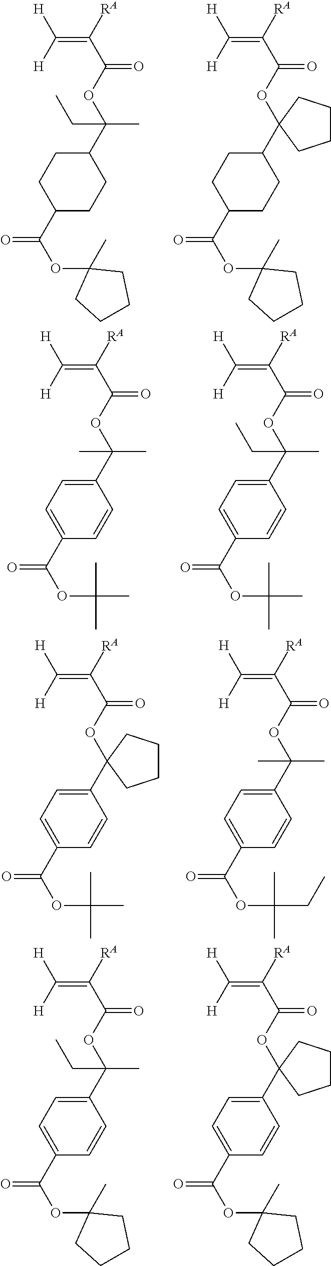

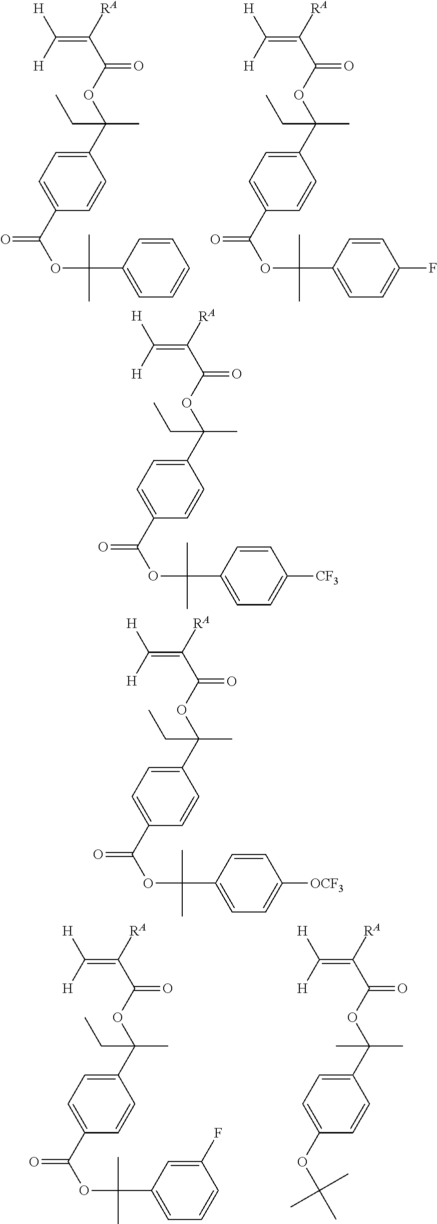

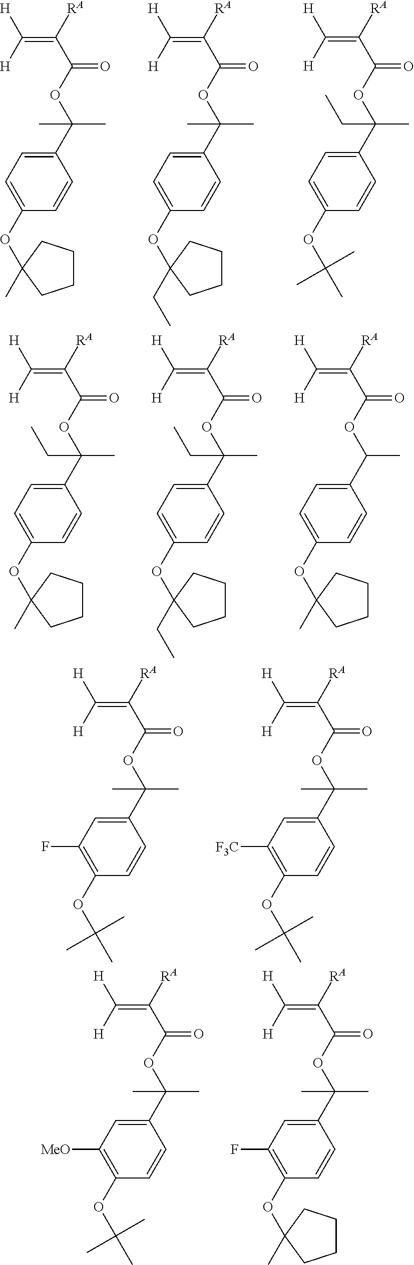

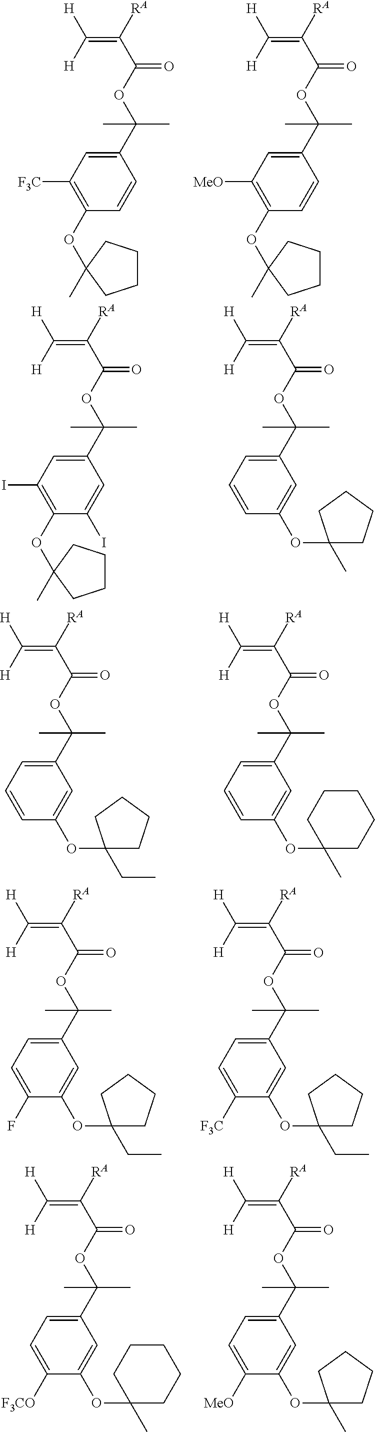

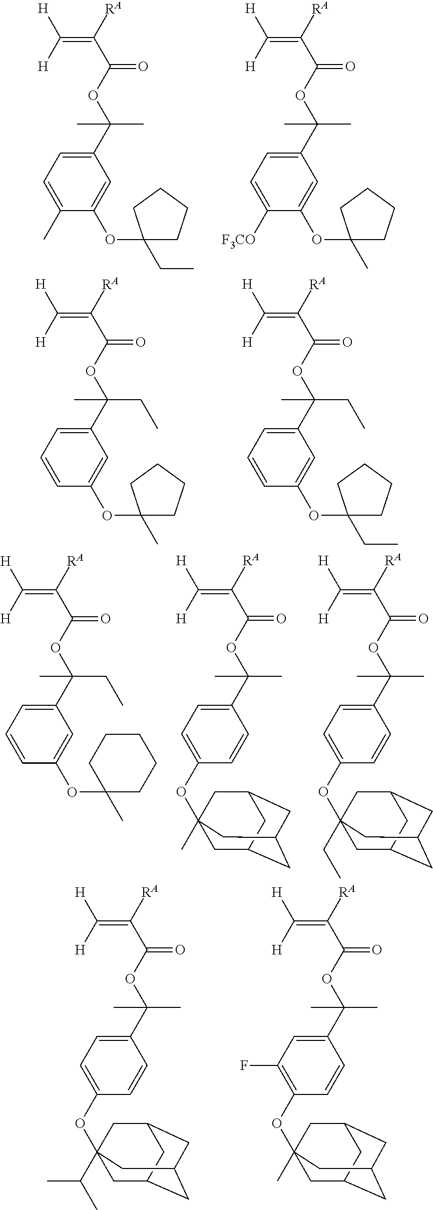

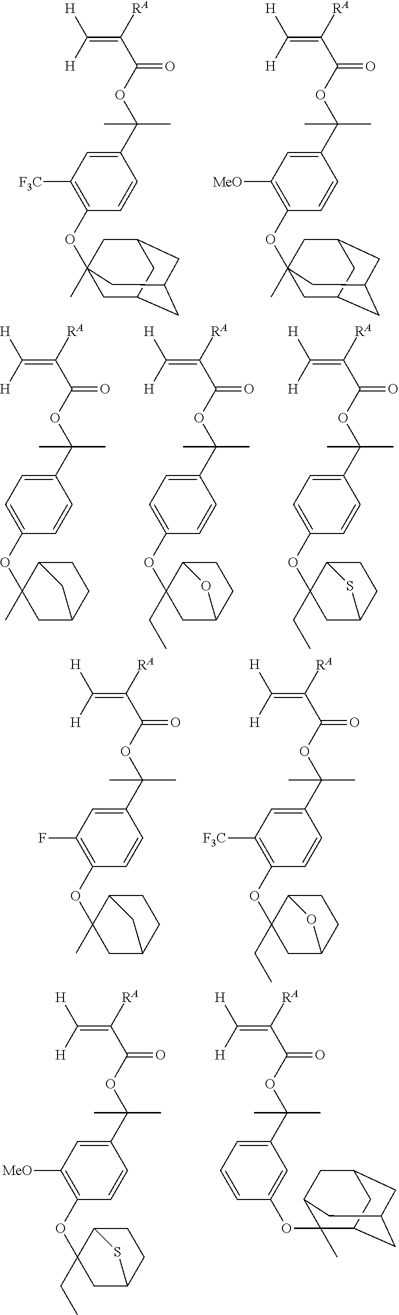

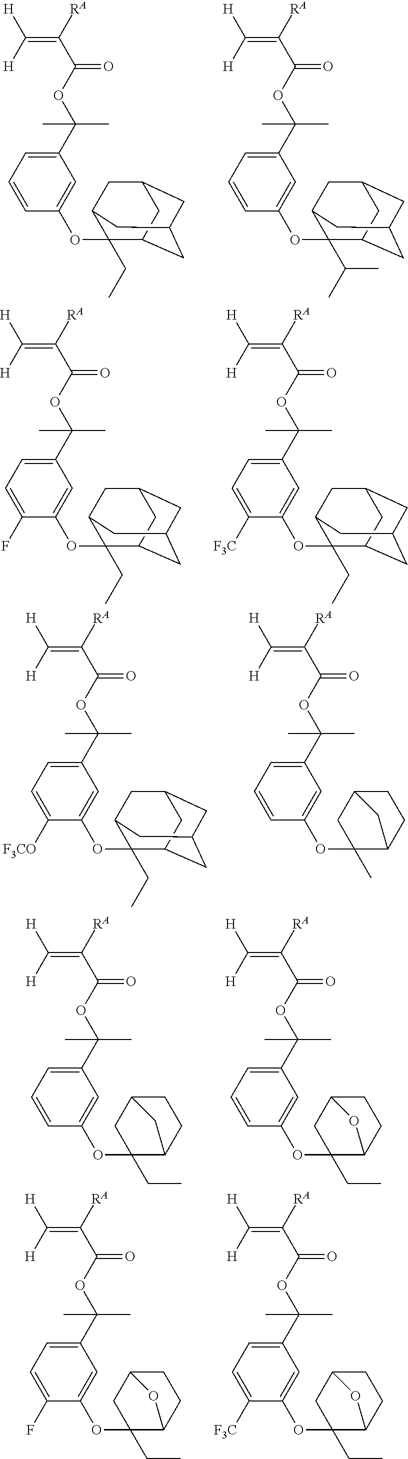

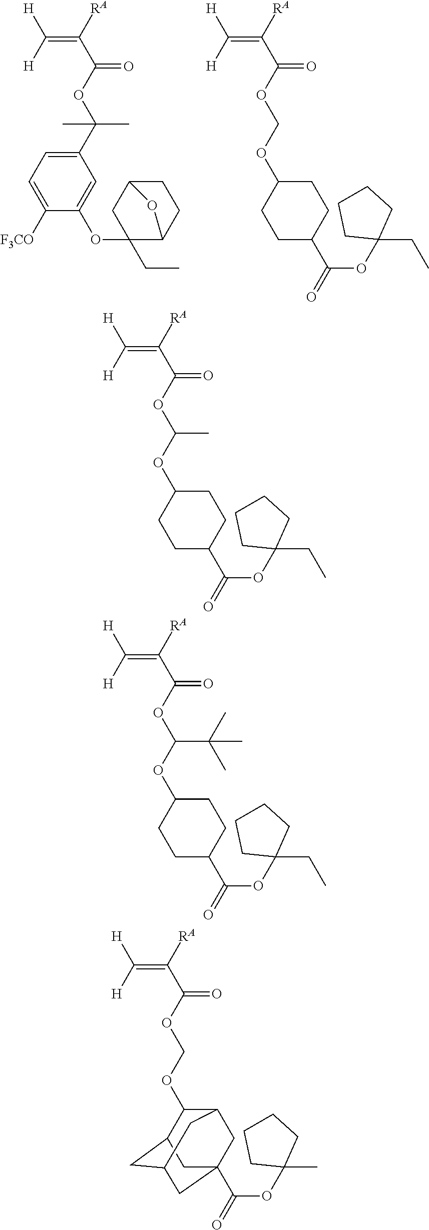

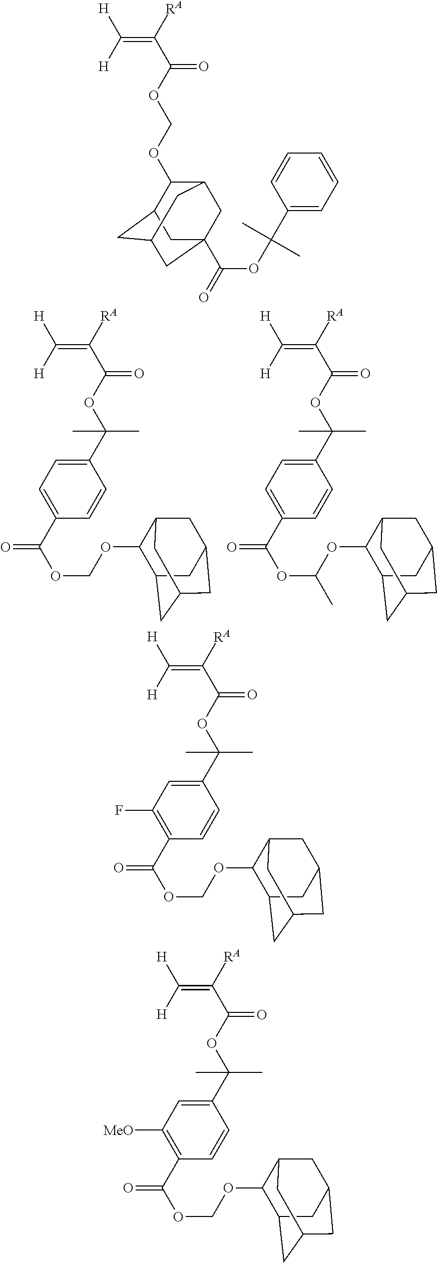

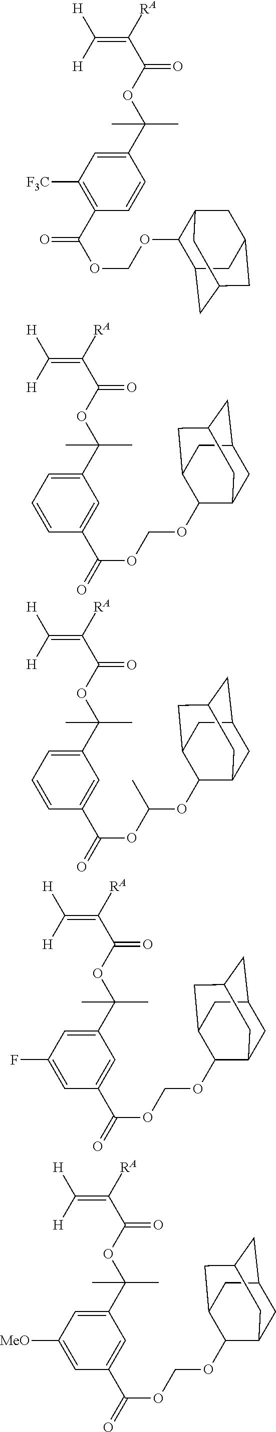

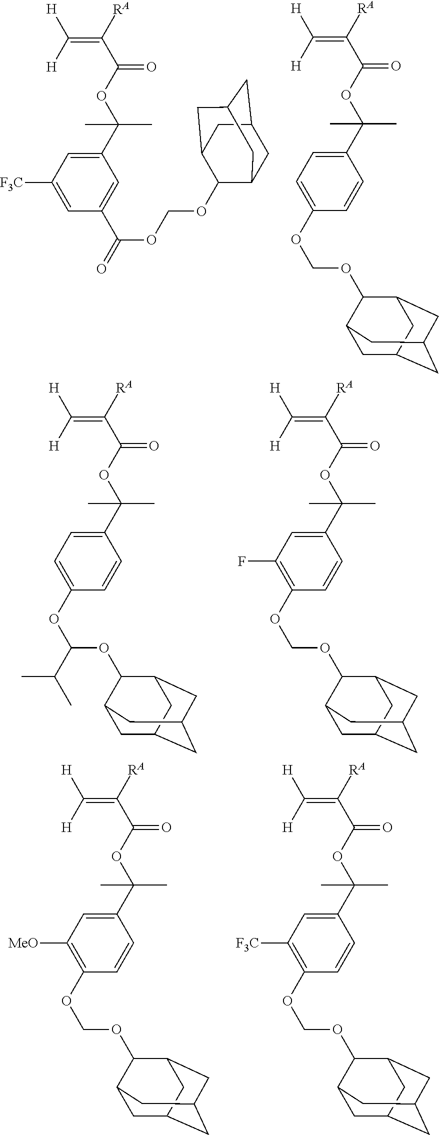

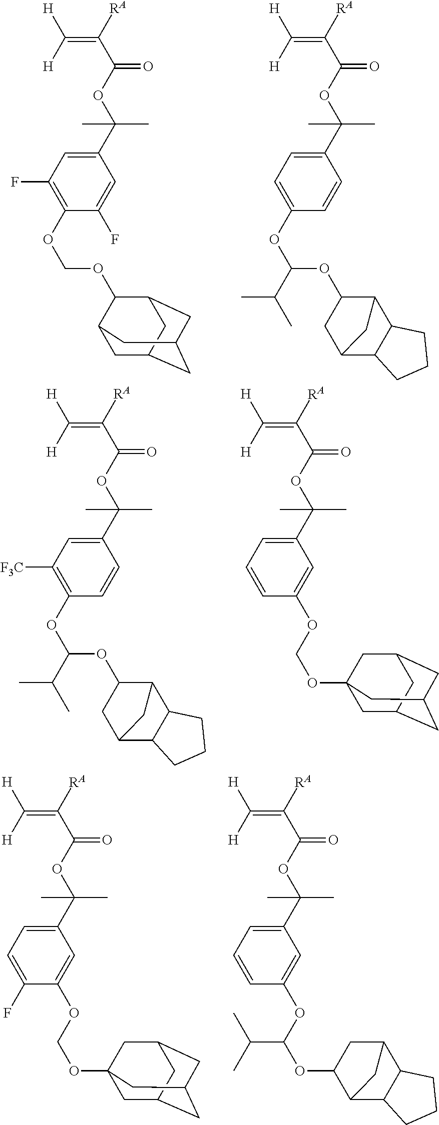

The inventive polymerizable monomer can be synthesized by a method fully using a known organic synthesis technique.

Particularly preferable structures for the inventive polymerizable monomer are those represented by the general formulae (A2-1) to (A2-4), having multiple cyclic groups, and reasons for this include the following.

It can be considered that, in repeating units obtained from polymerizable monomers represented by the general formulae (A2-1) to (A2-4) of the present invention contained in the base resin, the difference in the dissolution contrast in the polymer between before and after deprotection is increased, since a bulky acid-labile moiety, having multiple cyclic groups, is removed by exposure, contributing to the enhancement of resolution performance. As a similar approach, a resist composition having a bulky acid-labile group having a polycyclic hydrocarbon is already known (e. g. 3-ethyl-3-exotetracyclo[4.4.0.1^{2,5}.1^{7,10}]dodecanyl methacrylate disclosed in paragraph [0111] of JP 2005-320516 A). However, in the case of such an acid-labile group, the molecular weight and hydrophobicity of the removed compound are high, and therefore, the acid-labile group forms a residue after development in an alkaline development process. As a result, bridge defects appear on the underexposed side in positive resists, and the process window is degraded. On the other hand, in the inventive polymerizable monomer, it is conjectured that, since compounds that have undergone deprotection are further decomposed by an acid, low-molecular-weight and low-hydrophobicity compounds are formed in the end, and these are efficiently removed with an alkaline developer, widening the process window.

Note that, regarding unexposed portions, the inventive monomer has a structure having high hydrophobicity, and therefore, has high alkaline development resistance, so that breakage defects are reduced on the overexposed side. That is, a chemically-amplified resist composition containing a polymer obtained from the inventive polymerizable monomer has a wider margin on both the overexposed side and the underexposed side, so that the process window can be greatly improved, and it can be said that the composition is suitable as a positive resist material in fine patterning.

### [Chemically-Amplified Resist Composition]

The inventive chemically-amplified resist composition contains: (A) a base resin including a polymer including a repeating unit obtained from the inventive polymerizable monomer; and (B) an organic solvent.

The inventive chemically-amplified resist composition may further contain (C) a quencher or (D) a photo-acid generator as necessary, may further contain (E) a surfactant as necessary, and may further contain (F) other components as necessary. That is, it is preferable that the chemically-amplified resist composition further contains one or more selected from a quencher, a photo-acid generator, and a surfactant.

### [(A) Base Resin]

The base resin (A) contained in the inventive chemically-amplified resist composition includes a polymer including a repeating unit obtained from the inventive polymerizable monomer.

### [Polymer]

The inventive polymer includes a repeating unit obtained from the above-described polymerizable monomer. The polymerizable monomer can be used suitably as a constitutional unit of a polymer, being the base resin (A) in the inventive chemically-amplified resist composition (hereinafter, also referred to as repeating unit-A).

Regarding the repeating unit-A, two or more different kinds thereof may be copolymerized as a constitutional unit of the polymer.

The polymer preferably further includes a repeating unit represented by the following general formula (b1) (hereinafter, also referred to as repeating unit-b1).

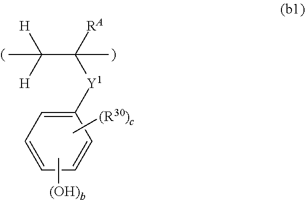

In the formula, R^{A} represents a hydrogen atom, a fluorine atom, a methyl group, or a trifluoromethyl group; Y¹ represents a single bond or *—C(═O)—O—; “*” represents an attachment point to the carbon atom of the main chain; R³⁰ represents a halogen atom, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and “b” represents an integer of 1 to 4 and “c” represents an integer of 0 to 4, provided that 1≤b+c≤5.

In the general formula (b1), R^{A} represents a hydrogen atom, a fluorine atom, a methyl group, or a trifluoromethyl group; Y¹ represents a single bond or *—C(═O)—O—; “*” represents an attachment point to the carbon atom of the main chain; R³⁰ represents a halogen atom, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and “b” represents an integer of 1 to 4 and “c” represents an integer of 0 to 4, provided that 1≤b+c≤5.

Specific examples of the repeating unit-b1 include the following, but are not limited thereto. Note that, in the following formulae, R^{A} is as defined above.

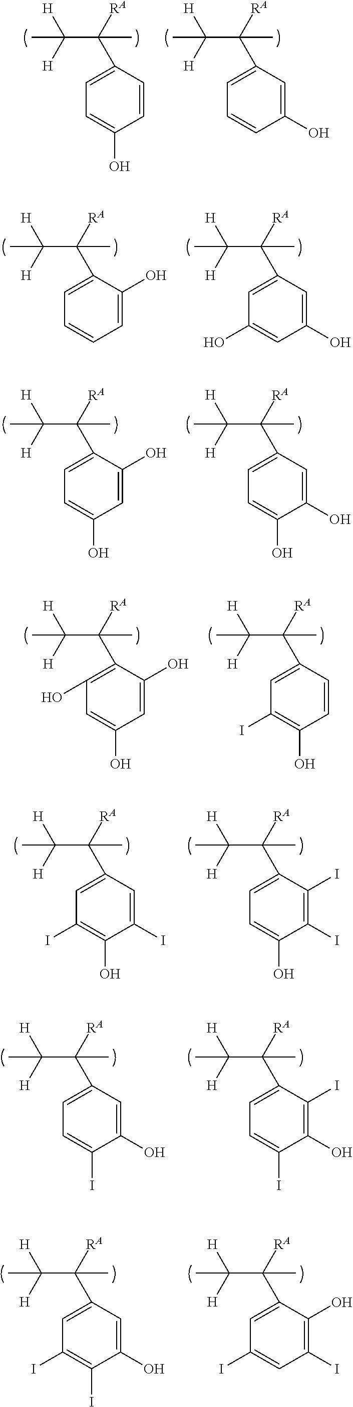

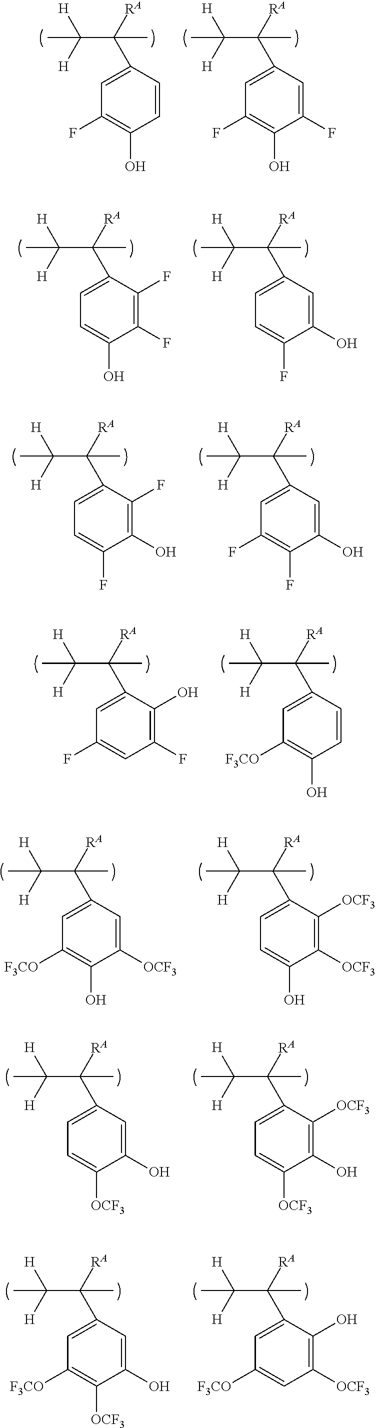

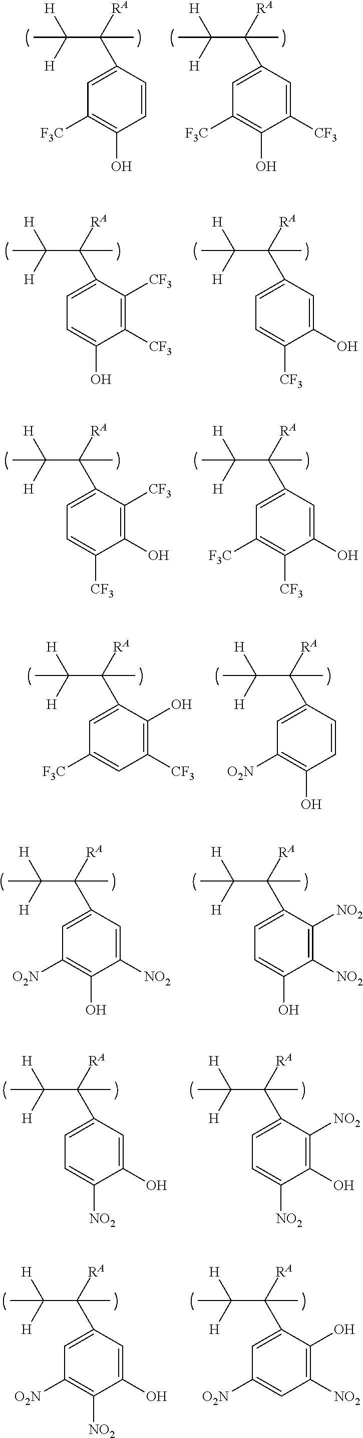

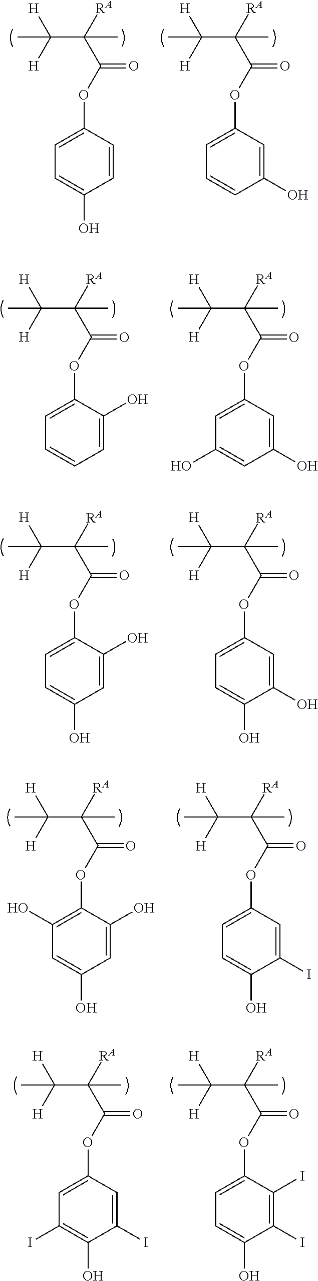

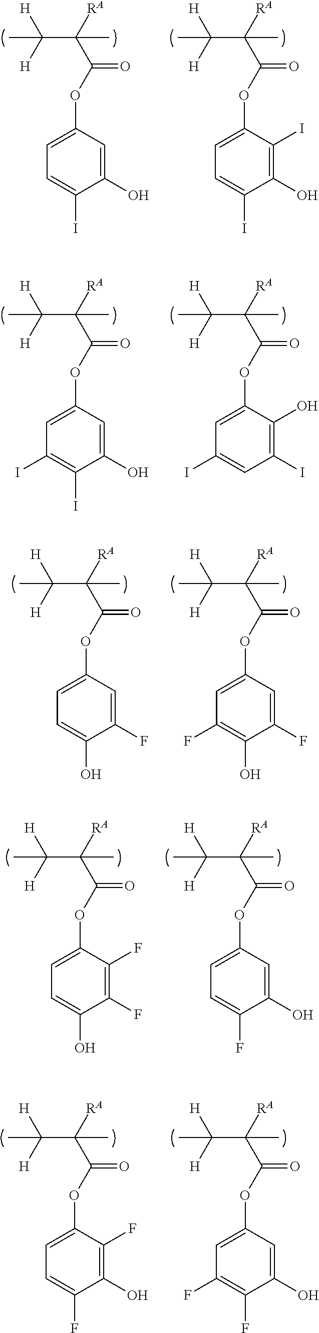

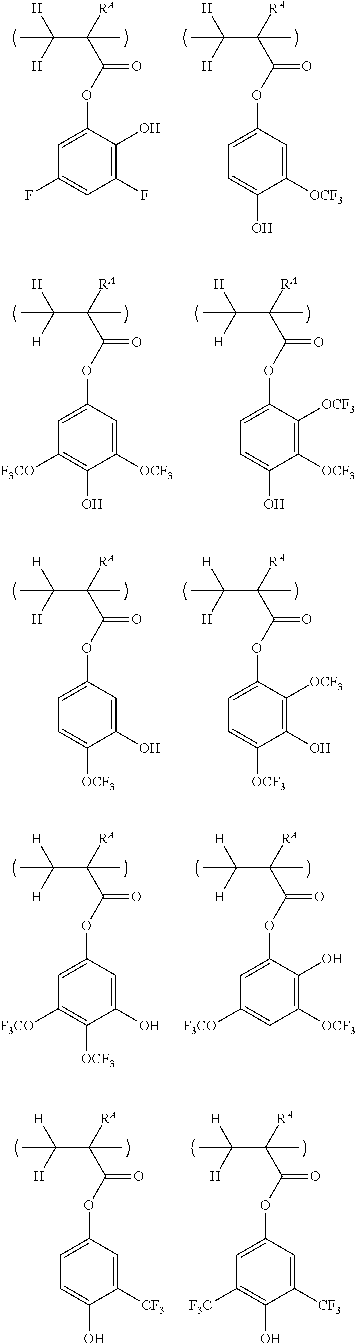

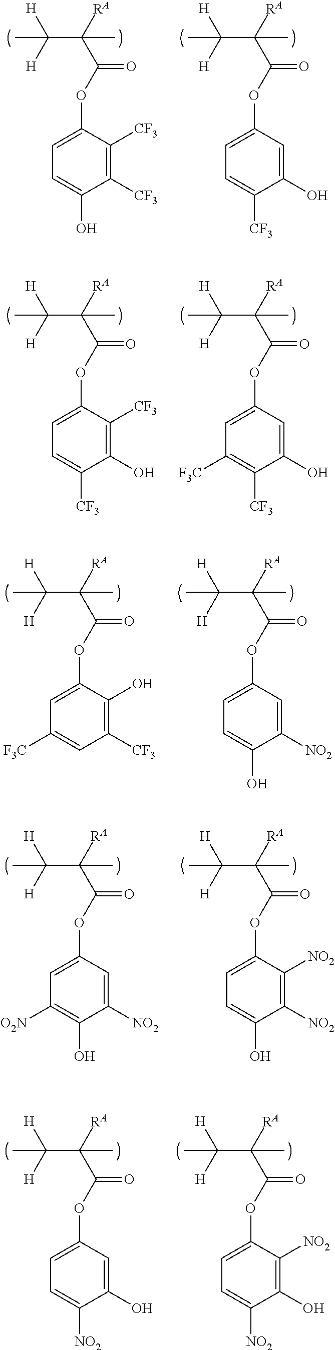

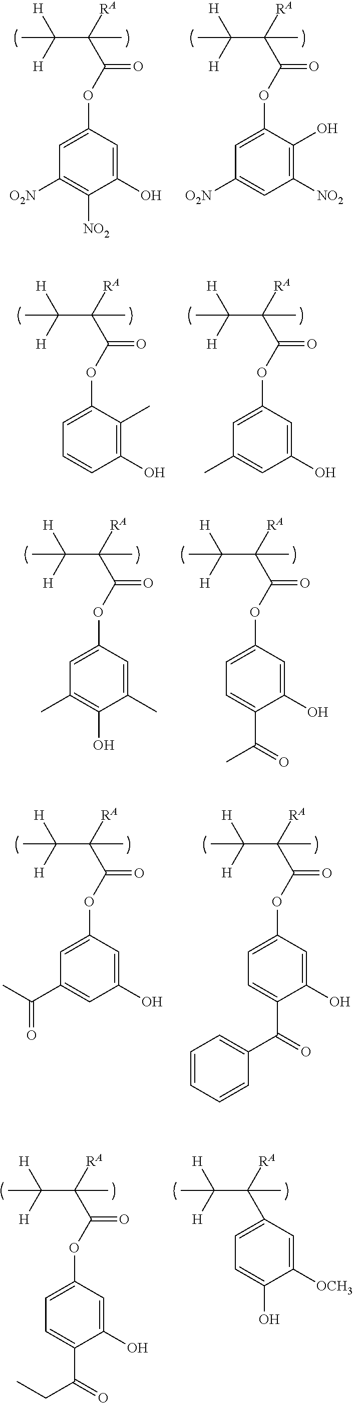

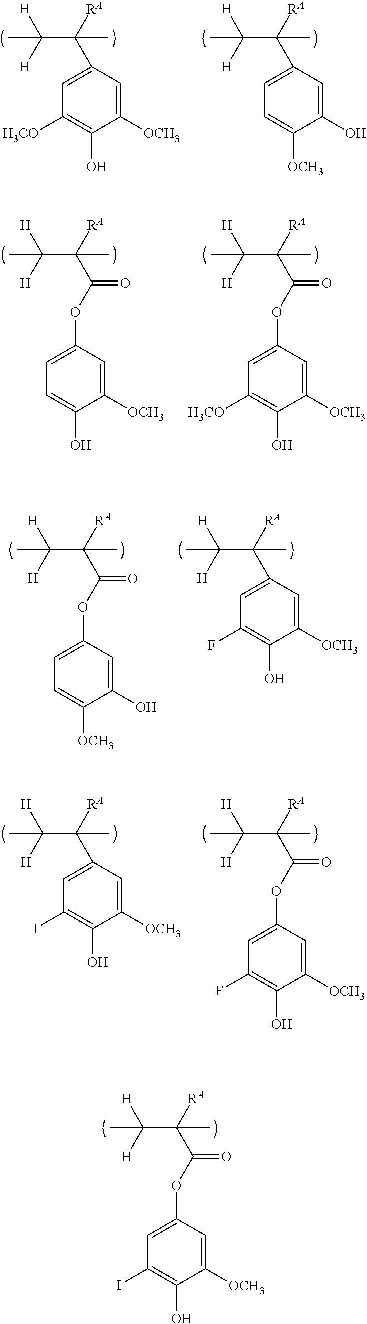

The polymer preferably further includes a repeating unit represented by the following general formula (b2) (hereinafter, also referred to as repeating unit-b2).

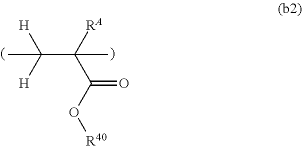

In the general formula (b2), R^{A} represents a hydrogen atom, a fluorine atom, a methyl group, or a trifluoromethyl group. R⁴⁰ represents a hydrogen atom or a group having 1 to 20 carbon atoms and including at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and carboxylic anhydride (—C(═O)—O—C(═O)—).

Specific examples of the repeating unit-b2 include the following, but are not limited thereto. Note that, in the following formulae, R^{A} is as defined above.

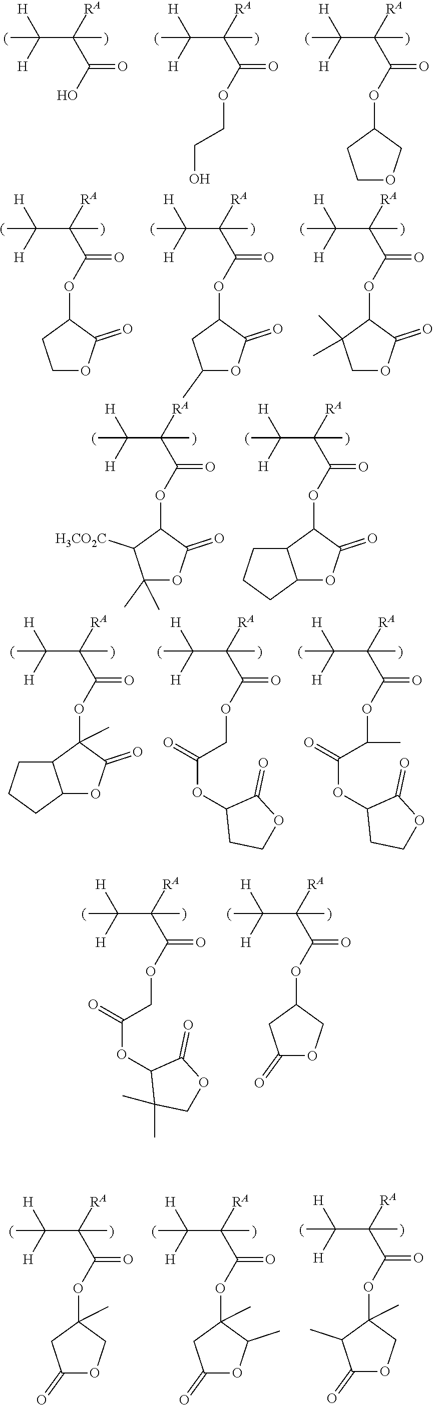

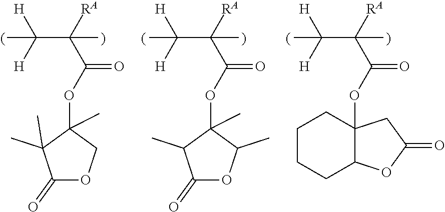

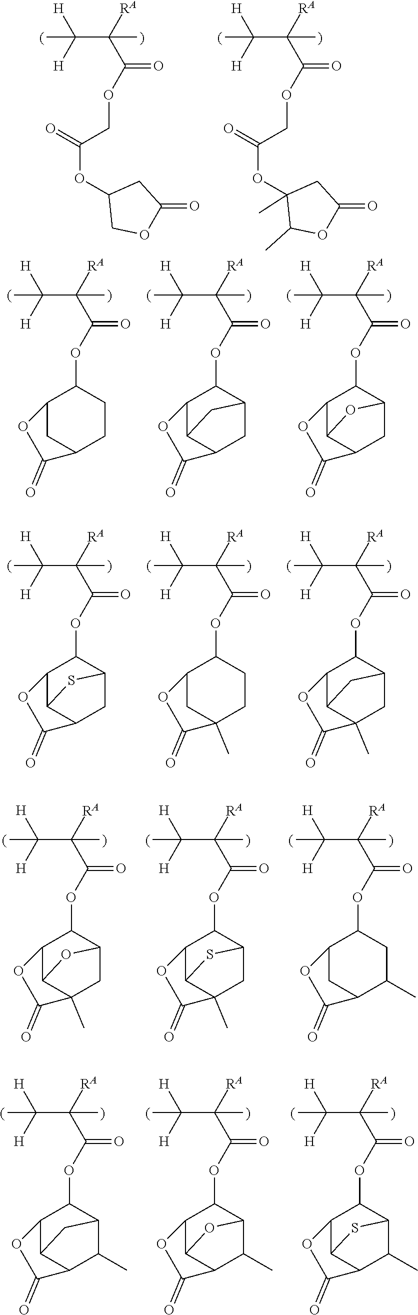

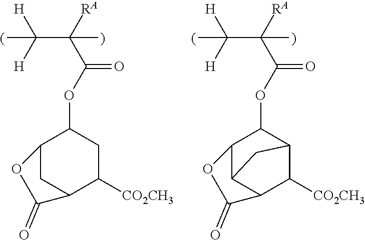

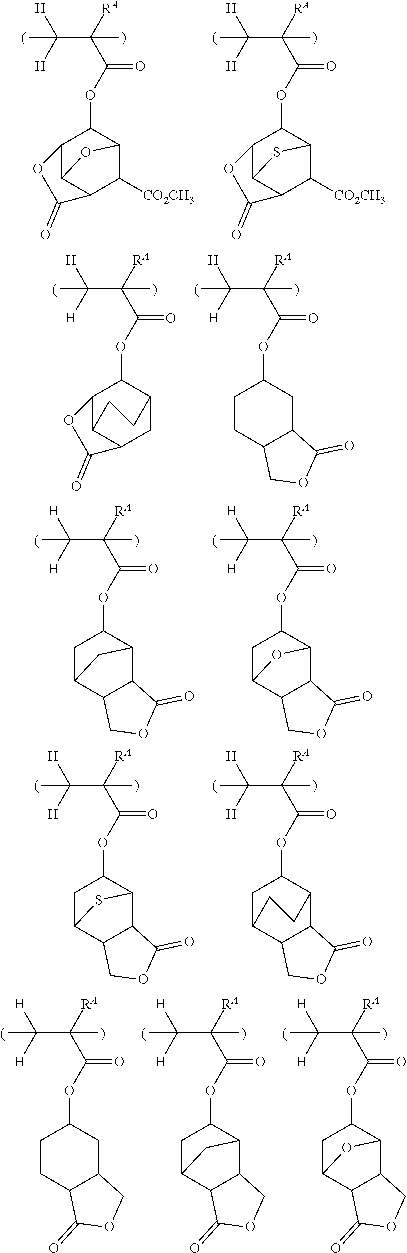

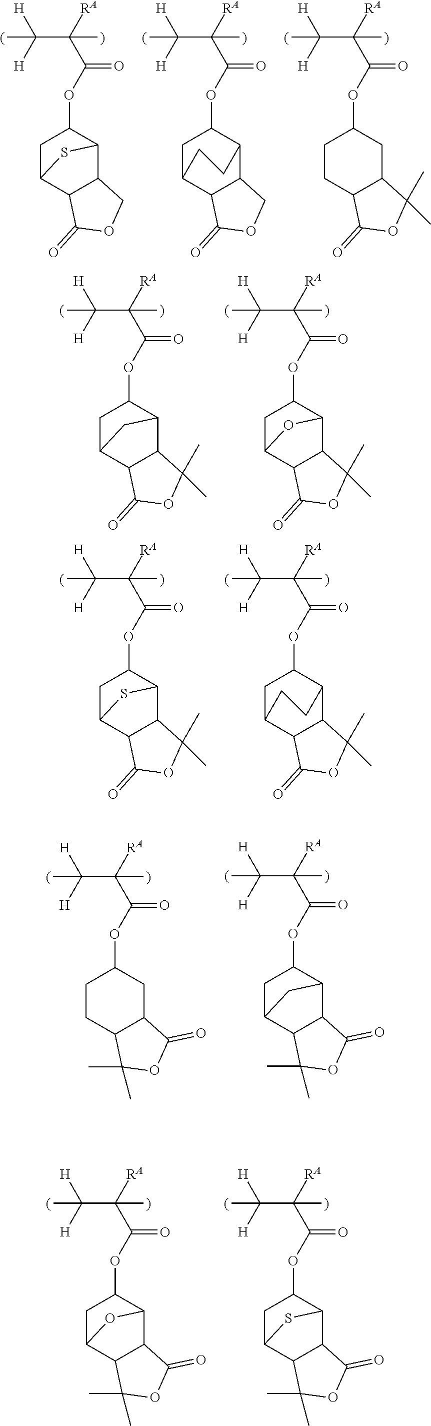

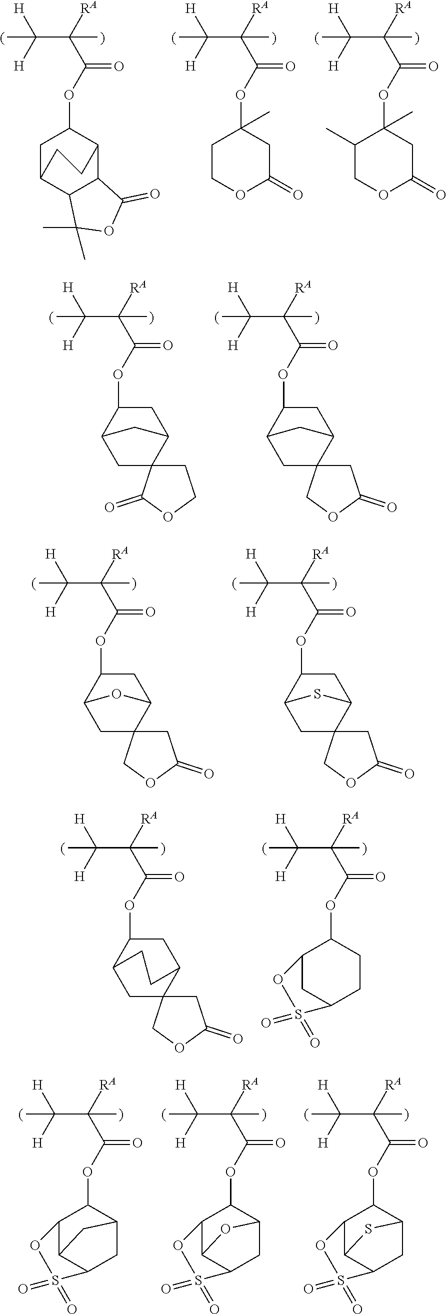

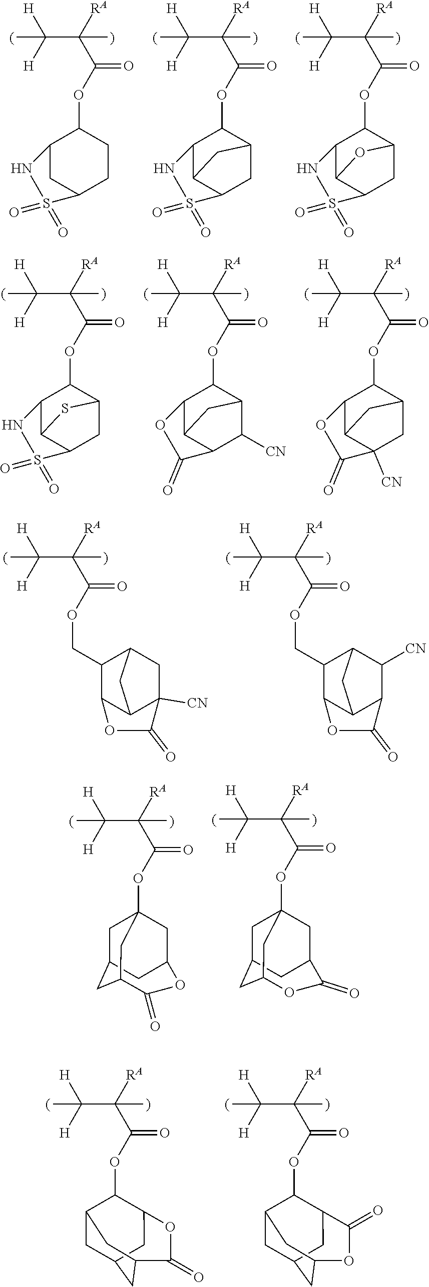

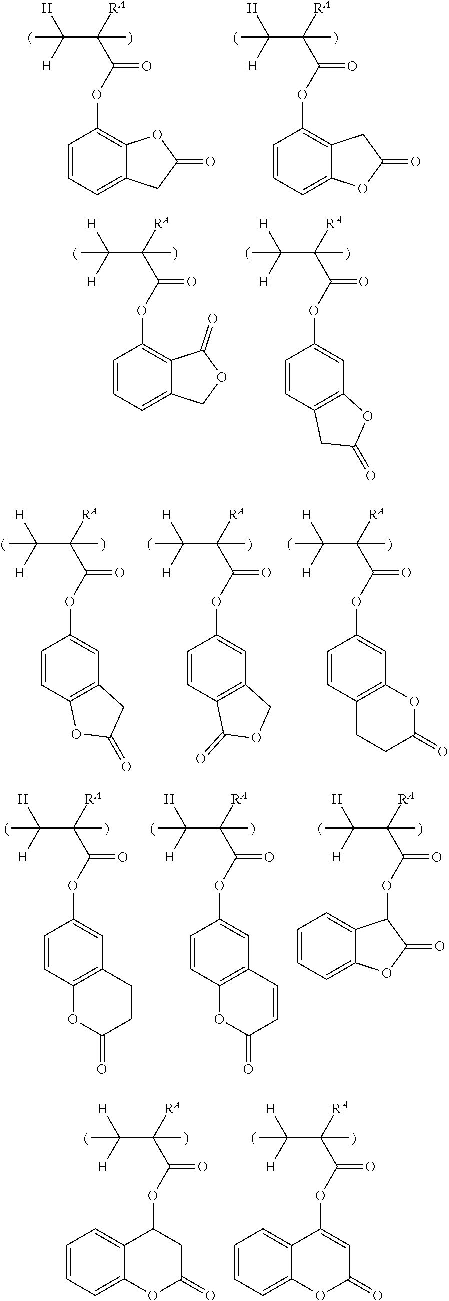

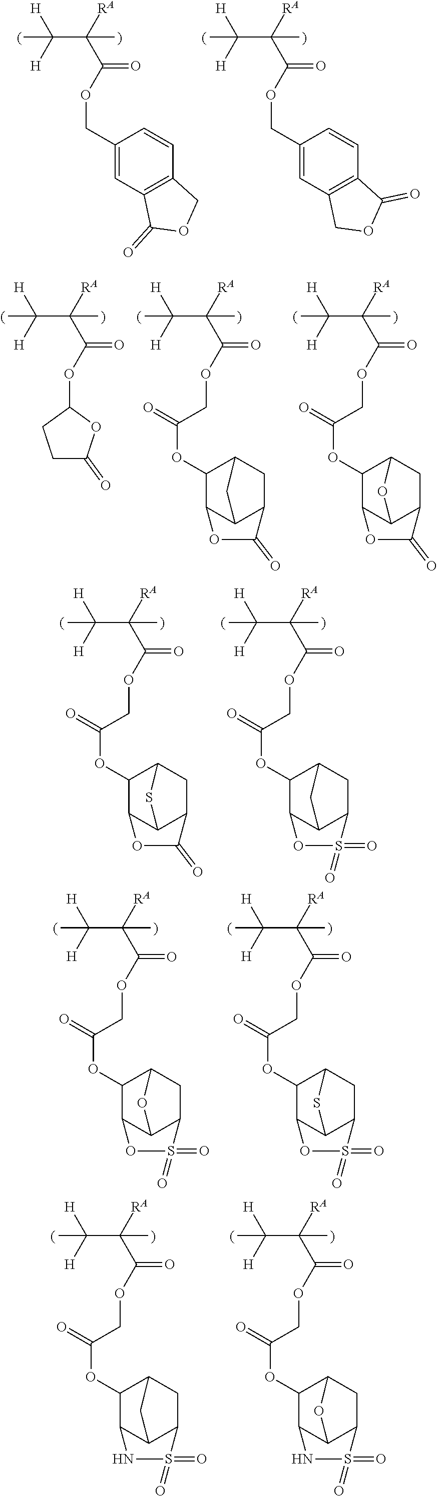

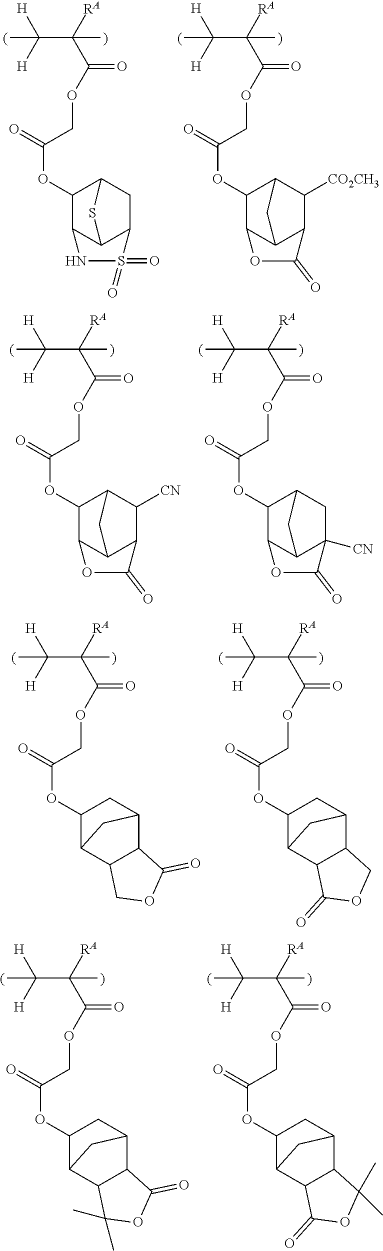

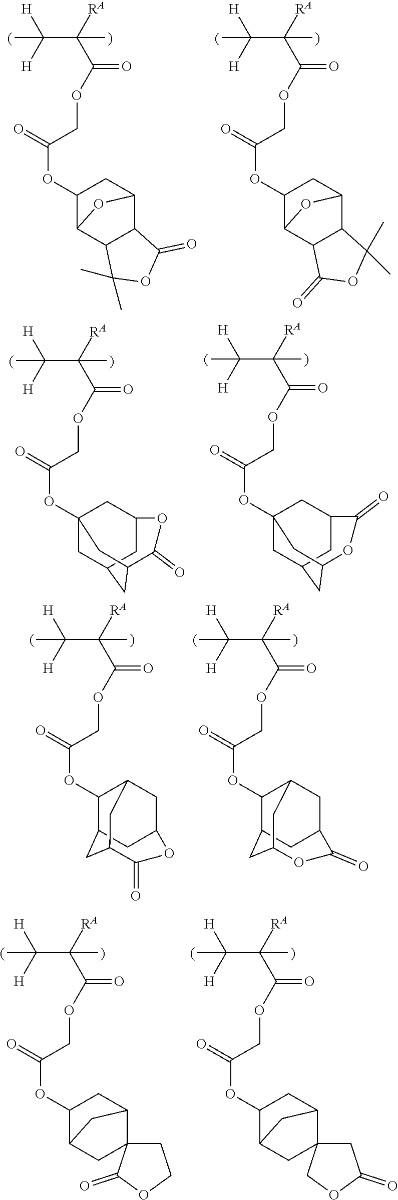

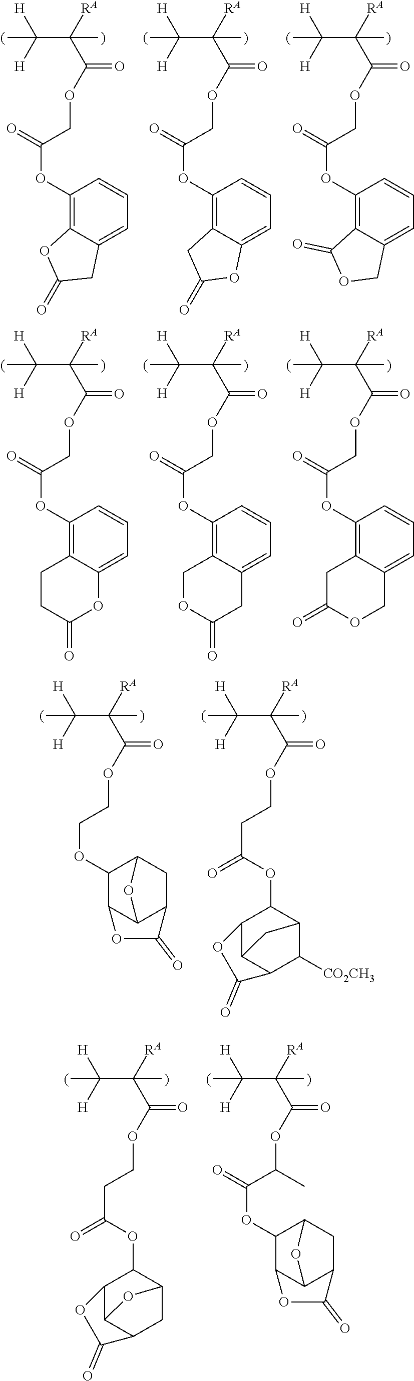

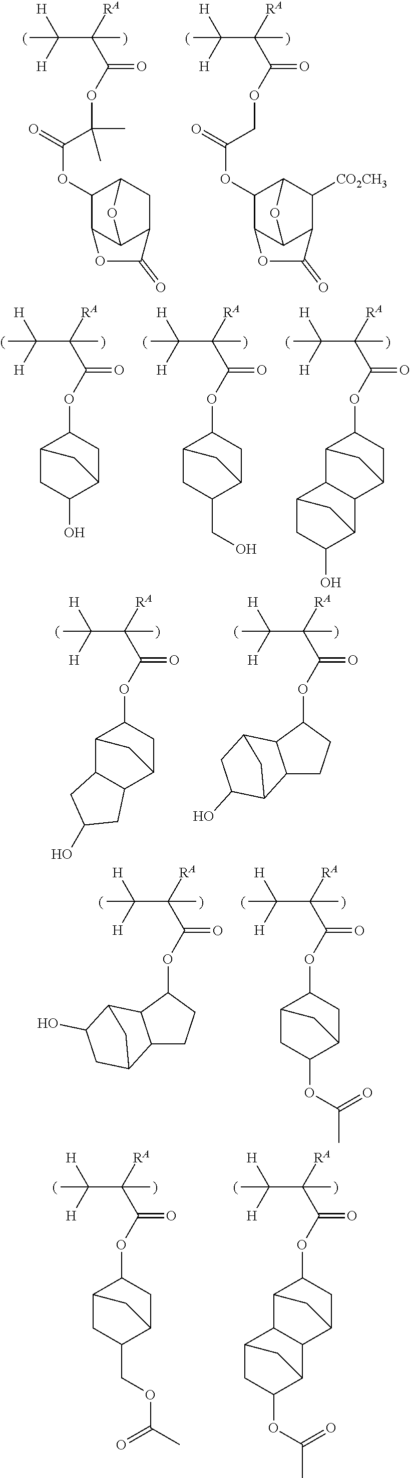

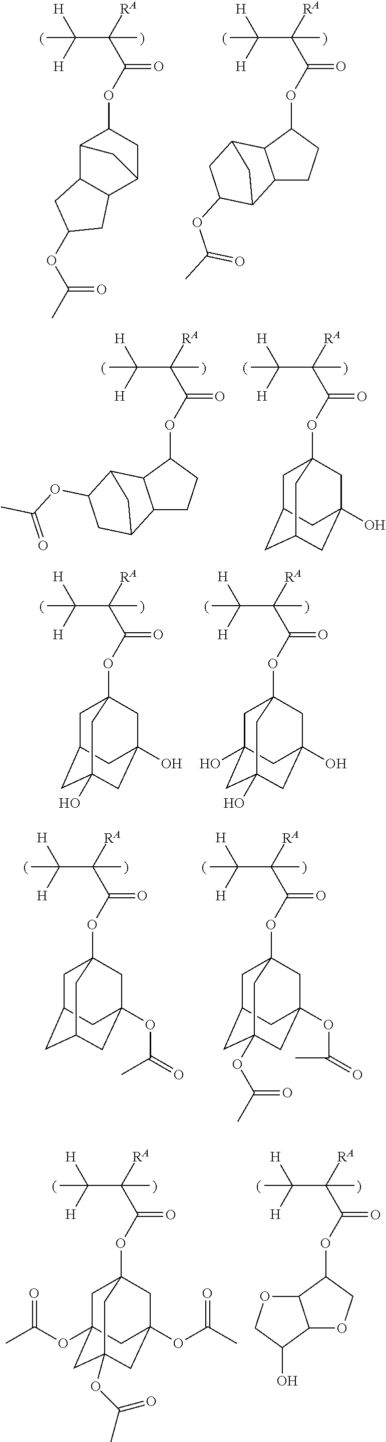

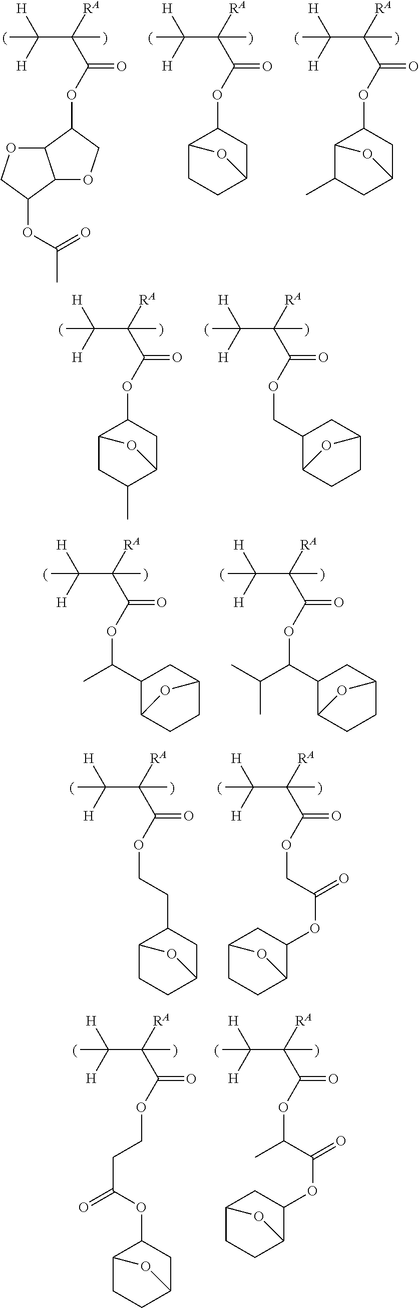

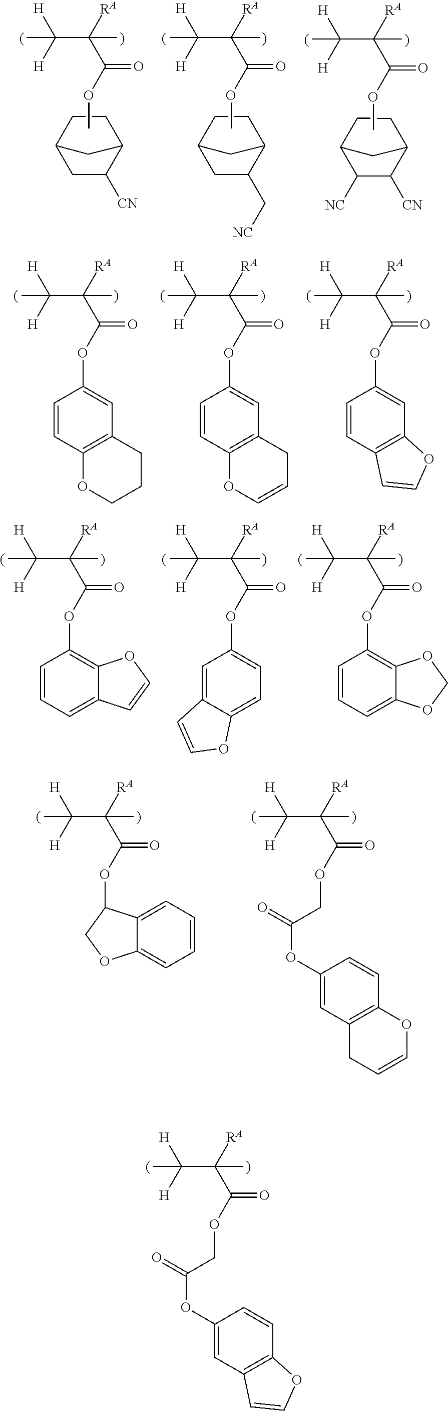

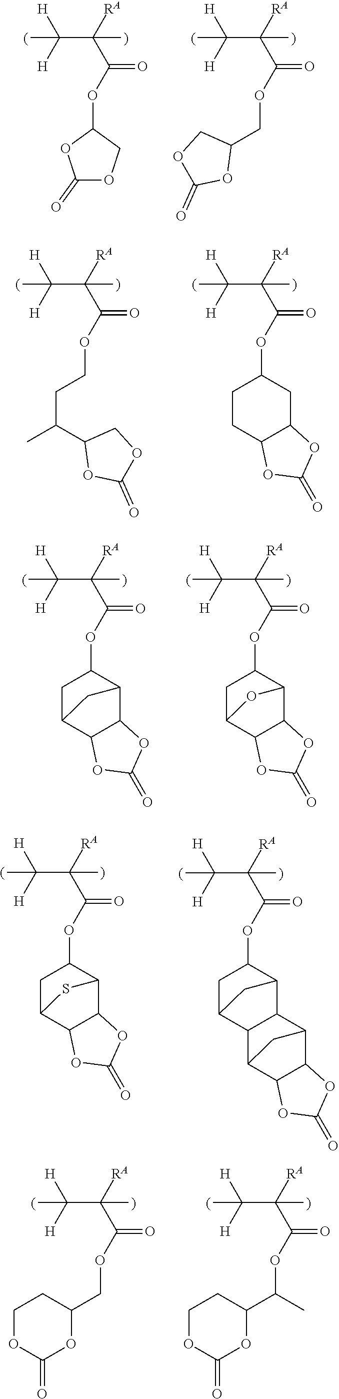

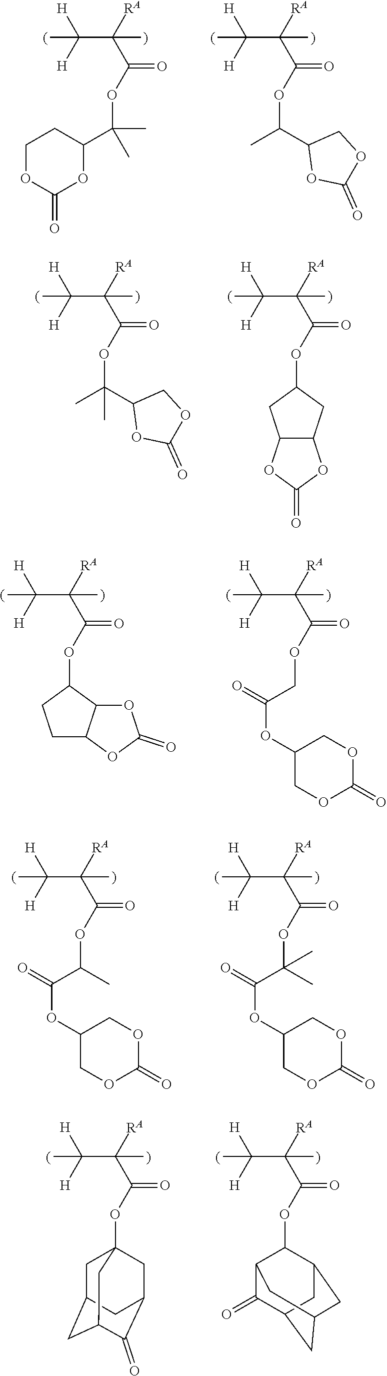

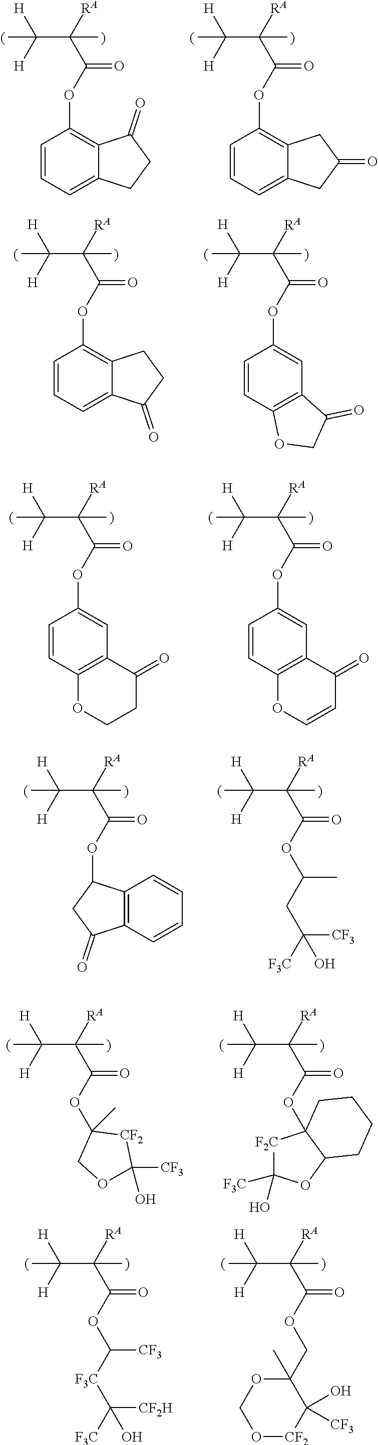

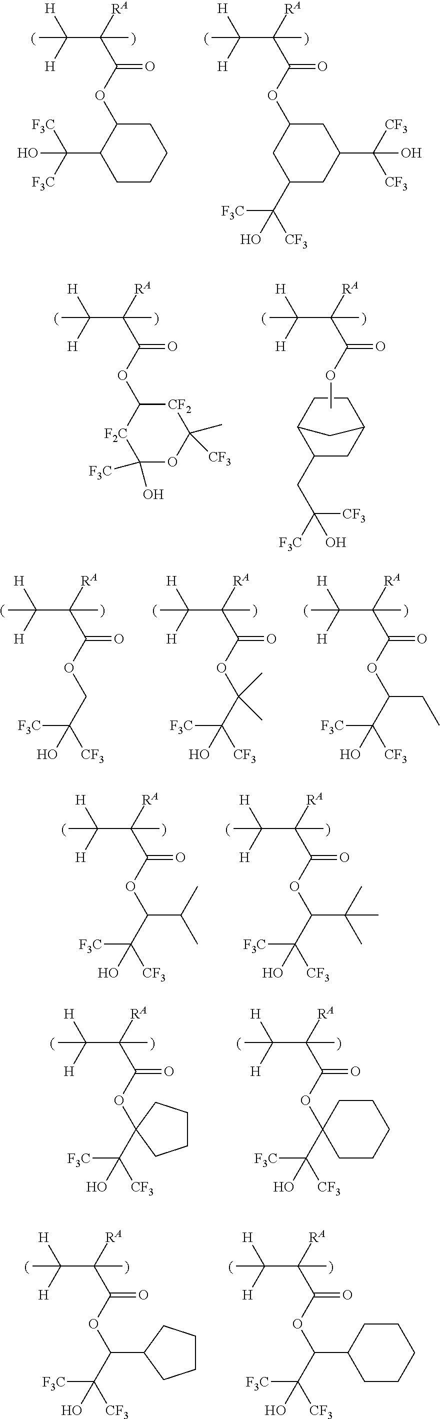

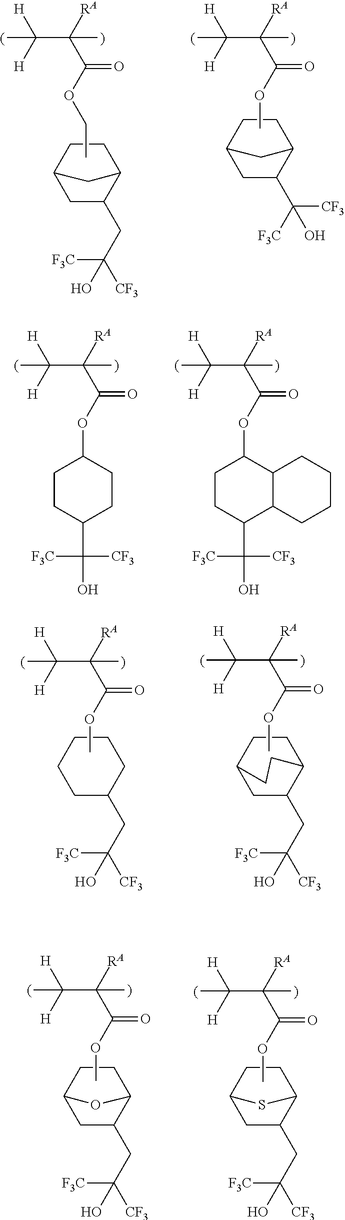

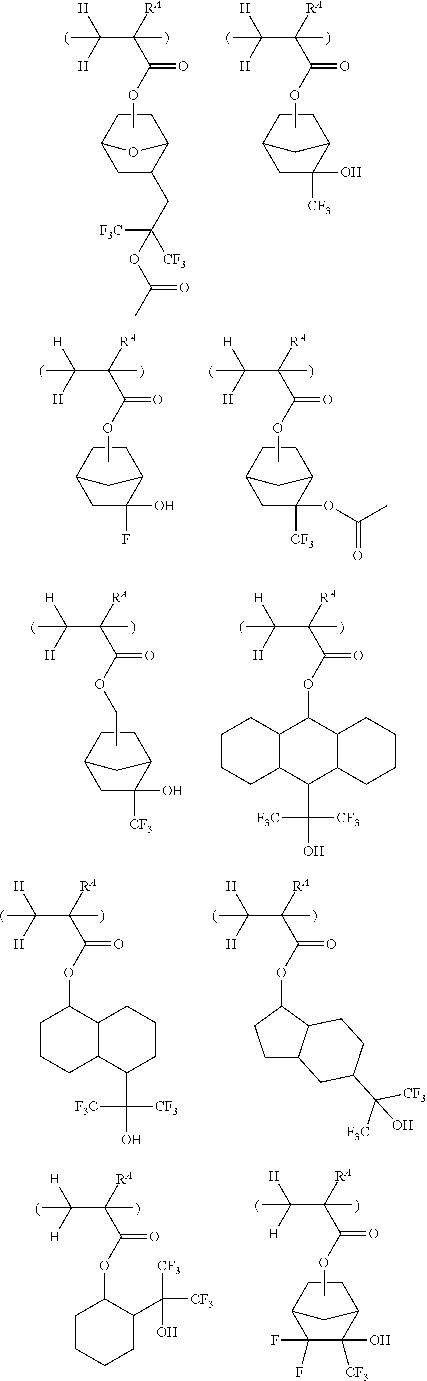

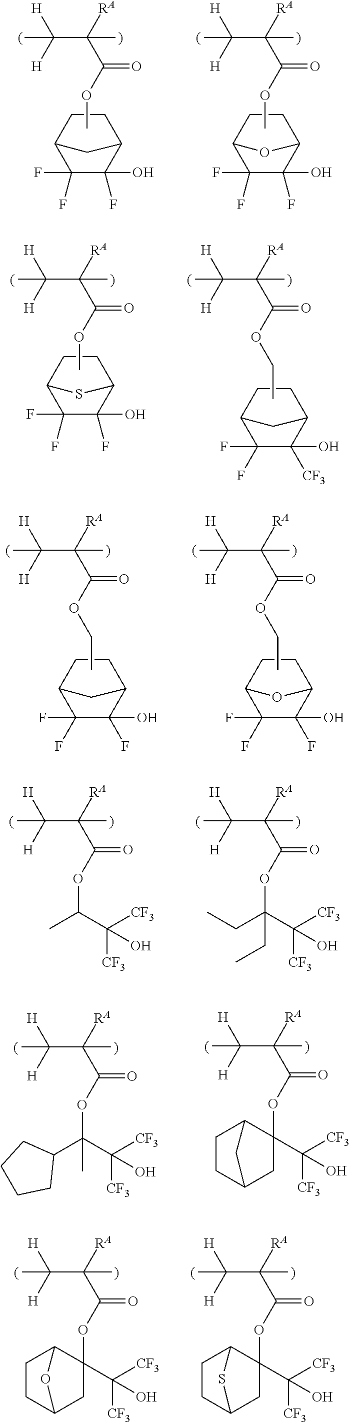

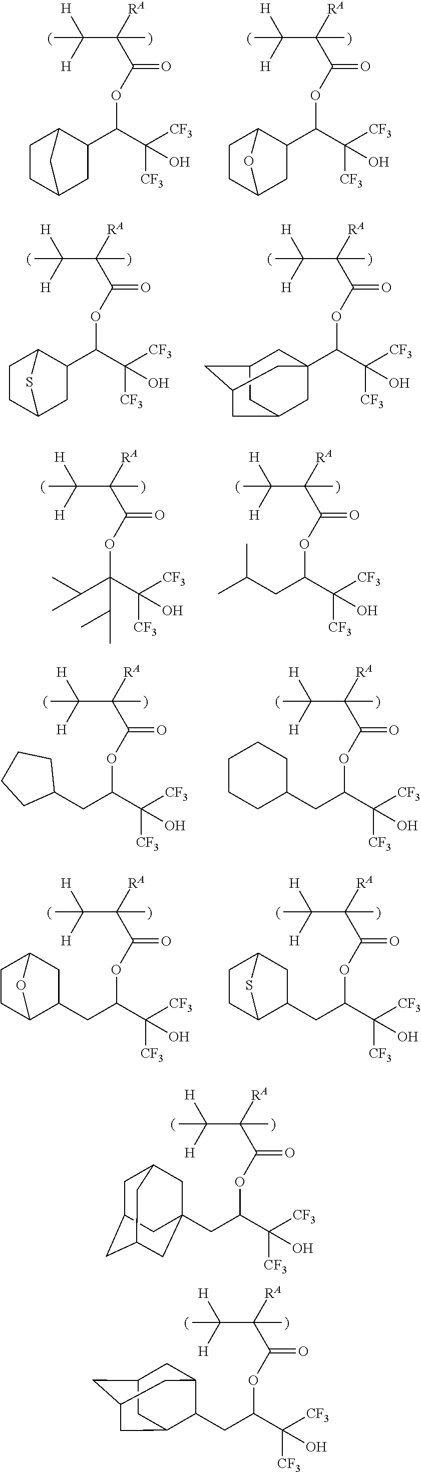

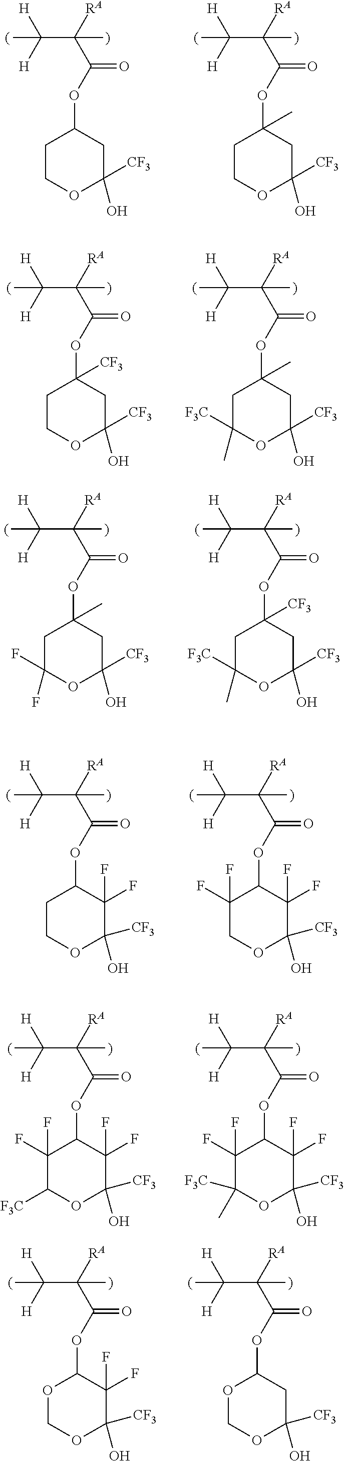

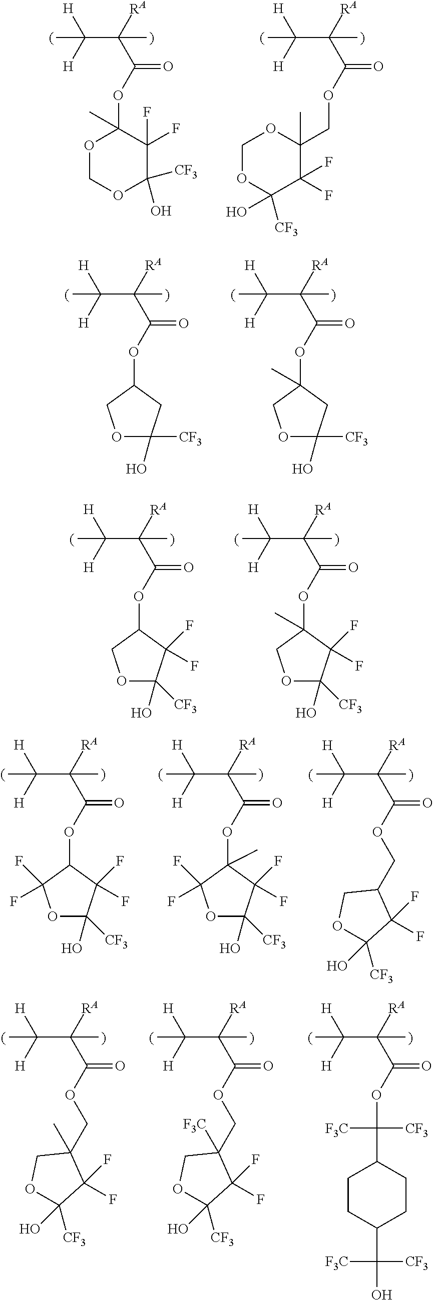

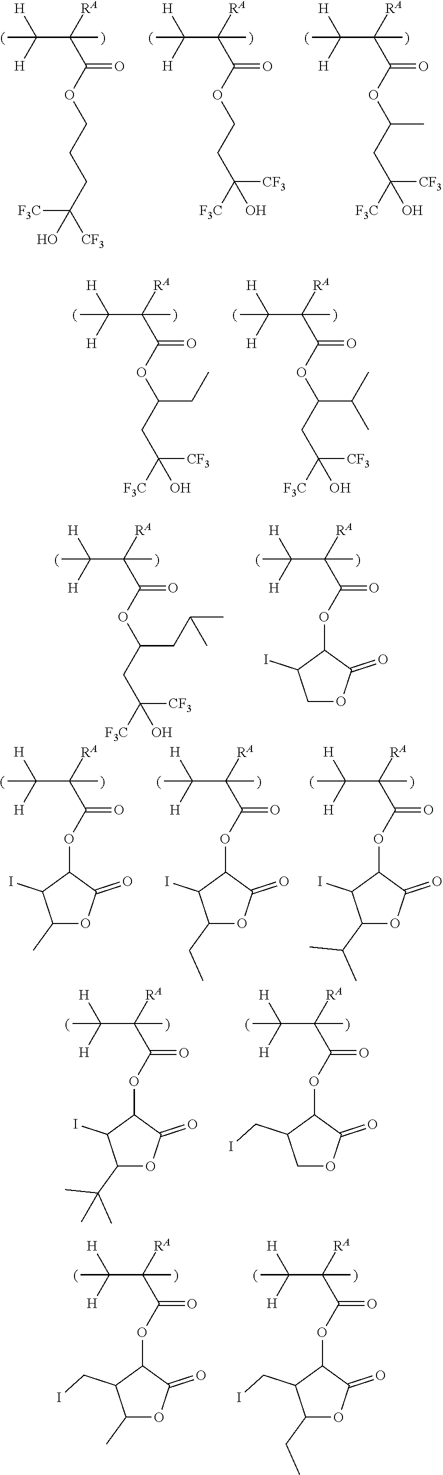

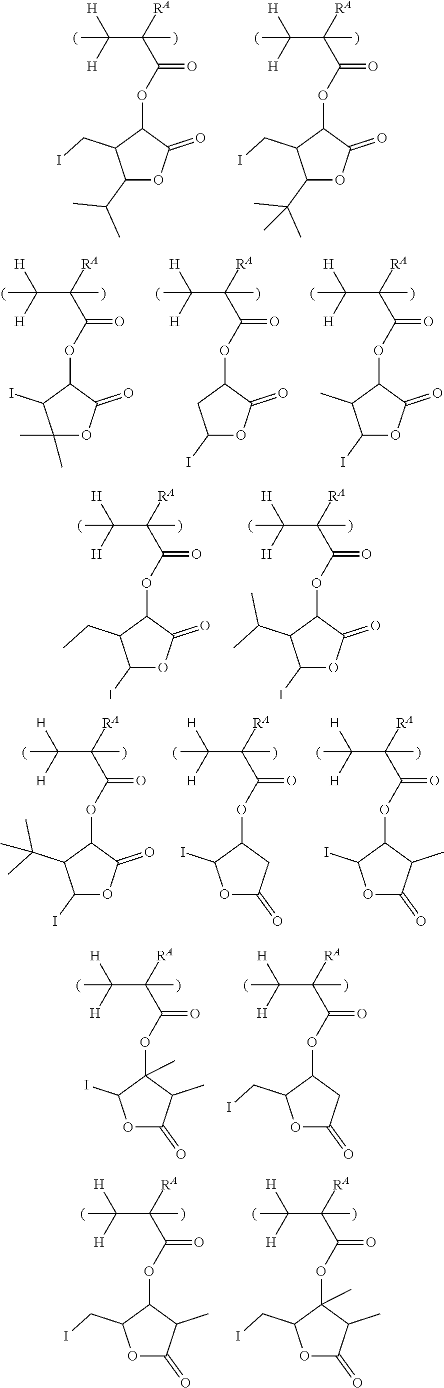

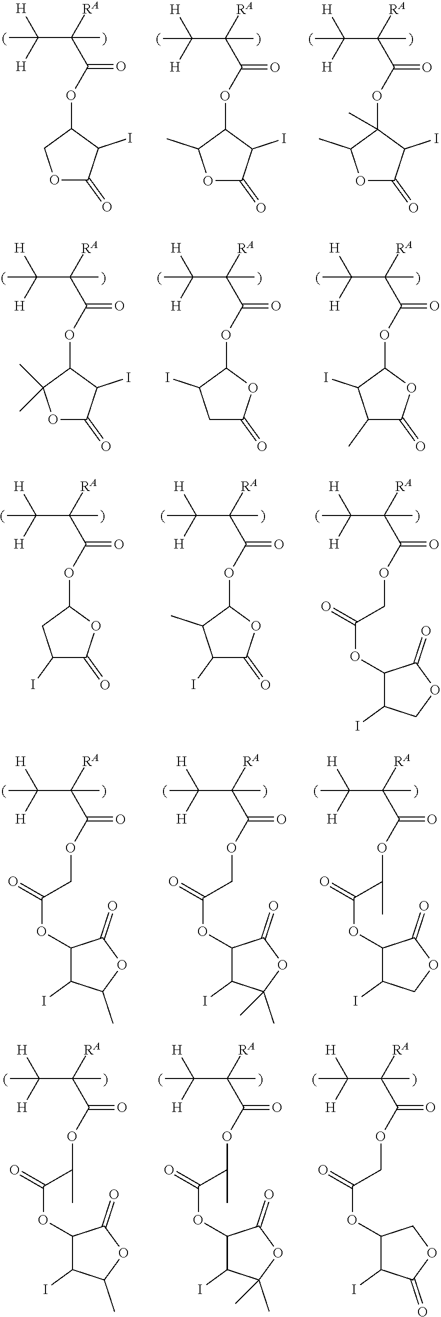

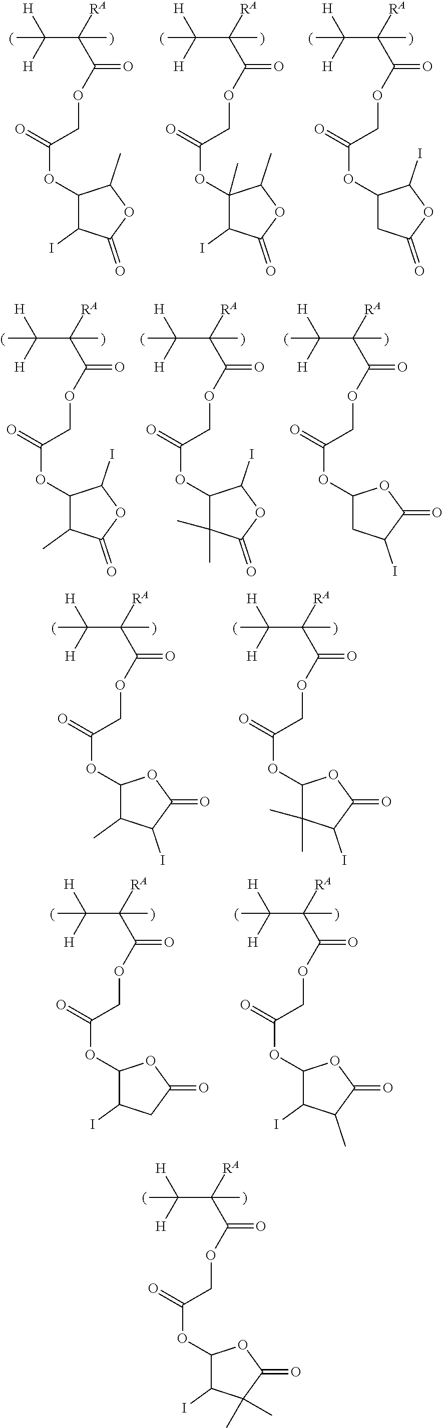

The repeating unit-b1 is particularly preferably used in EB lithography and EUV lithography, and as the repeating unit-b2, ones having a lactone ring as a polar group are particularly preferable in KrF lithography and ArF lithography.

The above-described polymer may further include at least one selected from a repeating unit represented by the following general formula (c1) (hereinafter, also referred to as repeating unit-c1), a repeating unit represented by the following general formula (c2) (hereinafter, also referred to as repeating unit-c2), a repeating unit represented by the following general formula (c3) (hereinafter, also referred to as repeating unit-c3), and a repeating unit represented by the following general formula (c4) (hereinafter, also referred to as repeating unit-c4). These repeating units function as photo-acid generators bonded to the polymer main chain.

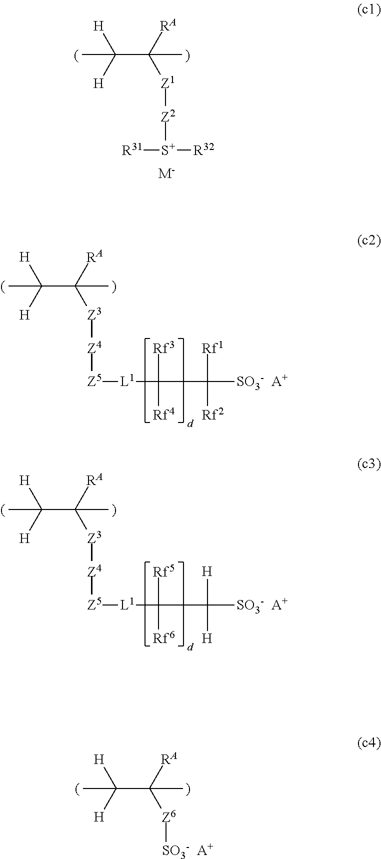

In the formulae, R^{A} represents a hydrogen atom, a fluorine atom, a methyl group, or a trifluoromethyl group; Z¹ represents a single bond or a phenylene group; Z² represents *—C(═O)—O—Z²¹—, *—C(═O)—NH—Z²¹—, or *—O—Z²¹—; Z²¹ represents an aliphatic hydrocarbylene group having 1 to 6 carbon atoms, a phenylene group, or a divalent group derived from a combination of these groups, and optionally contains a carbonyl group, an ester bond, an ether bond, or a hydroxy group; each Z³ independently represents a single bond, a phenylene group, a naphthylene group, or *—C(═O)—O—Z³¹—; Z³¹ represents an aliphatic hydrocarbylene group having 1 to 10 carbon atoms, a phenylene group, or a naphthylene group, the aliphatic hydrocarbylene group optionally containing a hydroxy group, an ether bond, an ester bond, or a lactone ring; each Z⁴ independently represents a single bond, **—Z⁴—C(═O)—O—, **—C(═O)—NH—Z⁴¹—, or **—O—Z⁴¹—; Z⁴¹ represents a hydrocarbylene group having 1 to 20 carbon atoms and optionally containing a heteroatom; each Z⁵ independently represents a single bond, ***—Z⁵¹—C(═O)—O—, ***—C(═O)—NH—Z⁵¹—, or ***—O—Z⁵¹—; Z⁵¹ represents a hydrocarbylene group having 1 to 20 carbon atoms and optionally containing a heteroatom; Z⁶ represents a single bond, a methylene group, an ethylene group, a phenylene group, a fluorinated phenylene group, a phenylene group having a substituent which is a trifluoromethyl group, *—C(═O)—O—Z⁶¹—, *—C(═O)—NH—Z⁶¹—, or *—O—Z⁶¹—; Z⁶¹ represents an aliphatic hydrocarbylene group having 1 to 6 carbon atoms, a phenylene group, a fluorinated phenylene group, or a phenylene group having a substituent which is a trifluoromethyl group, and optionally contains a carbonyl group, an ester bond, an ether bond, or a hydroxy group; “*” represents an attachment point to the carbon atom of the main chain; “**” represents an attachment point to Z³; “***” represents an attachment point to Z⁴; R³¹ and R³² each independently represent a hydrocarbyl group having 1 to 20 carbon atoms and optionally containing a heteroatom, the R³¹ and the R³² optionally being bonded to each other to form a ring together with the sulfur atom bonded thereto; L¹ represents a single bond, an ether bond, an ester bond, a carbonyl group, a sulfonic acid ester bond, a carbonate bond, or a carbamate bond; Rf¹ and Rf² each independently represent a fluorine atom or a fluorinated saturated hydrocarbyl group having 1 to 6 carbon atoms; Rf³ and Rf⁴ each independently represent a hydrogen atom, a fluorine atom, or a fluorinated saturated hydrocarbyl group having 1 to 6 carbon atoms; Rf⁵ and Rf⁶ each independently represent a hydrogen atom, a fluorine atom, or a fluorinated saturated hydrocarbyl group having 1 to 6 carbon atoms, provided that not all of the Rf⁵ and the Rf⁶ are hydrogen atoms simultaneously; M⁻ represents a non-nucleophilic counter ion; A⁺ represents an onium cation; and “d” represents an integer of 0 to 3.

In the general formulae (c1) to (c4), each R^{A} independently represents a hydrogen atom, a fluorine atom, a methyl group, or a trifluoromethyl group; Z¹ represents a single bond or a phenylene group; Z² represents *—C(═O)—O—Z²¹—, *—C(═O)—NH—Z²¹—, or *—O—Z²¹—; Z²¹ represents an aliphatic hydrocarbylene group having 1 to 6 carbon atoms, a phenylene group, or a divalent group derived from a combination of these groups, and optionally contains a carbonyl group, an ester bond, an ether bond, or a hydroxy group; each Z³ independently represents a single bond, a phenylene group, a naphthylene group, or *—C(═O)—O—Z³¹—; Z³¹ represents an aliphatic hydrocarbylene group having 1 to 10 carbon atoms, a phenylene group, or a naphthylene group, the aliphatic hydrocarbylene group optionally containing a hydroxy group, an ether bond, an ester bond, or a lactone ring; each Z⁴ independently represents a single bond, **—Z⁴¹—C(═O)—O—, **—C(═O)—NH—Z⁴¹—, or **—O—Z⁴—; Z⁴¹ represents a hydrocarbylene group having 1 to 20 carbon atoms and optionally containing a heteroatom; each Z⁵ independently represents a single bond, ***—Z⁵¹—C(═O)—O—, ***—C(═O)—NH—Z⁵¹—, or ***—O—Z⁵¹—; Z⁵¹ represents a hydrocarbylene group having 1 to 20 carbon atoms and optionally containing a heteroatom; Z⁶ represents a single bond, a methylene group, an ethylene group, a phenylene group, a fluorinated phenylene group, a phenylene group having a substituent which is a trifluoromethyl group, *—C(═O)—O—Z⁶¹—, *—C(═O)—NH—Z⁶¹—, or *—O—Z⁶¹—; Z⁶¹ represents an aliphatic hydrocarbylene group having 1 to 6 carbon atoms, a phenylene group, a fluorinated phenylene group, or a phenylene group having a substituent which is a trifluoromethyl group, and optionally contains a carbonyl group, an ester bond, an ether bond, or a hydroxy group; “*” represents an attachment point to the carbon atom of the main chain; “**” represents an attachment point to Z³; “***” represents an attachment point to Z⁴.

The aliphatic hydrocarbylene groups represented by Z²¹, Z³¹, and Z⁶¹ may be linear, branched, or cyclic, and specific examples thereof include: alkanediyl groups, such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, and a hexane-1,6-diyl group; cycloalkanediyl groups, such as a cyclopropanediyl group, a cyclobutanediyl group, a cyclopentanediyl group, and a cyclohexanediyl group; and groups which are combinations of these groups.

The hydrocarbylene groups represented by Z⁴¹ and Z⁵¹ may be saturated or unsaturated, and may be linear, branched, or cyclic. Specific examples thereof include the following, but are not limited thereto.

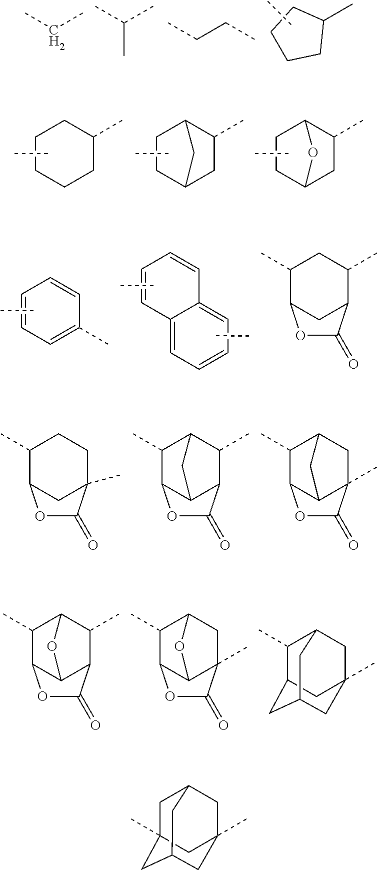

In the formulae, a broken line represents an attachment point.

In the general formula (c1), R³¹ and R³² each independently represent a hydrocarbyl group having 1 to 20 carbon atoms and optionally containing a heteroatom. The hydrocarbyl groups may be saturated or unsaturated, and may be linear, branched, or cyclic. Specific examples thereof include: alkyl groups having 1 to 20 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group, a naphthyl group, and a thienyl group; aralkyl groups having 7 to 20 carbon atoms, such as a benzyl group, a 1-phenylethyl group, and a 2-phenylethyl group; and groups which are combinations of these groups. Aryl groups are preferable. Moreover, part or all of the hydrogen atoms of the hydrocarbyl groups may be substituted with a group containing a heteroatom, such as an oxygen atom, a sulfur atom, a nitrogen atom, or a halogen atom, and part of the —CH₂— of the hydrocarbyl groups may be substituted with a group containing a heteroatom, such as an oxygen atom, a sulfur atom, or a nitrogen atom. Thus, the resulting hydrocarbyl groups may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, carboxylic anhydride (—C(═O)—O—C(═O)—), a haloalkyl group, etc.

Furthermore, the R³¹ and the R³² are optionally bonded to each other to form a ring together with the sulfur atom bonded thereto. In this case, examples of the ring include those given as examples of the ring that may be formed by R^{ct1} and R^{ct2} being bonded to each other together with the sulfur atom bonded thereto in the description of the general formula (cation-1) given later.

Specific examples of the cation of the repeating unit-c1 include the following, but are not limited thereto. Note that, in the following formulae, R^{A} is as defined above.

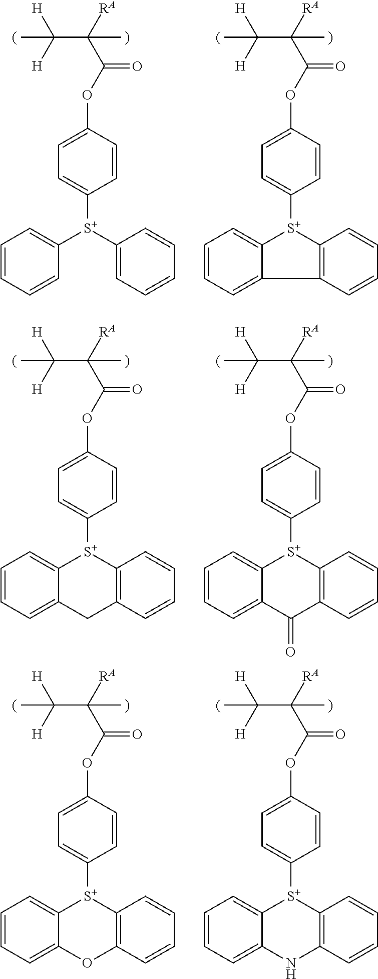

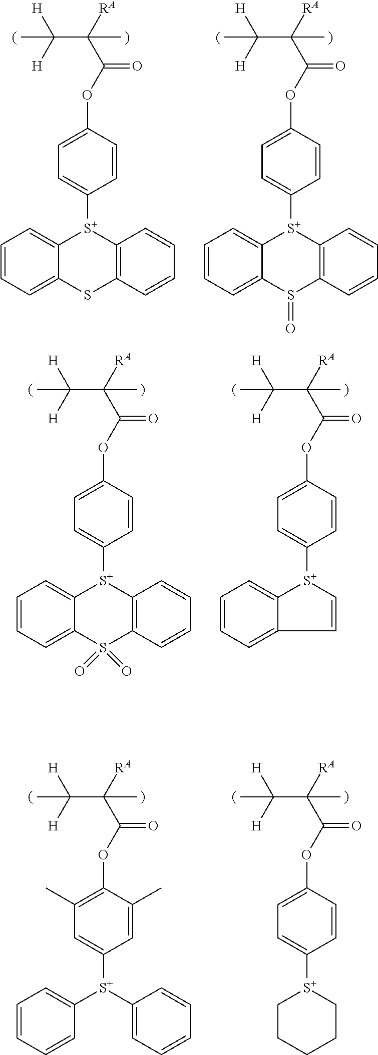

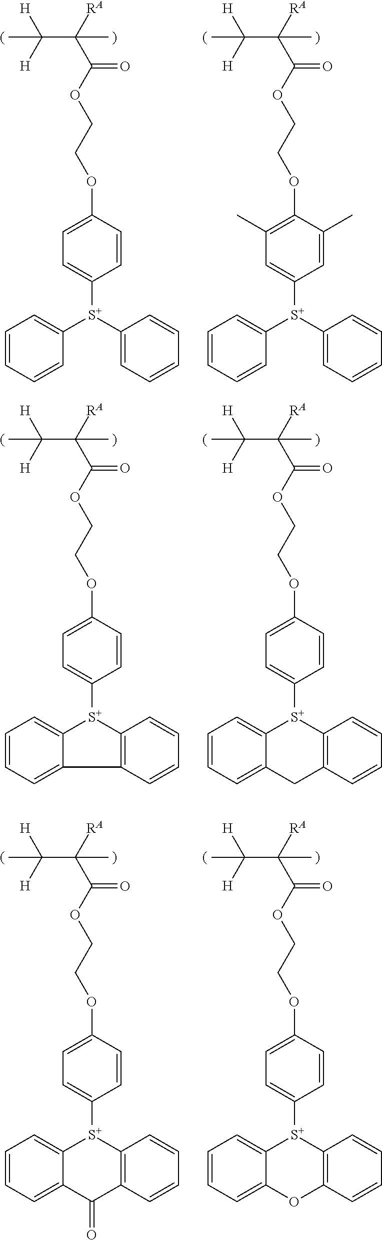

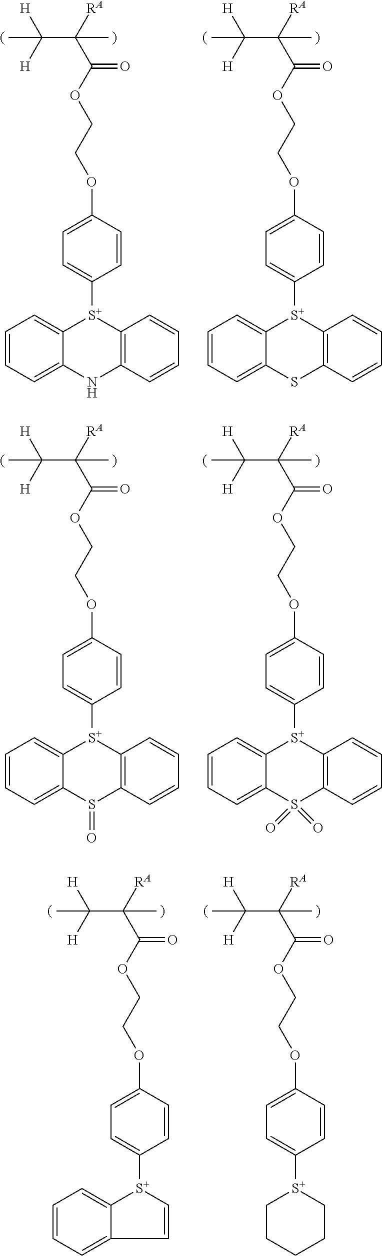

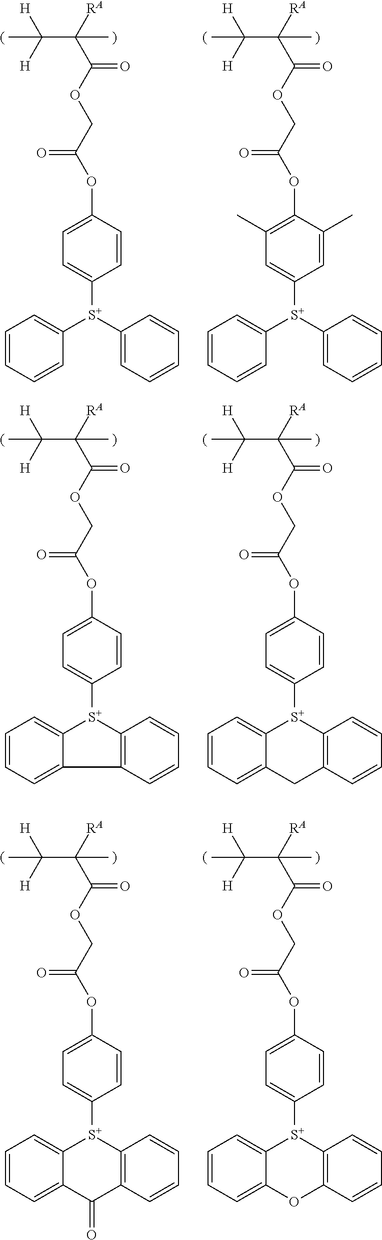

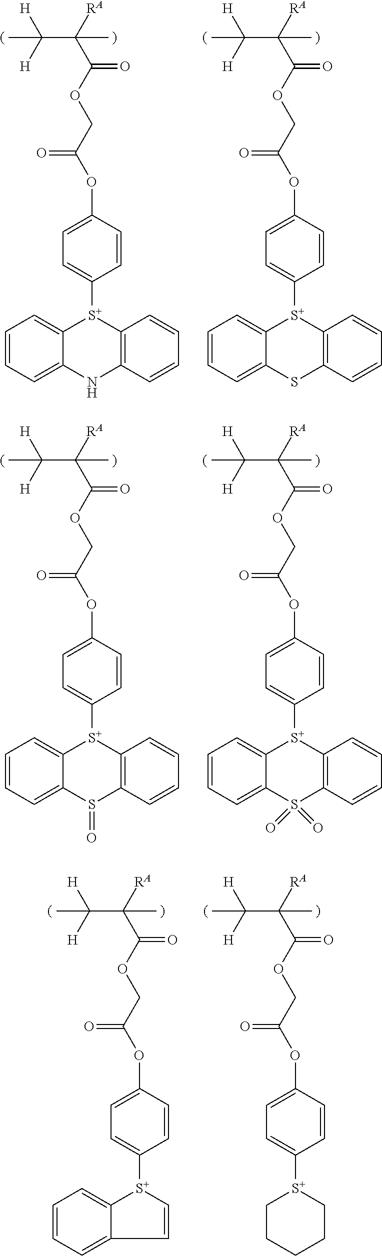

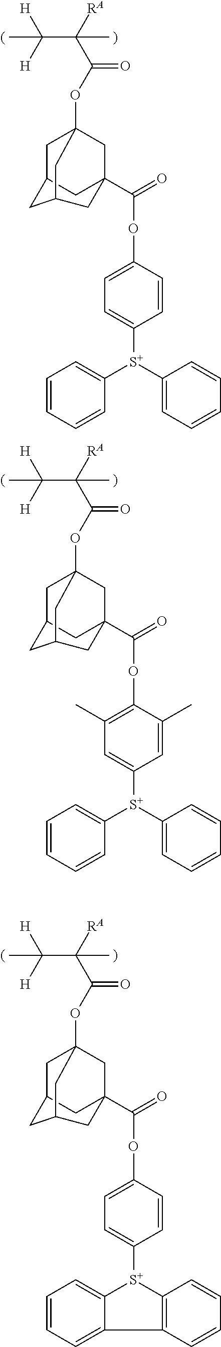

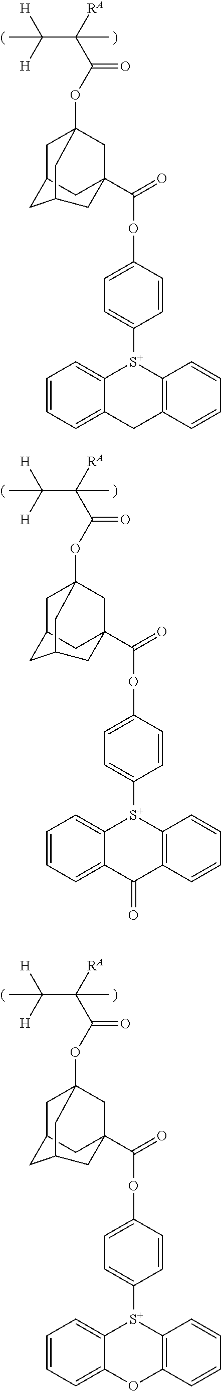

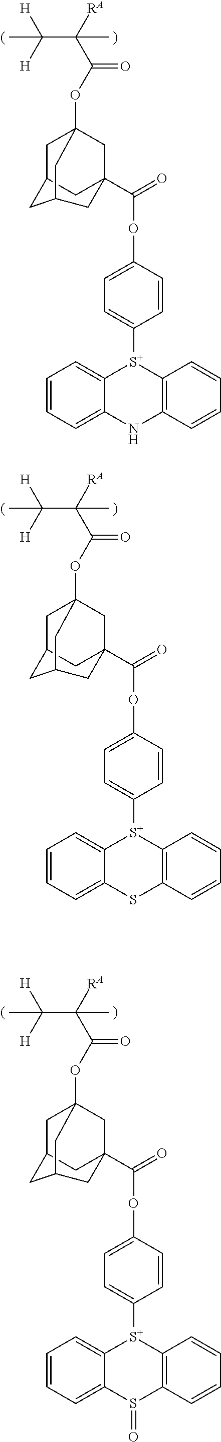

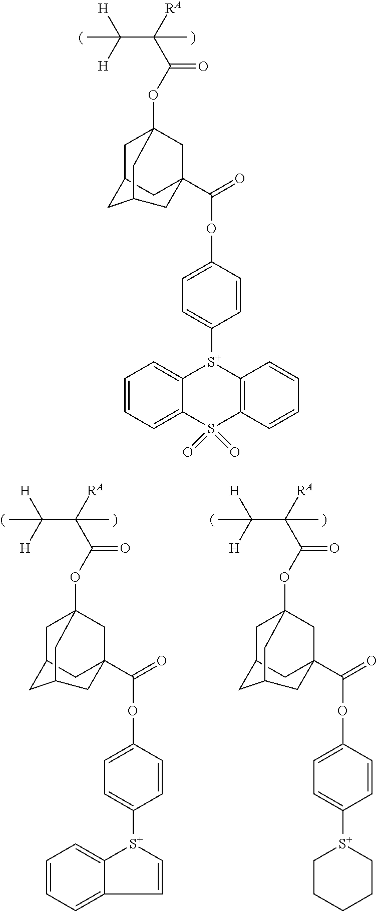

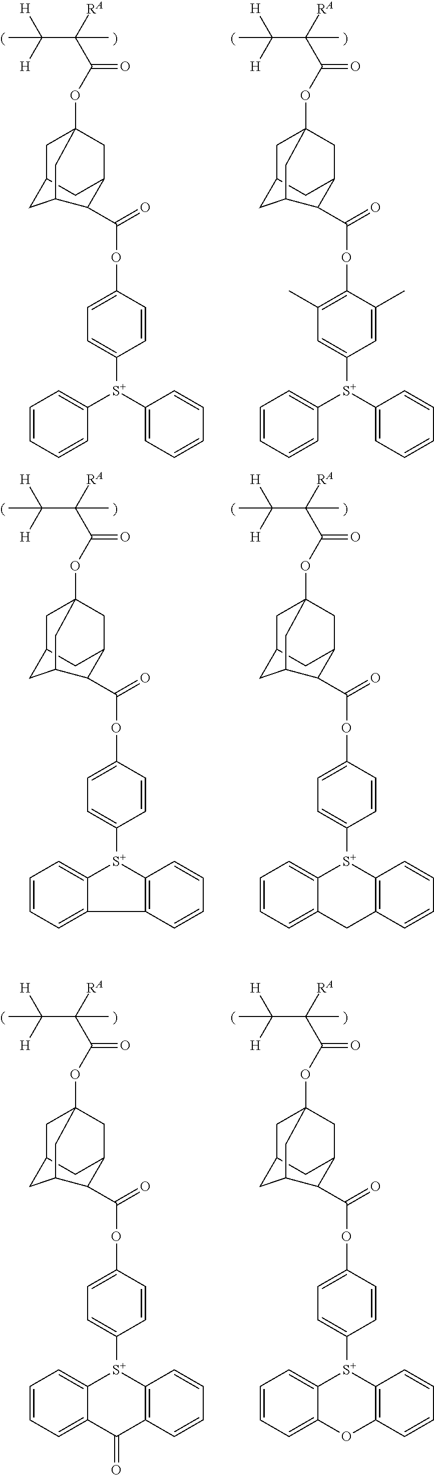

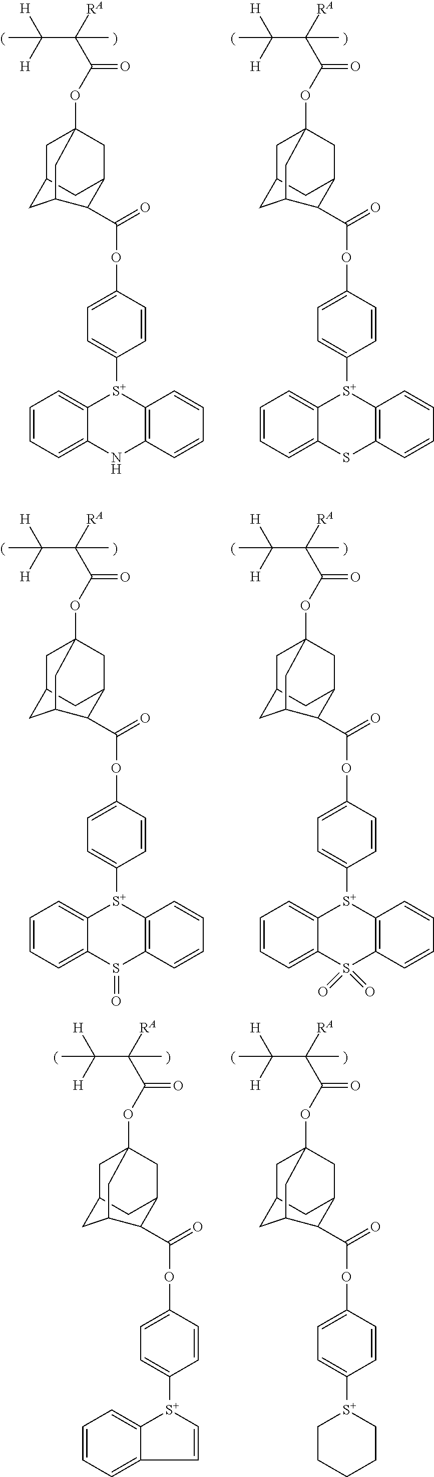

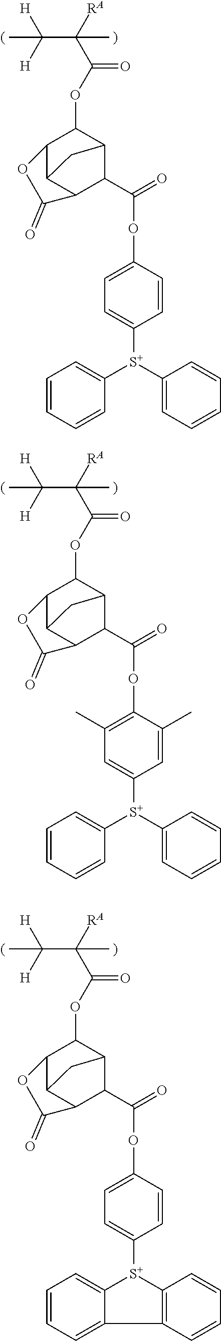

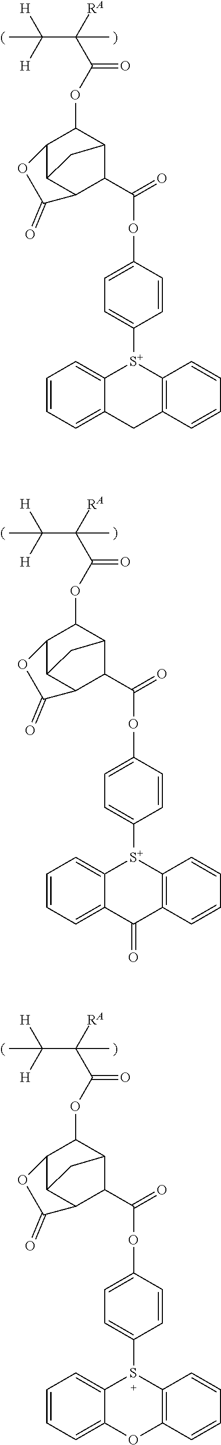

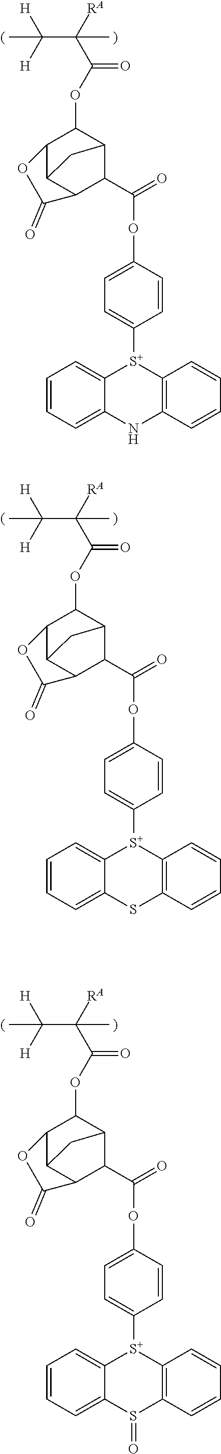

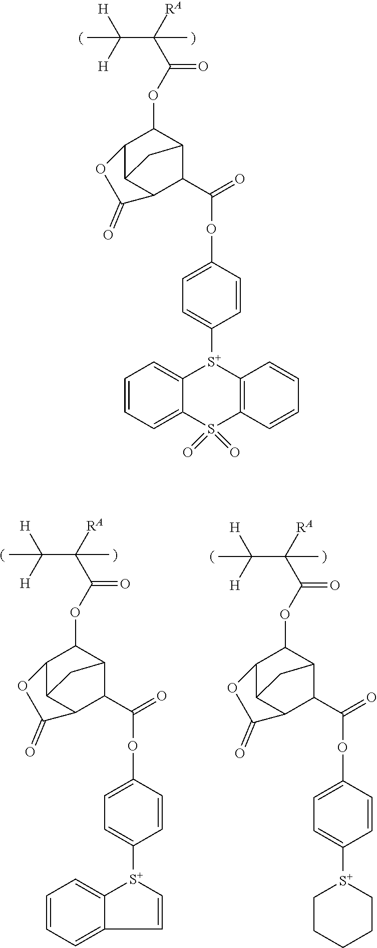

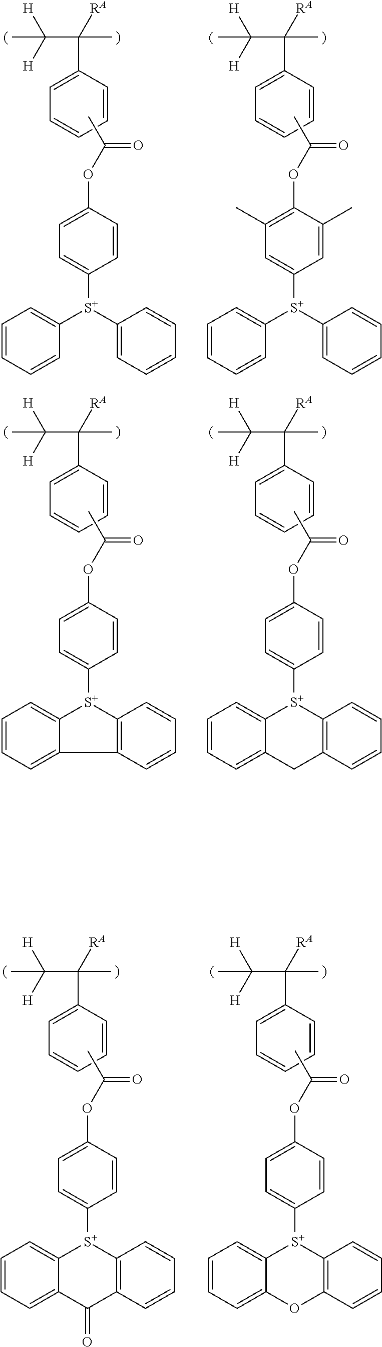

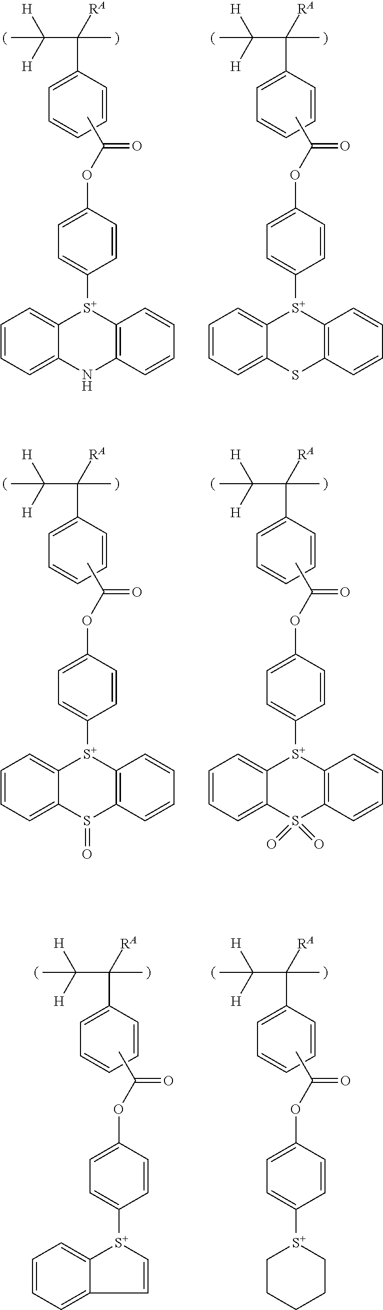

In the general formula (c1), M⁻ represents a non-nucleophilic counter ion. As the non-nucleophilic counter ion, halide ions, sulfonate anions, imidic acid anions, and methide acid anions are preferable. Specific examples of the halide ions include chloride and bromide ions. Specific examples of the sulfonate anions (sulfonate ions) include: fluoroalkylsulfonate ions, such as triflate, 1,1,1-trifluoroethanesulfonate, and nonafluorobutanesulfonate ions; arylsulfonate ions, such as tosylate, benzenesulfonate, 4-fluorobenzenesulfonate, and 1,2,3,4,5-pentafluorobenzenesulfonate ions; and alkylsulfonate ions, such as mesylate and butanesulfonate ions. Specific examples of the imidic acid anions (imide ions) include bis(trifluoromethylsulfonyl)imide, bis(perfluoroethylsulfonyl)imide, and bis(perfluorobutylsulfonyl)imide ions. Specific examples of the methide acid anions (methide ions) include tris(trifluoromethylsulfonyl)methide and tris(perfluoroethylsulfonyl)methide ions.

Other examples of the non-nucleophilic counter ion include anions represented by any of the following general formulae (c1-1) to (c1-4).

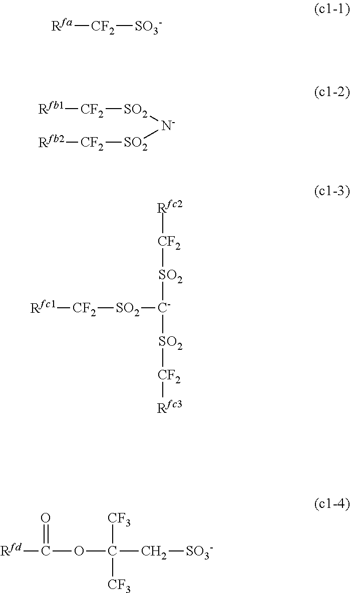

In the general formula (c1-1), R^{fa} represents a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the general formula (c1-1-1) described below.

As the anion represented by the general formula (c1-1), ones represented by the following general formula (c1-1-1) are preferable.

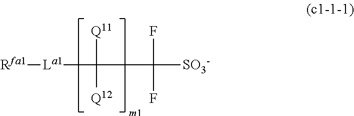

In the general formula (c1-1-1), Q¹¹ and Q¹² each independently represent a hydrogen atom, a fluorine atom, or a fluorinated saturated hydrocarbyl group having 1 to 6 carbon atoms, and for enhancing solvent solubility, it is preferable that at least one is a trifluoromethyl group. “ml” represents an integer of 0 to 4, and is particularly preferably 1. R^{fa1} represents a hydrocarbyl group having 1 to 35 carbon atoms and optionally containing a heteroatom. As the heteroatom, an oxygen atom, a nitrogen atom, a sulfur atom, a halogen atom, etc. are preferable, and an oxygen atom is more preferable. As the hydrocarbyl group, ones having 6 to 30 carbon atoms are particularly preferable from the viewpoint that high resolution can be achieved in fine patterning.

In the general formula (c1-1-1), the hydrocarbyl group represented by R^{fa1} having 1 to 35 carbon atoms may be saturated or unsaturated, and may be linear, branched, or cyclic. Specific examples thereof include: alkyl groups having 1 to 35 carbon atoms, such as a methyl group, an ethyl group, an n-propyl group, an isopropyl group, an n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups having 3 to 35 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; unsaturated aliphatic hydrocarbyl groups having 2 to 35 carbon atoms, such as an allyl group and a 3-cyclohexenyl group; aryl groups having 6 to 35 carbon atoms, such as a phenyl group, a 1-naphthyl group, a 2-naphthyl group, and a 9-fluorenyl group; aralkyl groups having 7 to 35 carbon atoms, such as a benzyl group and a diphenylmethyl group; and groups which are combinations of the groups.

Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, carboxylic anhydride (—C(═O)—O—C(═O)—), a haloalkyl group, etc. Examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, a 3-oxocyclohexyl group, etc.

In the general formula (c1-1-1), L^{a1} represents a single bond, an ether bond, an ester bond, a sulfonic acid ester bond, a carbonate bond, or a carbamate bond, preferably an ether bond or an ester bond from the viewpoint of synthesis, and further preferably an ester bond.

Specific examples of the anion represented by the general formula (c1-1-1) include the following, but are not limited thereto. Note that, in the following formulae, Q¹¹ is as defined above, and Ac represents an acetyl group.

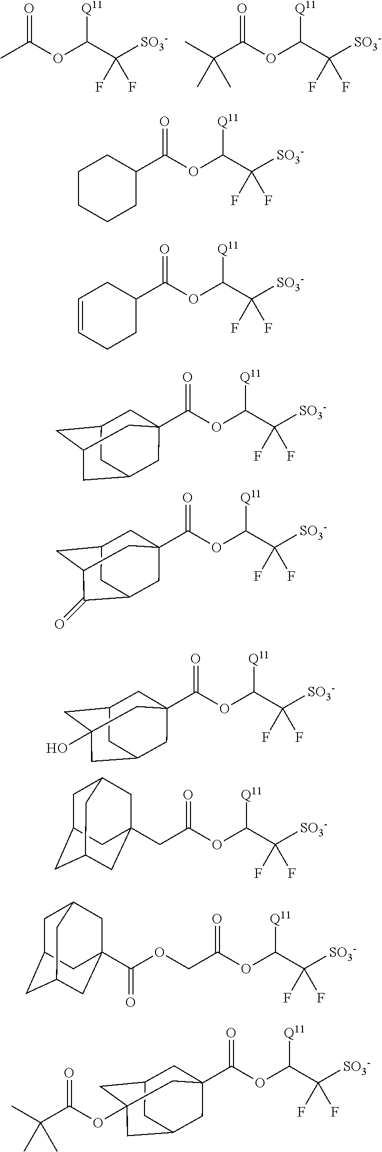

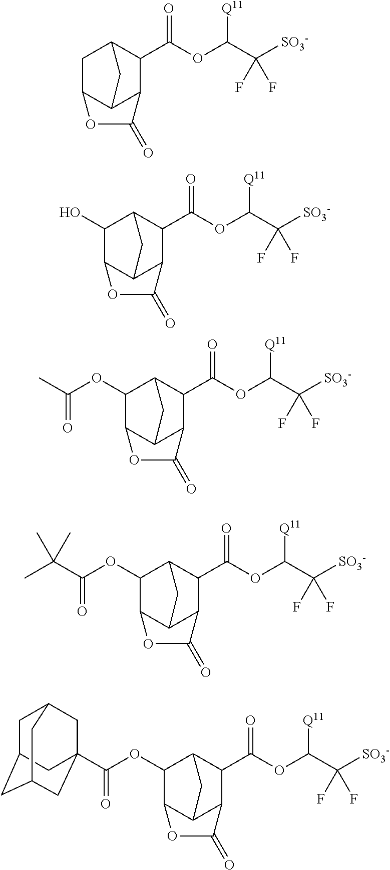

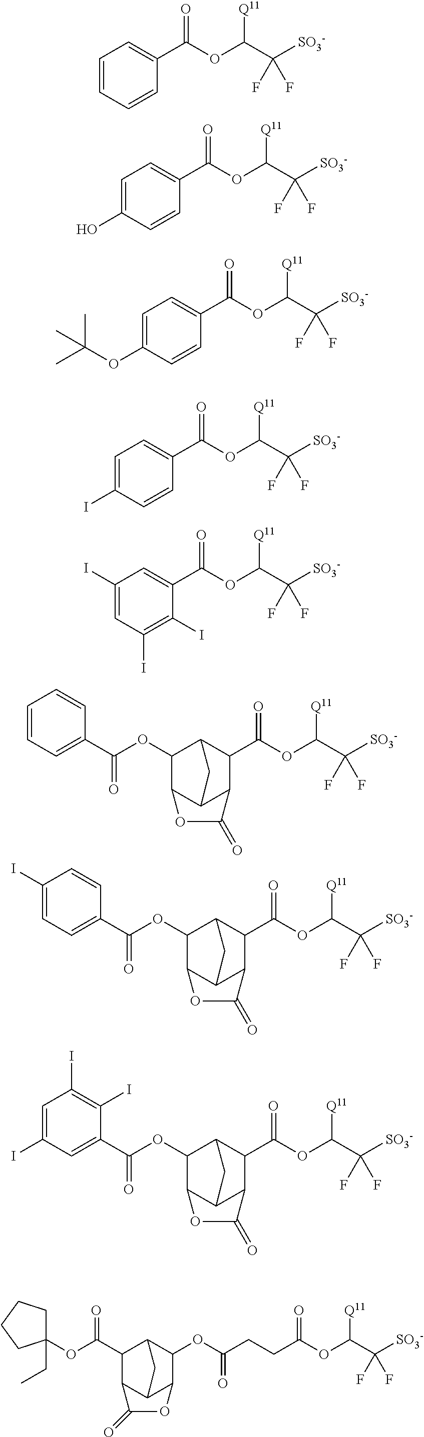

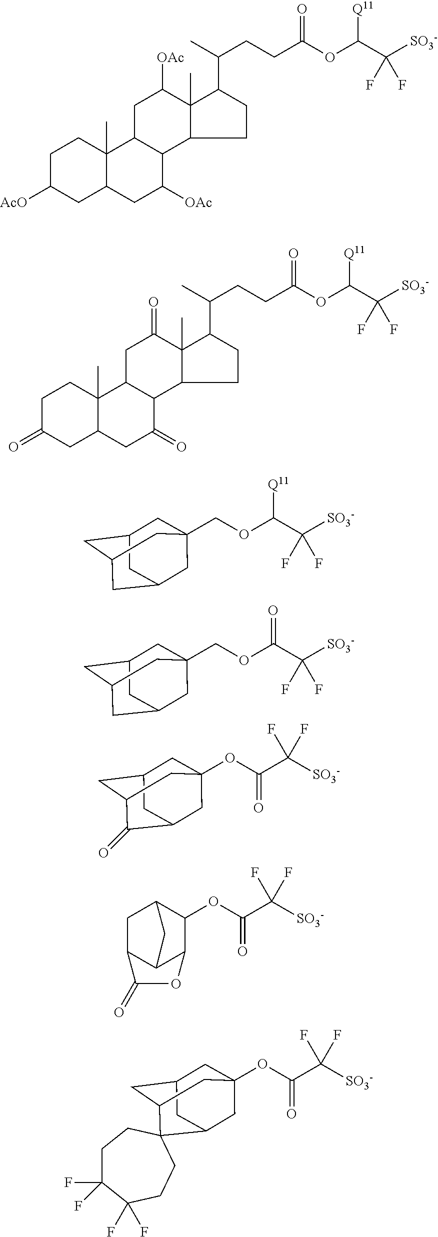

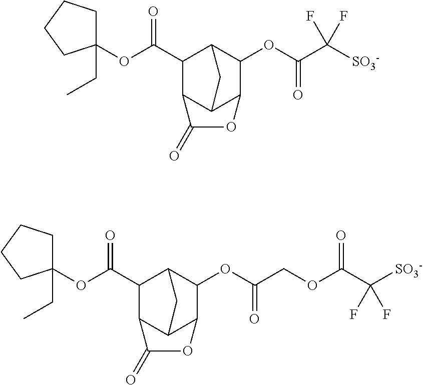

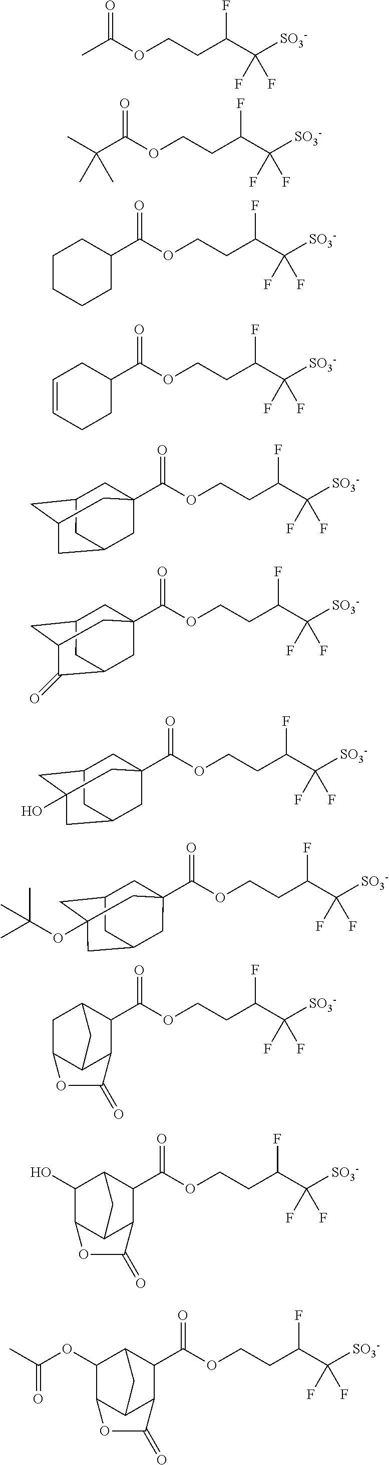

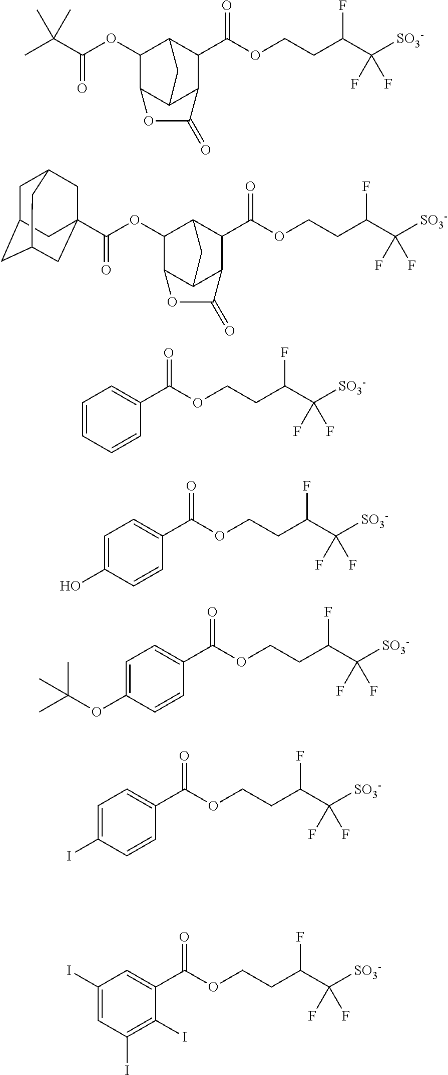

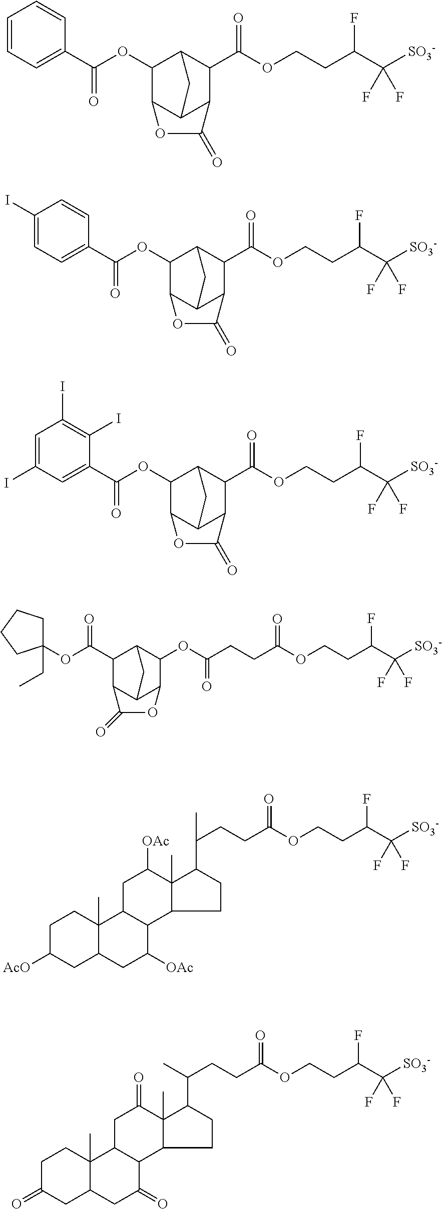

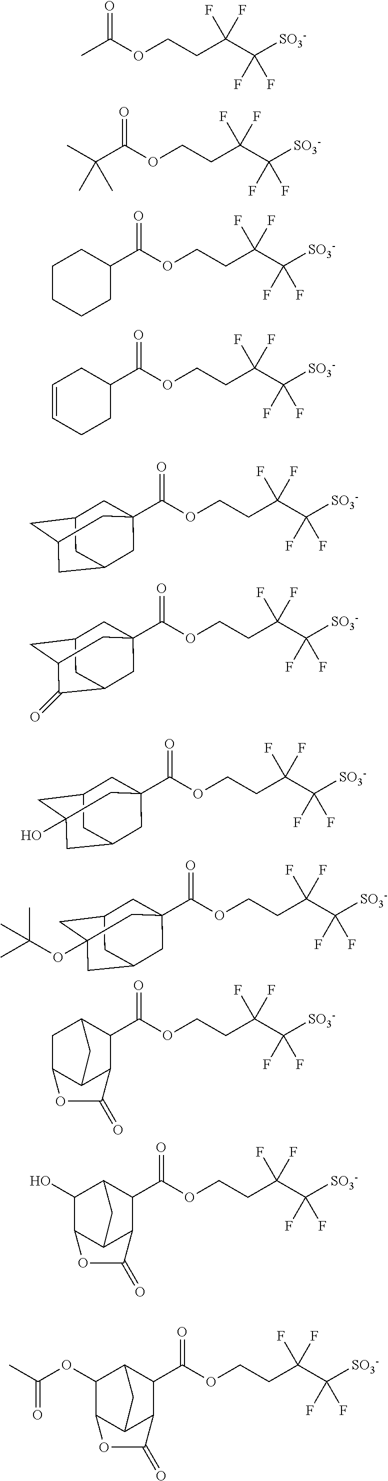

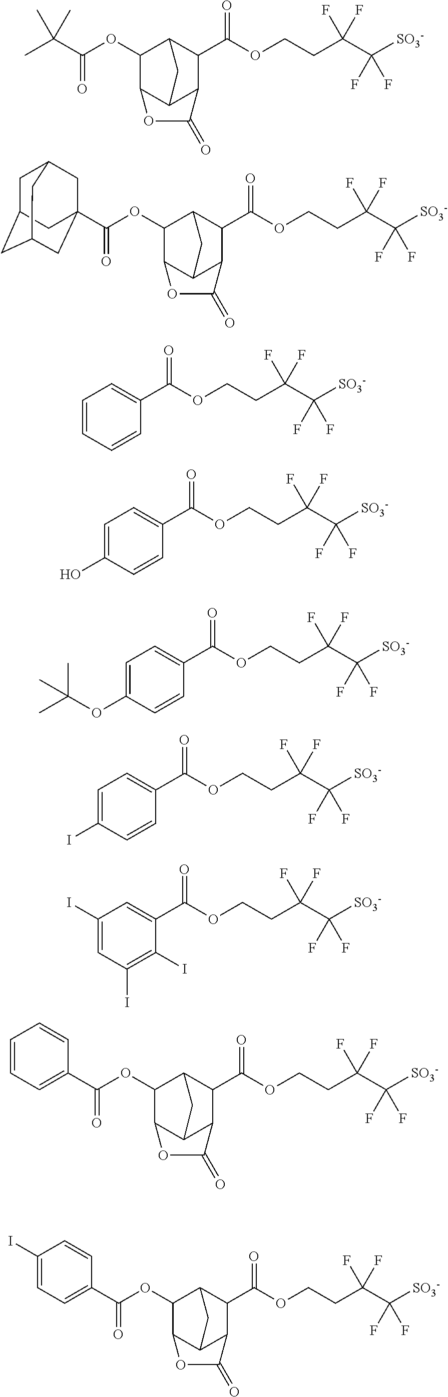

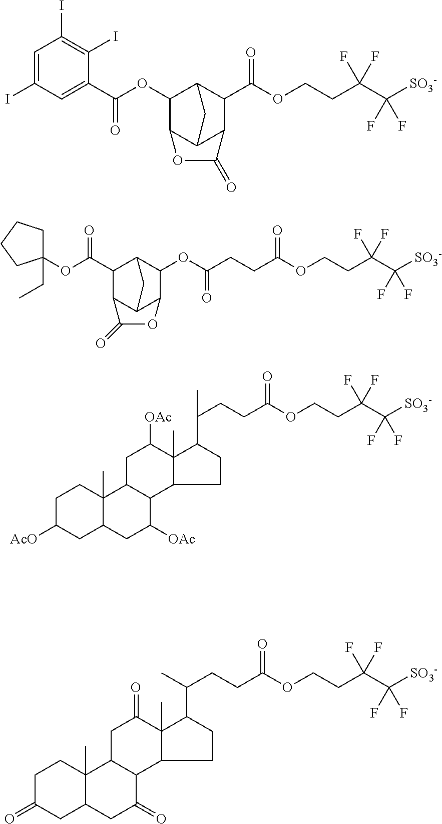

In the general formula (c1-2), R^{fb1} and R^{fb2} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the general formula (c1-1-1). R^{fb1} and R^{fb2} are preferably a fluorine atom or a linear fluorinated alkyl group having 1 to 4 carbon atoms. Alternatively, R^{fb1} and R^{fb2} may bond with each other to form a ring together with the group (—CF₂—SO₂—N—SO₂—CF₂—) bonded thereto. In this case, as the group obtained by R^{fb1} and R^{fb2} being bonded to each other, a fluorinated ethylene group or a fluorinated propylene group is preferable.

In the general formula (c1-3), R^{fc1}, R^{fc2}, and R^{fc3} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the general formula (c1-1-1). R^{fc1}, R^{fc2}, and R^{fc3} are preferably a fluorine atom or a linear fluorinated alkyl group having 1 to 4 carbon atoms. Alternatively, R^{fc1} and R^{fc2} may bond with each other to form a ring together with the group (—CF₂—SO₂—C—SO₂—CF₂—) bonded thereto. In this case, as the group obtained by R^{fc1} and R^{fc2} being bonded to each other, a fluorinated ethylene group or a fluorinated propylene group is preferable.

In the general formula (c1-4), R^{fd} represents a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the general formula (c1-1-1).

Specific examples of the anion represented by the general formula (c1-4) include the following, but are not limited thereto.

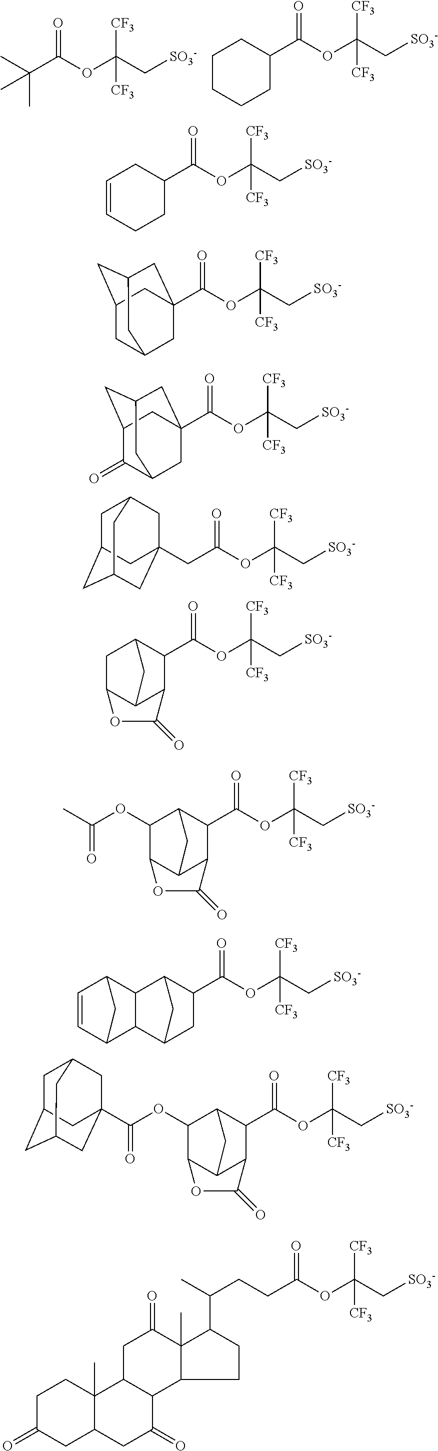

Examples of the non-nucleophilic counter ion further include anions having an aromatic ring having a substituent which is an iodine atom or a bromine atom. Specific examples of such anions include ones represented by the following general formula (c1-5).

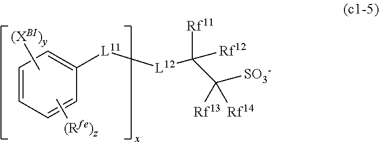

In the general formula (c1-5), “x” represents an integer that satisfies 1≤x≤3. “y” and “z” represent integers that satisfy 1≤y≤5, 0≤z≤3, and 1≤y+z≤5. “y” is preferably an integer that satisfies 1≤y≤3, more preferably 2 or 3. “z” is preferably an integer that satisfies 0 z 2.

In the general formula (c1-5), X^{BI} represents an iodine atom or a bromine atom, and when “x” and/or “y” is 2 or more, the X^{BI}s may be identical to or different from each other.

In the general formula (c1-5), L¹¹ represents a single bond, an ether bond, an ester bond, or a saturated hydrocarbylene group having 1 to 6 carbon atoms and optionally containing an ether bond or an ester bond. The saturated hydrocarbylene group may be linear, branched, or cyclic.

In the general formula (c1-5), Liz represents, when “x” is 1, a single bond or a divalent linking group having 1 to 20 carbon atoms, and when “x” is 2 or 3, an (x+1)-valent linking group having 1 to 20 carbon atoms. The linking group optionally contains an oxygen atom, a sulfur atom, or a nitrogen atom.

In the general formula (c1-5), R^{fe} represents a hydroxy group, a carboxy group, a fluorine atom, a chlorine atom, a bromine atom, or an amino group; a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, or a hydrocarbylsulfonyloxy group having 1 to 20 carbon atoms, each optionally containing a fluorine atom, a chlorine atom, a bromine atom, a hydroxy group, an amino group, or an ether bond; or —N(R^{feA}) (R^{feB}), —N(R^{feC})—C(═O)—R^{feD}, or —N(R^{feC})—C(═O)—O—R^{feD}. R^{feA} and R^{feB} each independently represent a hydrogen atom or a saturated hydrocarbyl group having 1 to 6 carbon atoms. R^{feC} represents a hydrogen atom or a saturated hydrocarbyl group having 1 to 6 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{feD} represents an aliphatic hydrocarbyl group having 1 to 16 carbon atoms, an aryl group having 6 to 12 carbon atoms, or an aralkyl group having 7 to 15 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When the “x” and/or “z” is 2 or more, the R^{fe}s may be identical to or different from each other.

Among the above, as R^{fe}, a hydroxy group, —N(R^{feC})—C(═O)—R^{feD}, —N(R^{feC})—C(═O)—O—R^{feD}, a fluorine atom, a chlorine atom, a bromine atom, a methyl group, a methoxy group, etc. are preferable.

In the general formula (c1-5), Rf¹¹ to Rf¹⁴ each independently represent a hydrogen atom, a fluorine atom, or a trifluoromethyl group, provided that at least one of Rf¹¹ to Rf¹⁴ is a fluorine atom or a trifluoromethyl group. In addition, Rf¹¹ and Rf¹² are optionally combined to form a carbonyl group. In particular, it is preferable that both Rf¹³ and Rf¹⁴ are fluorine atoms.

Specific examples of the anion represented by the general formula (c1-5) include the following, but are not limited thereto. Note that, in the following formulae, X^{BI} is as defined above.

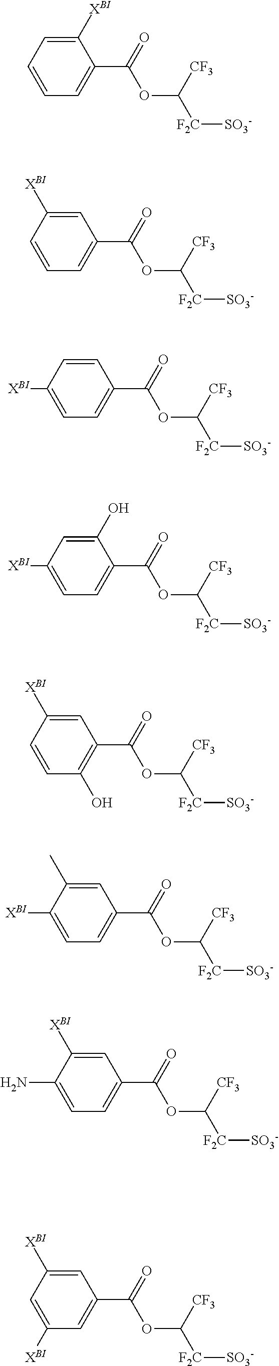

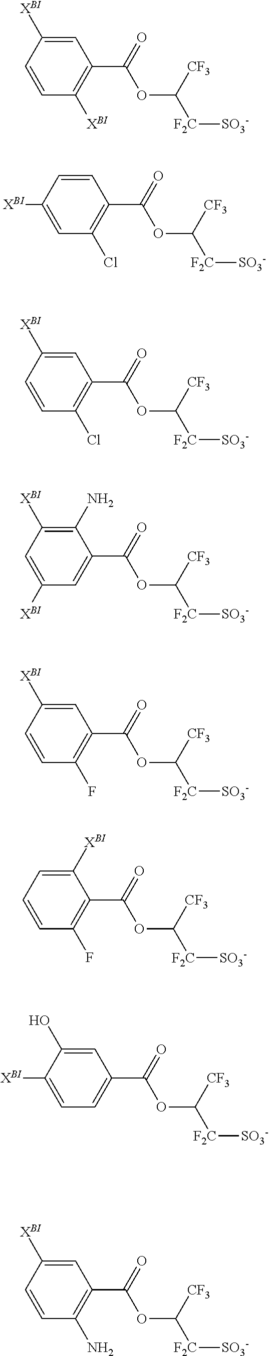

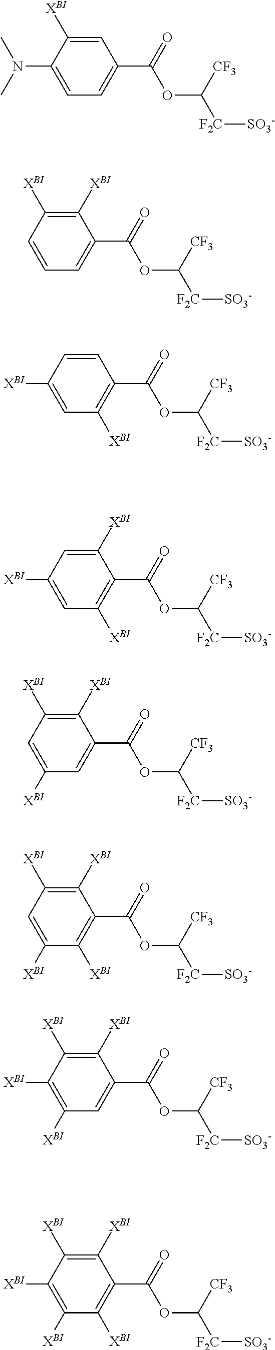

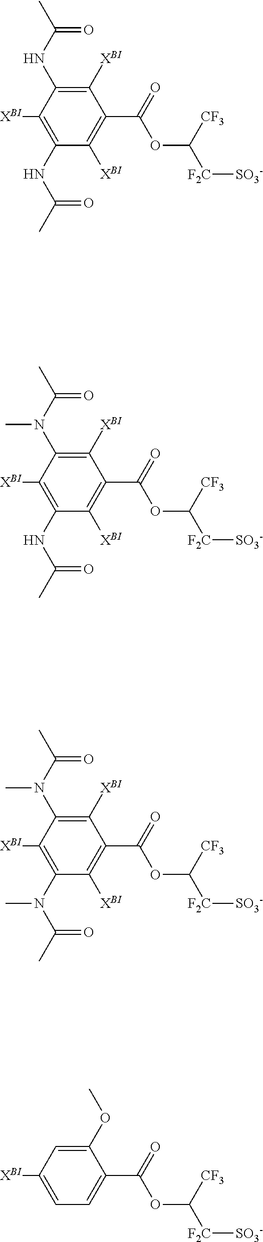

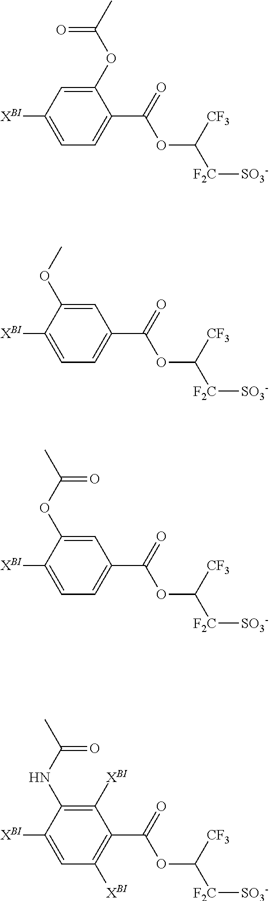

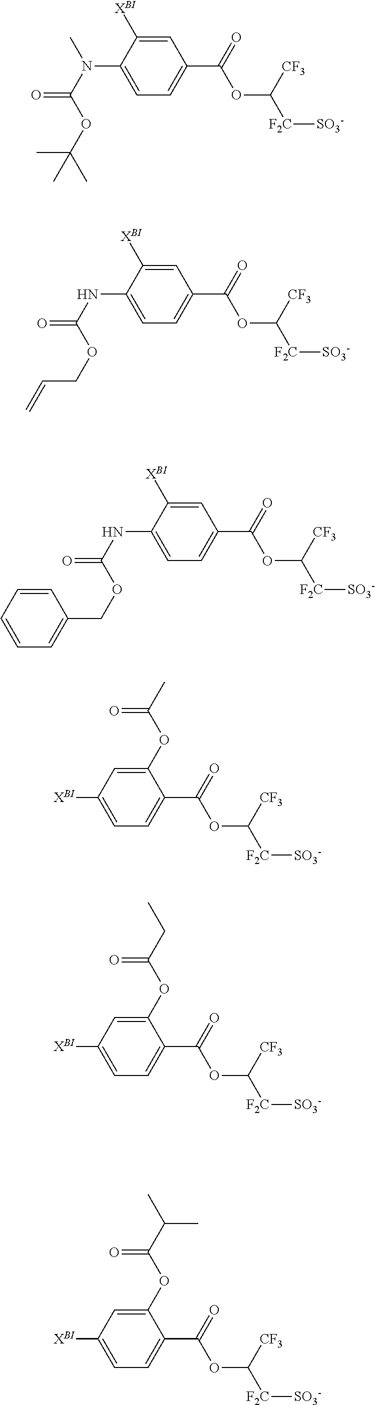

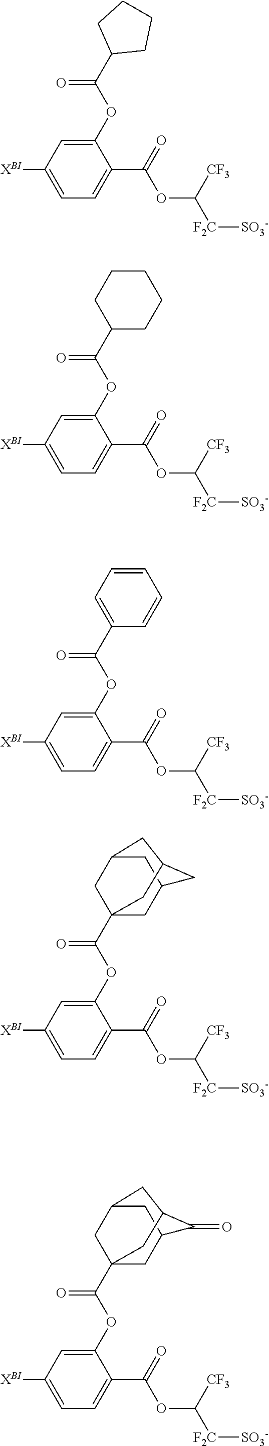

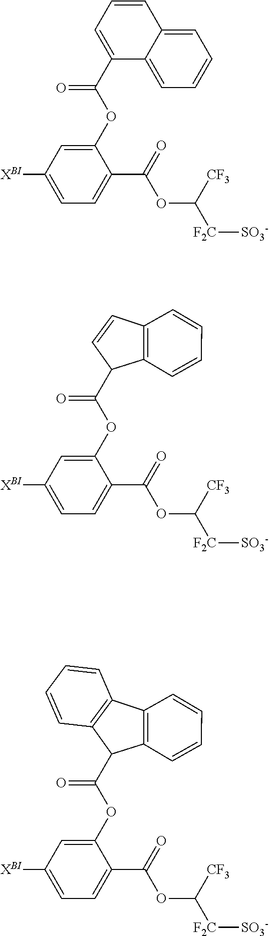

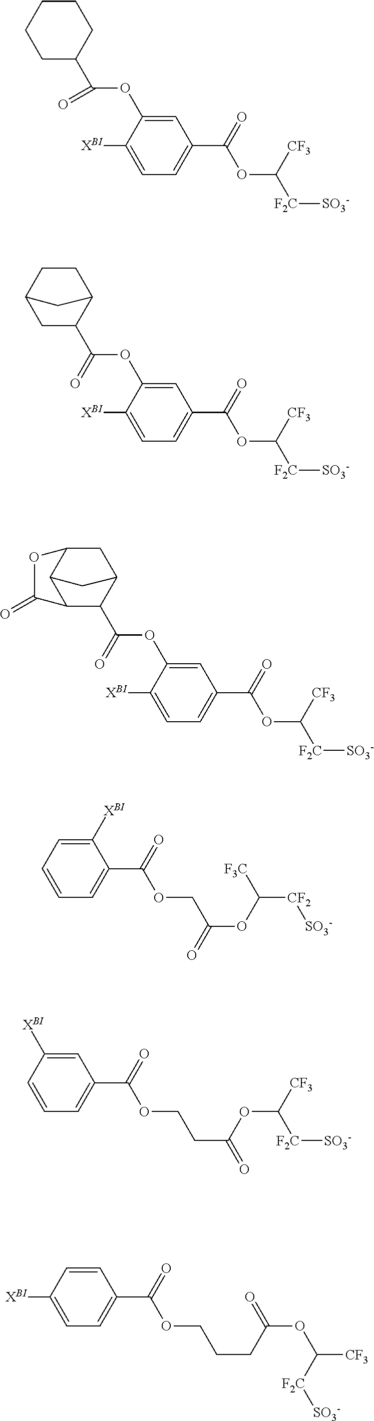

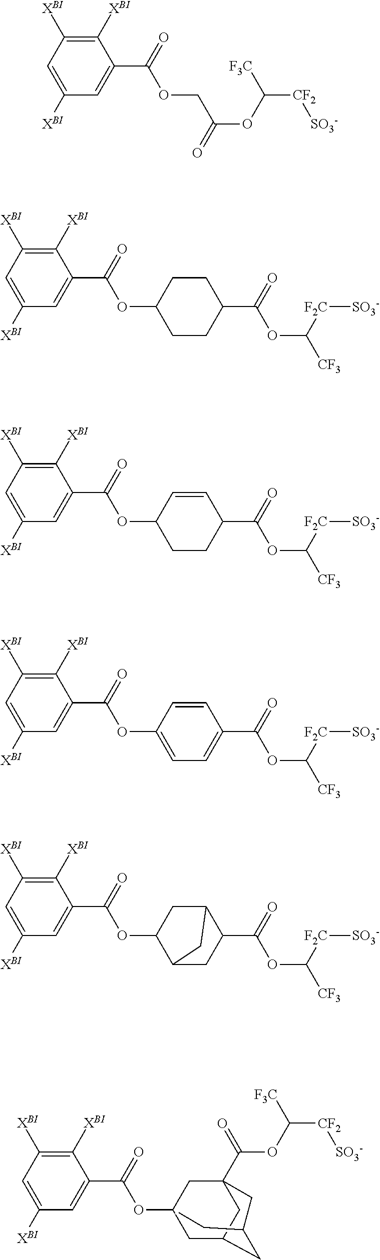

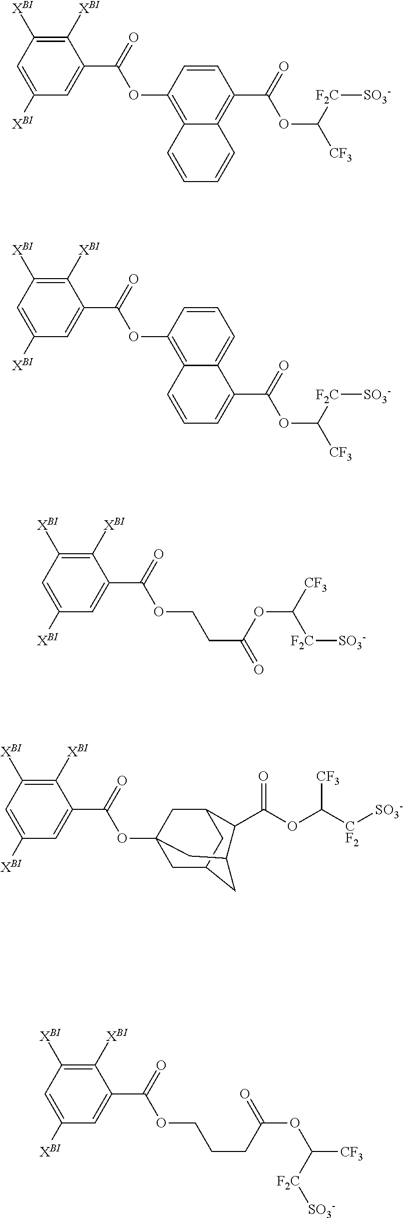

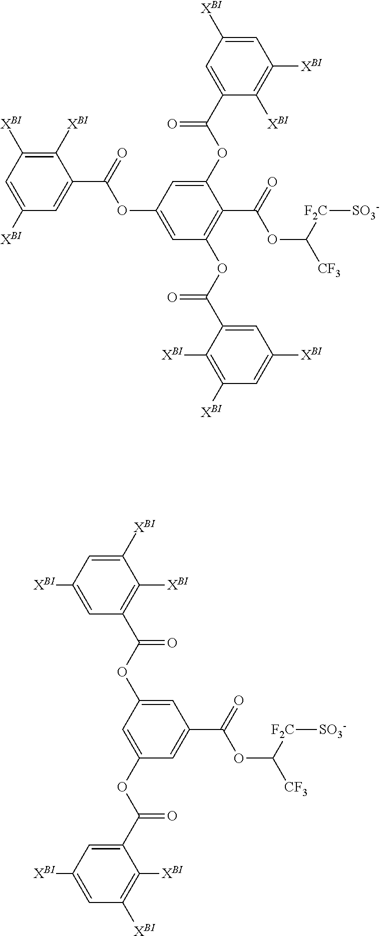

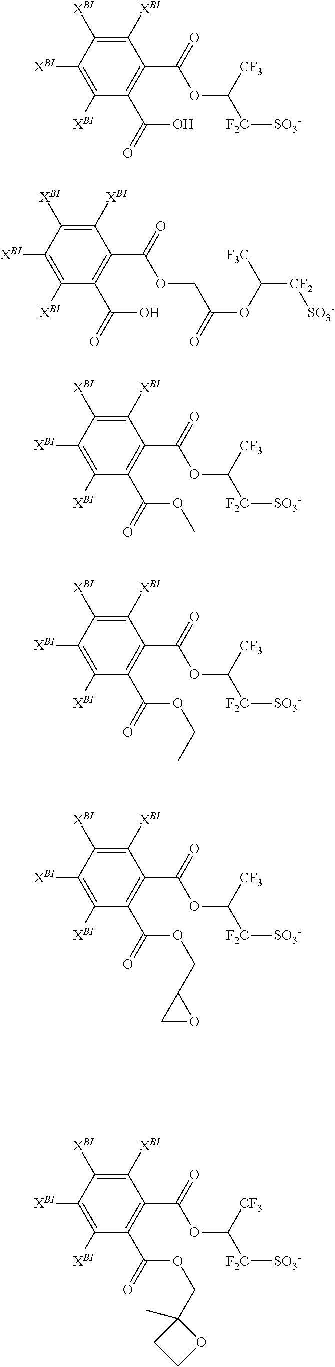

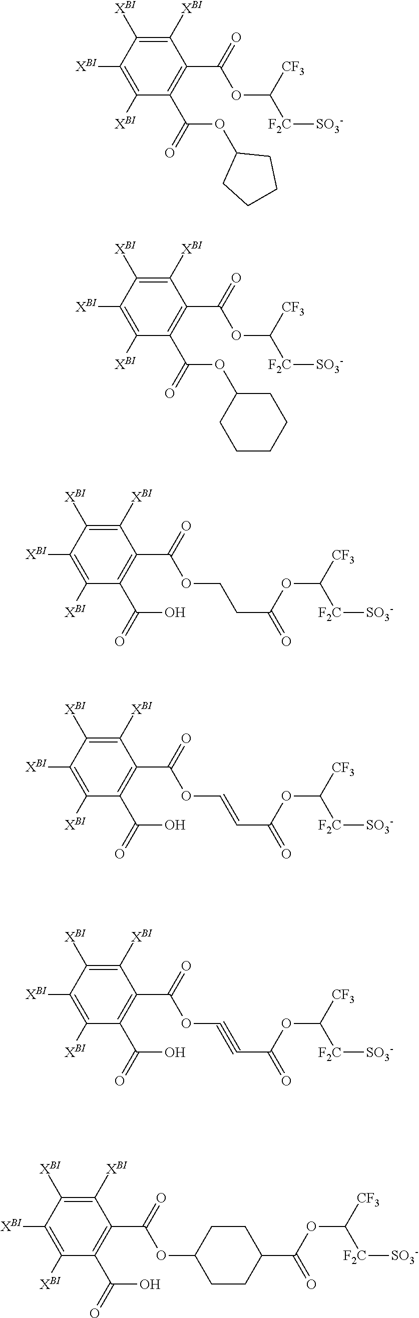

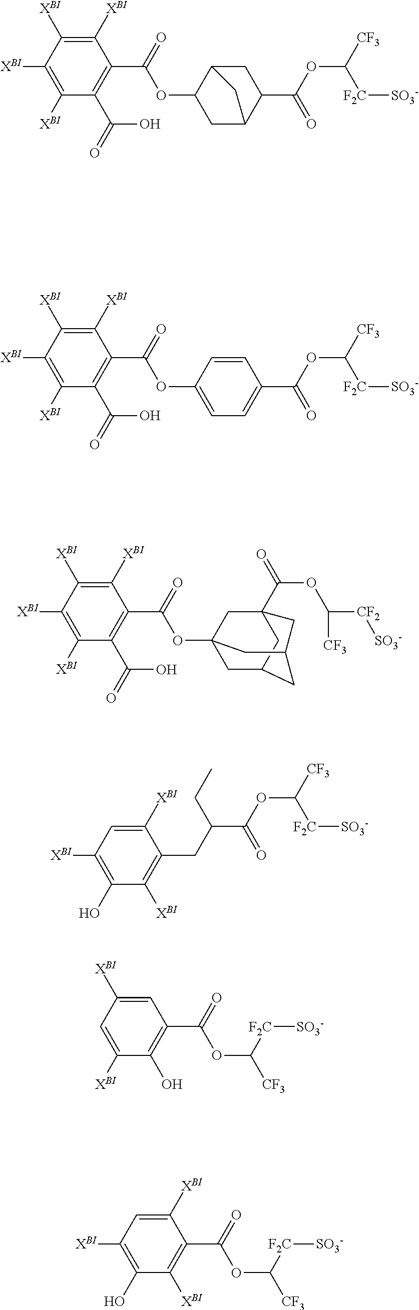

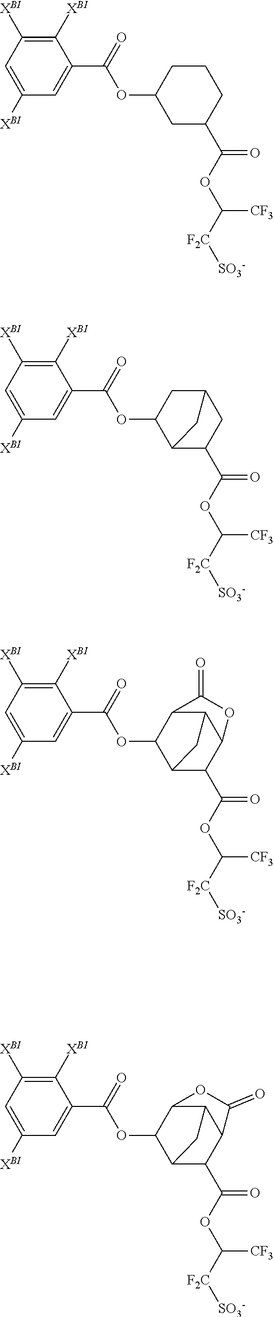

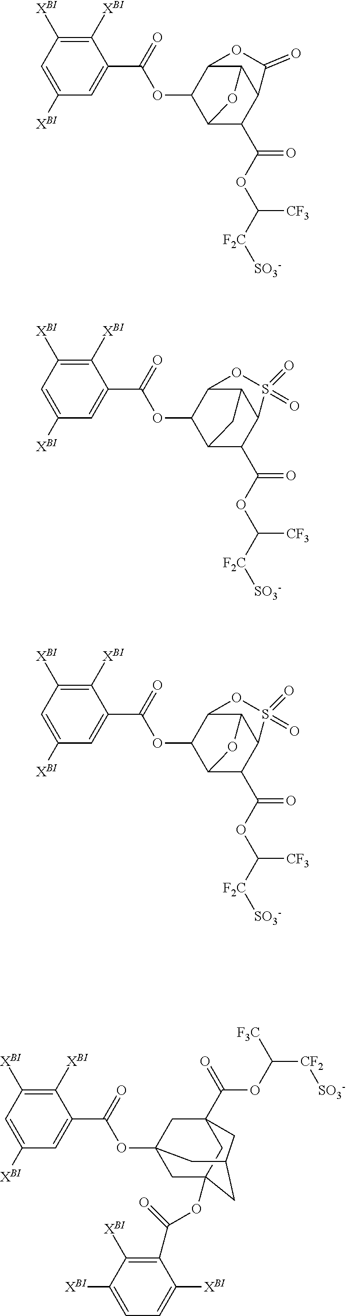

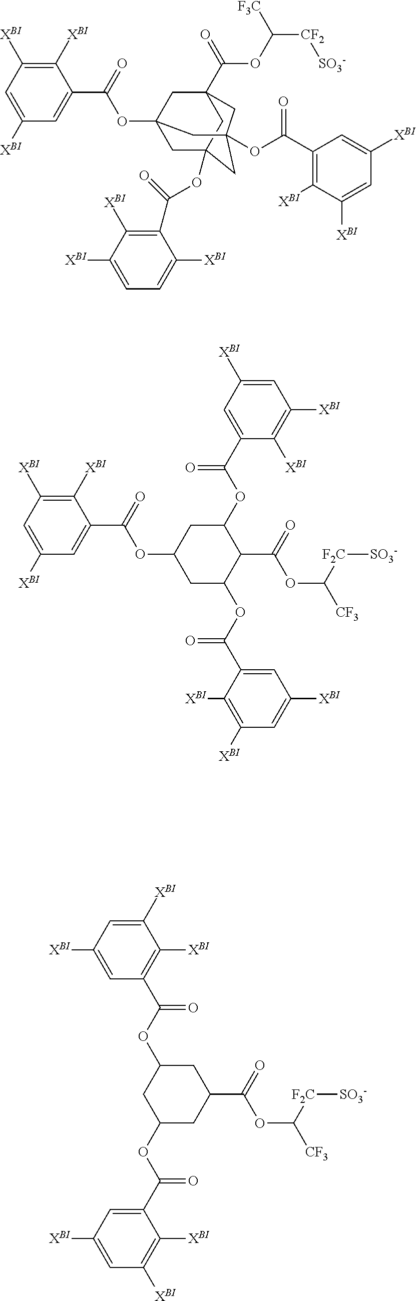

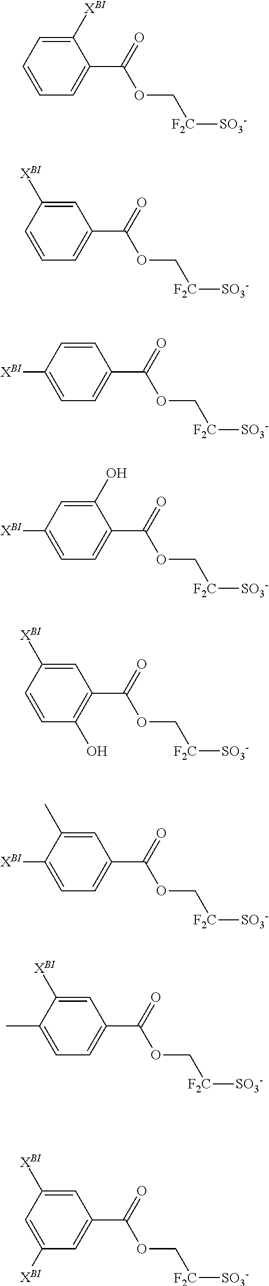

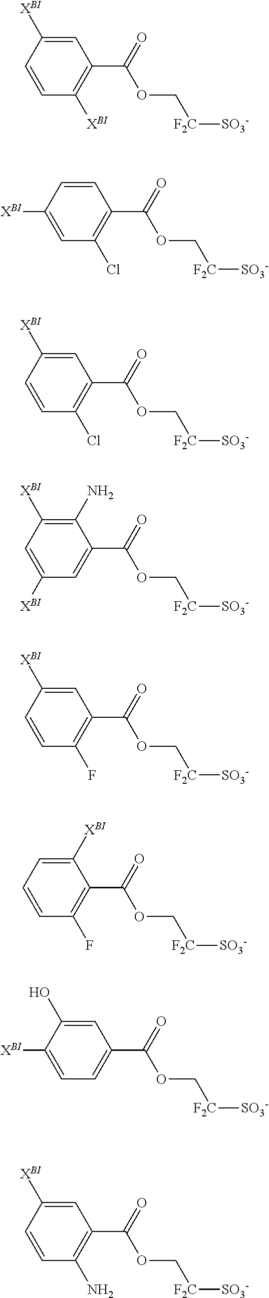

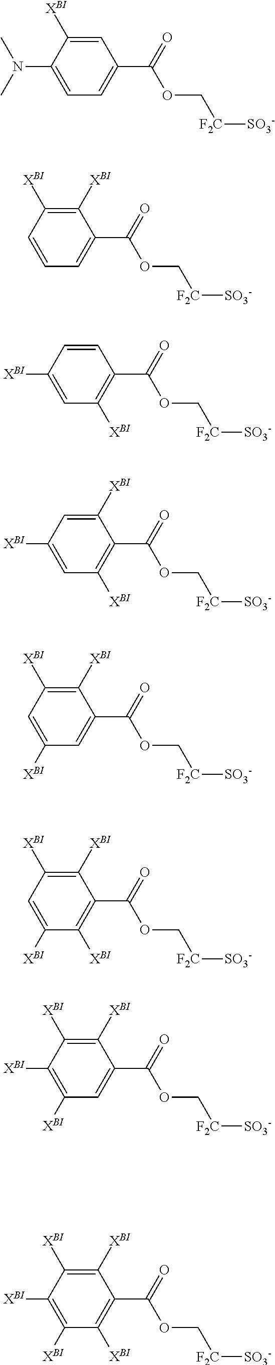

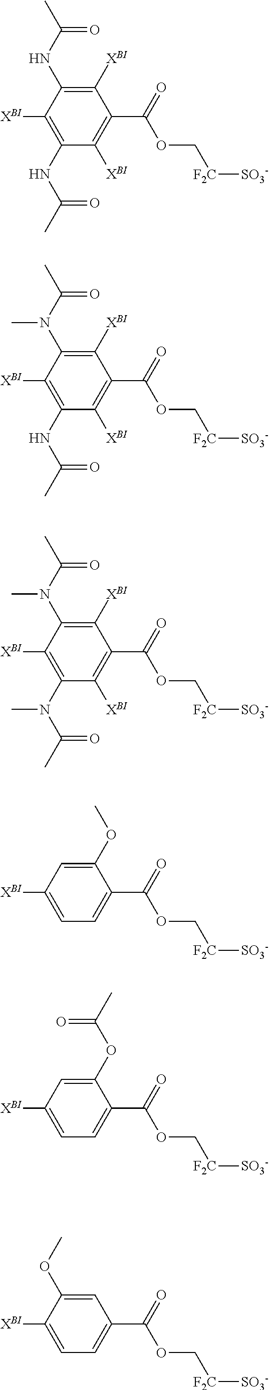

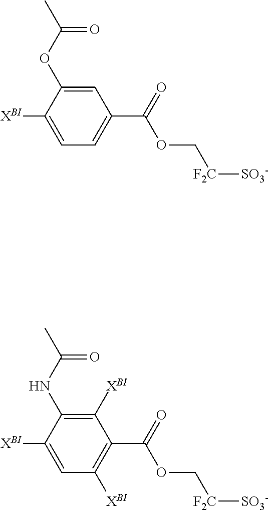

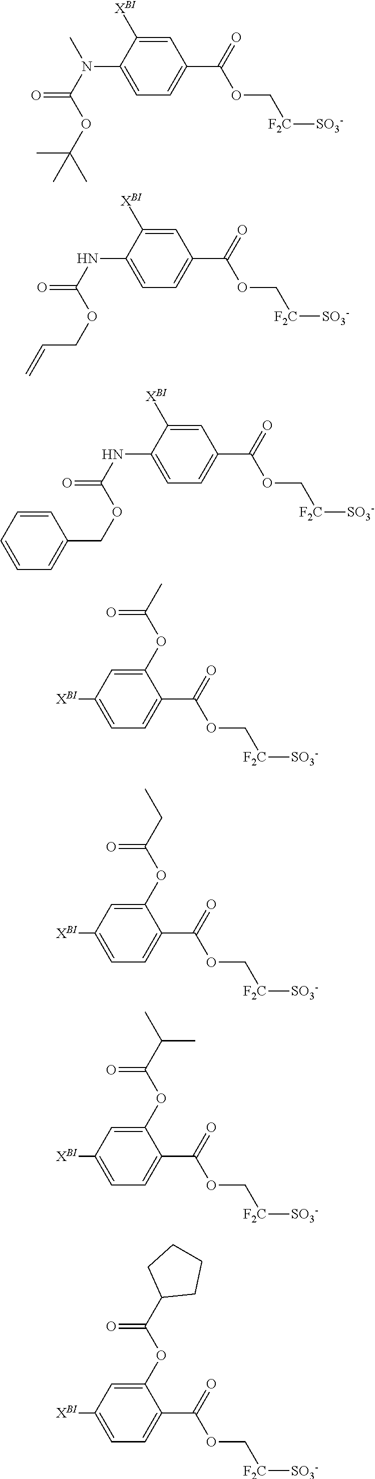

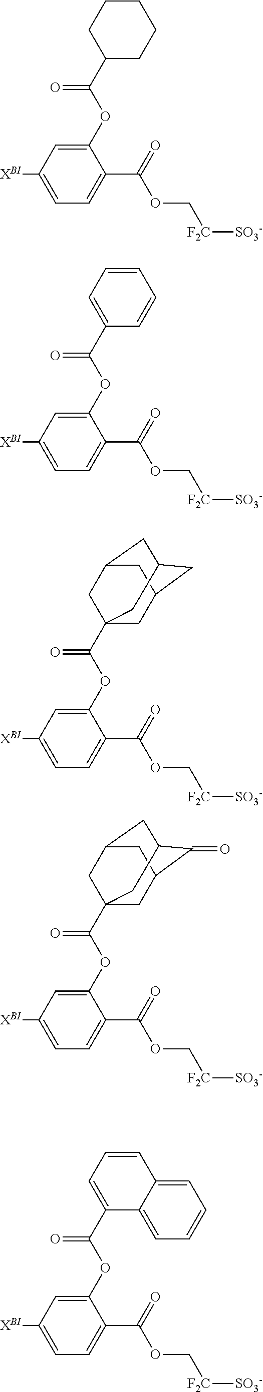

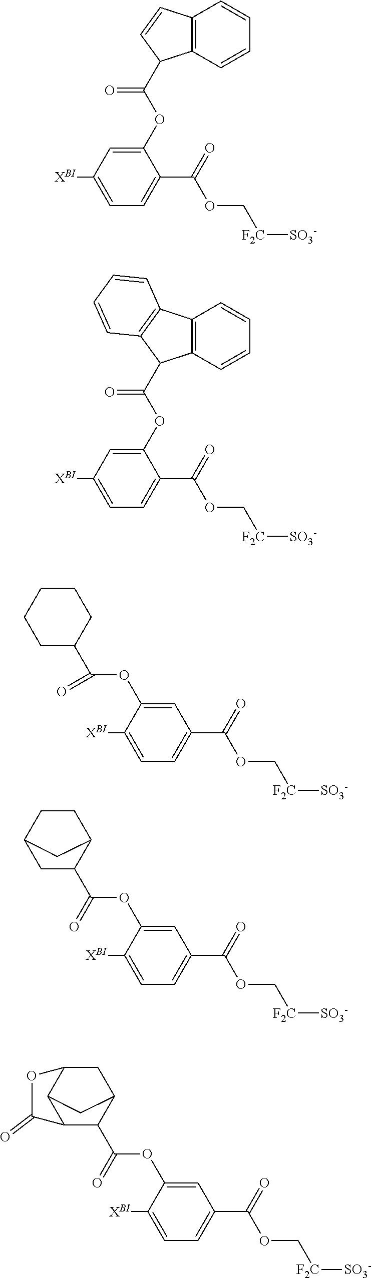

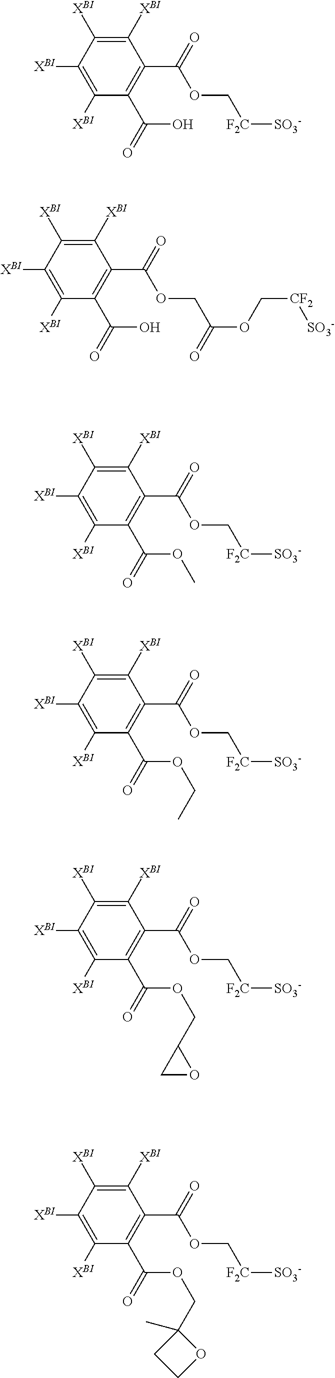

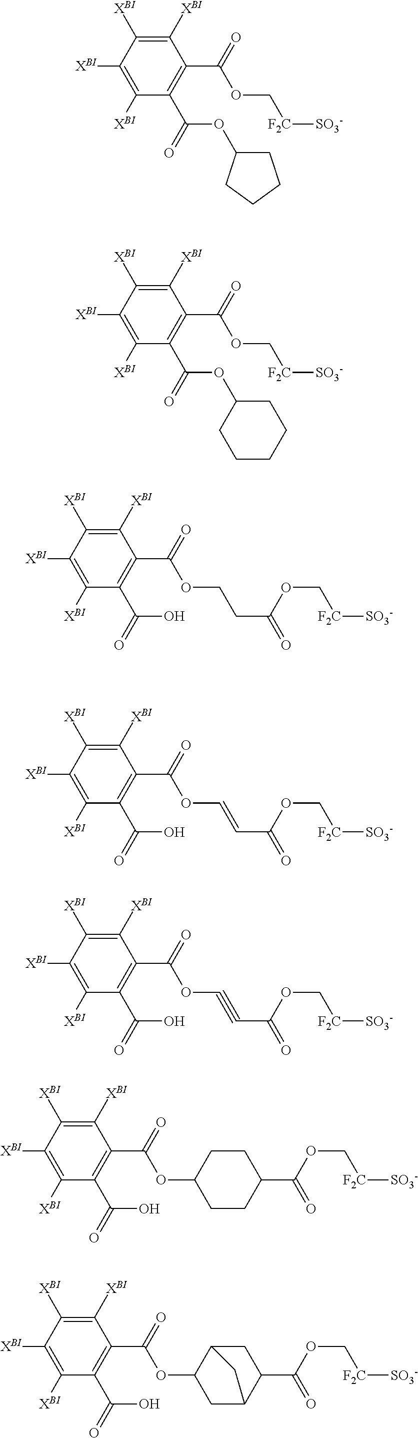

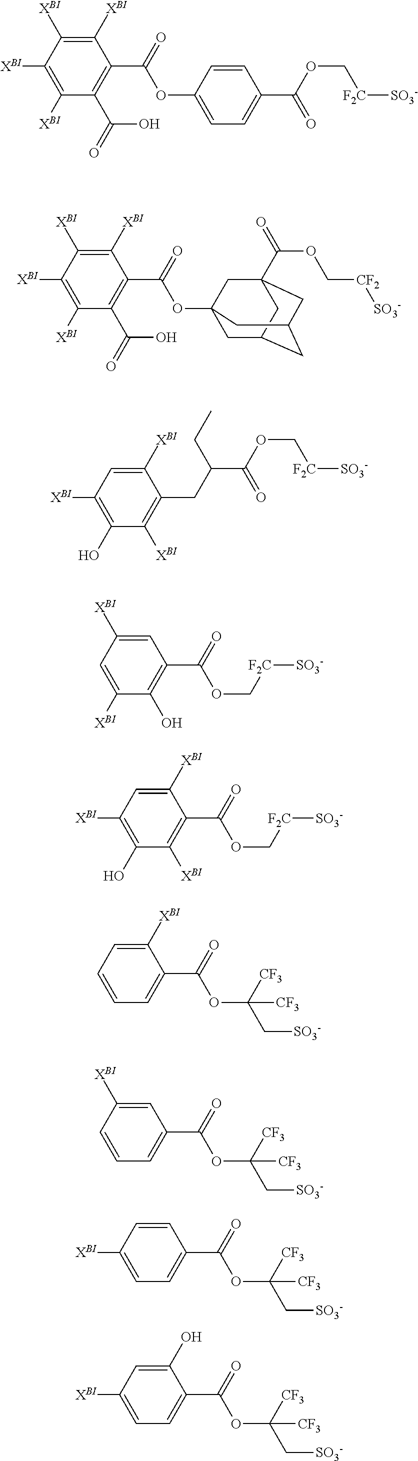

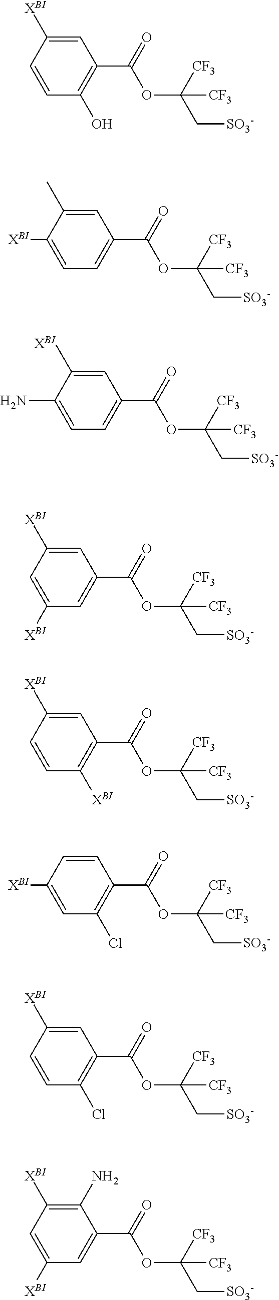

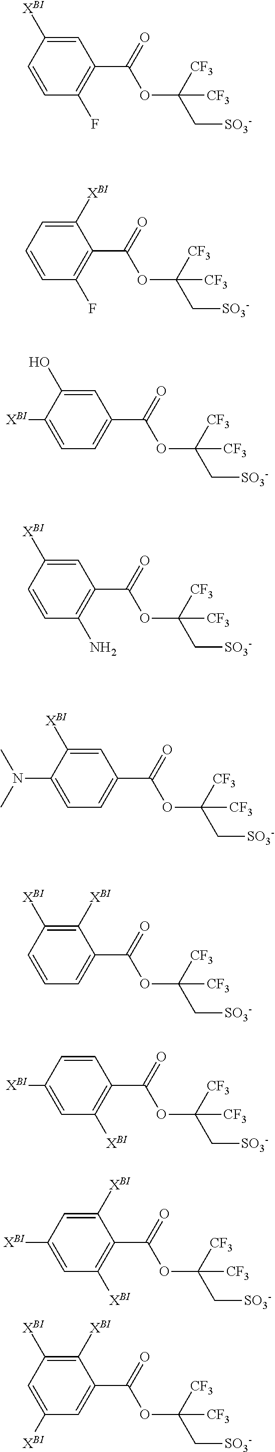

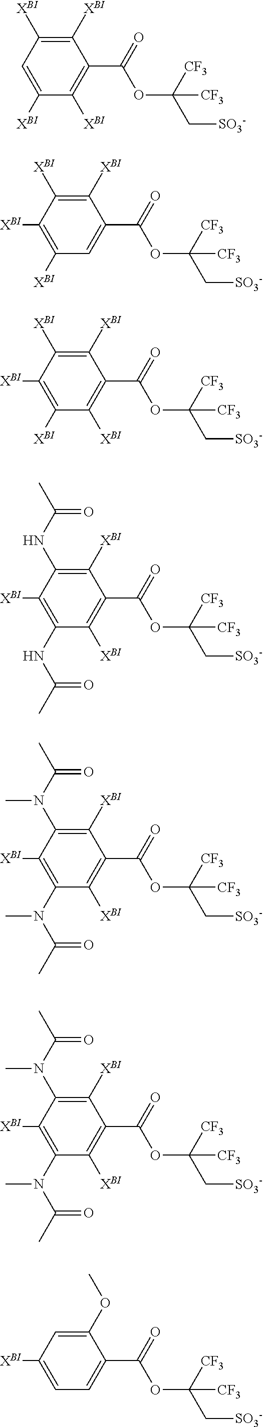

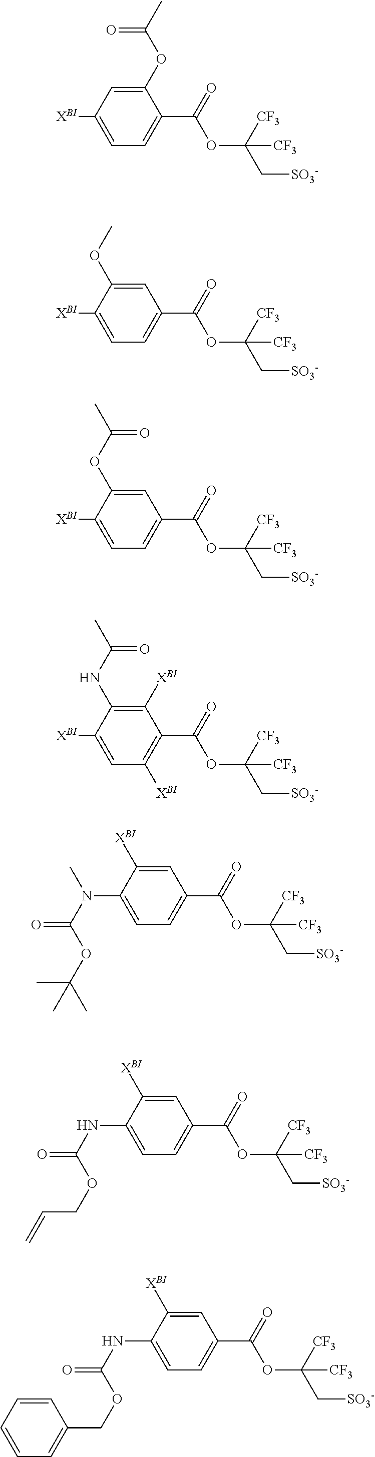

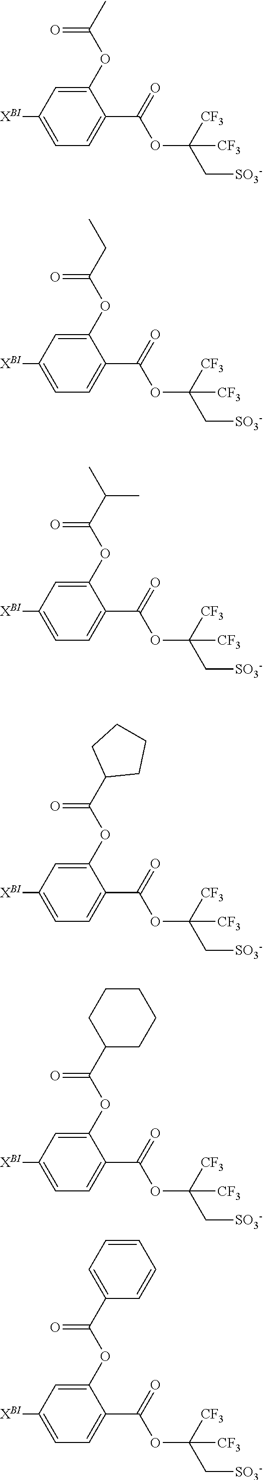

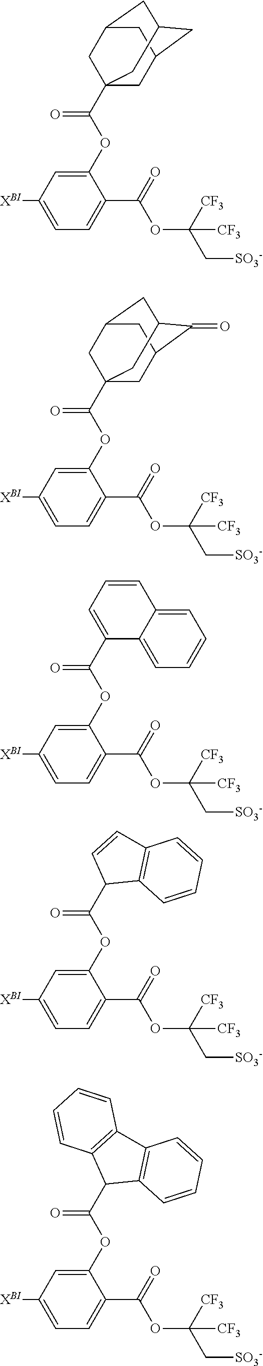

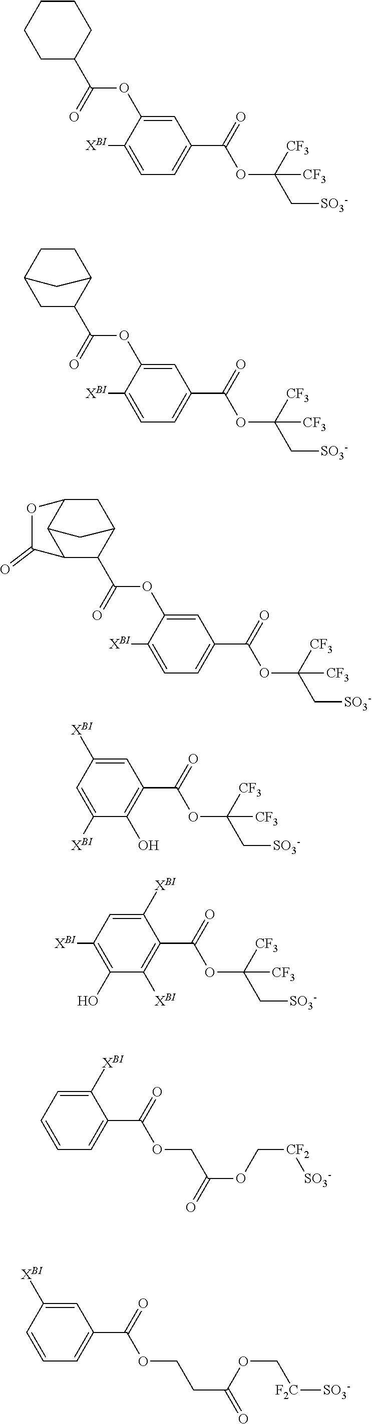

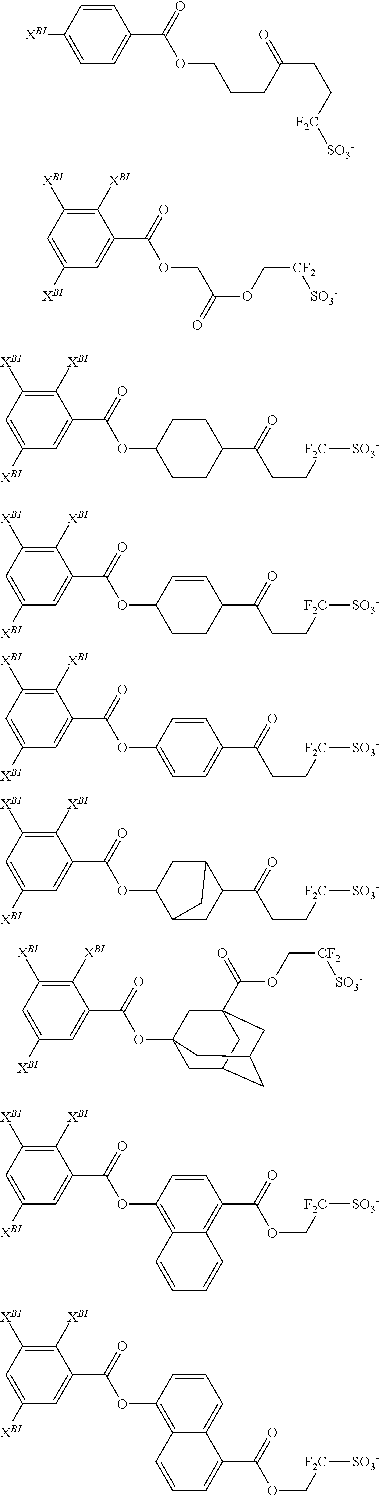

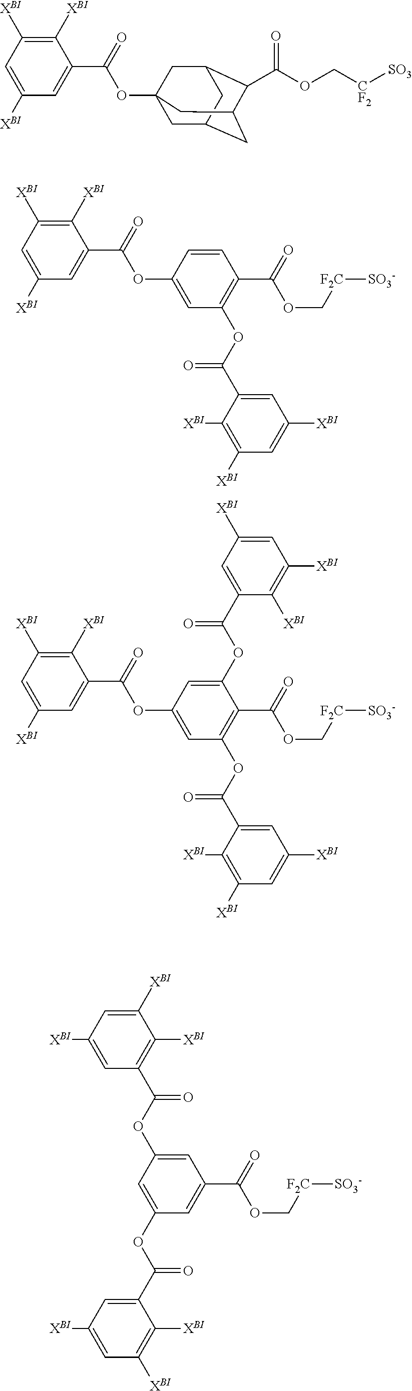

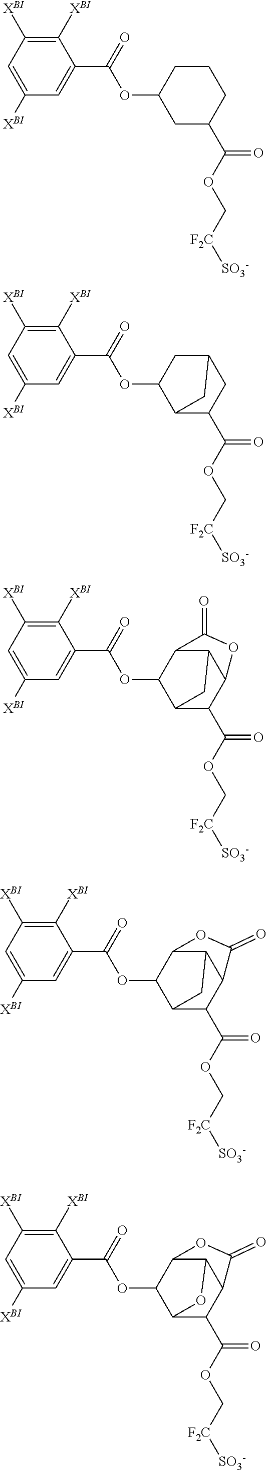

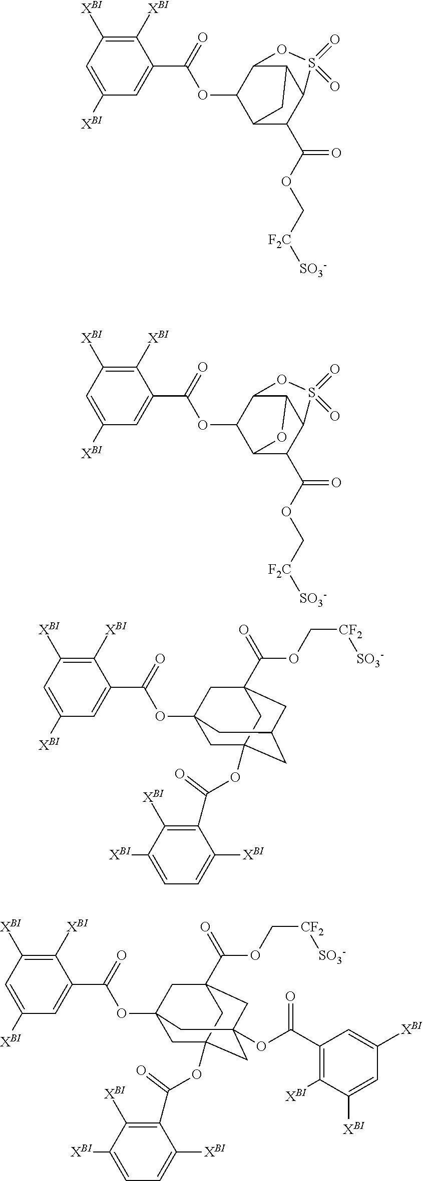

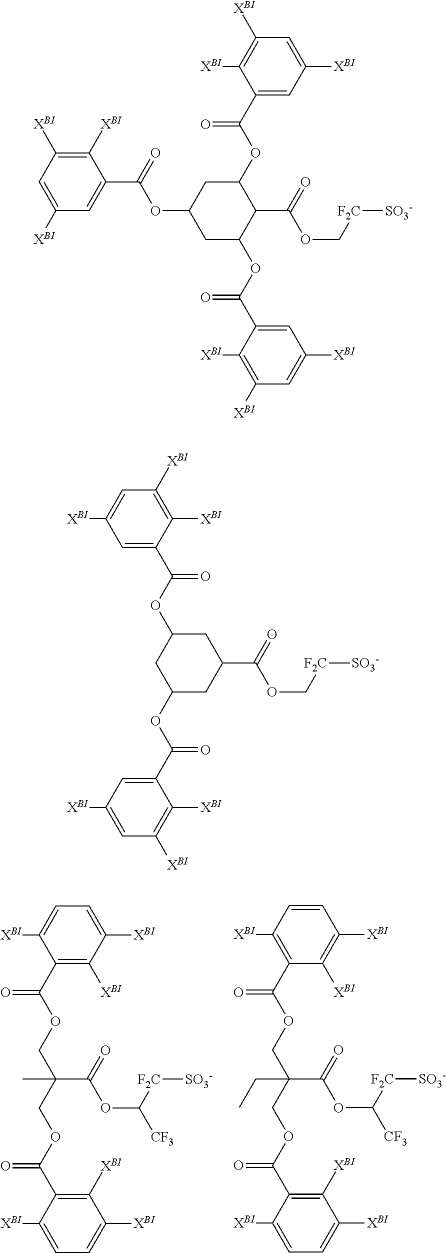

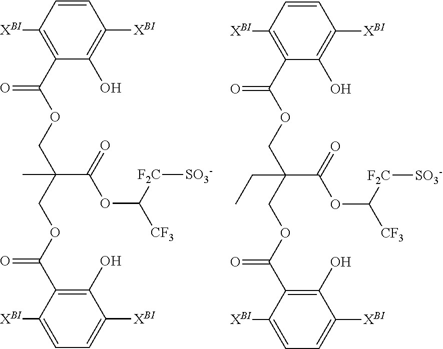

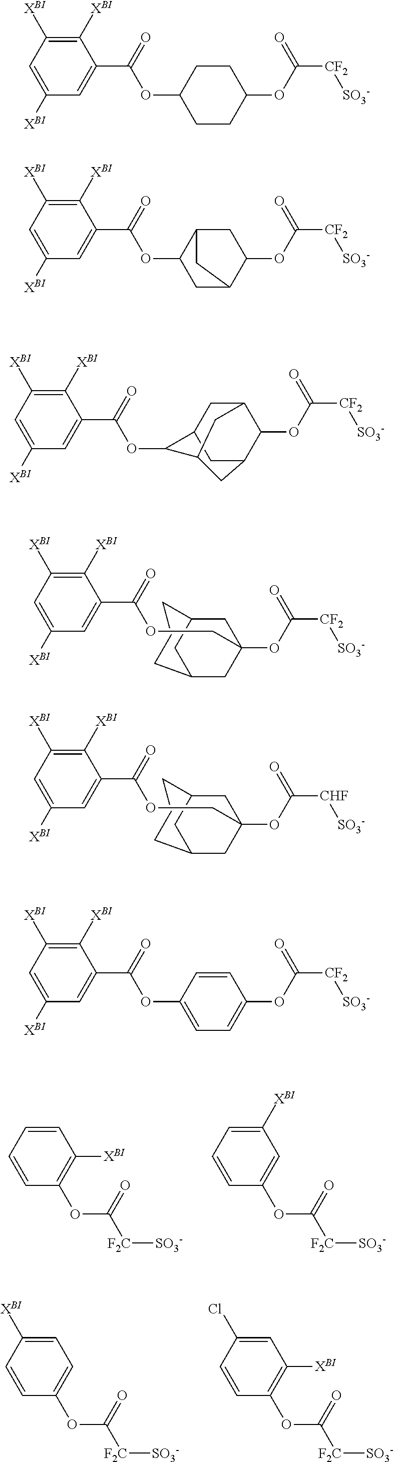

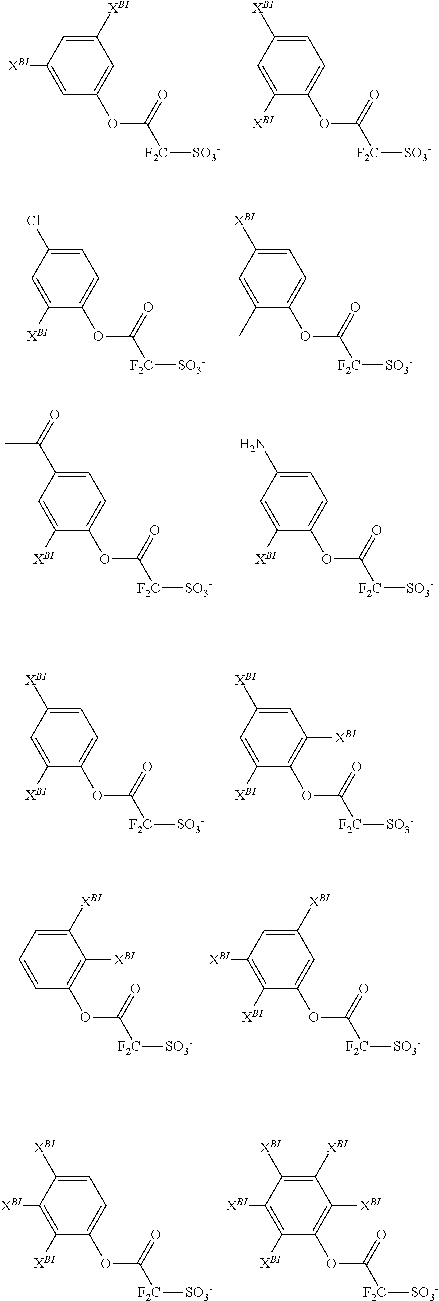

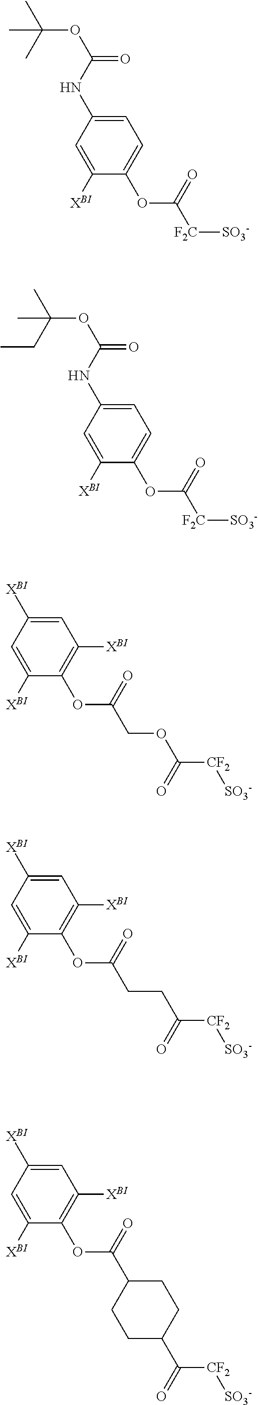

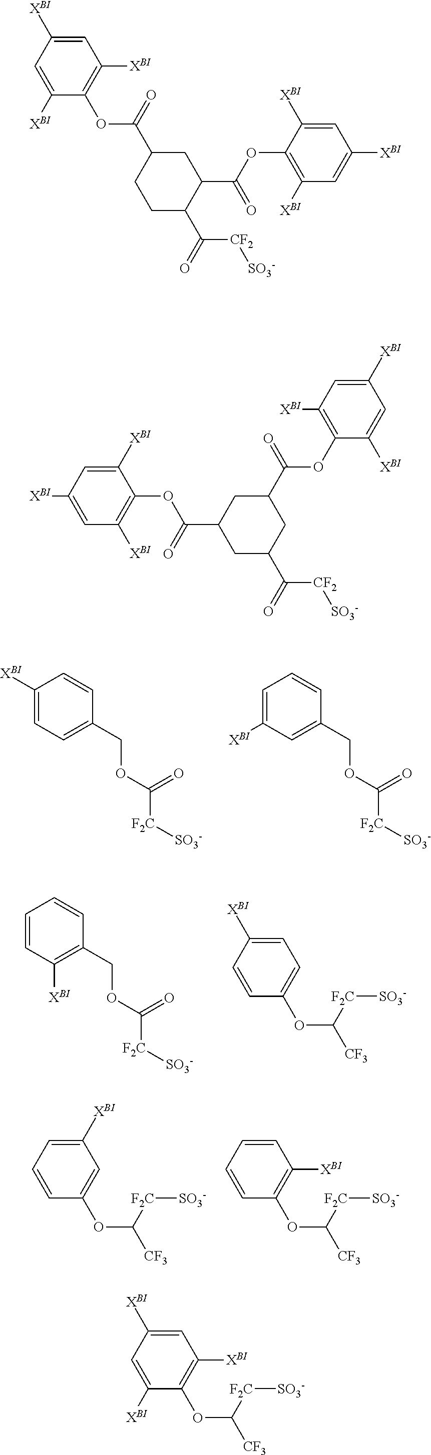

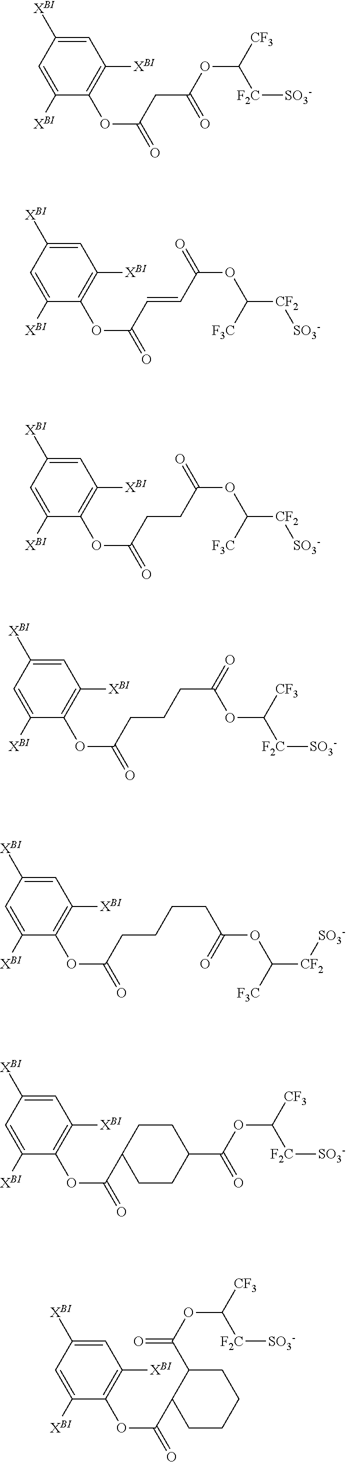

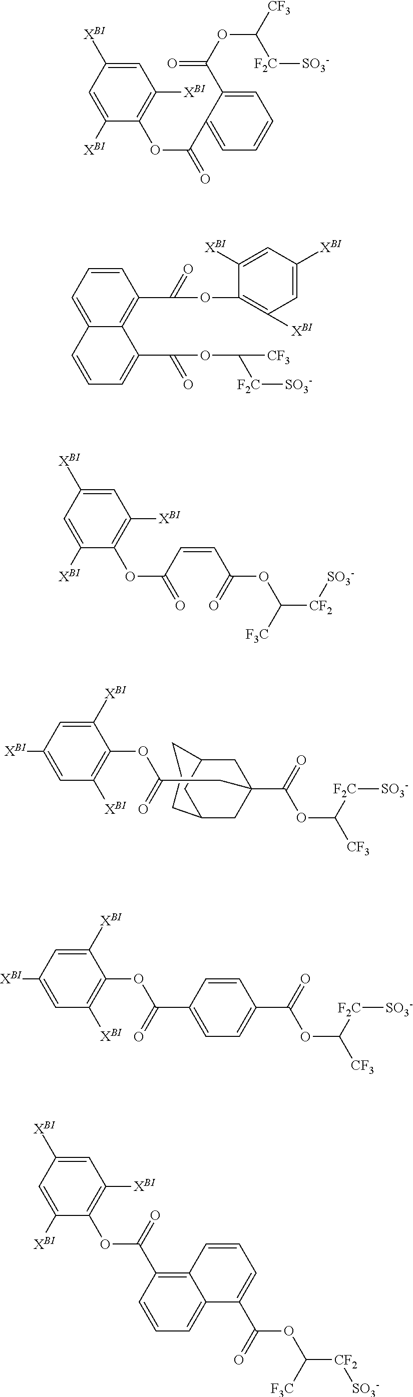

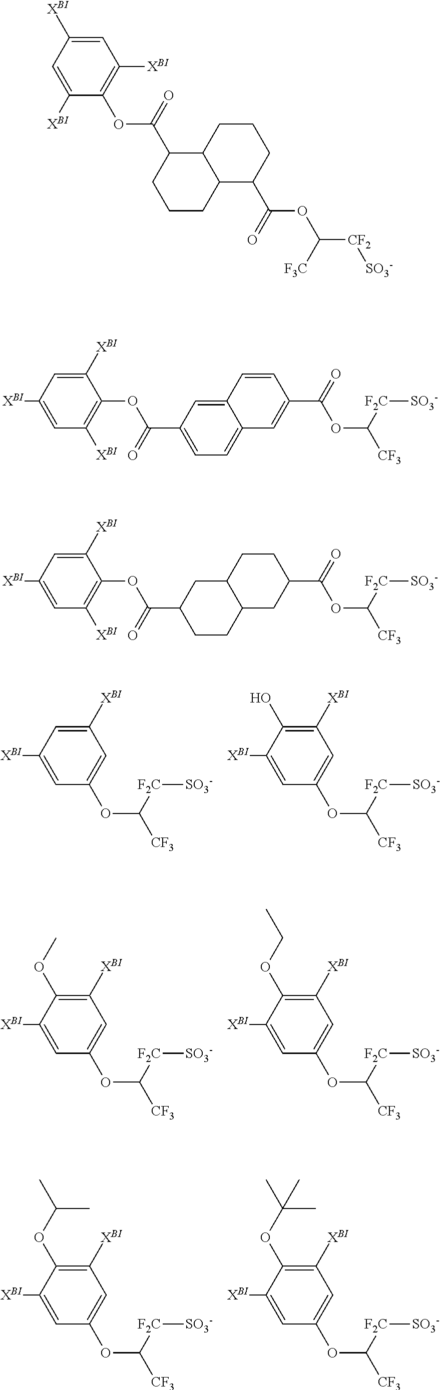

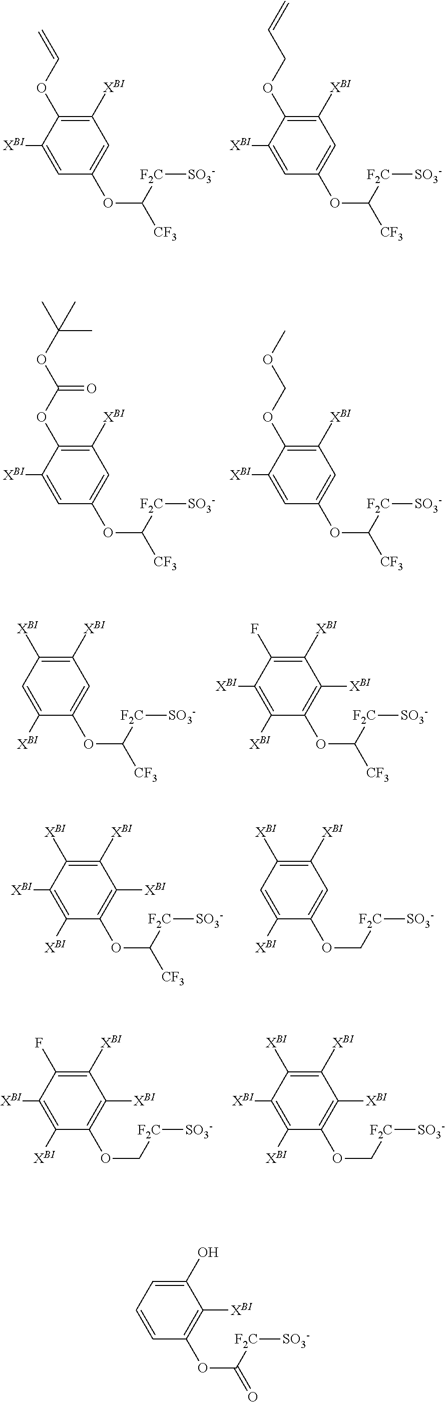

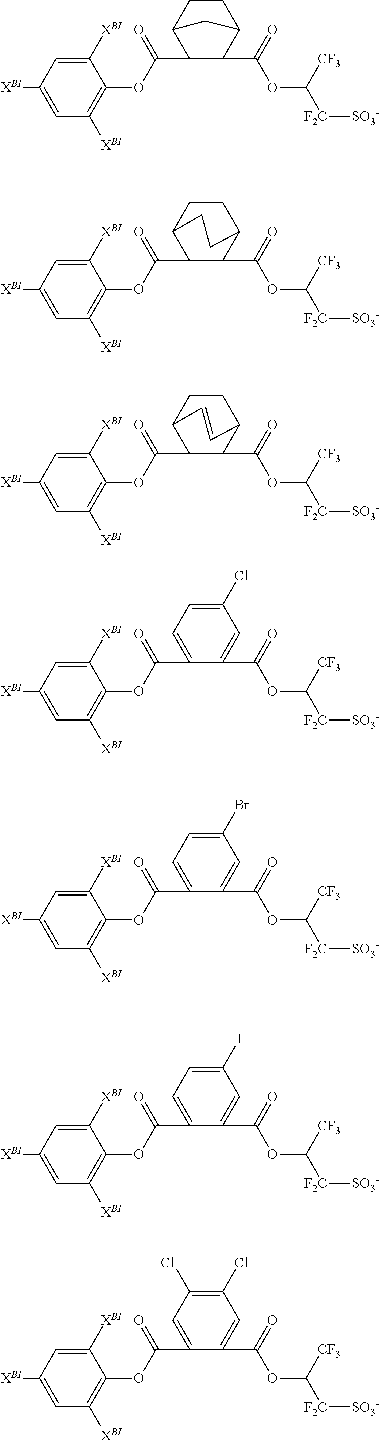

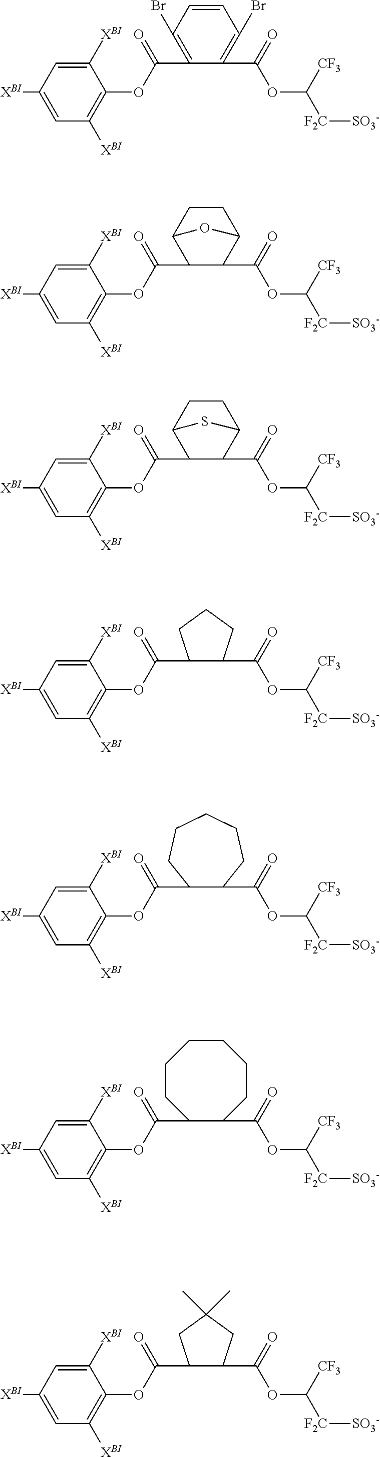

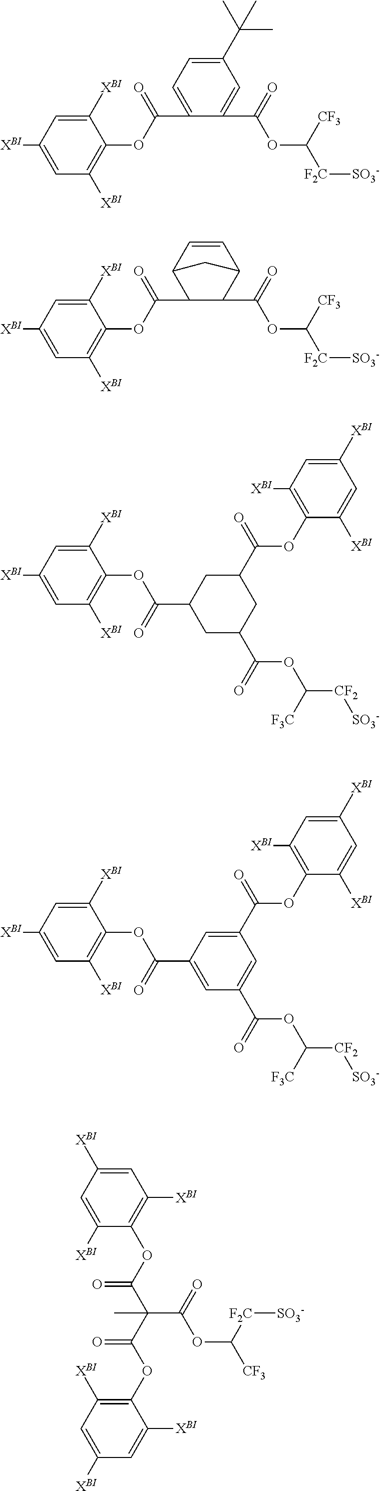

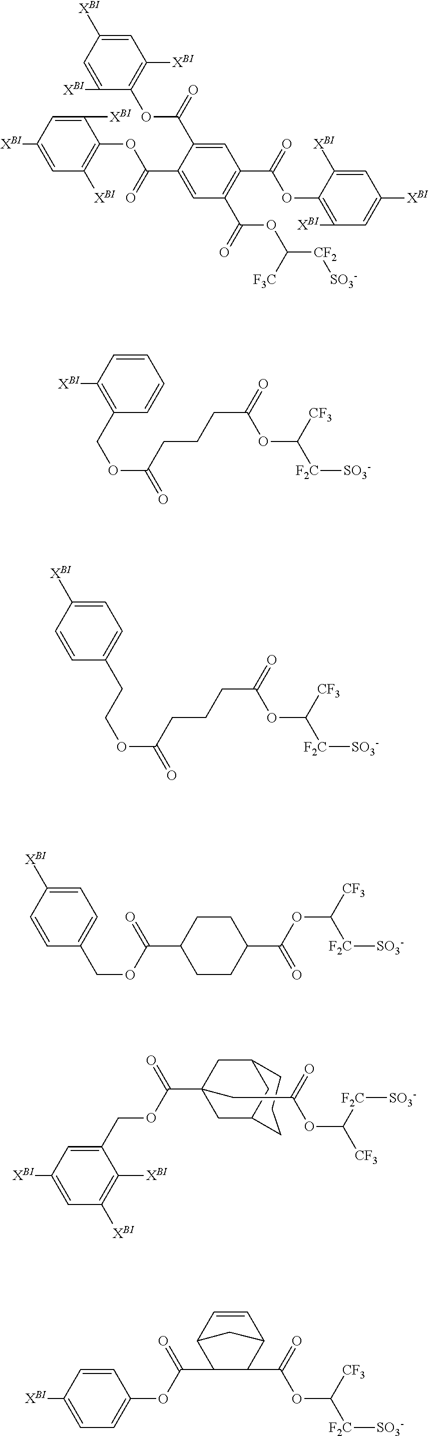

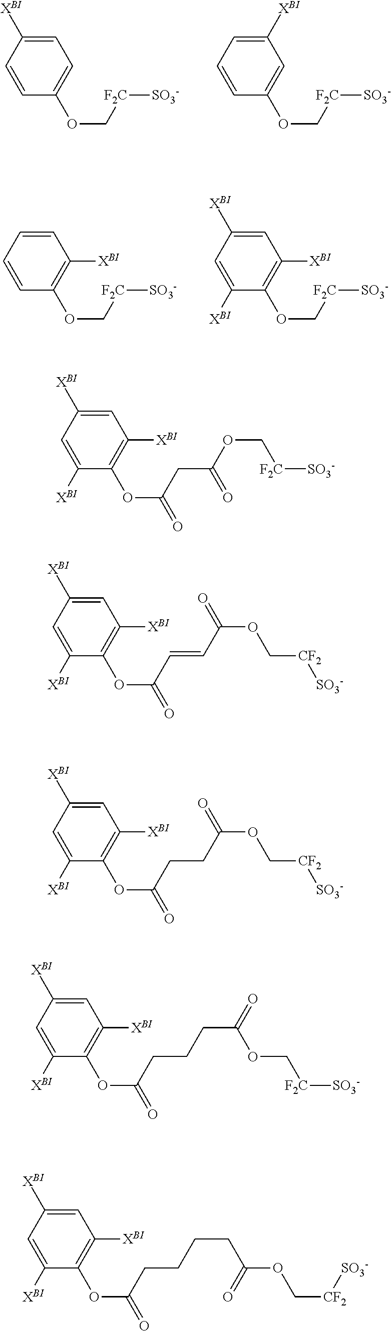

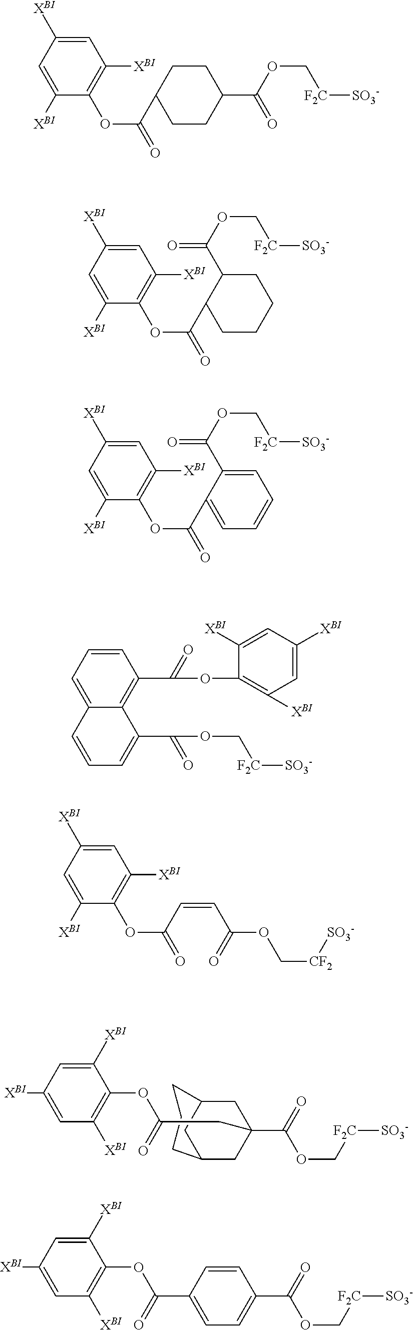

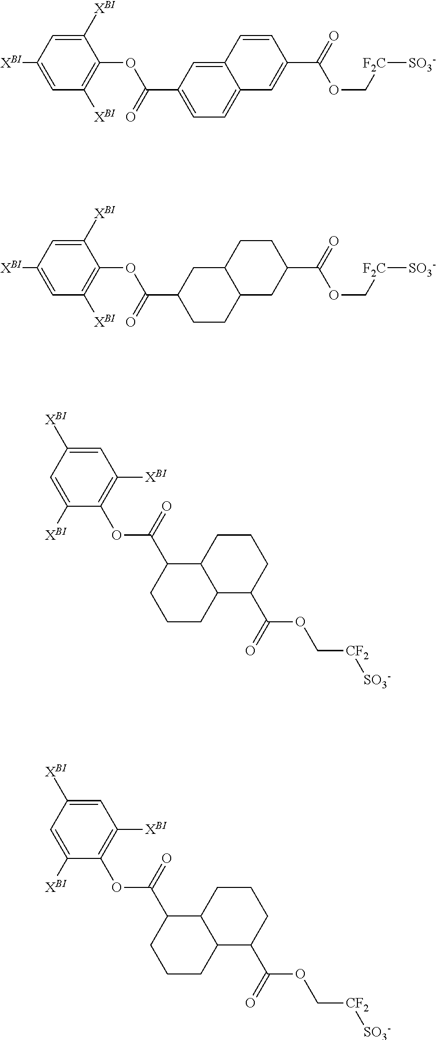

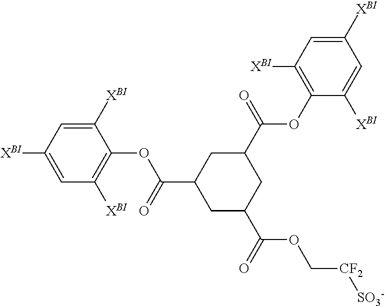

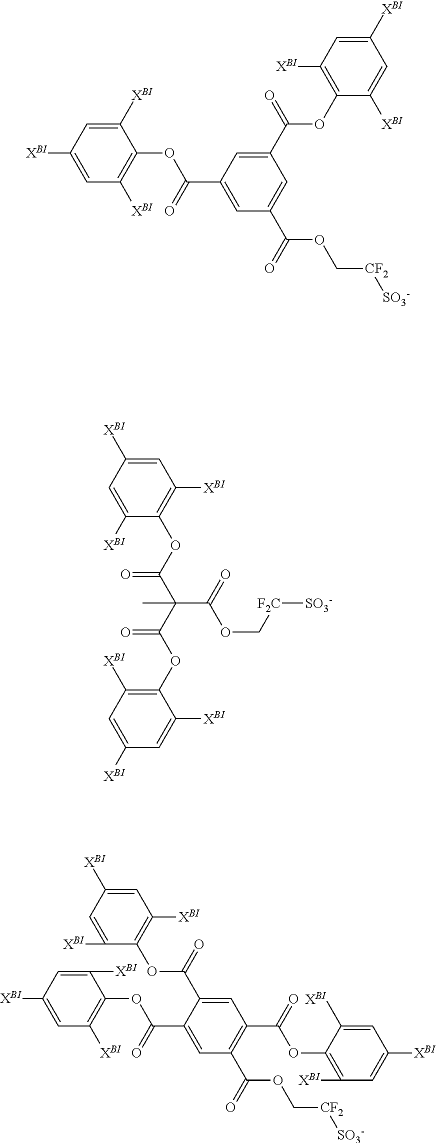

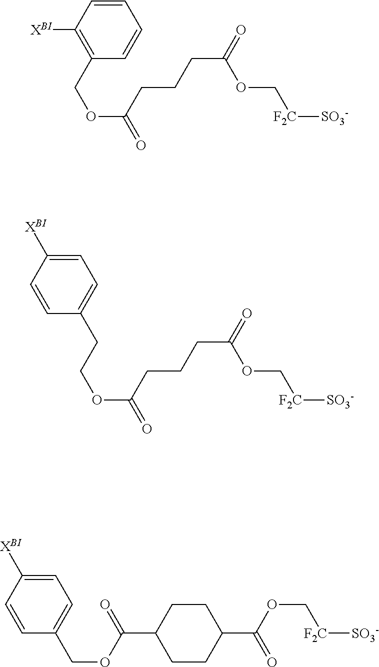

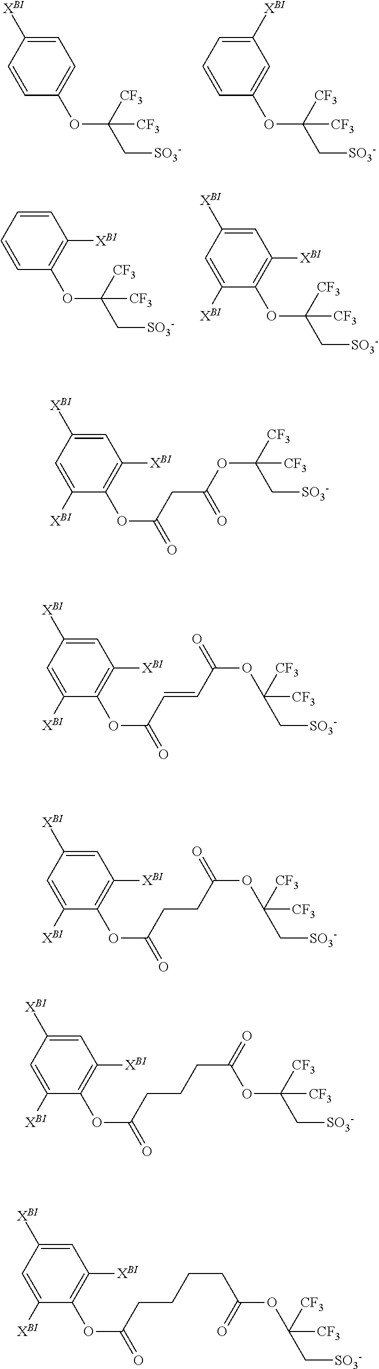

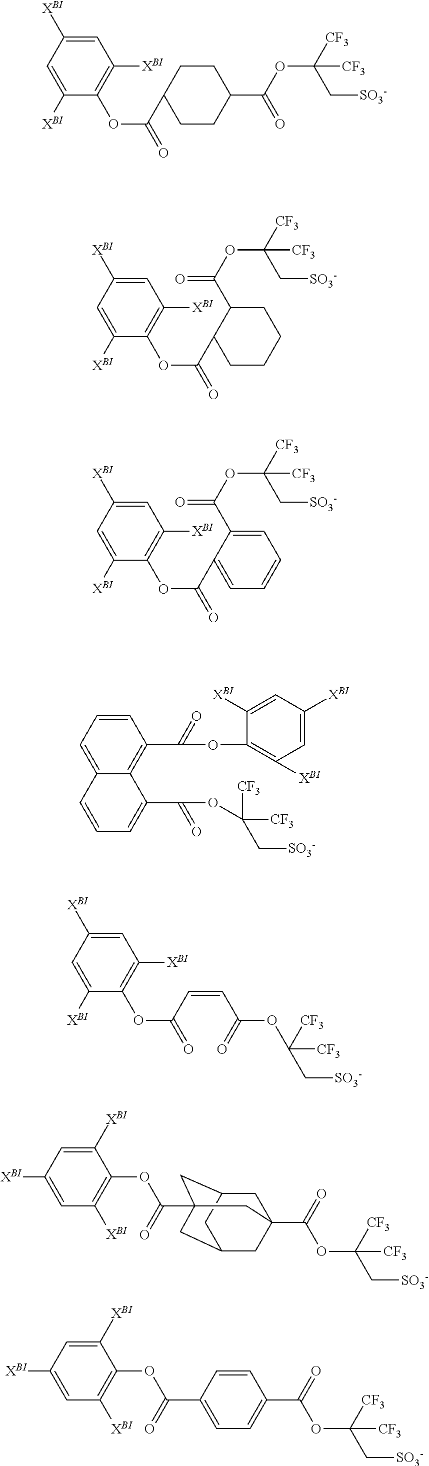

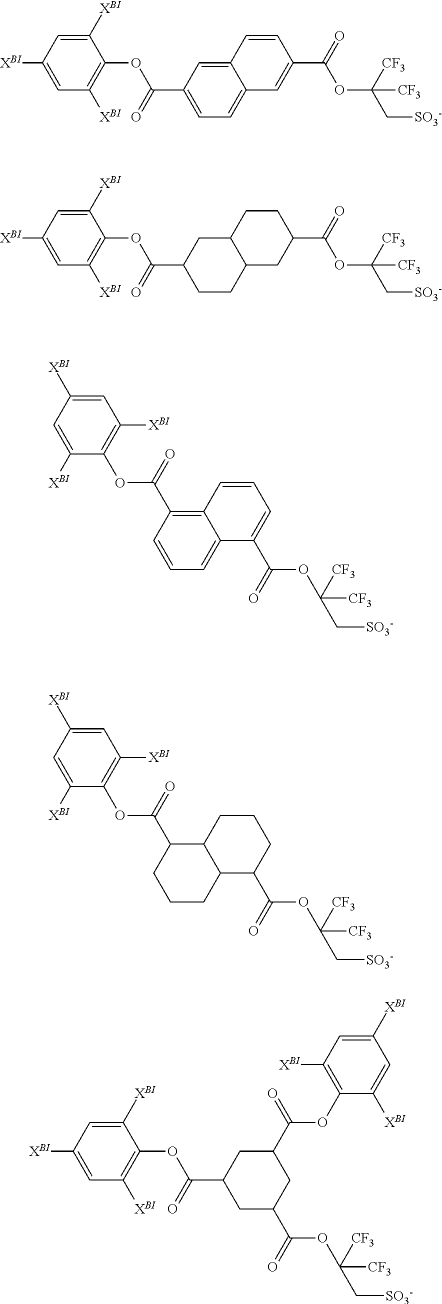

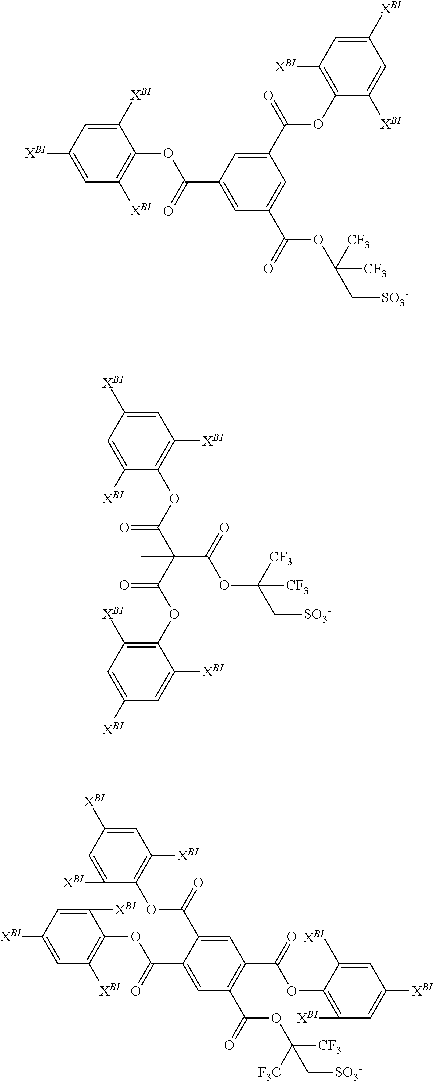

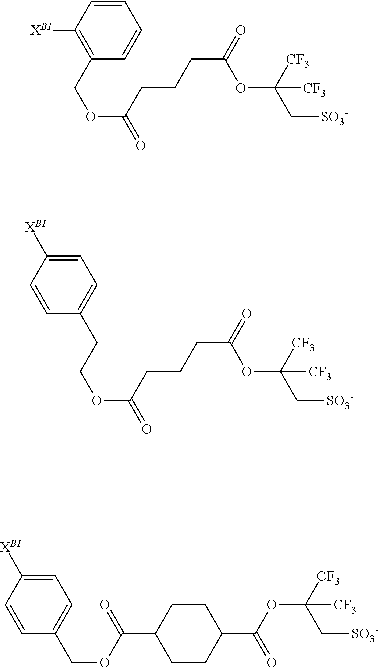

As the non-nucleophilic counter ion, it is also possible to use: a fluorobenzenesulfonate anion disclosed in JP 6648726 B2, bonded to an aromatic group containing an iodine atom; an anion disclosed in WO 2021/200056 A1 and JP 2021-070692 A, having a mechanism that the anion is decomposed by an acid; an anion disclosed in JP 2018-180525 A and JP 2021-035935 A, having a cyclic ether group; or an anion disclosed in JP 2018-092159 A.

Furthermore, as the non-nucleophilic counter ion, it is also possible to use: an anion disclosed in JP 2006-276759 A, JP 2015-117200 A, JP 2016-065016 A, and JP 2019-202974 A, being a bulky benzenesulfonate derivative containing no fluorine atoms; or a benzenesulfonate anion or alkylsulfonate anion disclosed in JP 6645464 B2, bonded to an aromatic group containing an iodine atom and not containing a fluorine atom.

Furthermore, as the non-nucleophilic counter ion, it is also possible to use: a bissulfonate anion disclosed in JP 2015-206932 A; a sulfonamide or sulfonimide anion disclosed in WO 2020/158366 A1, in which one is a sulfonic acid and the other is different from a sulfonic acid; or an anion disclosed in JP 2015-024989 A, in which one is a sulfonic acid and the other is a carboxylic acid.

In the general formulae (c2) and (c3), L¹ represents a single bond, an ether bond, an ester bond, a carbonyl group, a sulfonic acid ester bond, a carbonate bond, or a carbamate bond. Among these, from the viewpoint of synthesis, an ether bond, an ester bond, or a carbonyl group is preferable, and an ester bond or a carbonyl group is further preferable.

In the general formula (c2), Rf¹ and Rf² each independently represent a fluorine atom or a fluorinated saturated hydrocarbyl group having 1 to 6 carbon atoms. Among these, a fluorine atom is preferable as both Rf¹ and Rf² for enhancing the acidity of the generated acid. Rf³ and Rf⁴ each independently represent a hydrogen atom, a fluorine atom, or a fluorinated saturated hydrocarbyl group having 1 to 6 carbon atoms. Among these, it is preferable that at least one of Rf³ and Rf⁴ is a trifluoromethyl group for enhancing solvent solubility.

In the general formula (c3), Rf⁵ and Rf⁶ each independently represent a hydrogen atom, a fluorine atom, or a fluorinated saturated hydrocarbyl group having 1 to 6 carbon atoms, provided that not all of the Rf⁵ and the Rf⁶ are hydrogen atoms simultaneously. Among these, it is preferable that at least one of Rf⁵ and Rf⁶ is a trifluoromethyl group for enhancing solvent solubility.

In the general formulae (c2) and (c3), “d” represents an integer of 0 to 3, preferably 1.

Specific examples of the anion of the repeating unit-c2 include the following, but are not limited thereto. Note that, in the following formulae, R^{A} is as defined above, and Me represents a methyl group.

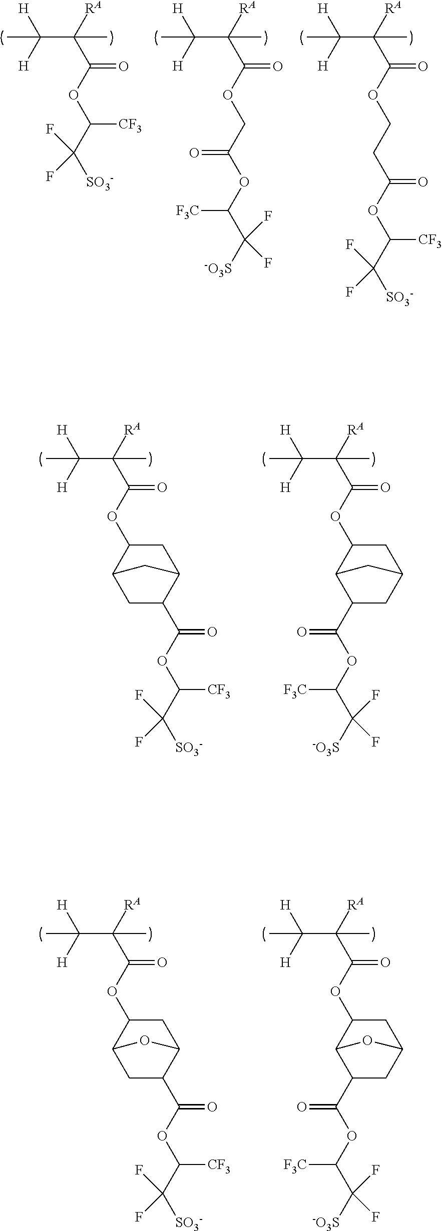

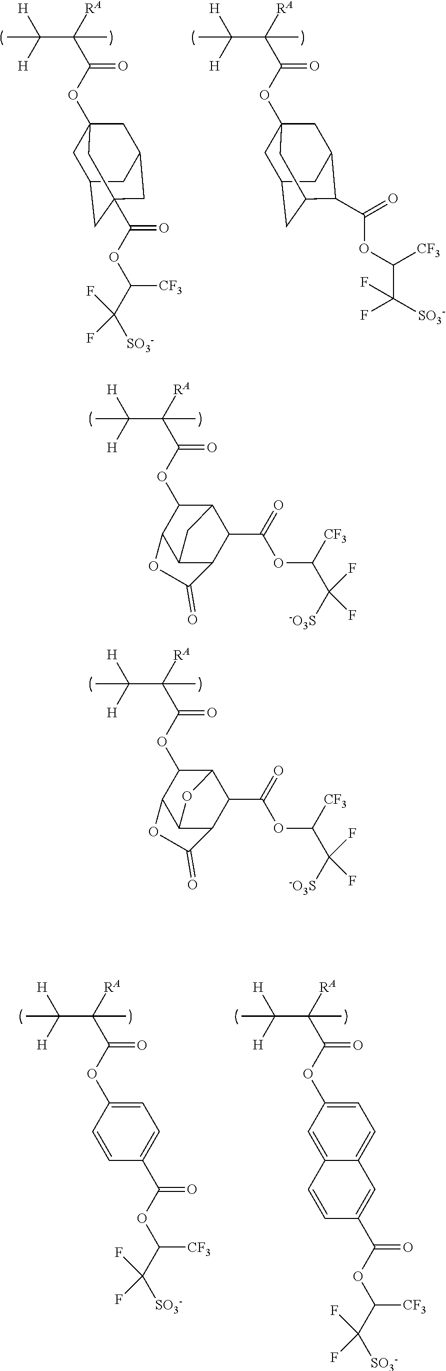

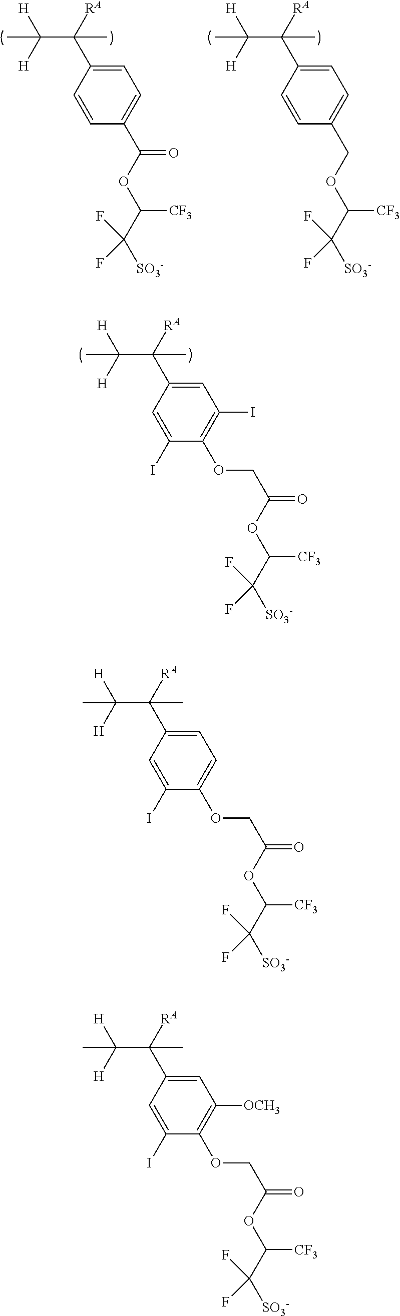

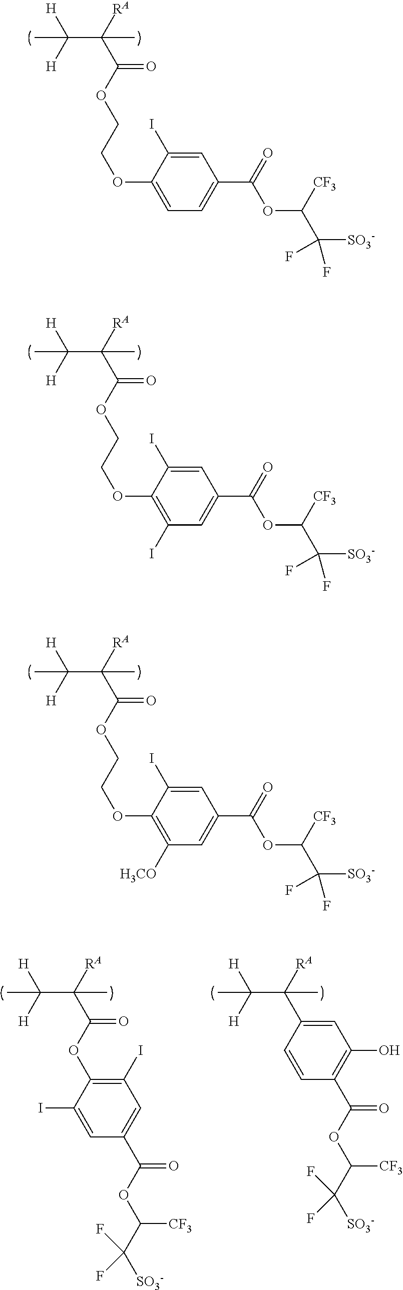

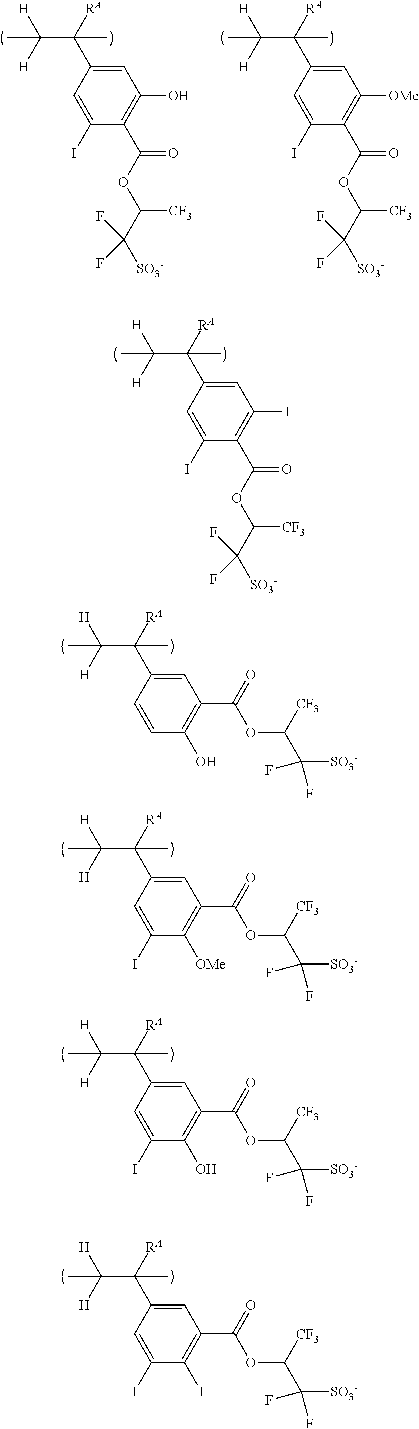

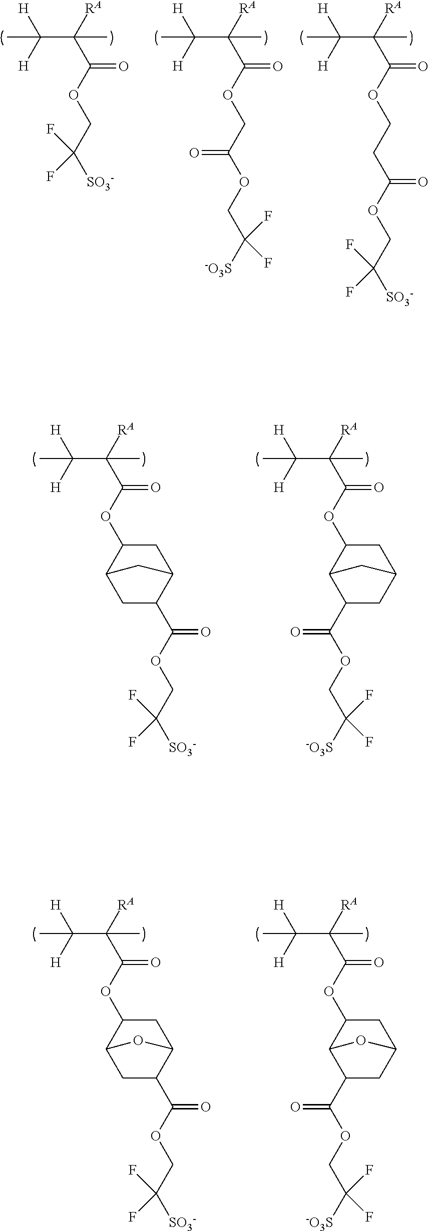

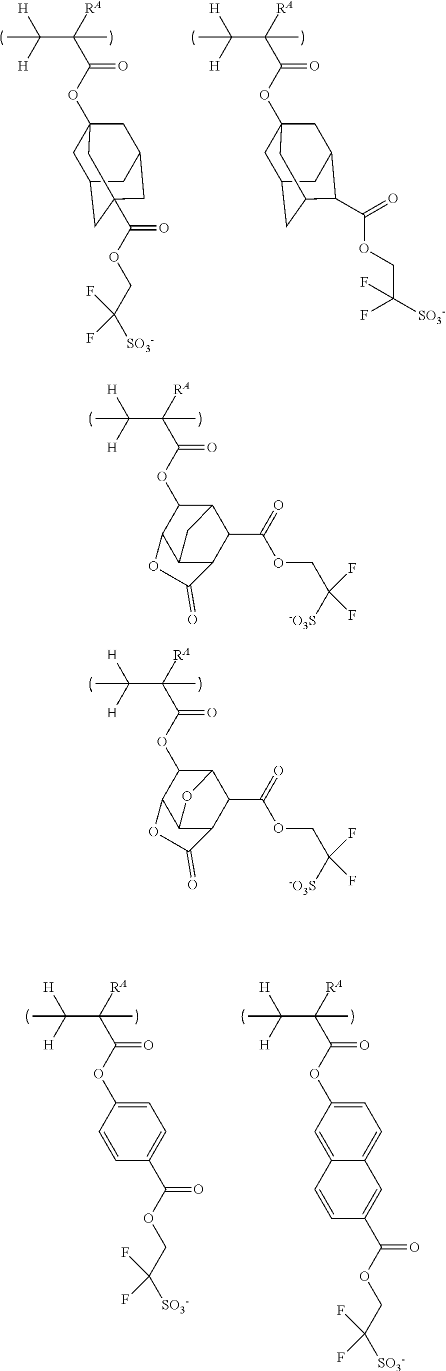

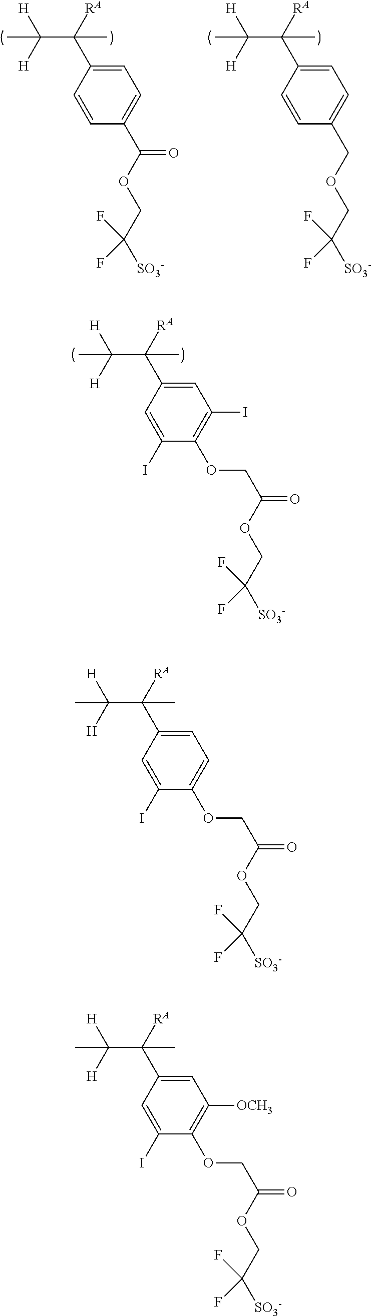

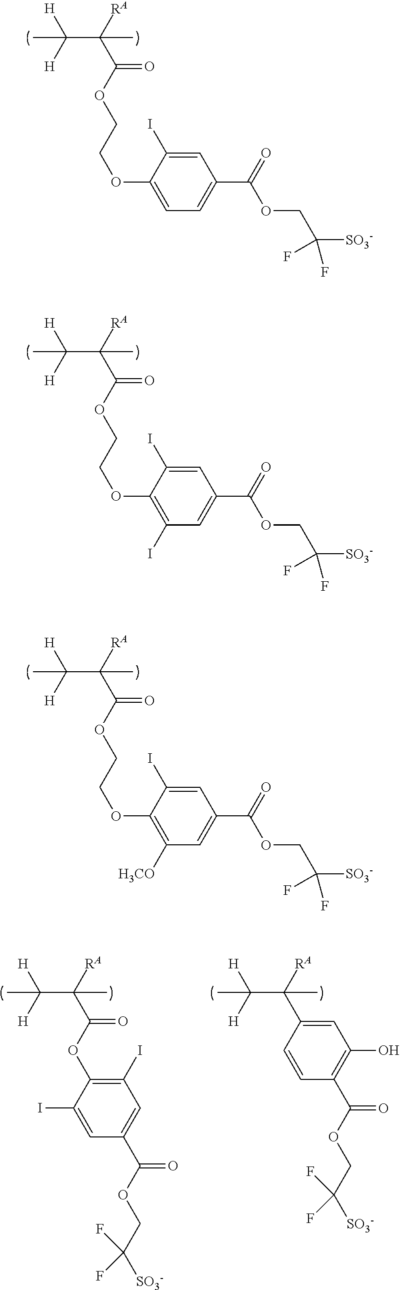

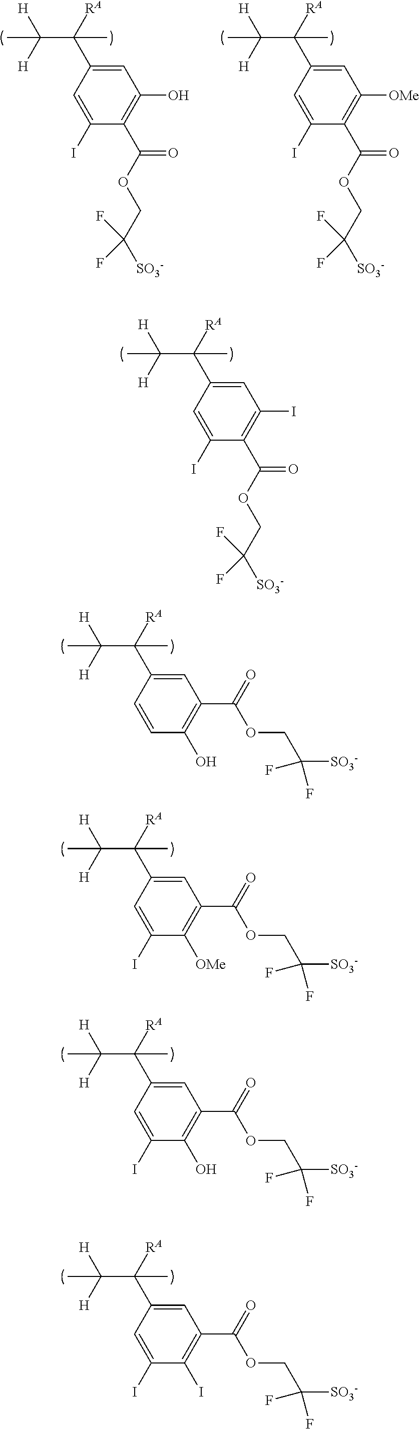

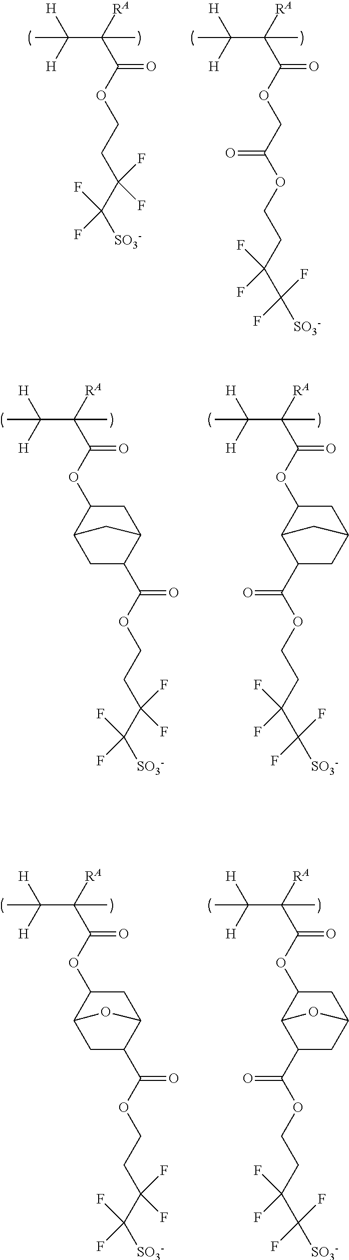

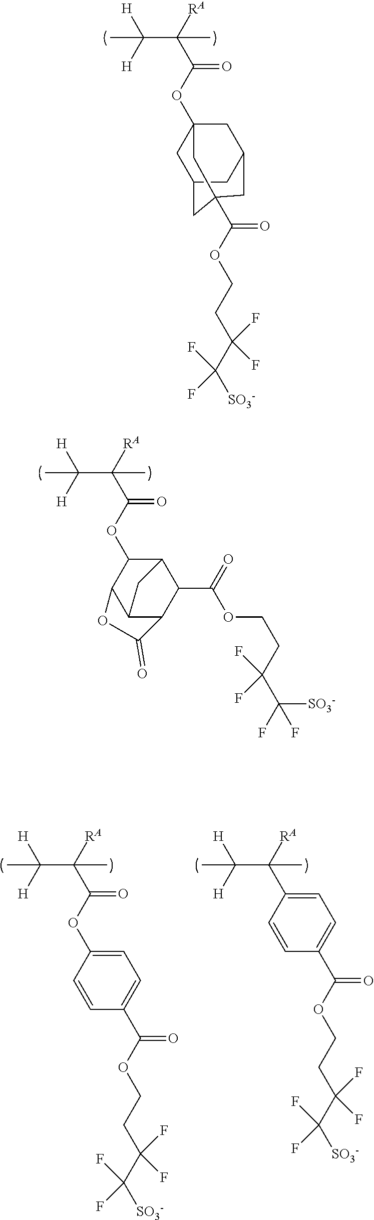

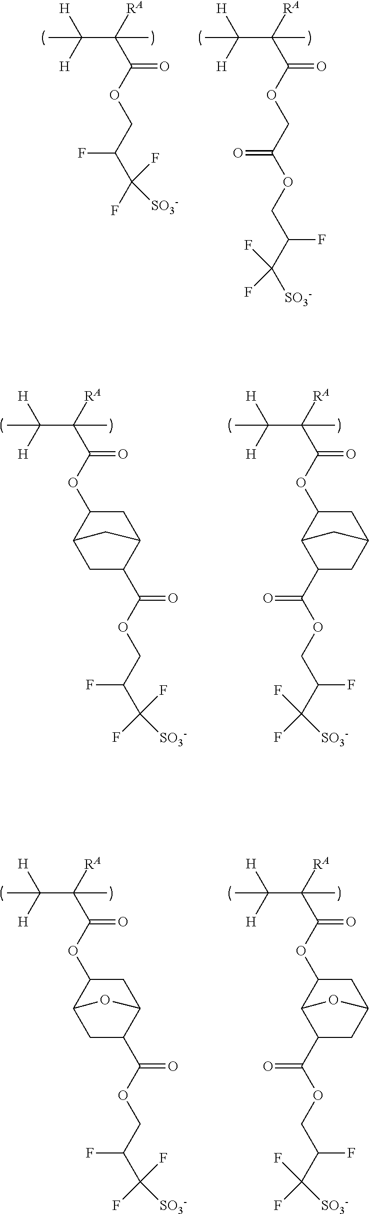

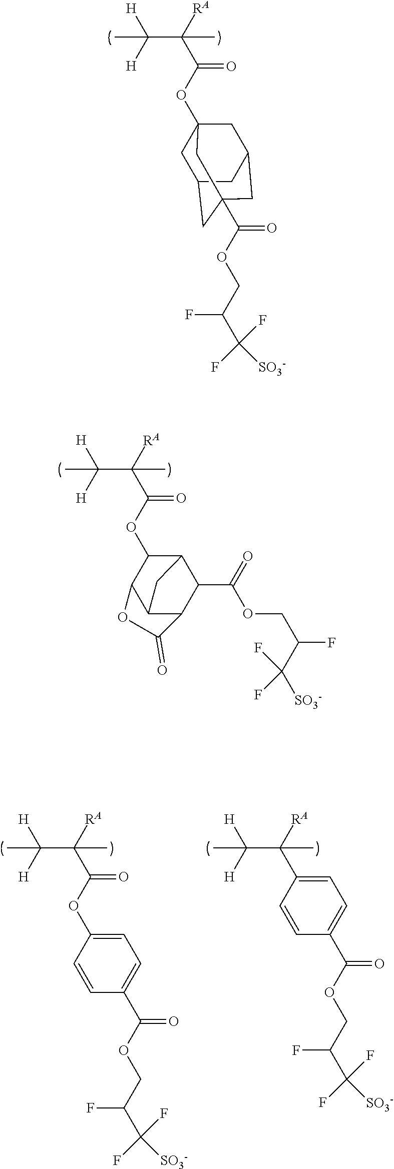

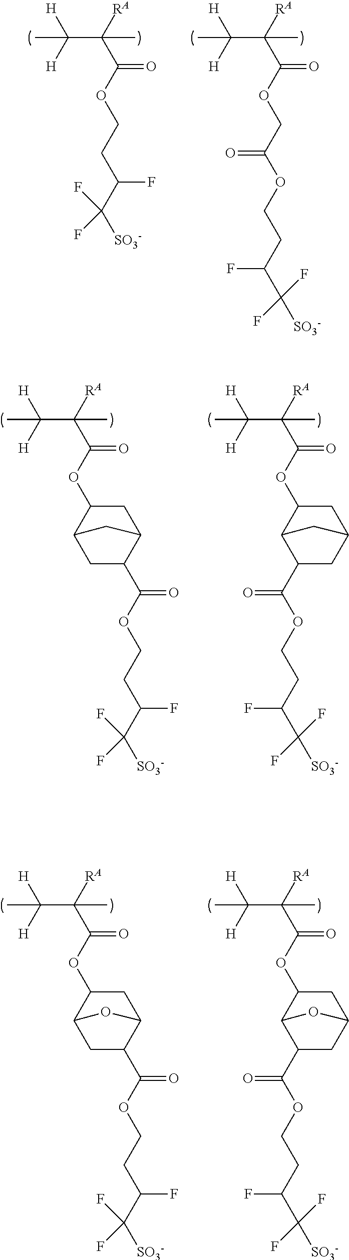

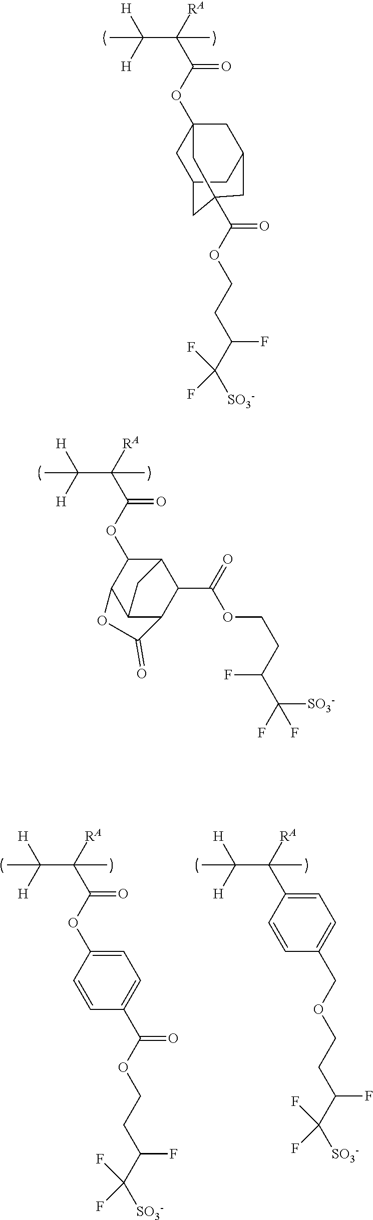

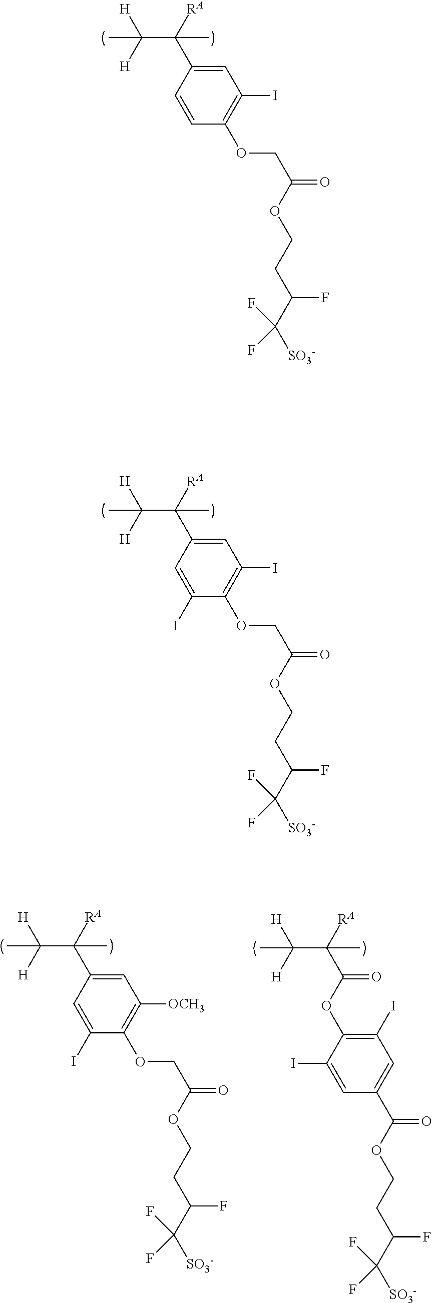

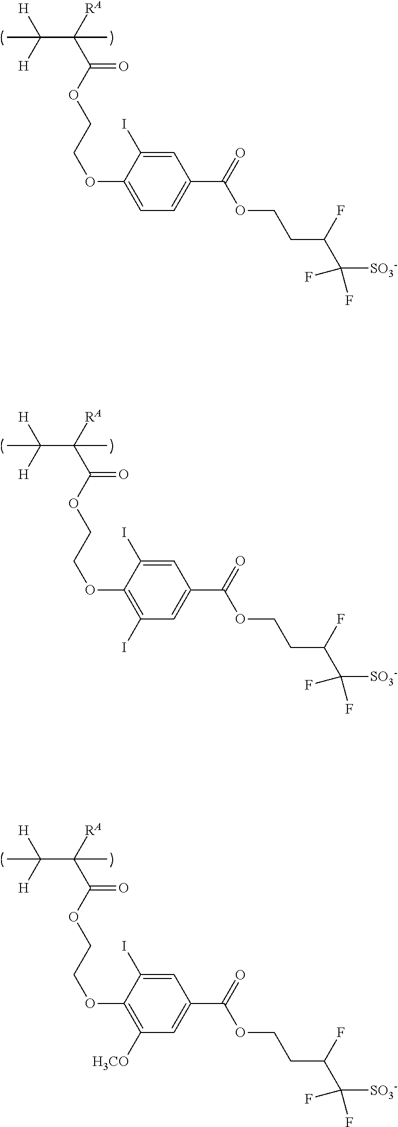

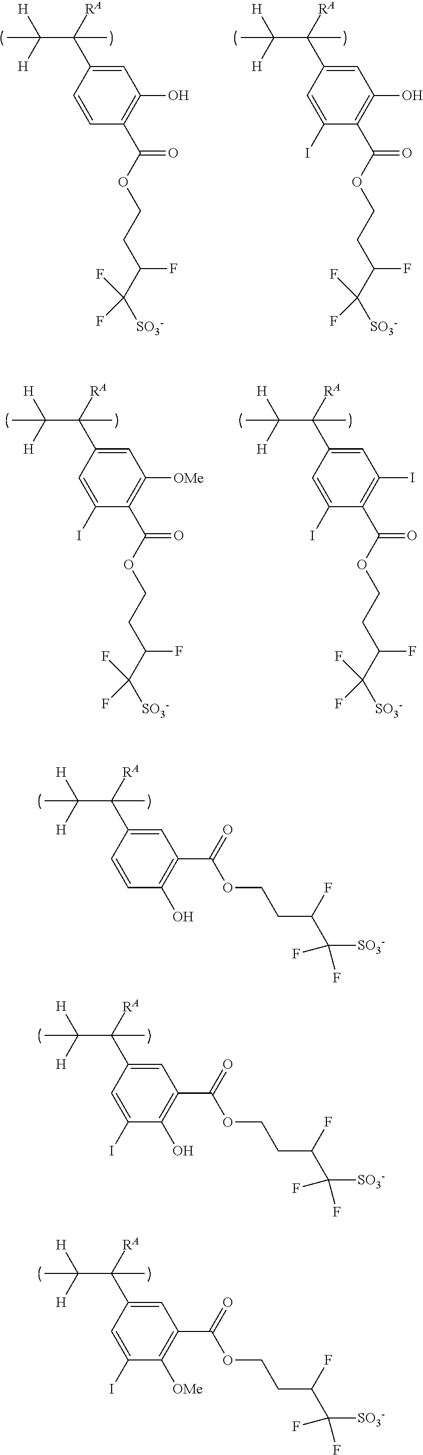

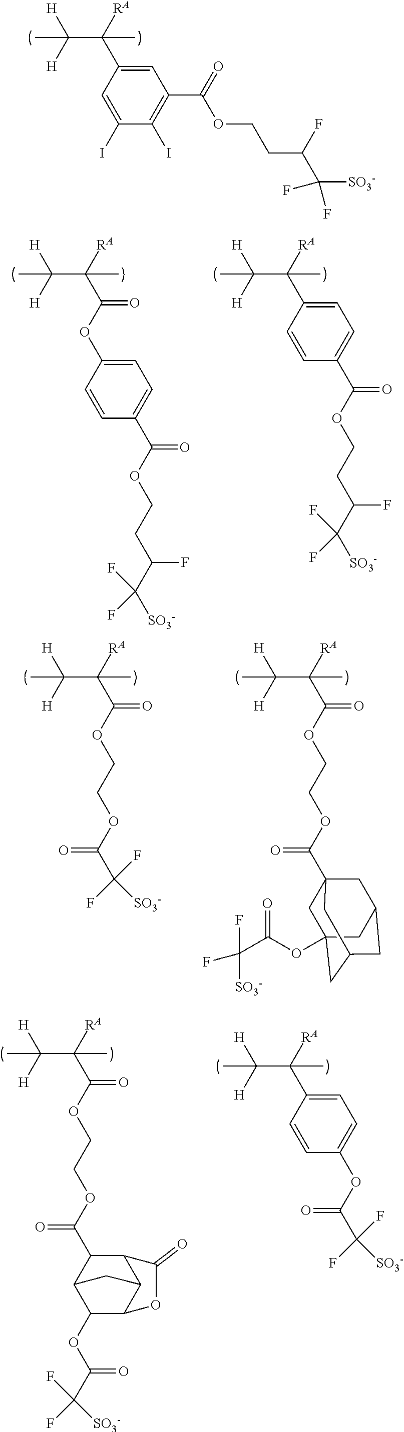

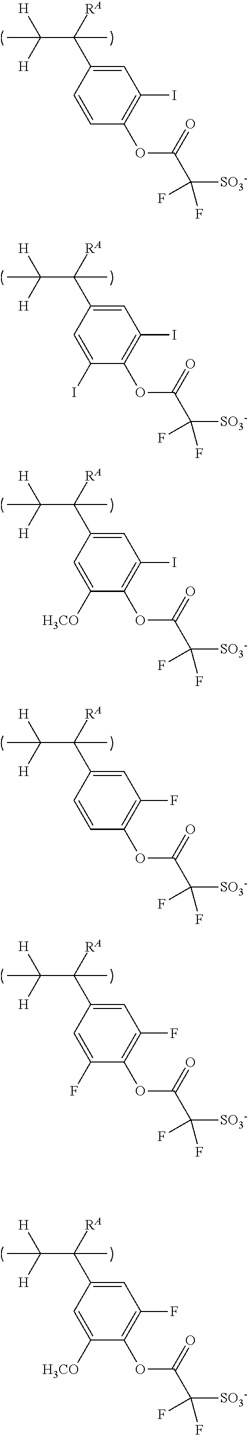

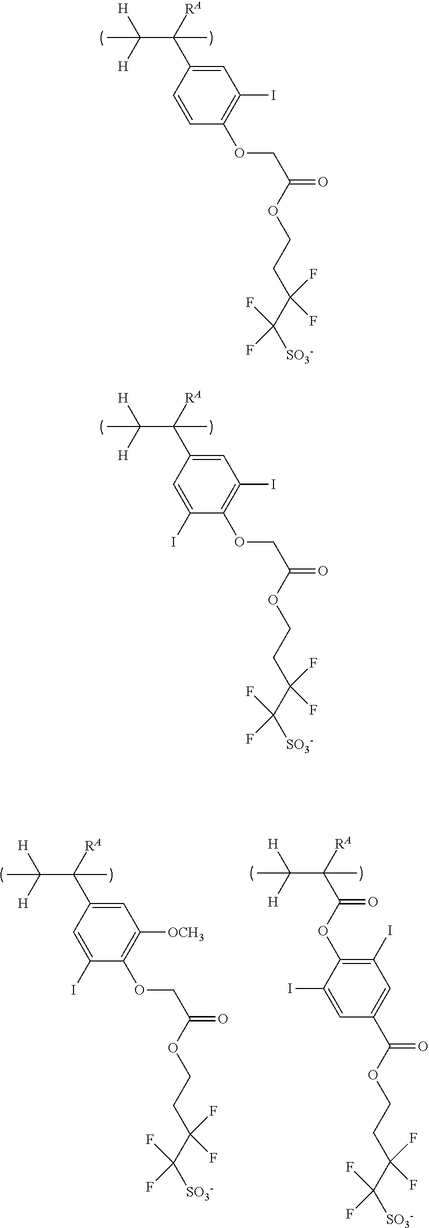

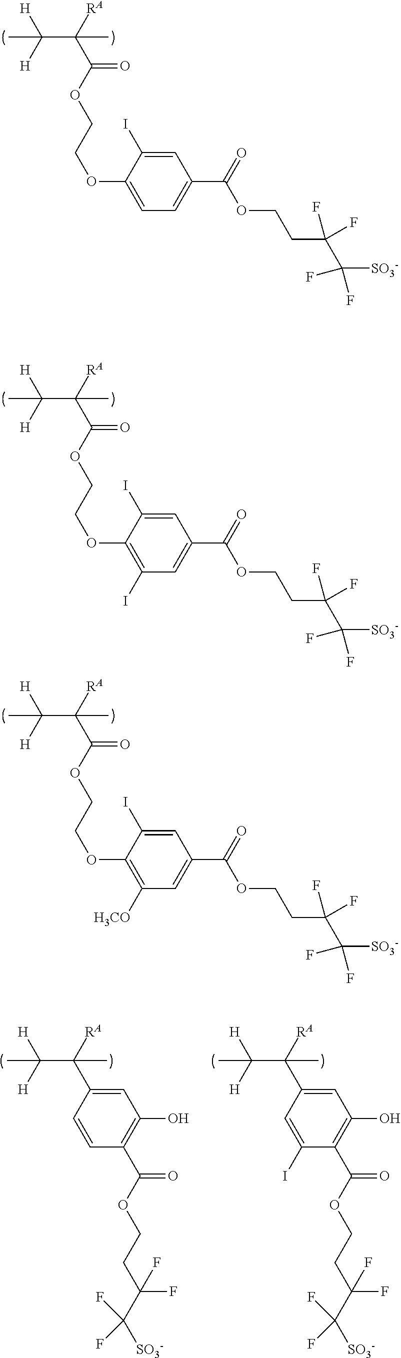

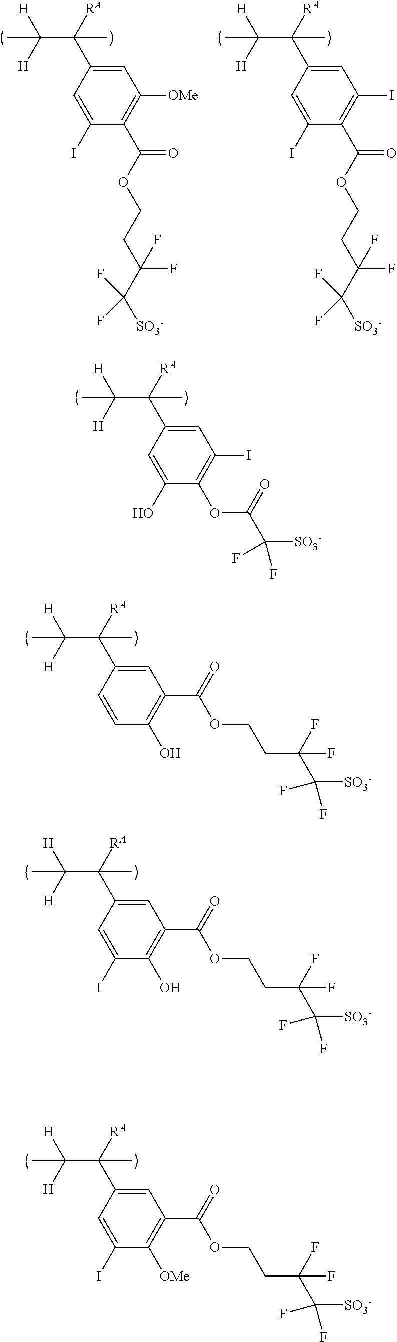

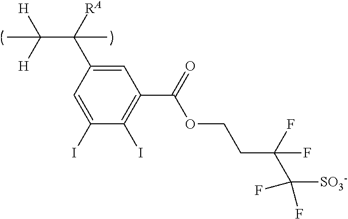

Specific examples of the anion of the repeating unit-c3 include the following, but are not limited thereto. Note that, in the following formulae, R^{A} is as defined above.

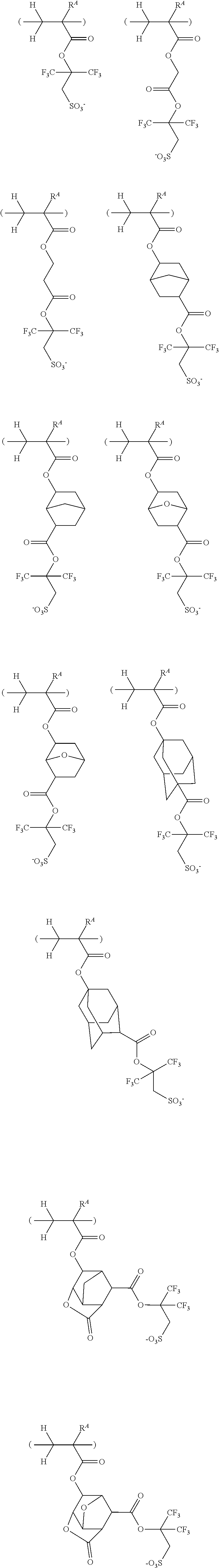

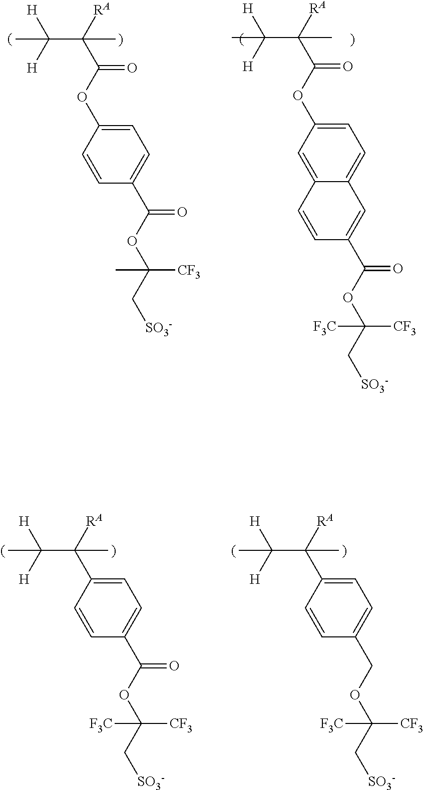

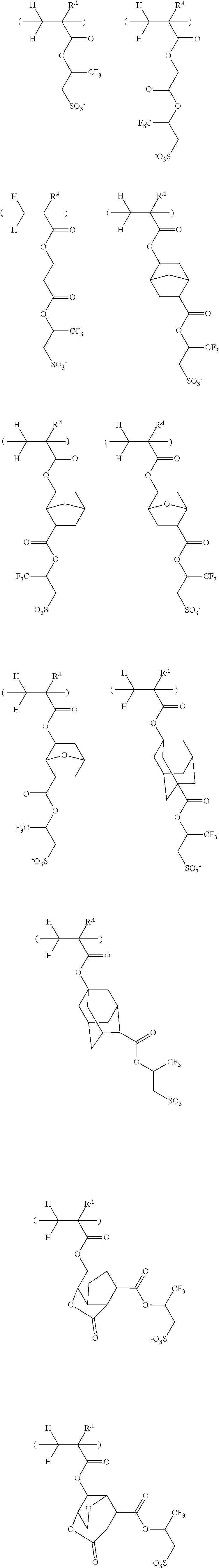

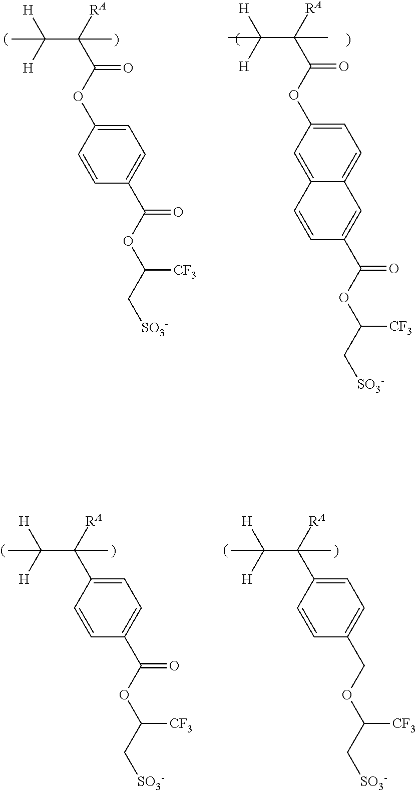

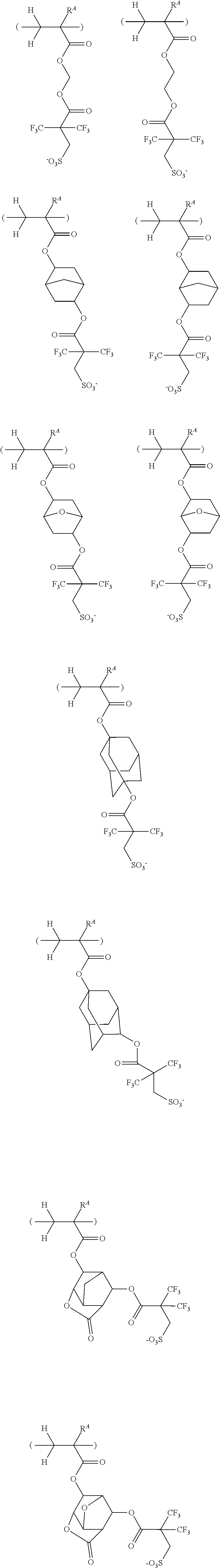

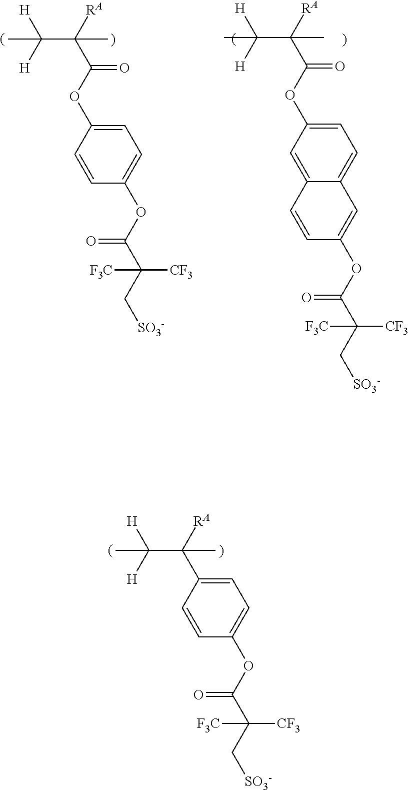

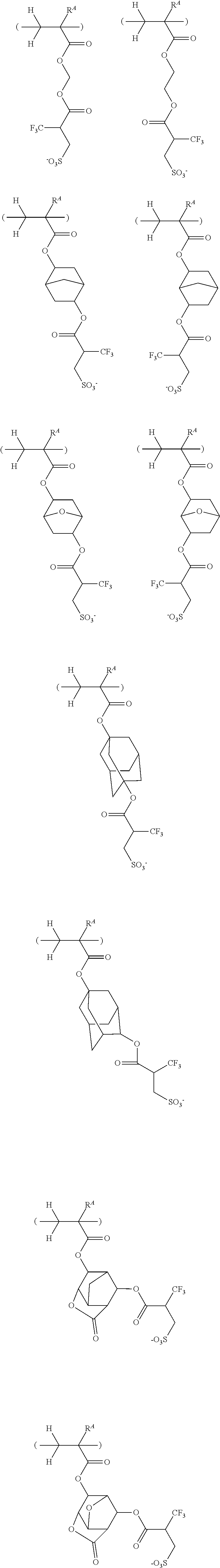

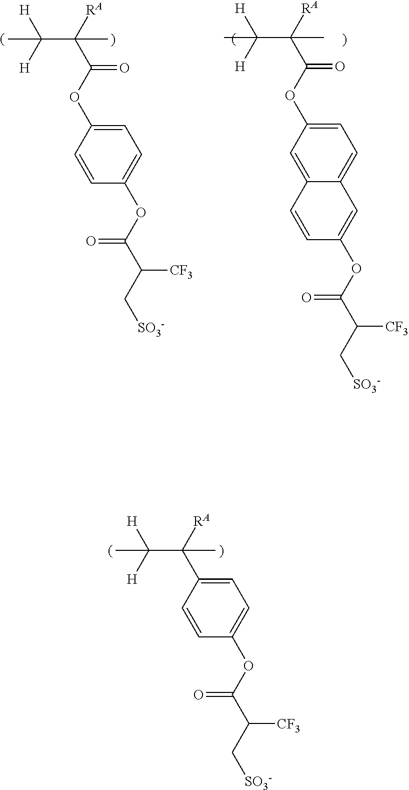

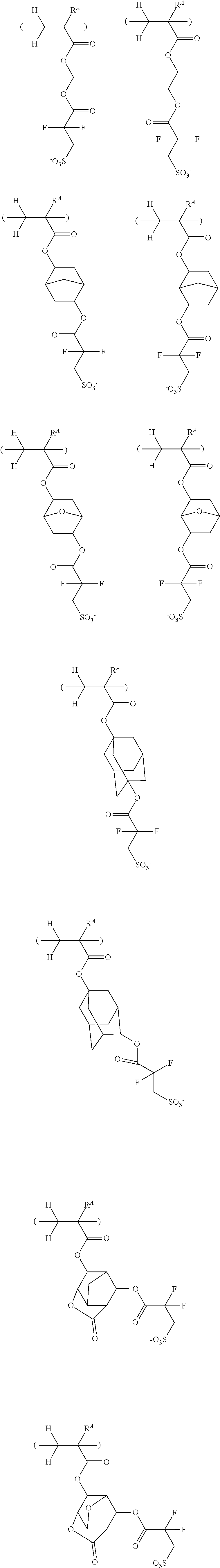

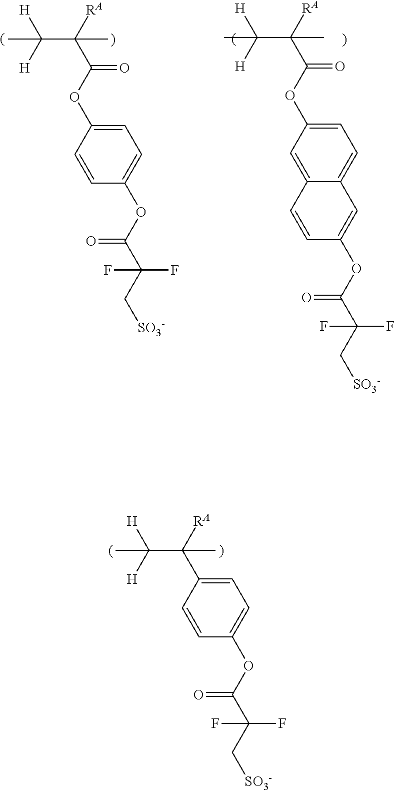

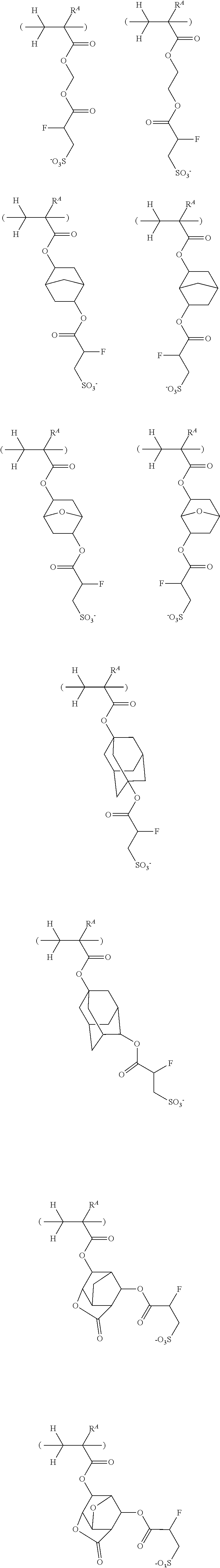

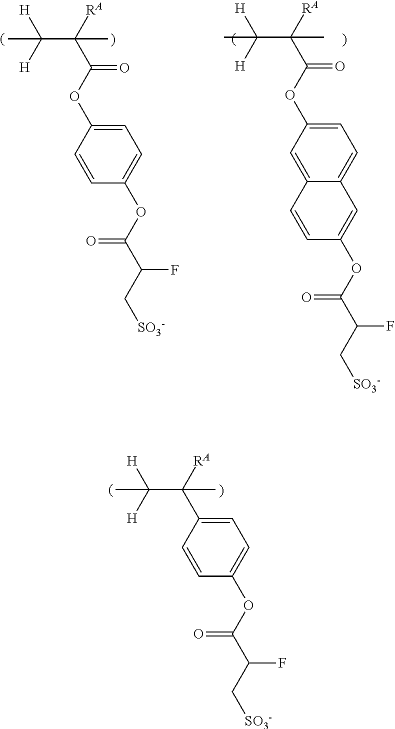

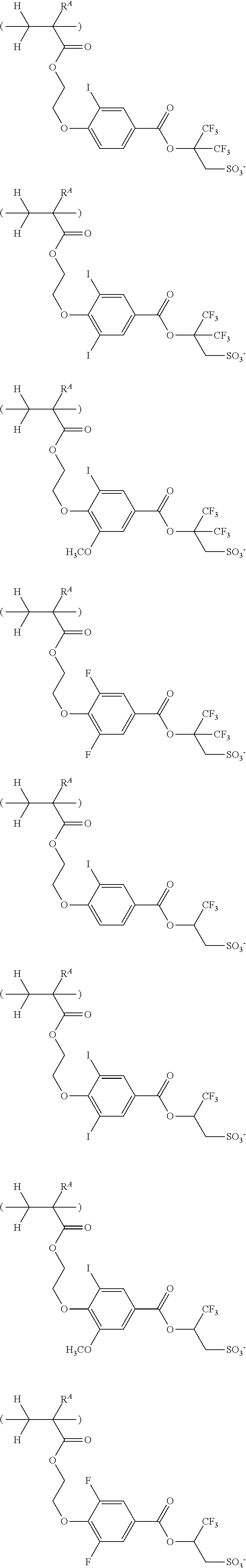

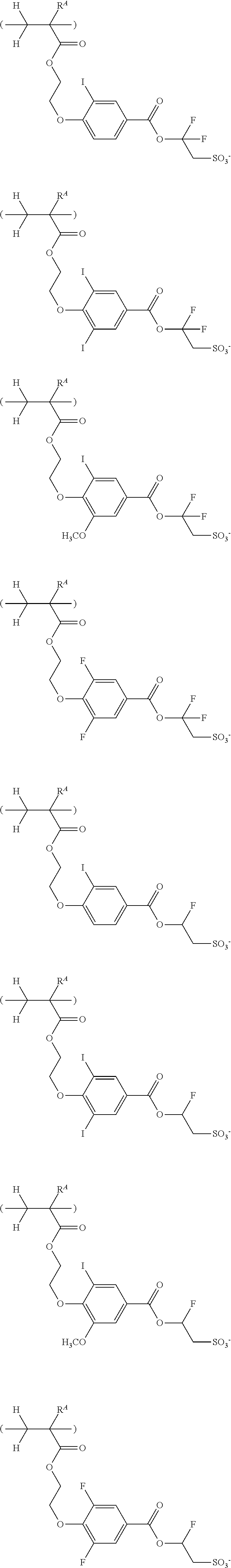

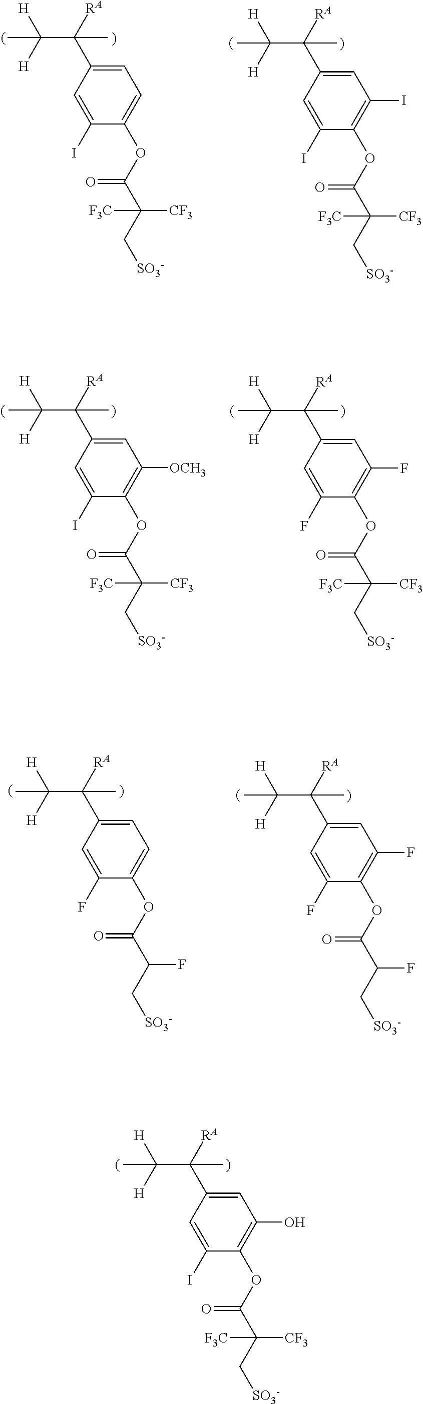

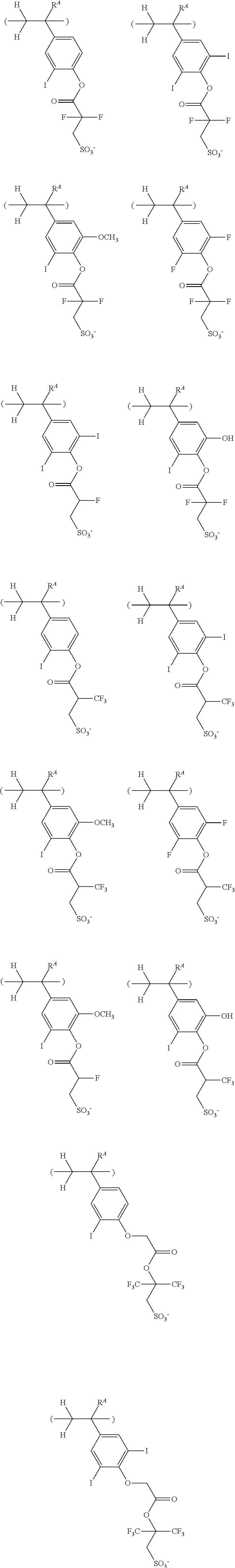

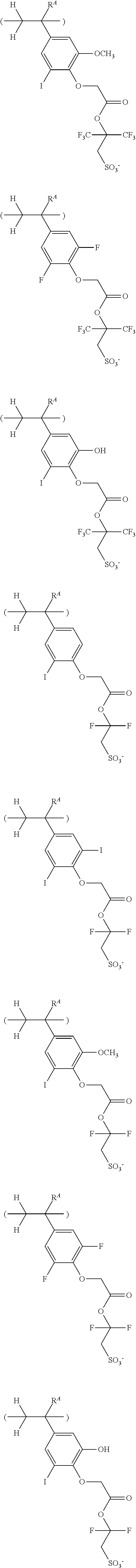

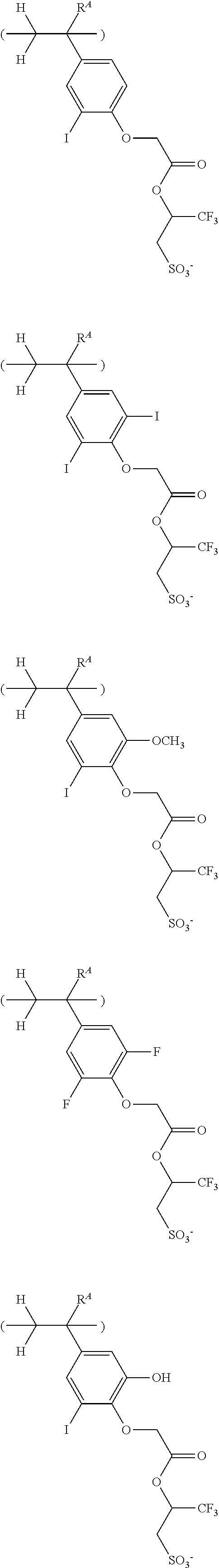

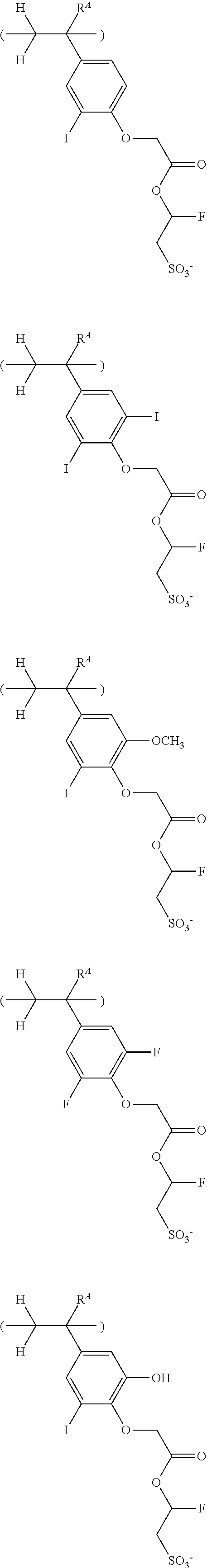

Specific examples of the anion of the repeating unit-c4 include the following, but are not limited thereto. Note that, in the following formulae, R^{A} is as defined above.

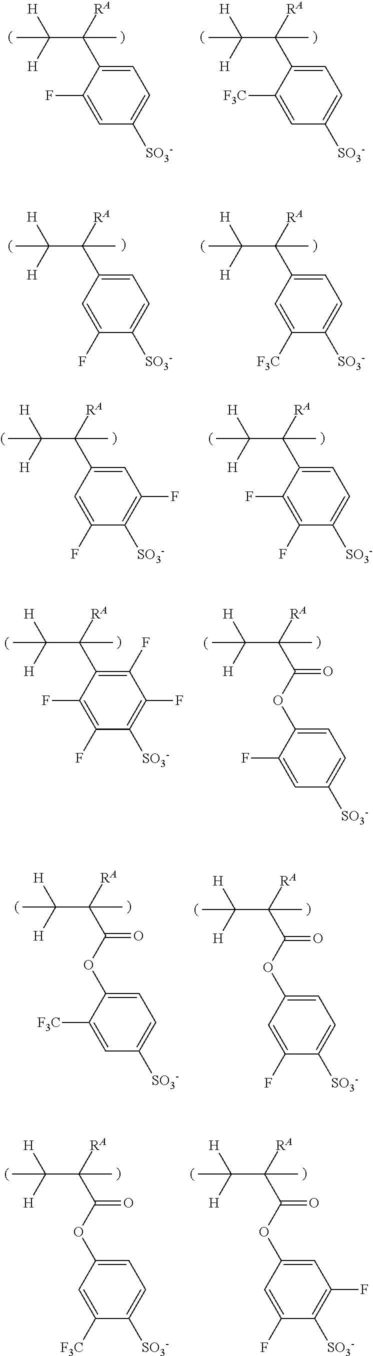

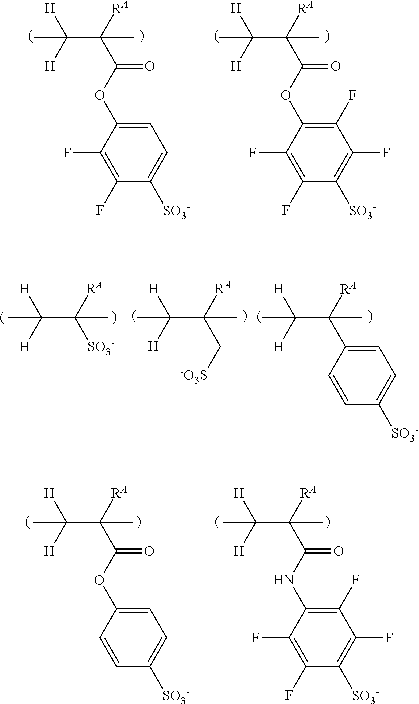

In the general formulae (c2) to (c4), A⁺ represents an onium cation. Examples of the onium cation include ammonium cations, sulfonium cations, and iodonium cations, and sulfonium cations and iodonium cations are preferable. As the onium cation, a sulfonium cation represented by the following general formula (cation-1) or an iodonium cation represented by the following general formula (cation-2) is preferable.

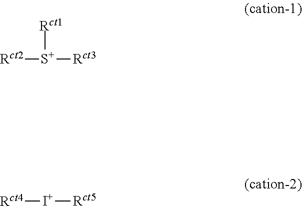

In the general formulae (cation-1) and (cation-2), R^{ct1} to R^{ct5} each independently represent a halogen atom or a hydrocarbyl group having 1 to 30 carbon atoms and optionally containing a heteroatom.

Examples of the halogen atom represented by R^{ct1} to R^{ct5} include a fluorine atom, a chlorine atom, a bromine atom, and an iodine atom.

The hydrocarbyl group represented by R^{ct1} to R^{ct5} having 1 to 30 carbon atoms may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, an allyl group, a propenyl group, a butenyl group, and a hexenyl group; cyclic unsaturated hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; groups which are combinations of these groups; etc. Aryl groups are preferable. Furthermore, part of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, R^{ct1} and R^{ct2} may be bonded to each other to form a ring together with the sulfur atom bonded thereto. In this case, examples of the structure of the ring include those represented by the following formulae.

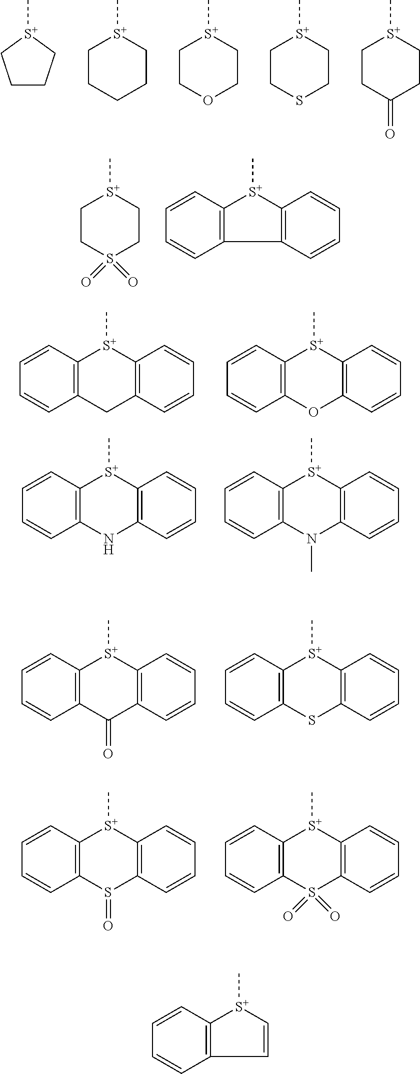

In the formulae, a broken line represents an attachment point to R^{ct3}.

Specific examples of the sulfonium cation represented by the general formula (cation-1) include the following, but are not limited thereto.

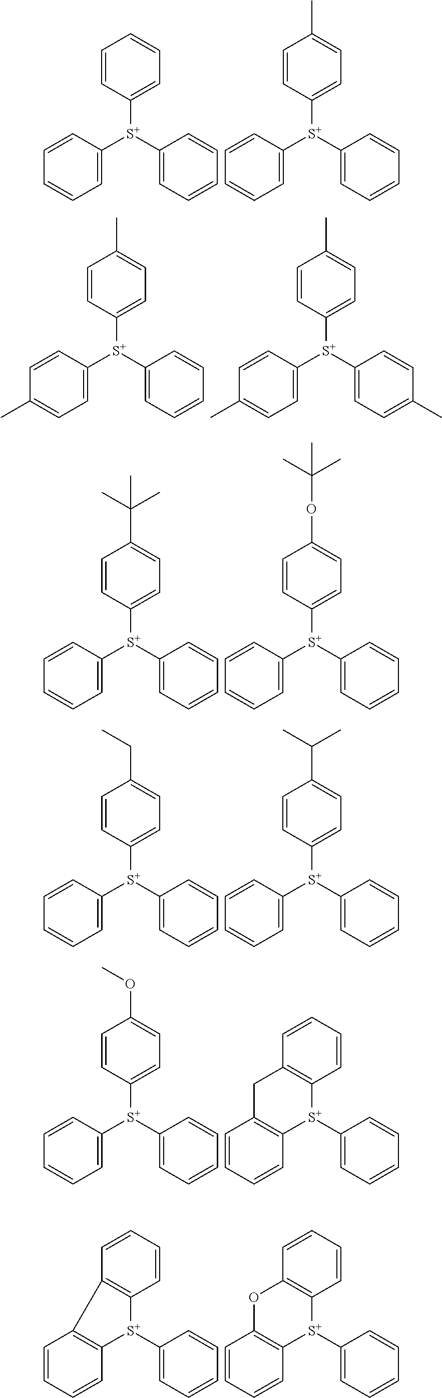

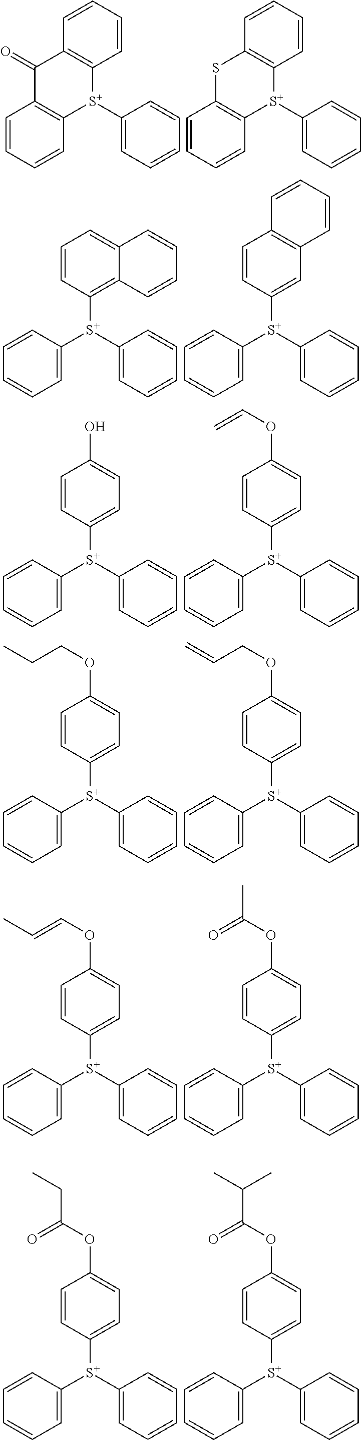

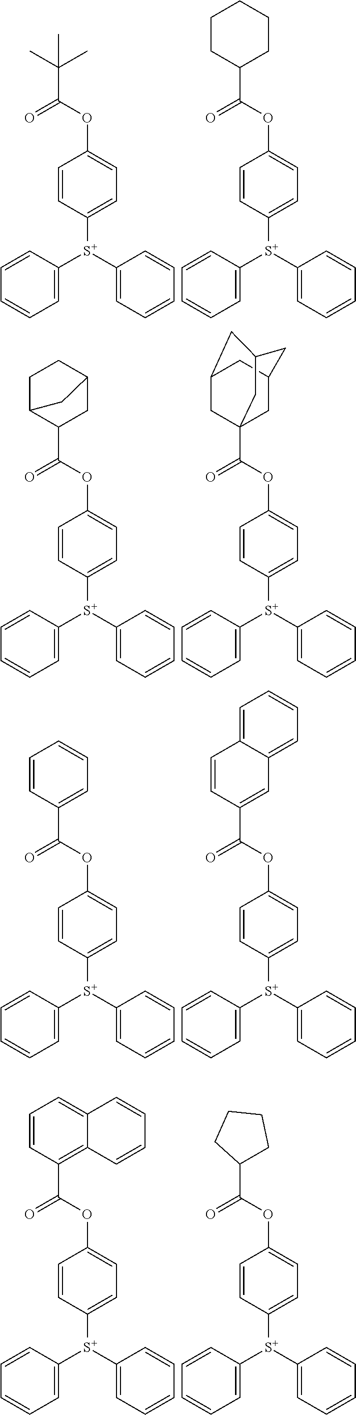

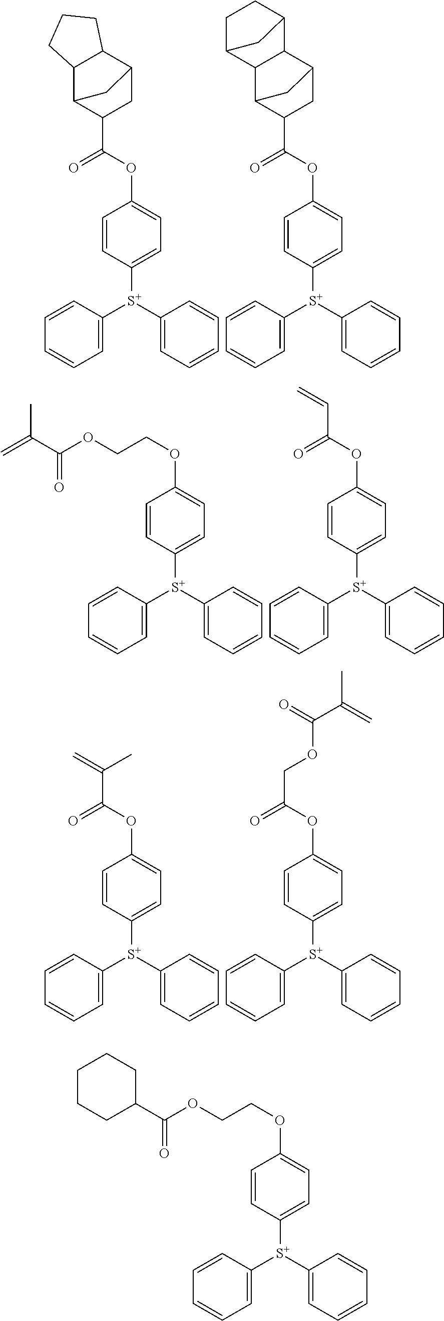

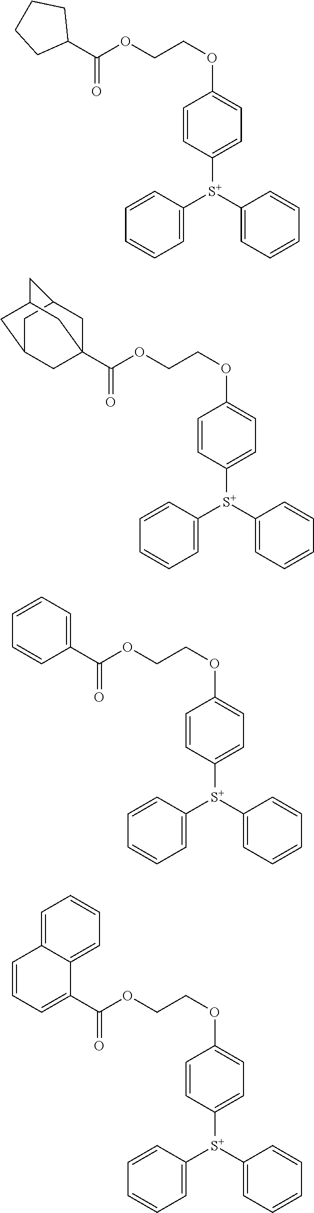

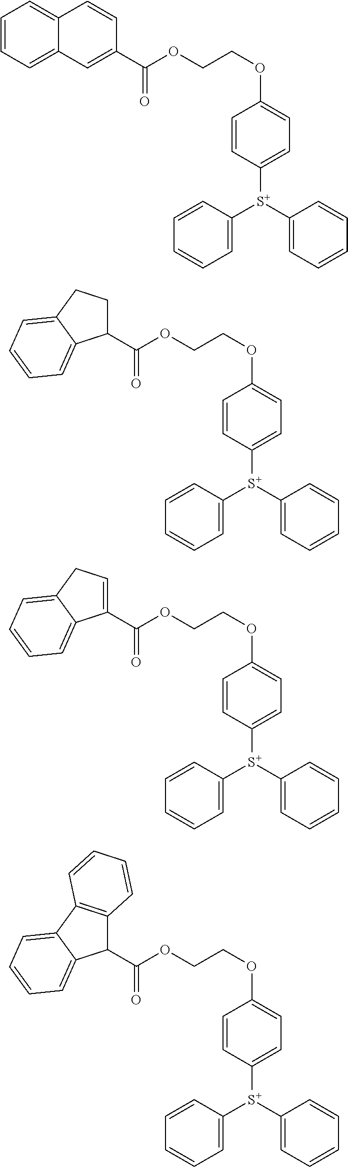

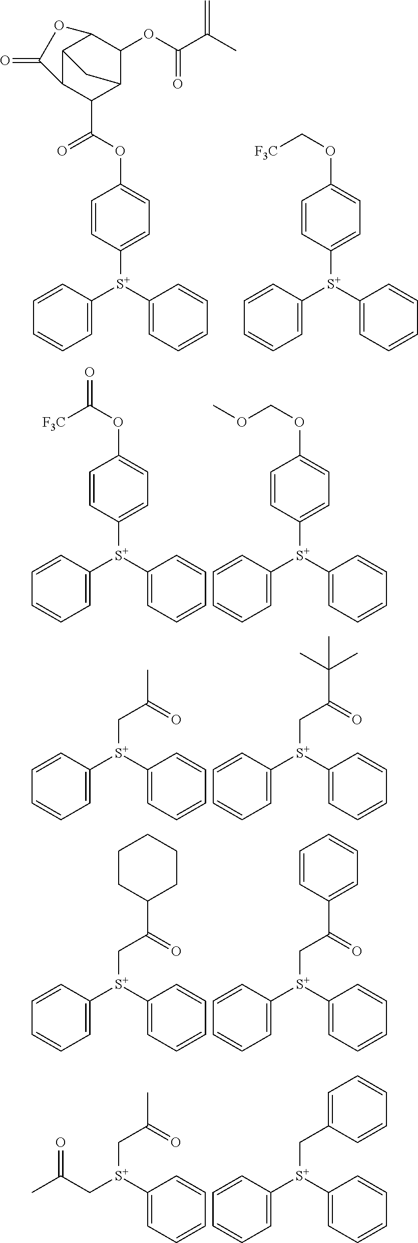

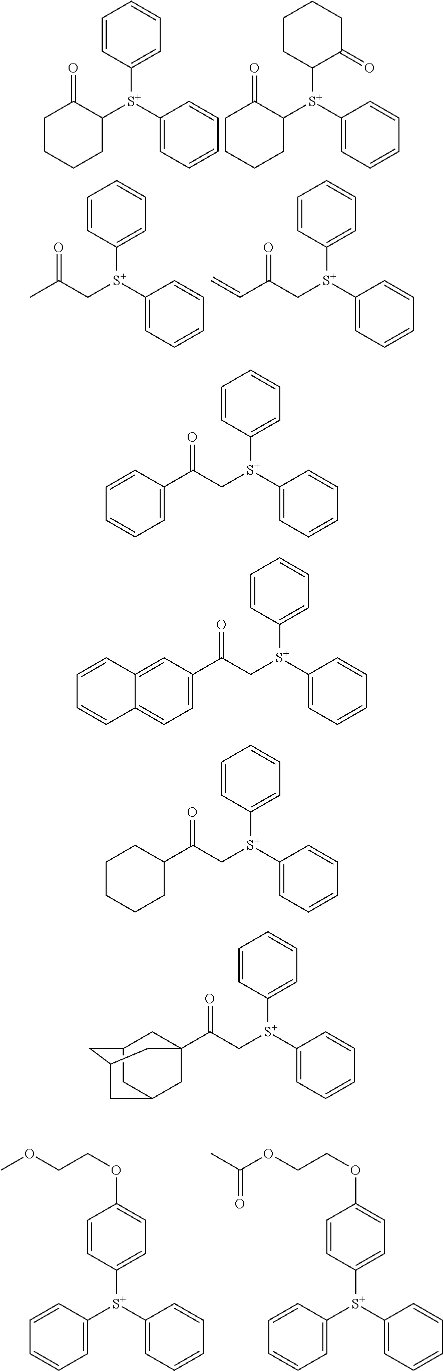

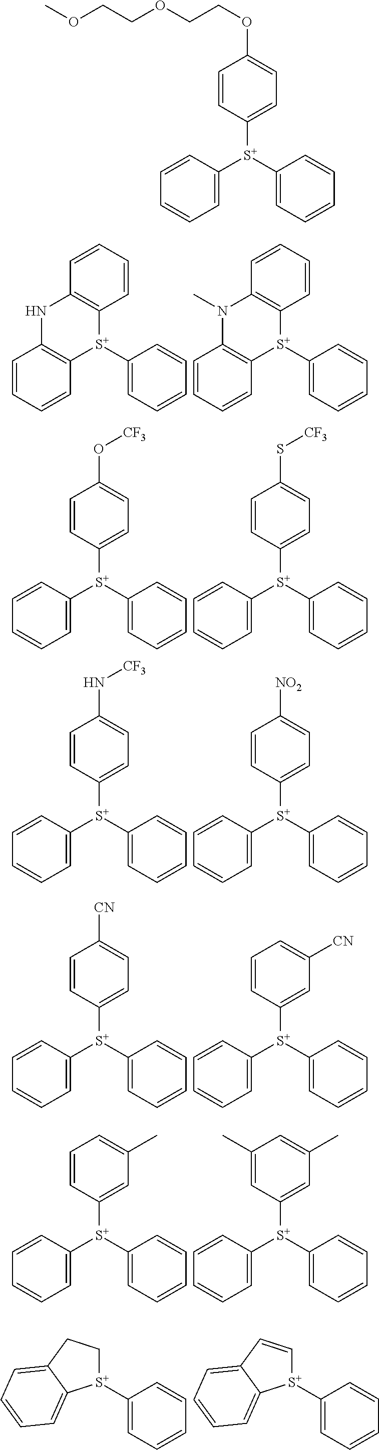

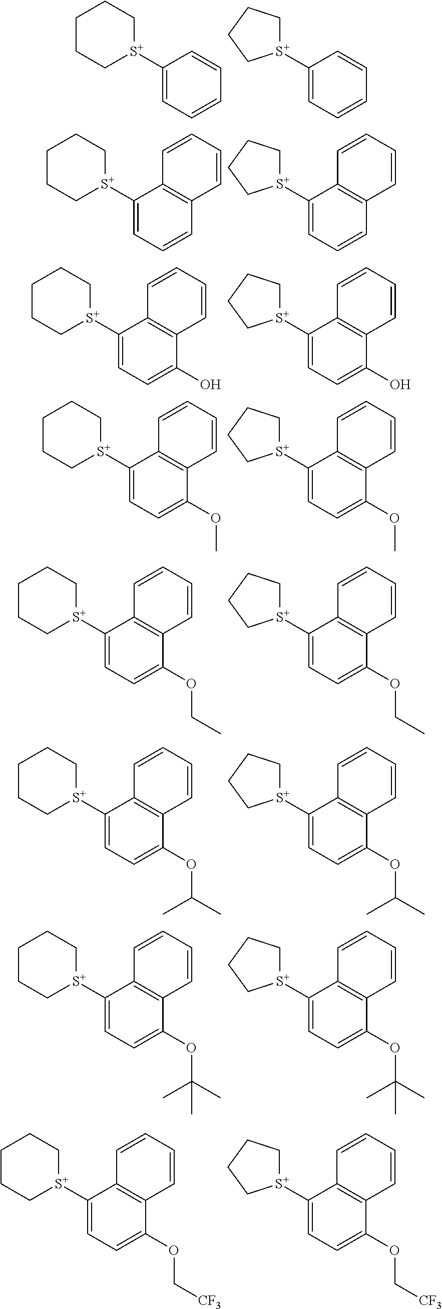

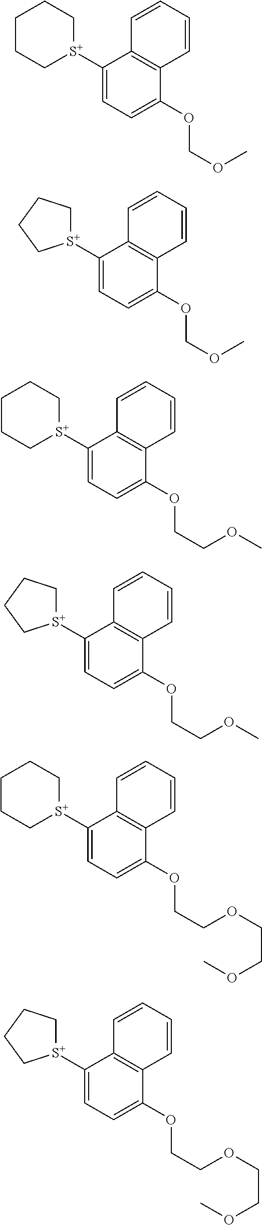

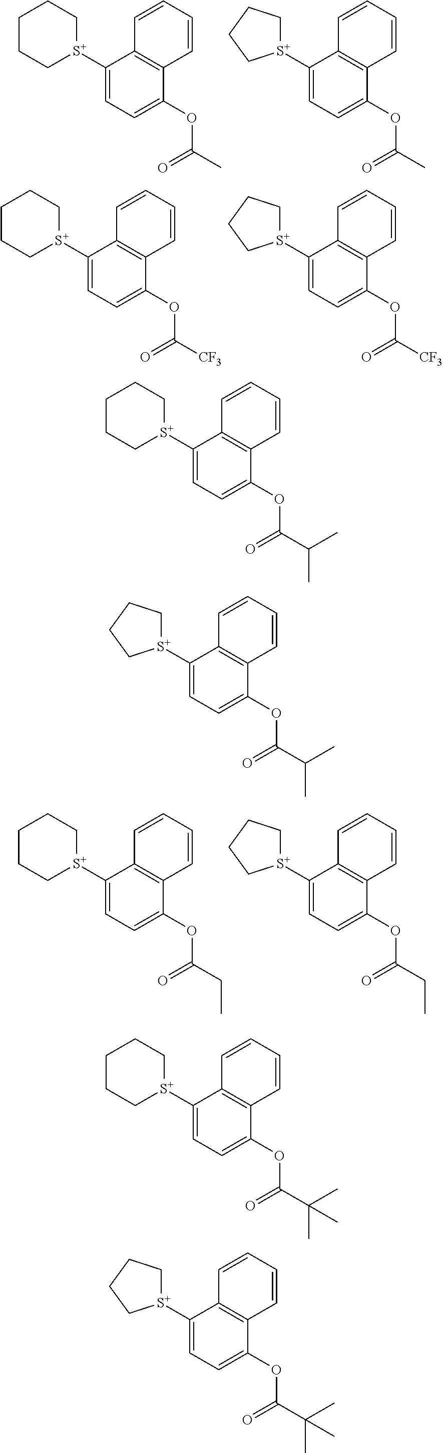

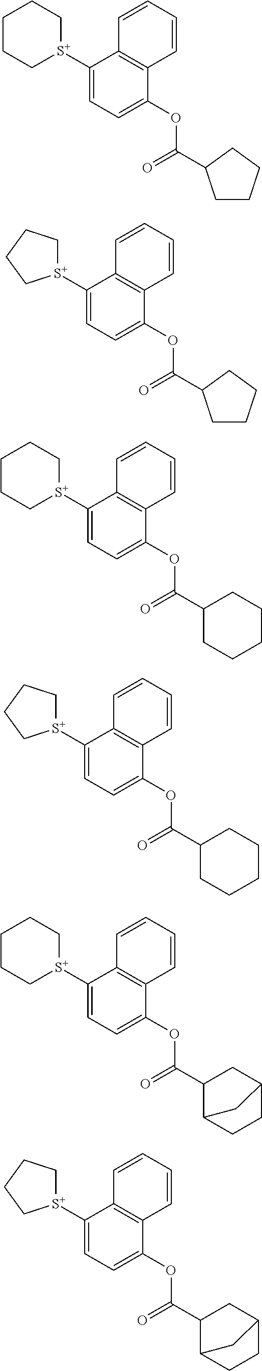

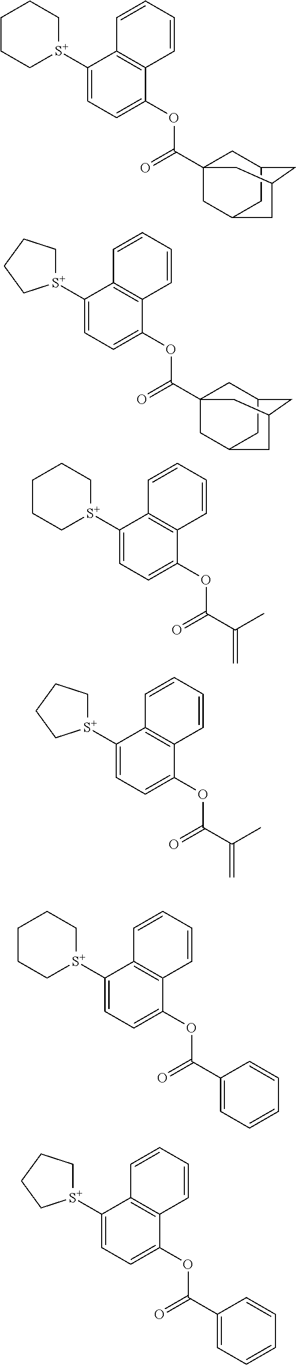

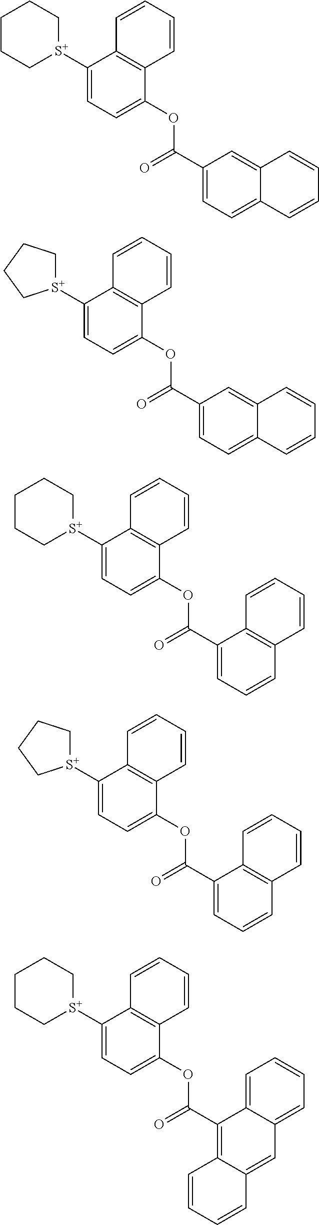

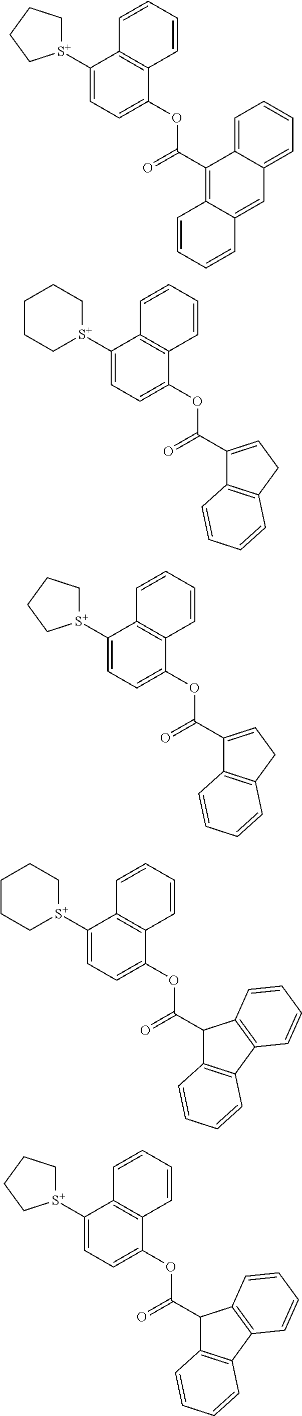

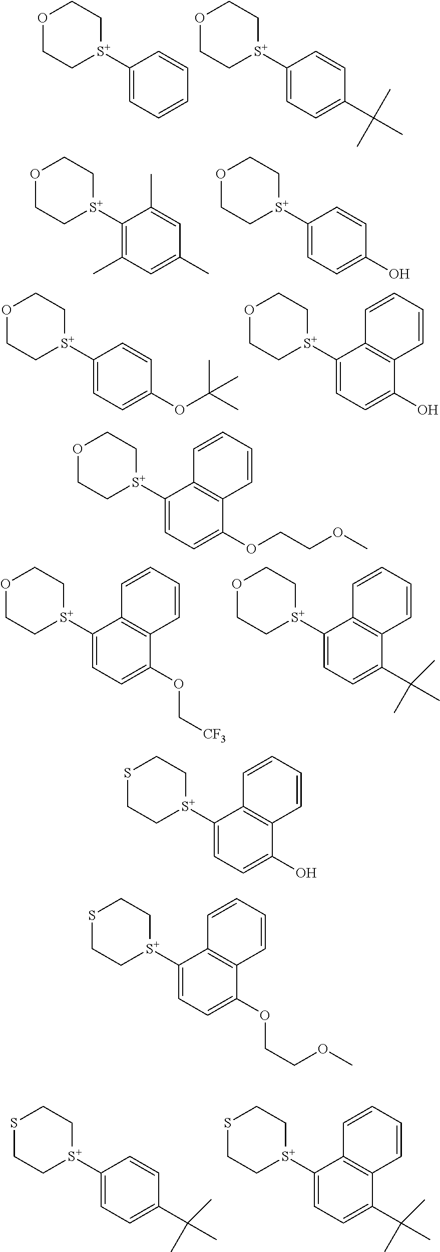

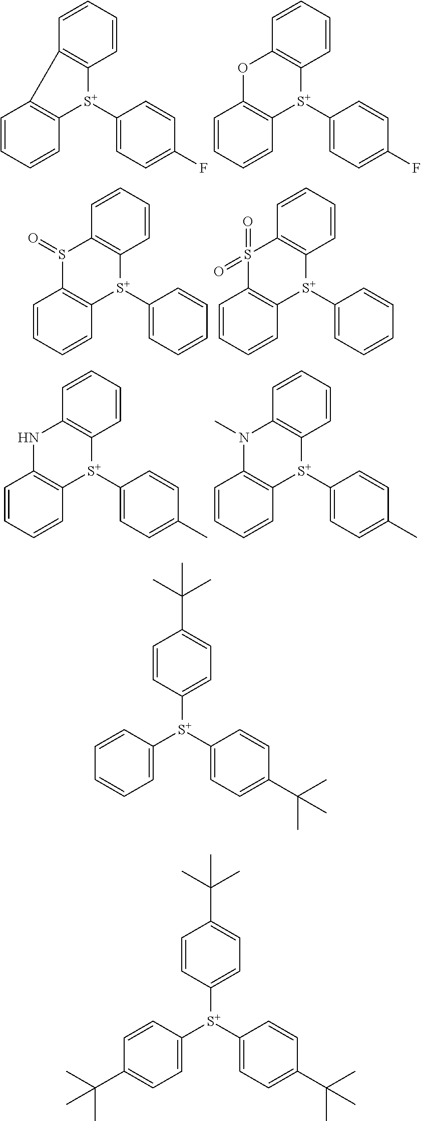

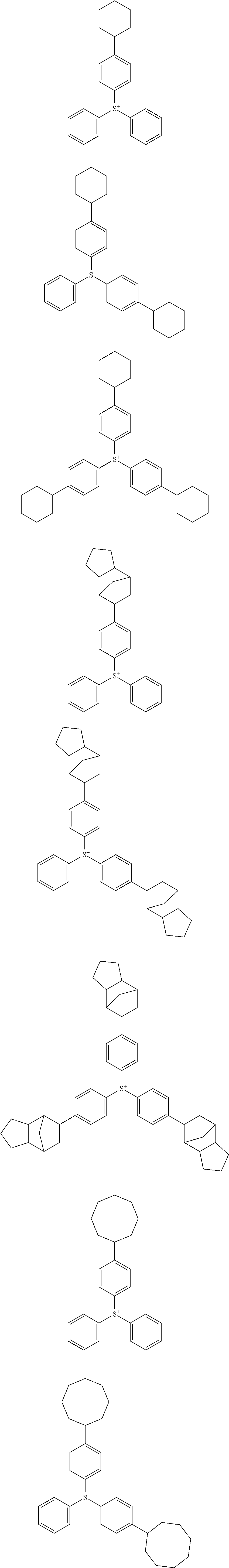

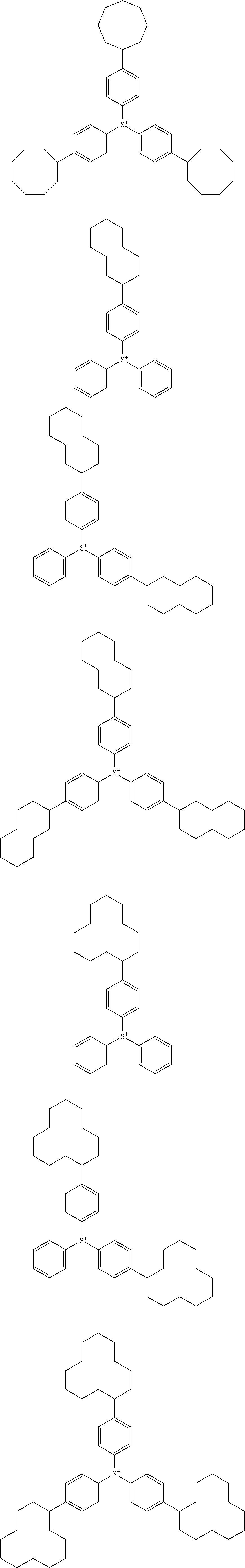

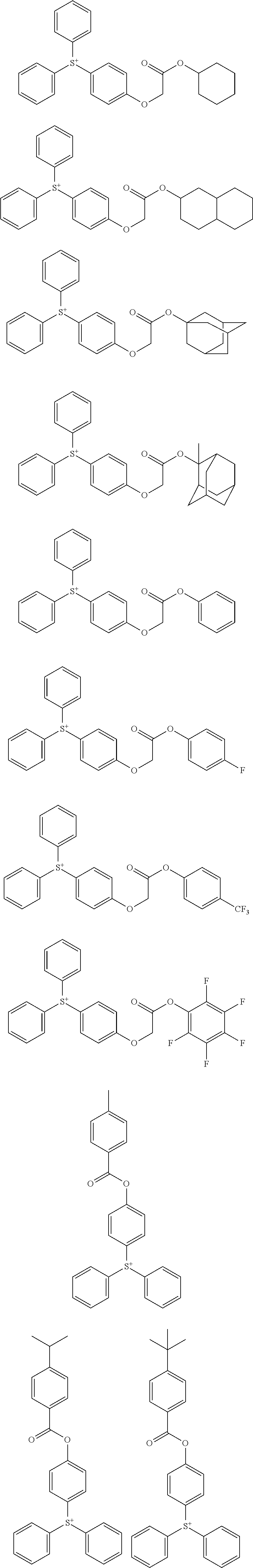

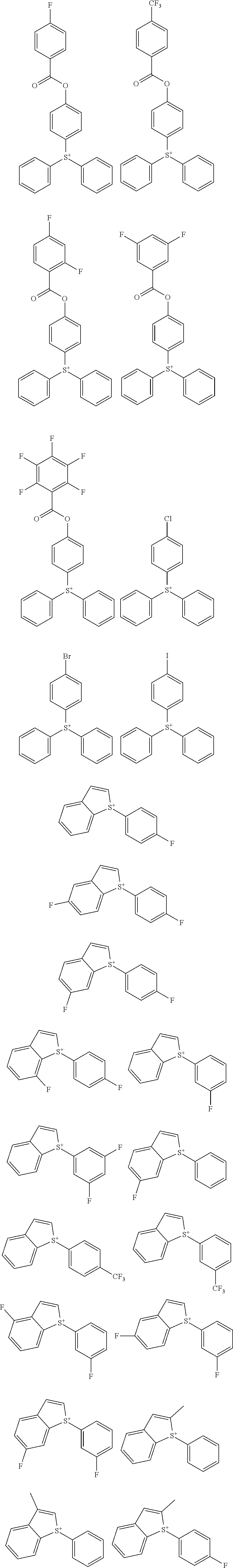

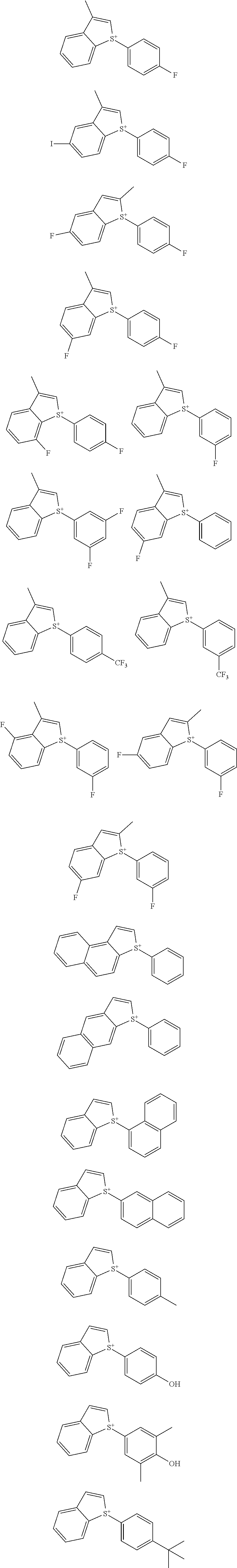

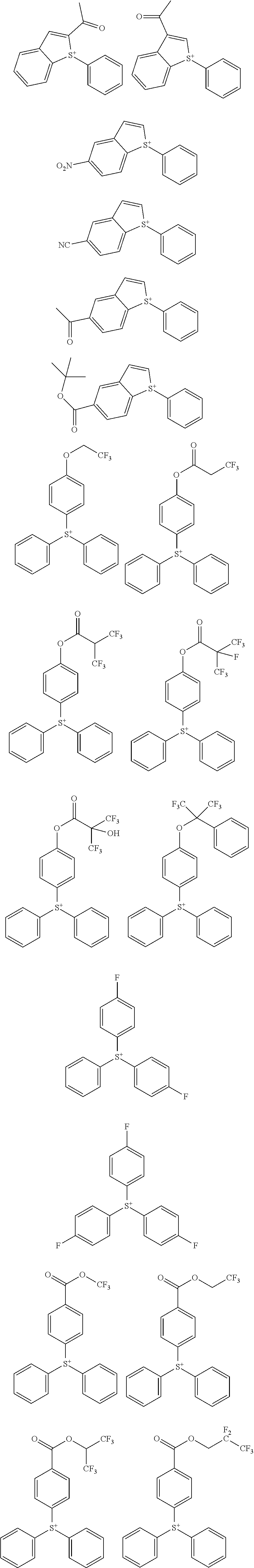

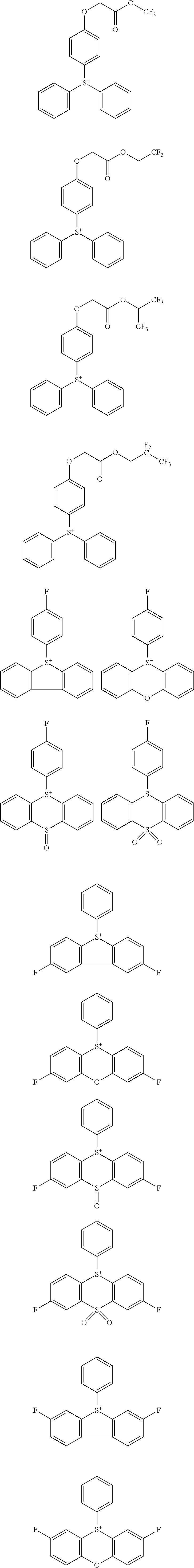

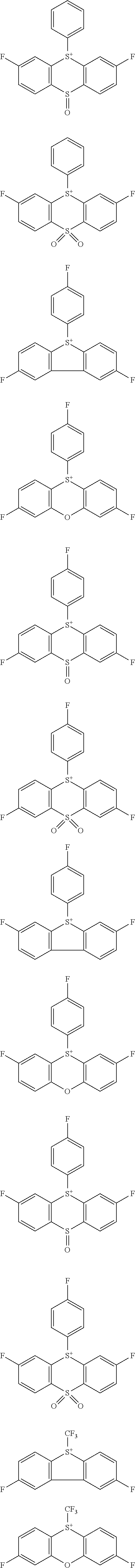

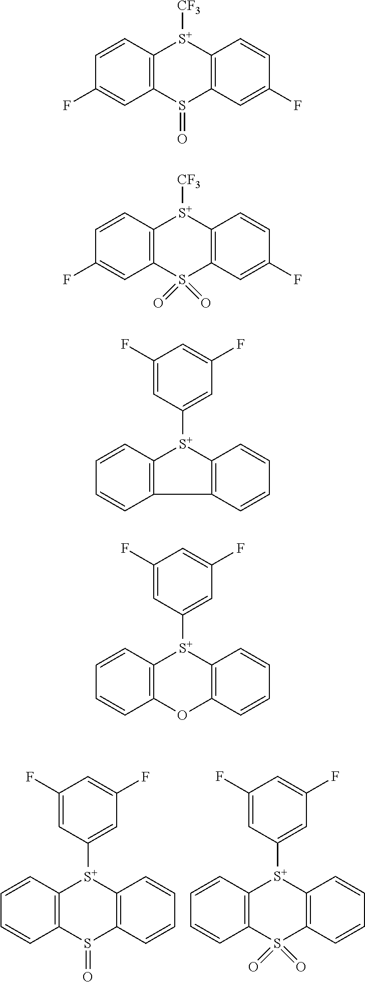

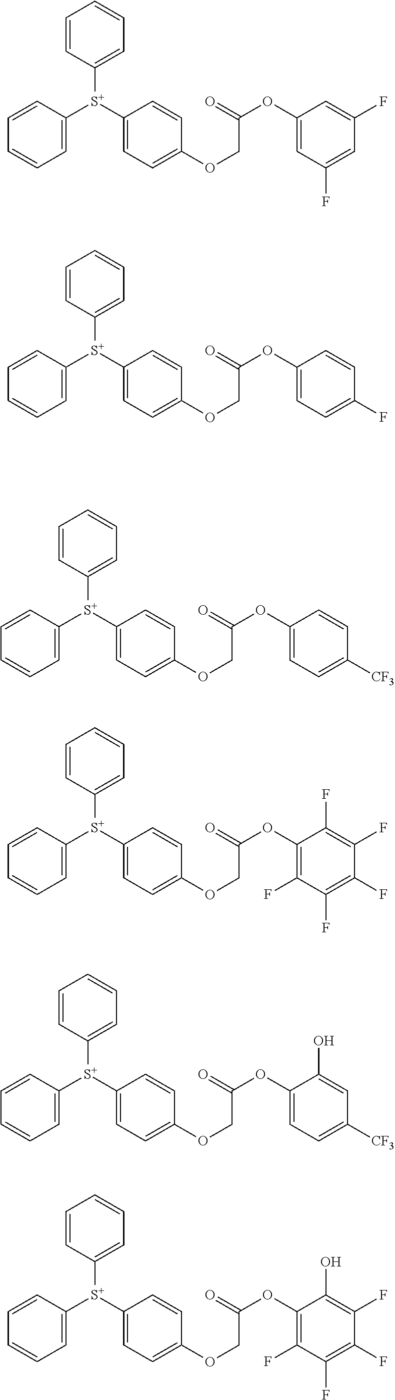

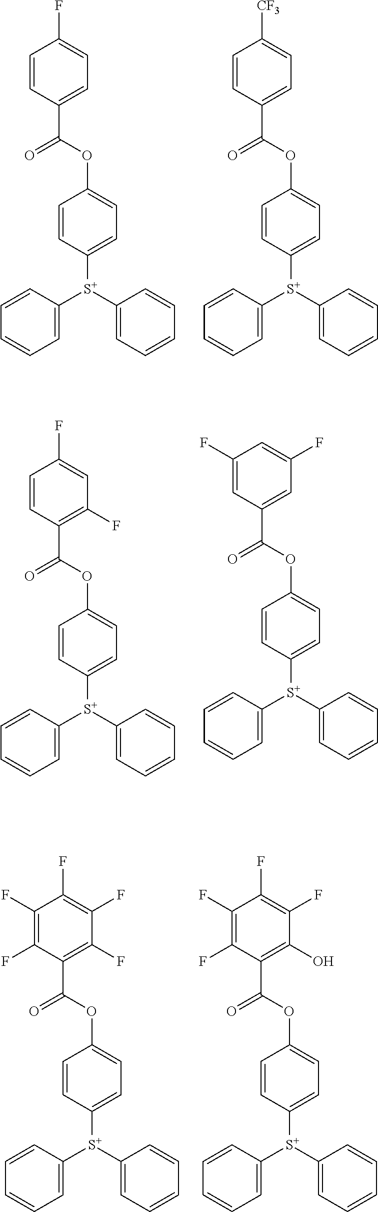

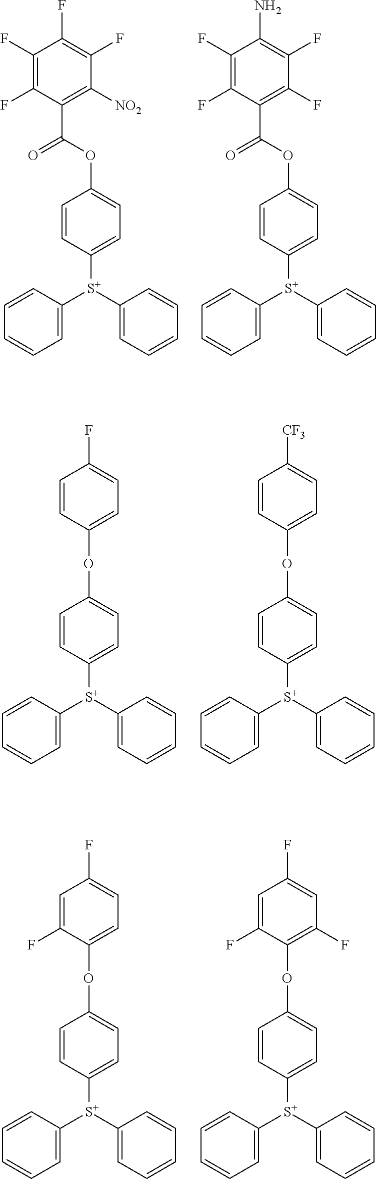

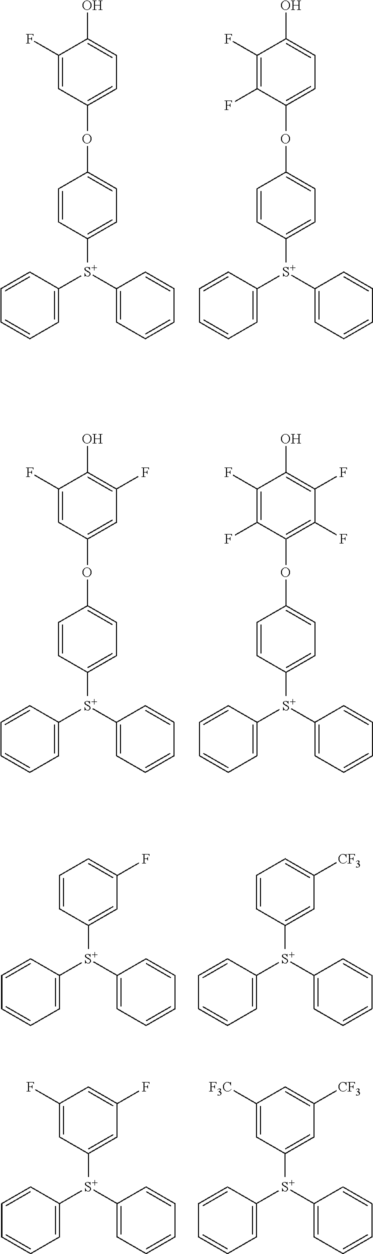

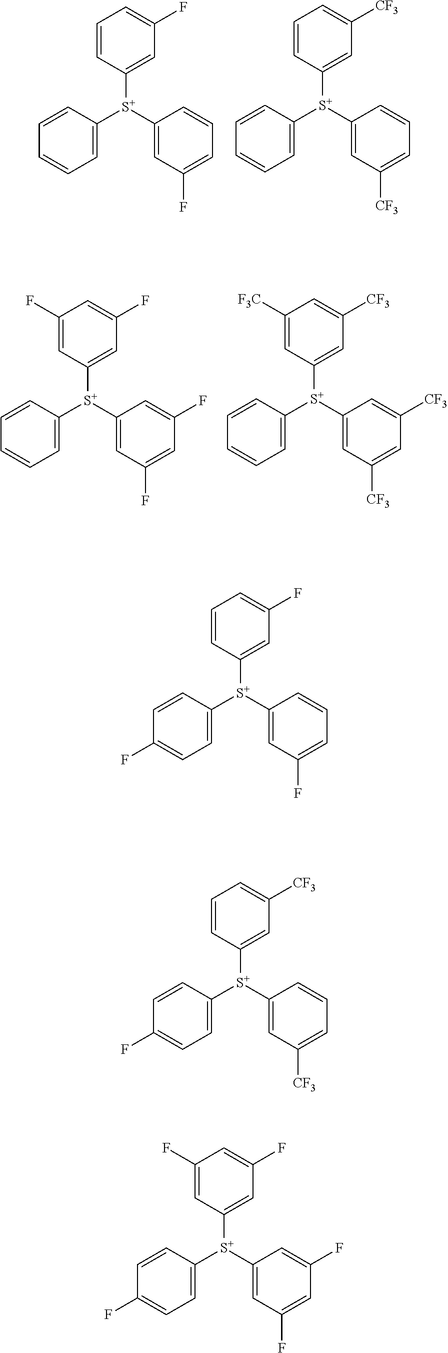

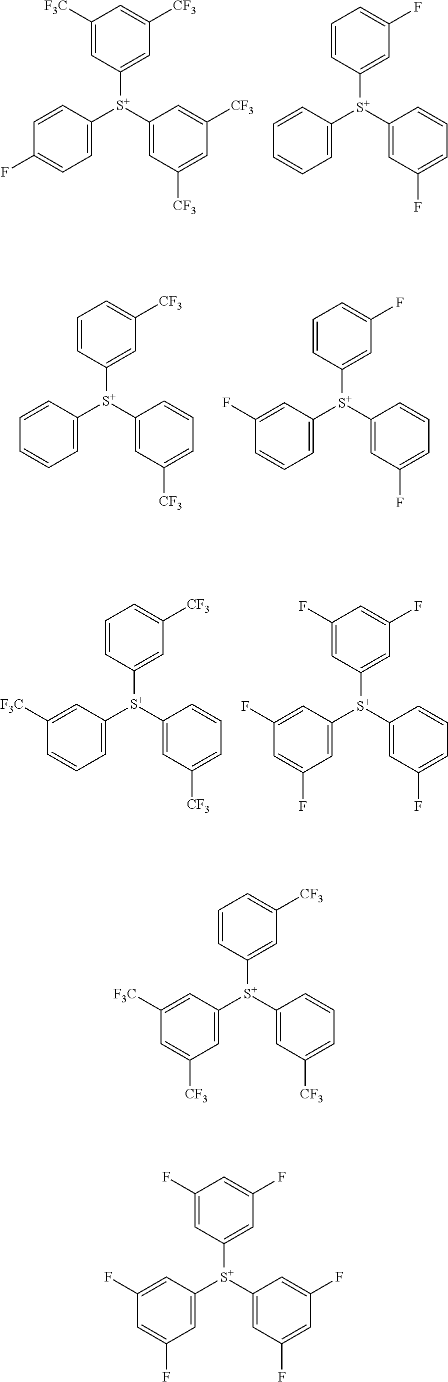

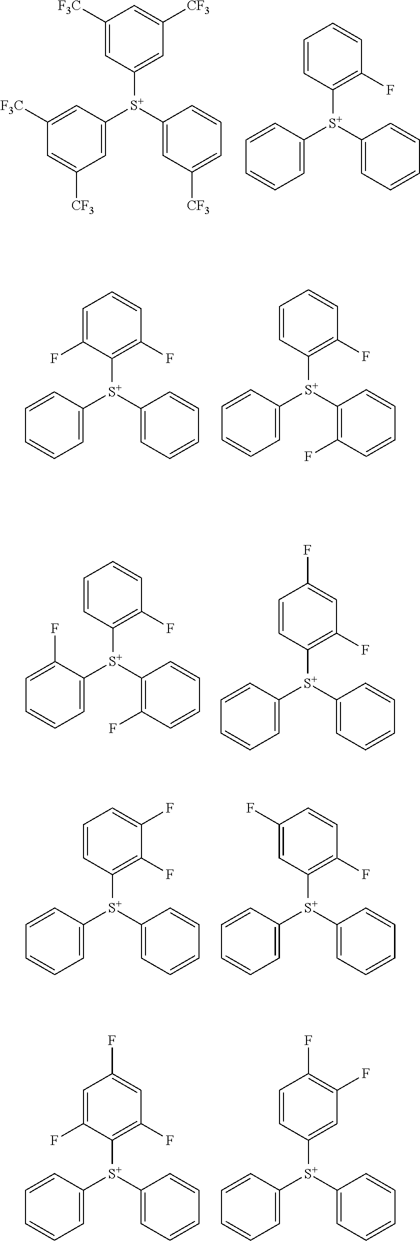

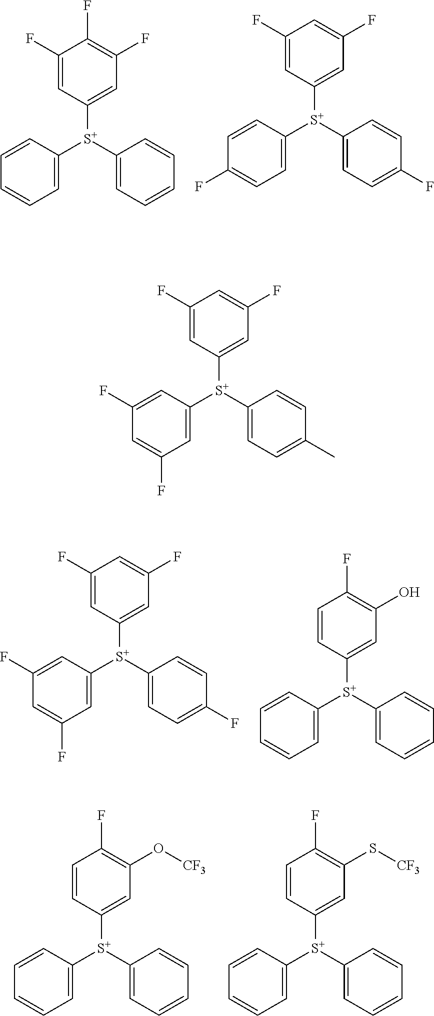

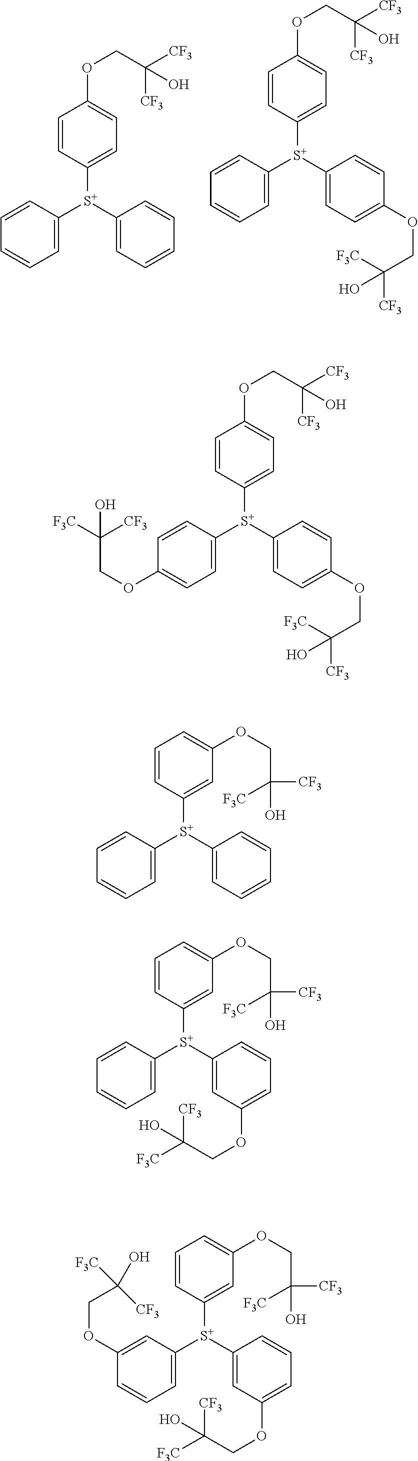

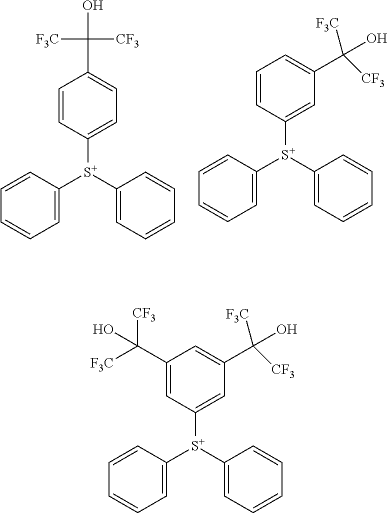

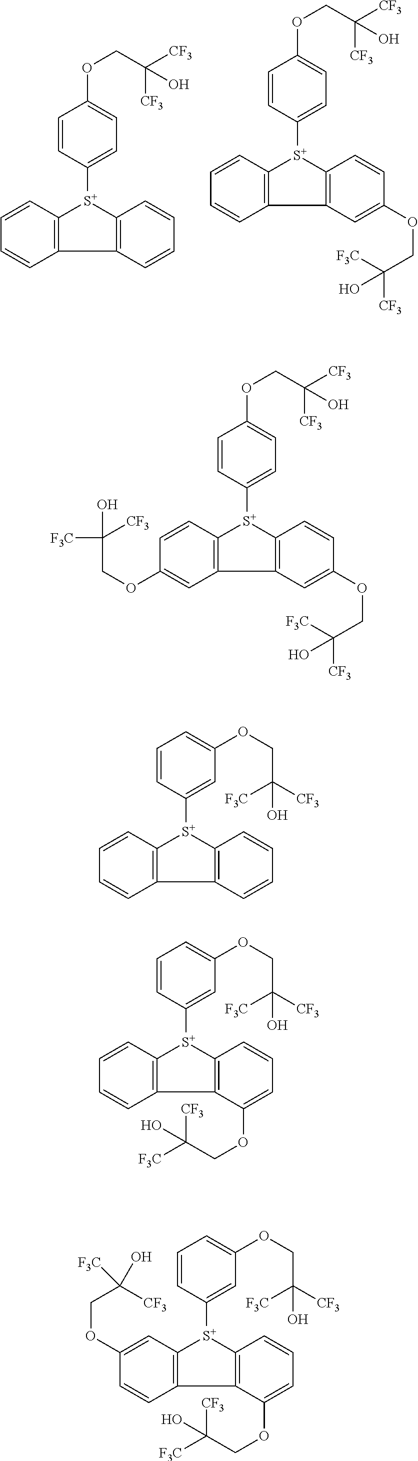

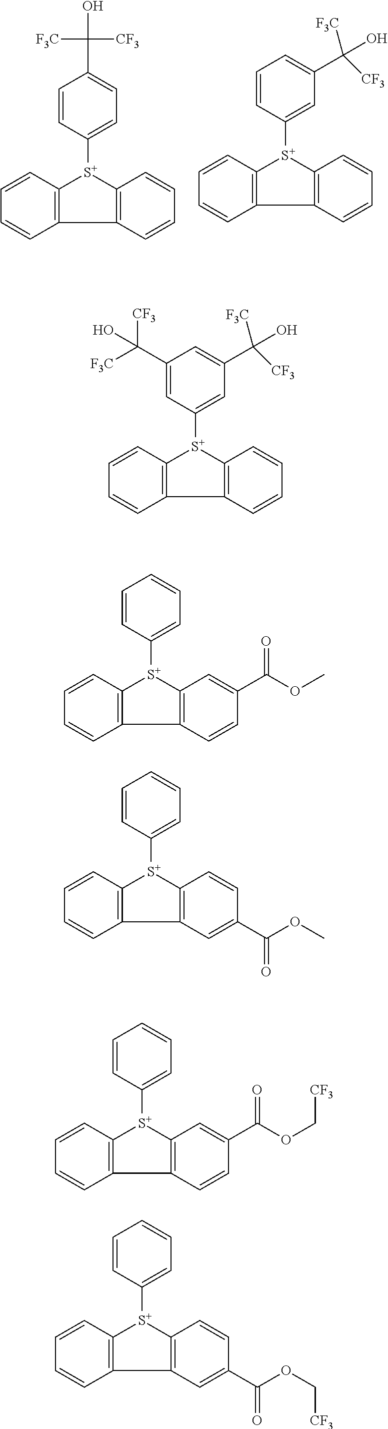

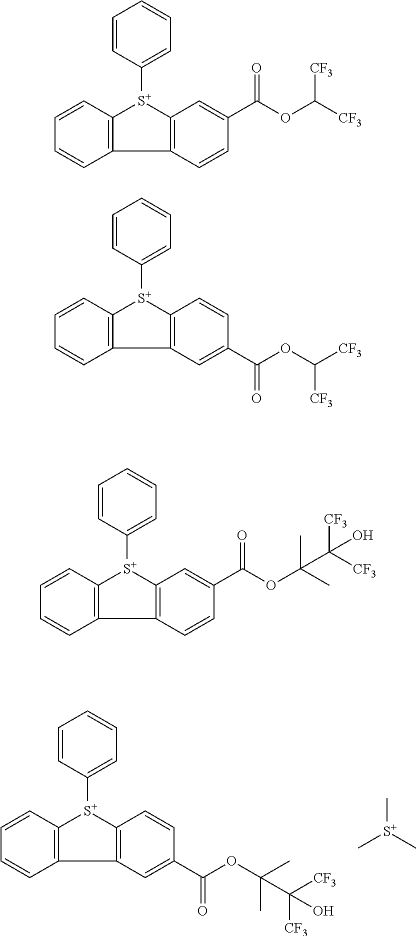

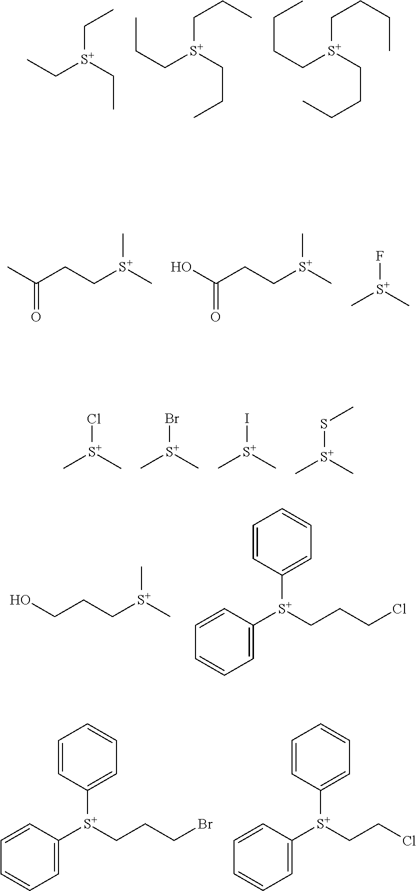

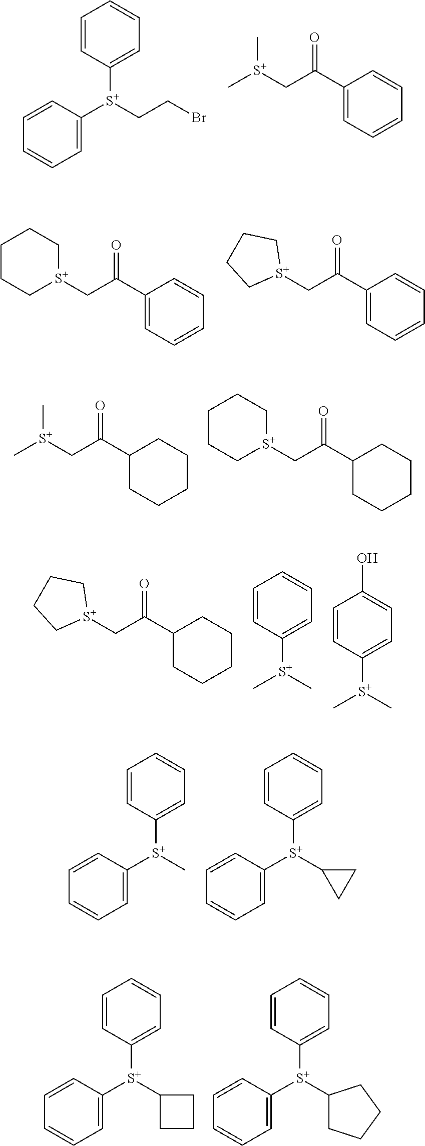

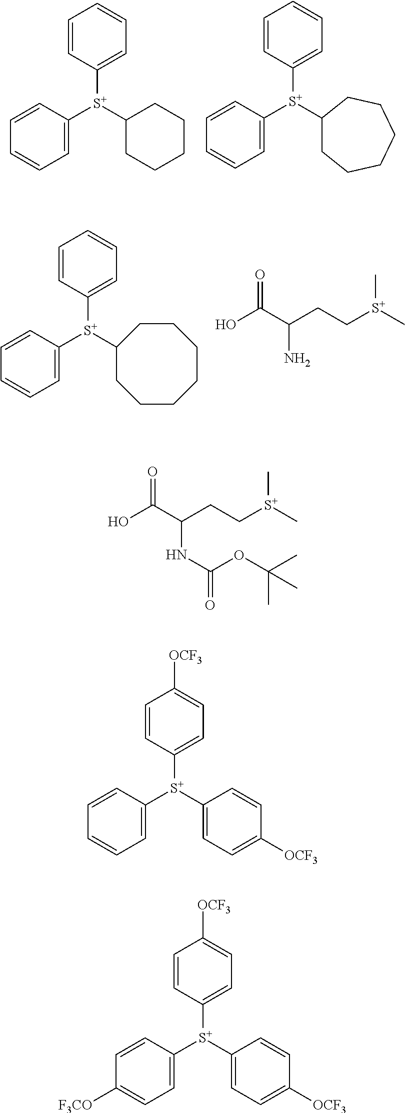

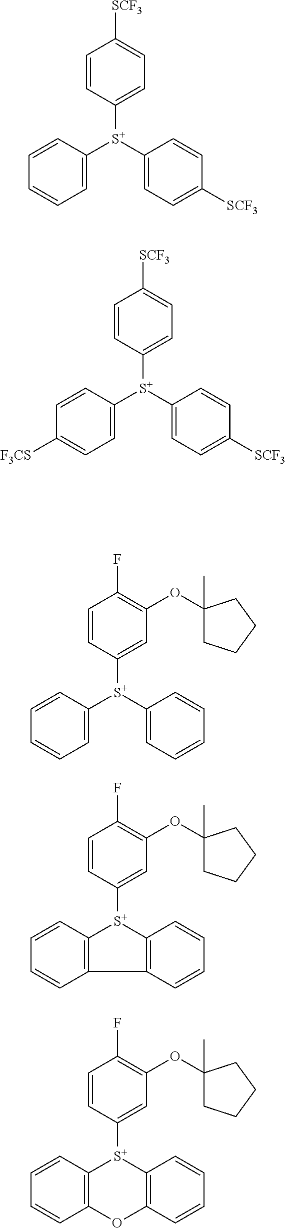

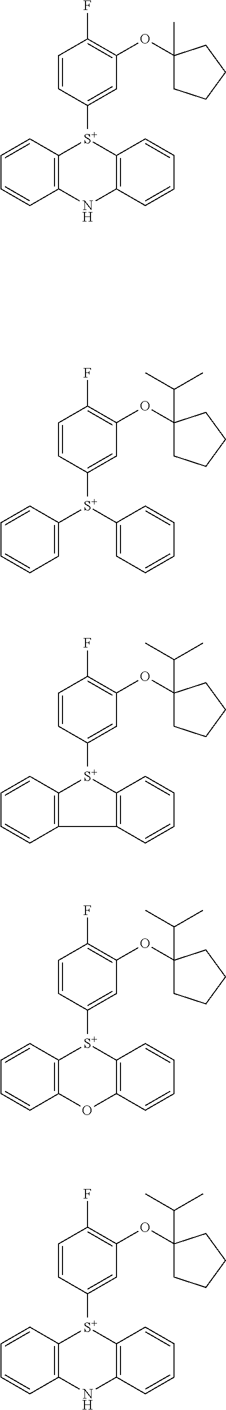

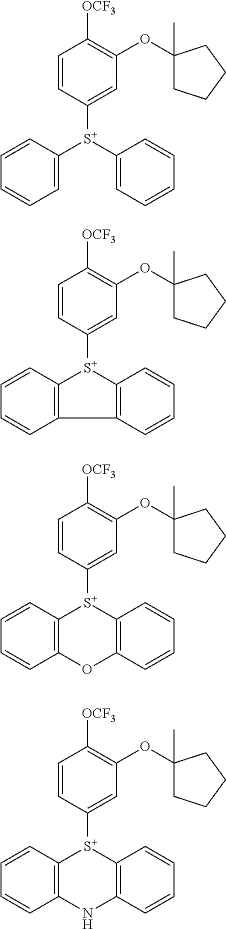

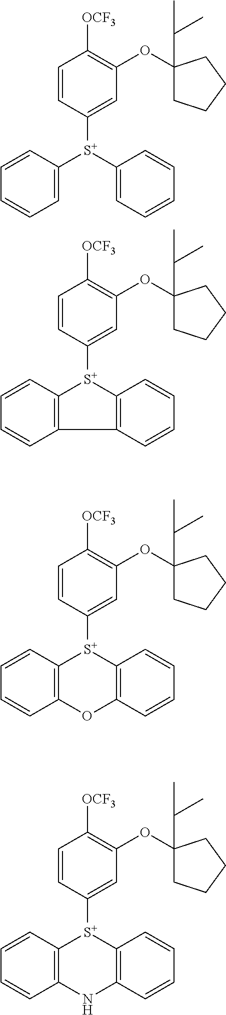

Specific examples of the iodonium cation represented by the general formula (cation-2) include the following, but are not limited thereto.

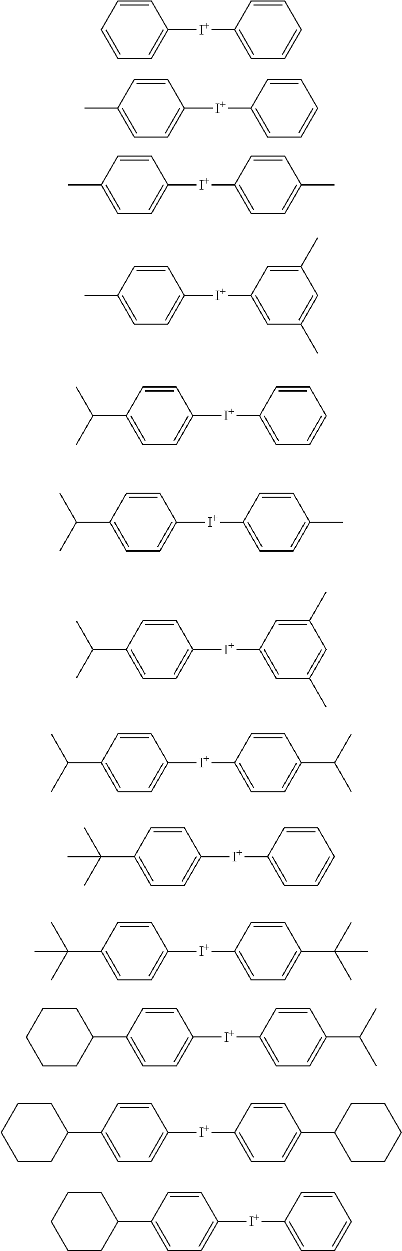

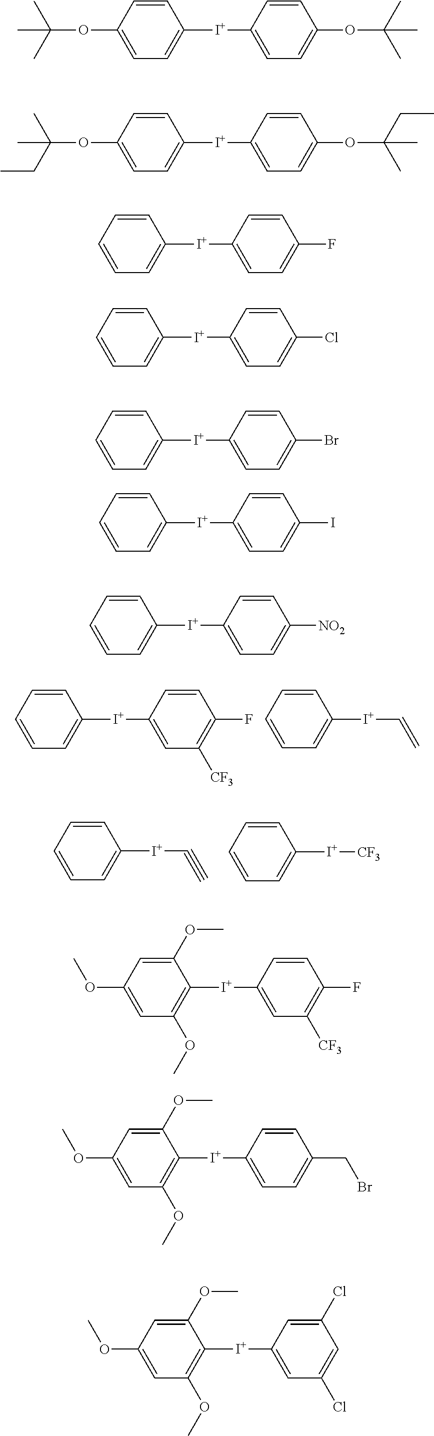

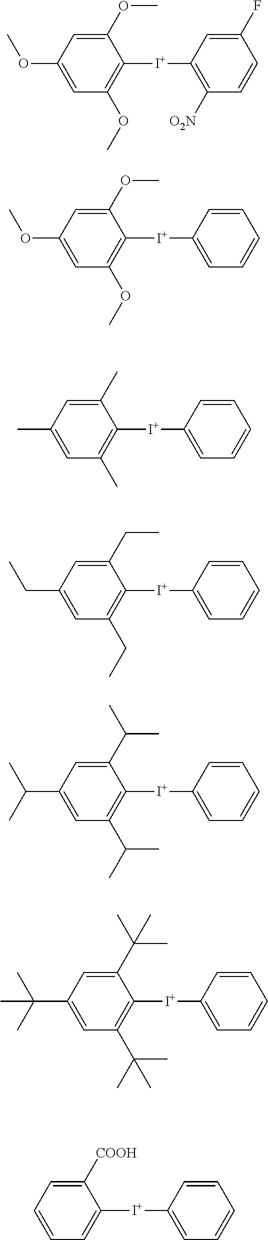

Examples of specific structures of the repeating units-c1 to -c4 include any combination of the above-described anions and cations.

Among the repeating units-c1 to -c4, the repeating units-c2, -c3, and -c4 are preferable from the viewpoint of controlling acid diffusion, the repeating units-c2 and -c4 are more preferable from the viewpoint of the acidity of the generated acid, and the repeating unit-c2 is further preferable from the viewpoint of solvent solubility.

The polymer may further include a repeating unit represented by the following general formula (a1) (hereinafter, also referred to as repeating unit-a1).

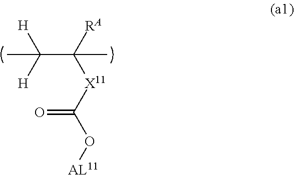

In the general formula (a1), R^{A} represents a hydrogen atom, a fluorine atom, a methyl group, or a trifluoromethyl group.

In the general formula (a1), X¹¹ represents a single bond, a phenylene group, a naphthylene group, or *—C(═O)—O—X¹¹¹—, and the phenylene group or the naphthylene group may have a substituent which is an alkoxy group having 1 to 10 carbon atoms and optionally containing a fluorine atom or is a halogen atom. X¹¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring. “*” represents an attachment point to the carbon atom of the main chain.

In the general formula (a1), AL¹¹ represents an acid-labile group. Examples of the acid-labile group include ones disclosed in JP 2013-080033 A and JP 2013-083821 A.

Typically, examples of the acid-labile group include those represented by the following general formulae (AL-11) to (AL-13).

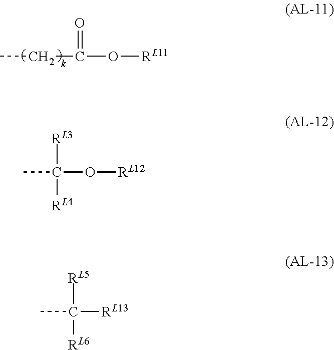

In the formulae, a broken line represents an attachment point.

In the general formulae (AL-11) and (AL-12), R^{L11} and R^{L12} each independently represent a hydrocarbyl group 1 to 40 carbon atoms, and optionally contain a heteroatom such as an oxygen atom, a sulfur atom, a nitrogen atom, or a fluorine atom. The hydrocarbyl groups may be saturated or unsaturated, and may be linear, branched, or cyclic. As the hydrocarbyl groups ones having 1 to 20 carbon atoms are preferable.

In the general formula (AL-11), “k” represents an integer of 0 to 10, preferably an integer of 1 to 5.

In the general formula (AL-12), R^{L13} and R^{L14} each independently represent a hydrogen atom or a hydrocarbyl group having 1 to 20 carbon atoms, and optionally contain a heteroatom such as an oxygen atom, a sulfur atom, a nitrogen atom, or a fluorine atom. The hydrocarbyl group may be saturated or unsaturated, and may be linear, branched, or cyclic. As the hydrocarbyl group ones having 1 to 20 carbon atoms are preferable. Furthermore, any two of R^{L12}, R^{L13}, and R^{L14} may be bonded to each other to form a ring having 3 to 20 carbon atoms together with the carbon atoms or the carbon atom and the oxygen atom bonded thereto. As the ring, a ring having 4 to 16 carbon atoms is preferable, and an alicyclic group is particularly preferable.

In the general formula (AL-13), R^{L15}, R^{L16}, and R^{L17} each independently represent a hydrocarbyl group having 1 to 20 carbon atoms, and optionally contain a heteroatom such as an oxygen atom, a sulfur atom, a nitrogen atom, or a fluorine atom. The hydrocarbyl group may be saturated or unsaturated, and may be linear, branched, or cyclic. As the hydrocarbyl group, ones having 1 to 20 carbon atoms are preferable. Furthermore, any two of R^{L15}, R^{L16}, and R^{L17} may be bonded to each other to form a ring having 3 to 20 carbon atoms together with the carbon atom bonded thereto. As the ring, a ring having 4 to 16 carbon atoms is preferable, and an alicyclic group is particularly preferable.

Specific examples of the repeating unit-a1 include the following, but are not limited thereto. Note that, in the following formulae, R^{A} and AL¹¹ are as defined above.

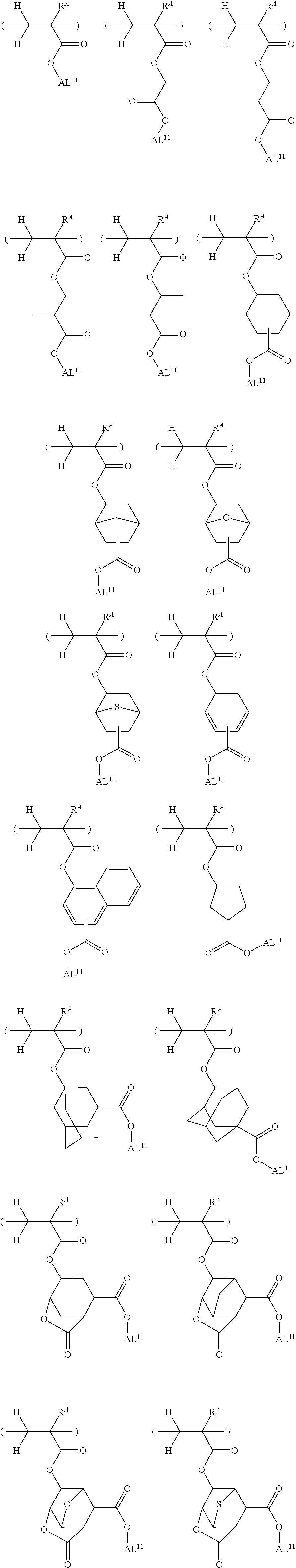

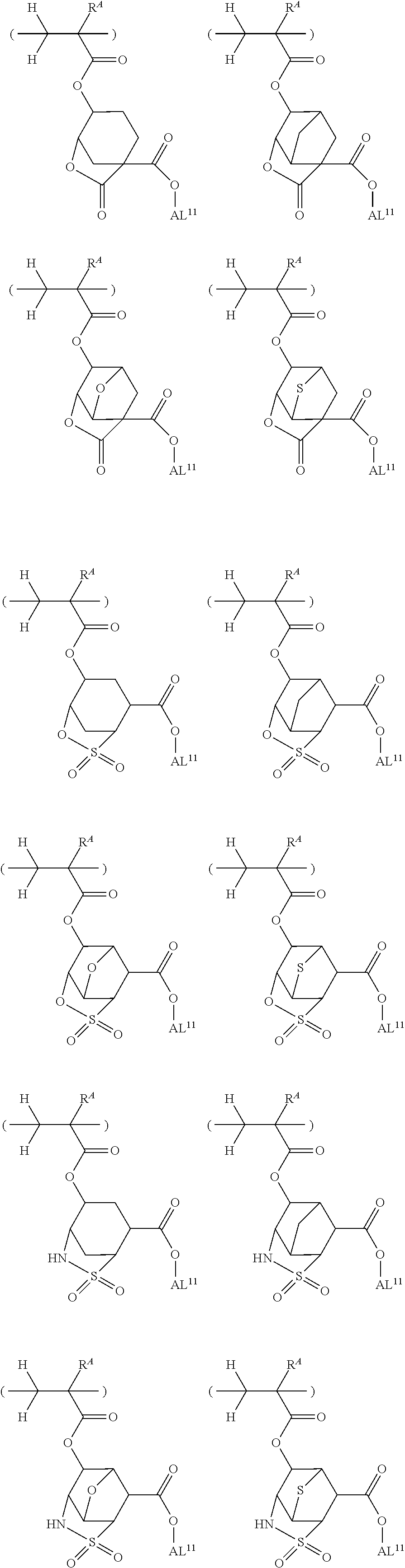

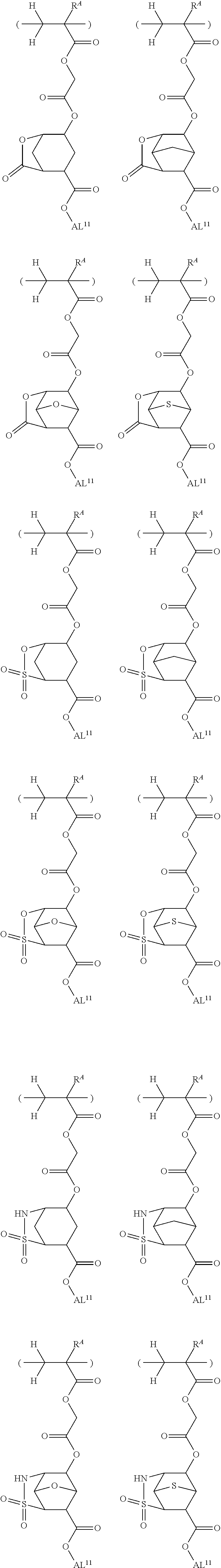

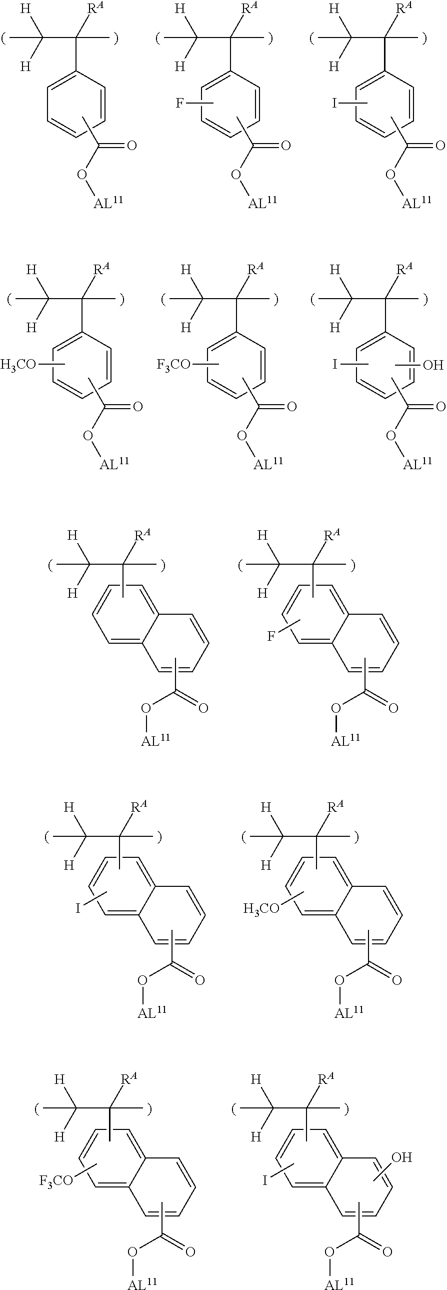

The polymer may further include a repeating unit represented by the following general formula (a2) (hereinafter, also referred to as repeating unit-a2).

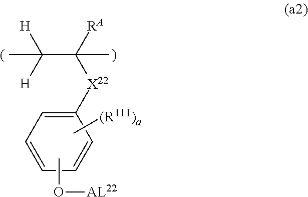

In the general formula (a2), R^{A} represents a hydrogen atom, a fluorine atom, a methyl group, or a trifluoromethyl group. X²² represents a single bond or *—C(═O)—O—. “*” represents an attachment point to the carbon atom of the main chain. R¹¹¹ represents a halogen atom, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. AL²² represents an acid-labile group. Examples of the acid-labile group include those given as examples of the acid-labile group represented by AL¹¹. “a” represents an integer of 0 to 4, preferably 0 or 1.

Specific examples of the repeating unit-a2 include the following, but are not limited thereto. Note that, in the following formulae, R^{A} and AL²² are as defined above.

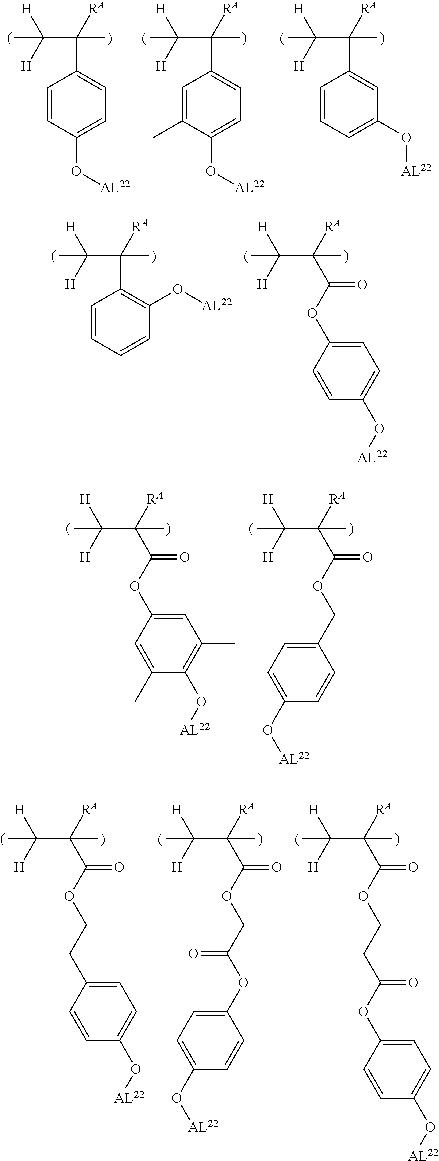

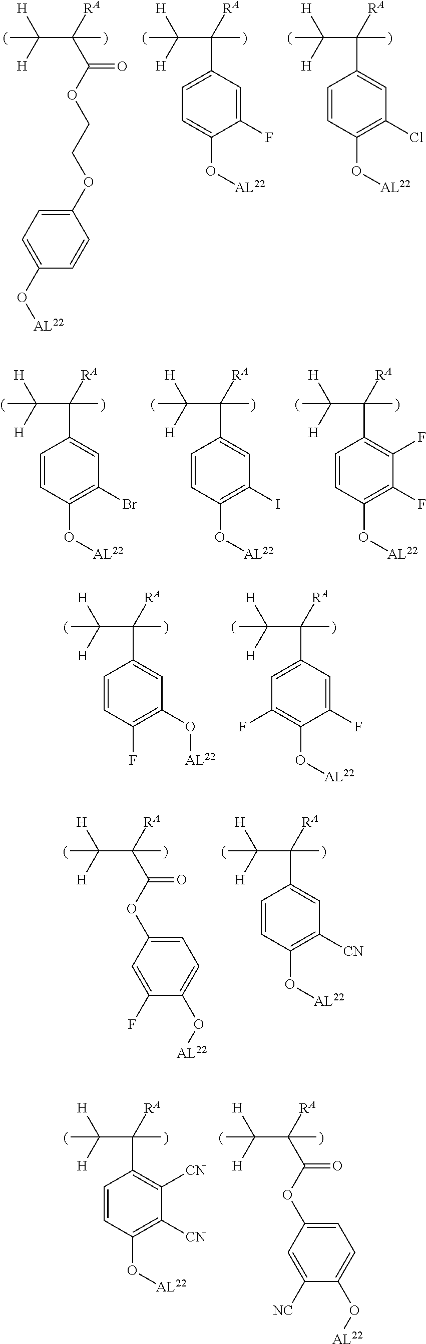

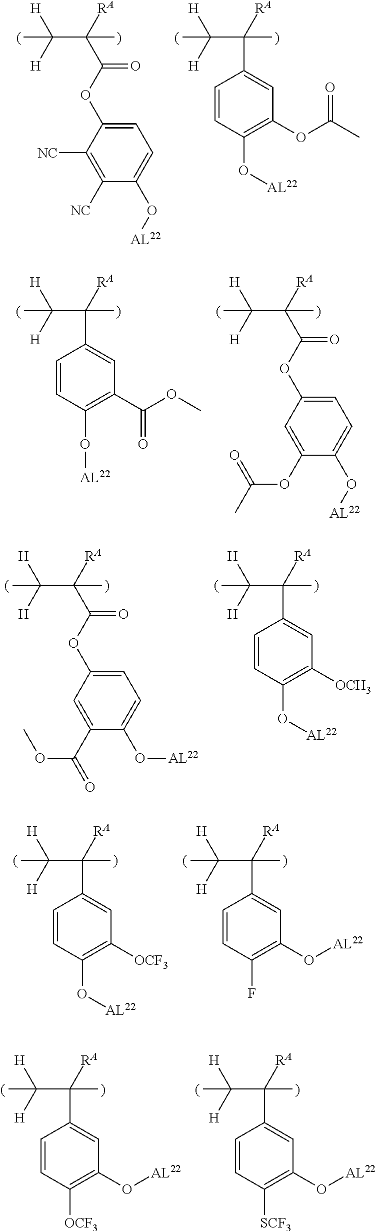

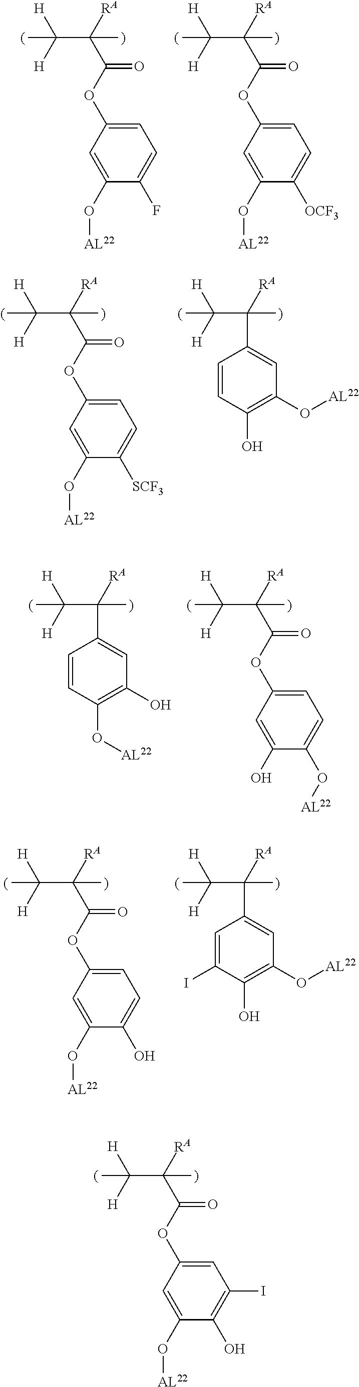

The polymer may further include a repeating unit having a structure where a hydroxy group is protected with an acid-labile group (hereinafter, also referred to as repeating unit-d). The repeating unit-d is not particularly limited as long as it has one or more structures where a hydroxy group is protected, and the protecting group is decomposed by the action of an acid to generate a hydroxy group, but ones represented by the following general formula (d1) are preferable.

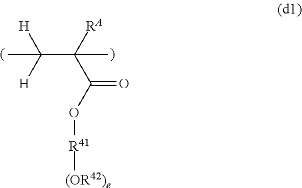

In the general formula (d1), R^{A} is as defined above. R⁴¹ represents an (e+1)-valent hydrocarbon group having 1 to 30 carbon atoms and optionally containing a heteroatom. R⁴² represents an acid-labile group. “e” represents an integer of 1 to 4.

In the general formula (d1), the acid-labile group represented by R⁴² may be deprotected by the action of an acid and generate a hydroxy group. The structure of R⁴² is not particularly limited, but an acetal structure, a ketal structure, an alkoxycarbonyl group, an alkoxymethyl group represented by the following general formula (d2), etc. are preferable, and an alkoxymethyl group represented by the following general formula (d2) is particularly preferable.

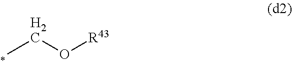

In the formula, “*” represents an attachment point. R⁴³ represents a hydrocarbyl group having 1 to 15 carbon atoms.

Specific examples of the acid-labile group represented by R⁴², the alkoxymethyl group represented by the general formula (d2), and the repeating unit-d include those given as examples in the description of the repeating unit-d disclosed in JP 2020-111564 A.

The polymer may further include a repeating unit-e derived from indene, benzofuran, benzothiophene, acenaphthylene, chromone, coumarin, norbornadiene, or a derivative thereof. Specific examples of a monomer to give the repeating unit-e include the following, but are not limited thereto.

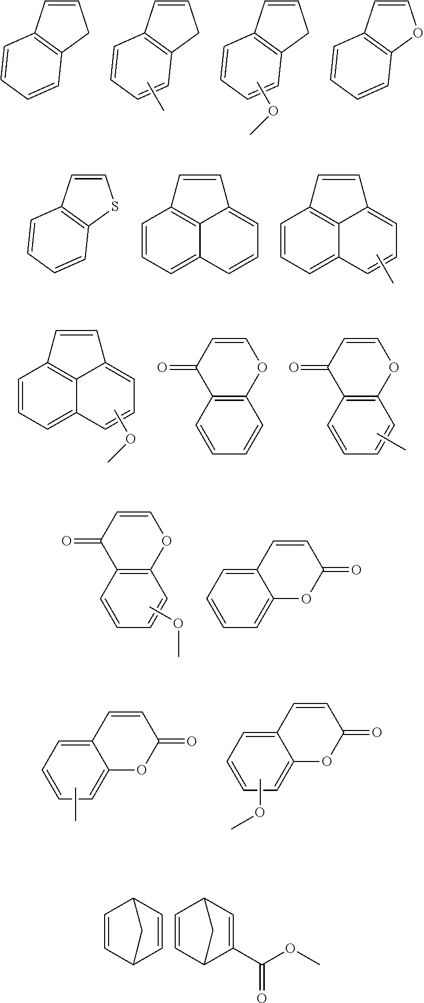

The polymer may further include a repeating unit-f derived from indane, vinylpyridine, or vinylcarbazole.

In the inventive polymer, the content ratios of the repeating units-A, -a1, -a2, -b1, -b2, -c1 to -c4, -d, -e, and -f are preferably 0<A≤0.8, 0≤a1≤0.8, 0≤a2≤0.8, 0≤b1≤0.6, 0≤b2≤0.6, 0≤c1≤0.4, 0≤c2≤0.4, 0≤c3≤0.4, 0≤c4≤0.4, 0≤d≤0.5, 0≤e≤0.3, and 0≤f≤0.3; more preferably 0<A≤0.5, 0≤a1≤0.5, 0≤a2≤0.5, 0≤b1≤0.5, 0≤b2≤0.5, 0≤c1≤0.3, 0≤c2≤0.3, 0≤3≤0.3, 0≤c4≤0.3, 0≤d≤0.3, 0≤e≤0.3, and 0≤f≤0.3, provided that A+a1+a2+b1+b2+c1+c2+c3+c4+d+e+f=1.

The polymer preferably has a weight-average molecular weight (Mw) of 1,000 to 500,000, more preferably 3,000 to 100,000. When the Mw is in these ranges, sufficient etching resistance can be achieved, and there is no risk of resolution being degraded by a difference in dissolution rate between before and after exposure not being ensured. Note that, in the present invention, Mw is a value measured in terms of polystyrene by gel permeation chromatography (GPC) using tetrahydrofuran (THF) or N,N-dimethylformamide (DMF) as an eluent.

Furthermore, regarding the molecular weight distribution (Mw/Mn) of the polymer, as pattern rule is miniaturized, the influence of Mw/Mn is likely to be greater, and therefore, to obtain a chemically-amplified resist composition suitably used for a fine pattern size, the Mw/Mn is preferably 1.0 to 2.0, a narrow dispersity. Within this range, there are few polymers having a molecular weight lower or higher than Mw, and there are no risks of foreign substances being found on the pattern or the pattern shape being degraded after exposure.

To synthesize the polymer, for example, the monomers to give the repeating units described above can be subjected to heat polymerization in an organic solvent to which a radical polymerization initiator has been added.

Examples of the organic solvent used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator include 2,2′-azobisisobutyronitrile (AIBN), 2,2′-azobis(2,4-dimethylvaleronitrile), dimethyl-2,2′-azobis(2-methylpropionate), 1,1′-azobis(1-acetoxy-1-phenylethane), benzoyl peroxide, and lauroyl peroxide. The amount of the polymerization initiator to be added is preferably 0.01 to 25 mol % of the total amount of the monomers to be polymerized. The reaction temperature is preferably 50 to 150° C., more preferably 60 to 100° C. The reaction time is preferably 2 to 24 hours, and from the viewpoint of production efficiency, more preferably 2 to 12 hours.

The polymerization initiator may be added to the solution of the monomer and supplied to the reaction vessel, or a solution of the polymerization initiator may be prepared separately from the solution of the monomer, and each may be supplied to the reaction vessel independently. There is a possibility that the polymerization reaction may progress due to radicals generated from the polymerization initiator during waiting time and an ultra-high molecular weight polymer may be generated, and therefore, from the viewpoint of quality control, it is preferable to prepare each of the monomer solution and the polymerization initiator solution independently and add the solutions dropwise. An acid-labile group introduced into the monomer may be used as it is, or may be protected or partially protected after polymerization. Furthermore, to adjust the molecular weight, a known chain transfer agent, such as dodecyl mercaptan and 2-mercaptoethanol may also be used. In this case, the amount of the chain transfer agent to be added is preferably 0.01 to 20 mol % of the total amount of the monomers to be polymerized.

In the case of a monomer containing a hydroxy group, the process may include: substituting the hydroxy group with an acetal group susceptible to deprotection with acid, such as an ethoxyethoxy group, prior to the polymerization; and performing the deprotection with weak acid and water after the polymerization. Alternatively, the process may include: substituting the hydroxy group with an acetyl group, a formyl group, a pivaloyl group, or the like prior to the polymerization; and performing alkaline hydrolysis after the polymerization.

In a case where hydroxystyrene or hydroxyvinylnaphthalene is copolymerized, hydroxystyrene or hydroxyvinylnaphthalene and other monomers may be subjected to heat polymerization in an organic solvent to which a radical polymerization initiator has been added; alternatively, acetoxystyrene or acetoxyvinylnaphthalene may be used, and after the polymerization, the acetoxy group may be deprotected by the alkaline hydrolysis to convert the acetoxystyrene or acetoxyvinylnaphthalene to polyhydroxystyrene or hydroxypolyvinylnaphthalene.

As examples of the base in the alkaline hydrolysis, ammonia water, triethylamine, etc. are usable. The reaction temperature is preferably −20 to 100° C., more preferably 0 to 60° C. The reaction time is preferably 0.2 to 100 hours, more preferably 0.5 to 20 hours.

Incidentally, the amount of each monomer in the monomer solution can, for example, be set appropriately to achieve the above-described preferable content ratios of the repeating units.

As for the polymer obtained in the above-described manufacturing method, a reaction solution obtained by the polymerization reaction may be a final product. Alternatively, a powder obtained via a purification step, such as a reprecipitation method in which the reaction solution is added into a poor solvent to obtain a powder, may be treated as a final product. From the viewpoints of operation efficiency and quality stabilization, the powder obtained in the purification step is preferably dissolved in a solvent to form a polymer solution to be operated as a final product.

Specific examples of the solvent used in this case include solvents described in paragraphs [0144] to [0145] in JP 2008-111103 A, and include: ketones, such as cyclohexanone and methyl-2-n-pentylketone; alcohols, such as 3-methoxybutanol, 3-methyl-3-methoxybutanol, 1-methoxy-2-propanol, and 1-ethoxy-2-propanol; ethers, such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as GBL; alcohols, such as diacetone alcohol (DAA); alcoholic solvents having a high boiling point, such as diethylene glycol, propylene glycol, glycerin, 1,4-butanediol, and 1,3-butanediol; and mixed solvents thereof.

In the polymer solution, the concentration of the polymer is preferably 0.01 to 30 mass %, more preferably 0.1 to 20 mass %.

The reaction solution and the polymer solution are preferably filtered with a filter. The filtration can remove foreign substances and gel, which may cause defects, and is effective in terms of quality stabilization.

Examples of a material of the filter used for the filtration include fluorocarbon, cellulose, nylon, polyester, and hydrocarbon materials. In the step of filtering the resist composition, preferable is a filter formed with a fluorocarbon, so-called Teflon (registered trademark), a hydrocarbon, such as polyethylene and polypropylene, or nylon. A pore size of the filter can be appropriately selected depending on target cleanliness, and is preferably 100 nm or smaller, and more preferably 20 nm or smaller. One of these filters may be used alone, or a plurality of these filters may be used in combination. As for the filtration method, the solution may be passed through the filter only once, but the solution is preferably circulated to be filtered a plurality of times. In the step for producing the polymer, the filtration step may be performed in any order any number of times, but the reaction solution after the polymerization reaction, the polymer solution, or both thereof are preferably filtered.

One kind of the base resin (A) may be used, or two or more kinds thereof that differ in composition ratio, Mw, and/or Mw/Mn may be used in combination. Furthermore, the base resin (A) may include, in addition to the above-described polymer, a hydrogenated ring-opening metathesis polymer, and regarding this polymer, those disclosed in JP 2003-066612 A can be used.

### [(B) Organic Solvent]

The inventive chemically-amplified resist composition contains an organic solvent as a component (B). The organic solvent (B) is not particularly limited as long as it is capable of dissolving the above-described components and the components described later. Examples of such an organic solvent include: ketones, such as cyclopentanone, cyclohexanone, and methyl-2-n-pentyl ketone; alcohols, such as 3-methoxybutanol, 3-methyl-3-methoxybutanol, 1-methoxy-2-propanol, and 1-ethoxy-2-propanol; ketoalcohols, such as DAA; ethers, such as PGME, ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as GBL; and mixed solvents thereof.

Among these organic solvents (B), 1-ethoxy-2-propanol, PGMEA, cyclohexanone, GBL, DAA, and mixed solvents thereof, which are particularly excellent in the solubility of the component (A) base resin, are preferable.

In the inventive chemically-amplified resist composition, the organic solvent (B) is preferably contained in an amount of 200 to 5,000 parts by mass, more preferably 400 to 3,500 parts by mass based on 80 parts by mass of the base resin (A). One kind of the organic solvent (B) may be used, or two or more kinds thereof may be used in mixture.

### [(C) Quencher]

The inventive chemically-amplified resist composition may contain a quencher as a component (C). Note that, in the present invention, a quencher means a material for trapping an acid generated from the photo-acid generator in the chemically-amplified resist composition to prevent diffusion to unexposed portions and forming a desired pattern.

Examples of the quencher (C) include onium salts represented by the following general formula (2) or (3).

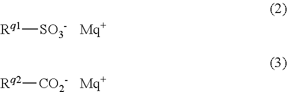

In the general formula (2), R^{q1} represents a hydrogen atom or a hydrocarbyl group having 1 to 40 carbon atoms and optionally containing a heteroatom, excluding hydrocarbyl groups in which the hydrogen atom bonded to the carbon atom in the a position of the sulfo group is substituted with a fluorine atom or a fluoroalkyl group. In the general formula (3), R^{q2} represents a hydrogen atom or a hydrocarbyl group having 1 to 40 carbon atoms and optionally containing a heteroatom.

Specific examples of the hydrocarbyl group represented by R^{q1} having 1 to 40 carbon atoms include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; aryl groups having 6 to 40 carbon atoms, such as a phenyl group, a naphthyl group, and an anthracenyl group; etc. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Specific examples of the hydrocarbyl group represented by R^{q2} also include, besides the substituents given as specific examples of R^{q1}, fluorinated saturated hydrocarbyl groups, such as a trifluoromethyl group and a trifluoroethyl group, and fluorinated aryl groups, such as a pentafluorophenyl group and a 4-trifluoromethylphenyl group.

Specific examples of the anion of the onium salt represented by the general formula (2) include the following, but are not limited thereto.

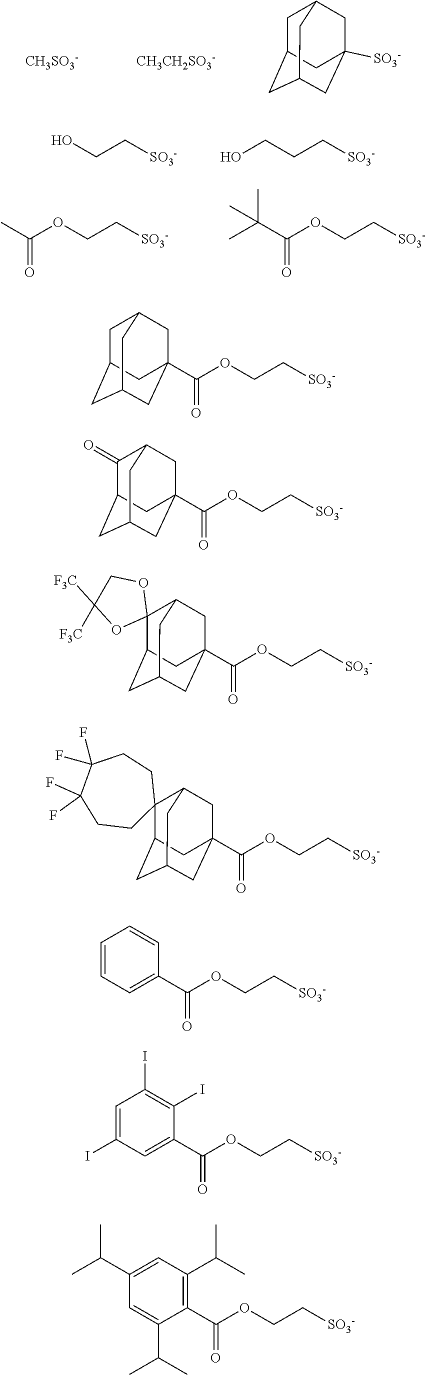

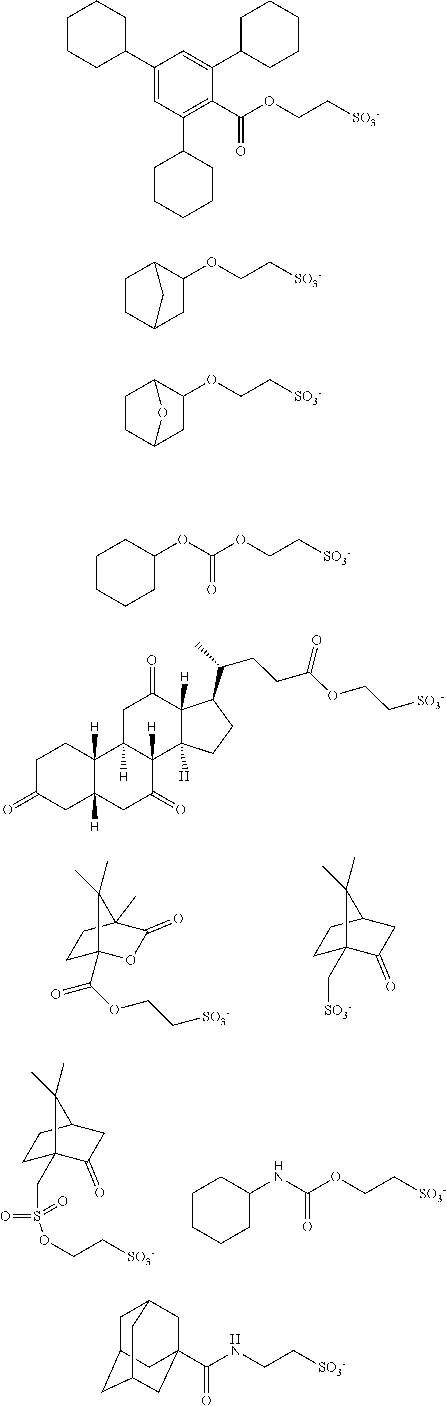

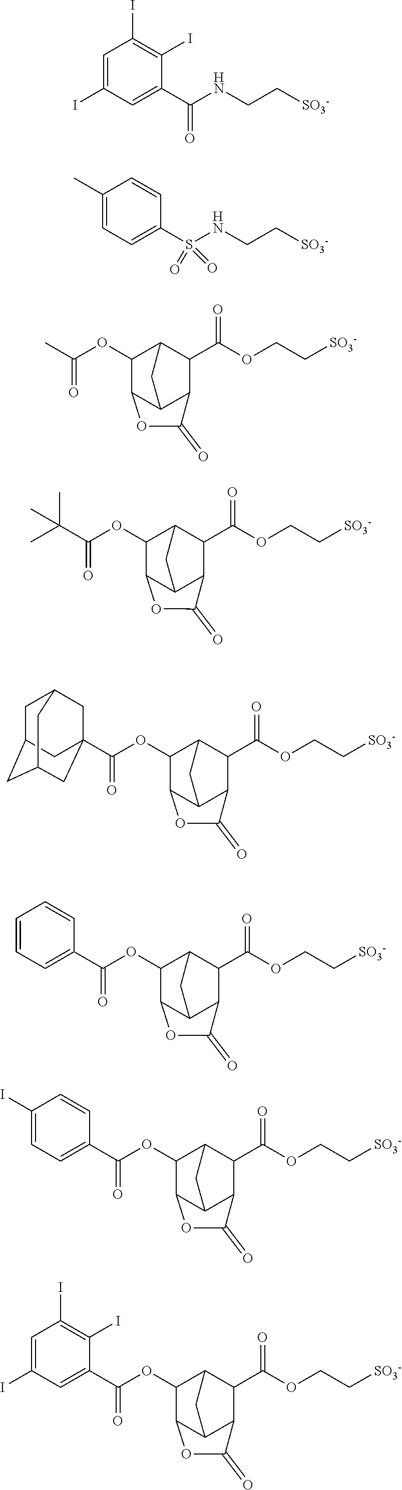

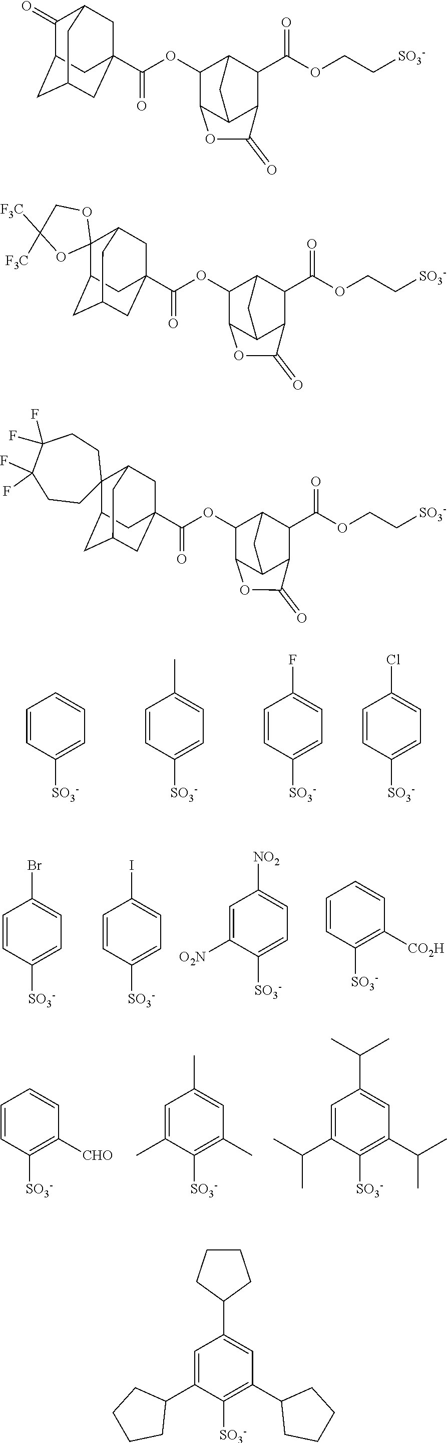

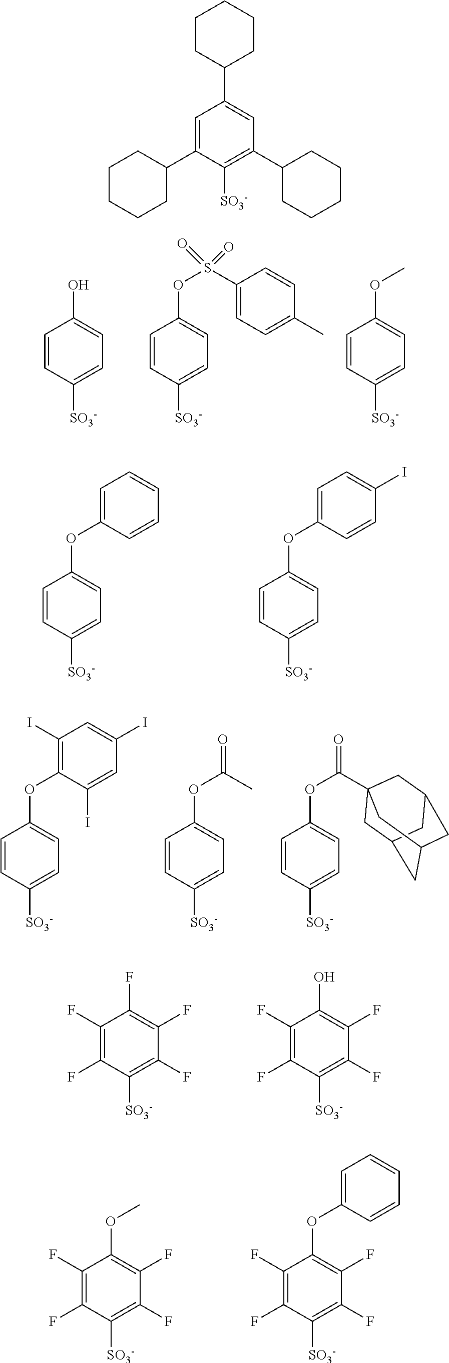

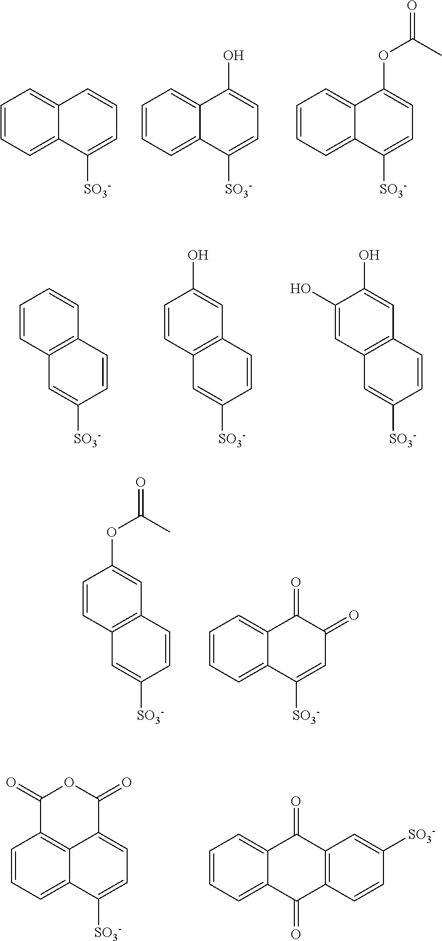

Specific examples of the anion of the onium salt represented by the general formula (3) include the following, but are not limited thereto.

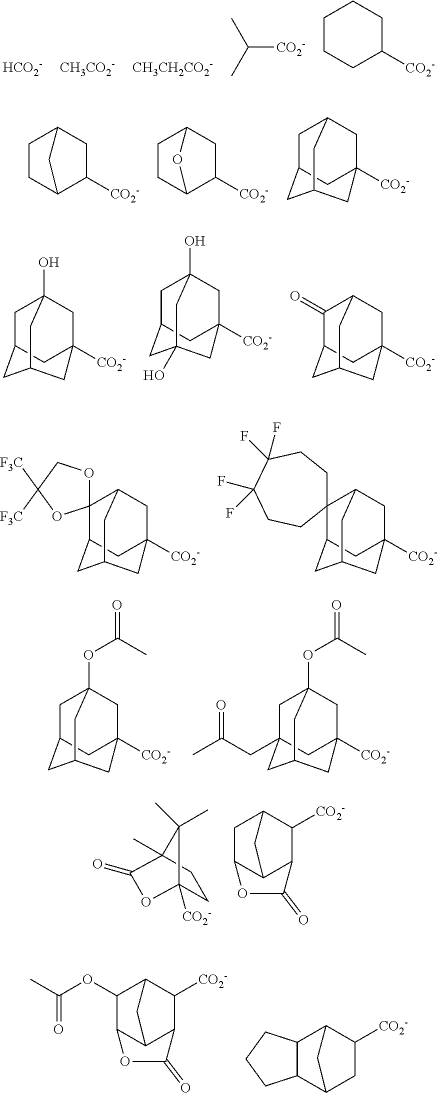

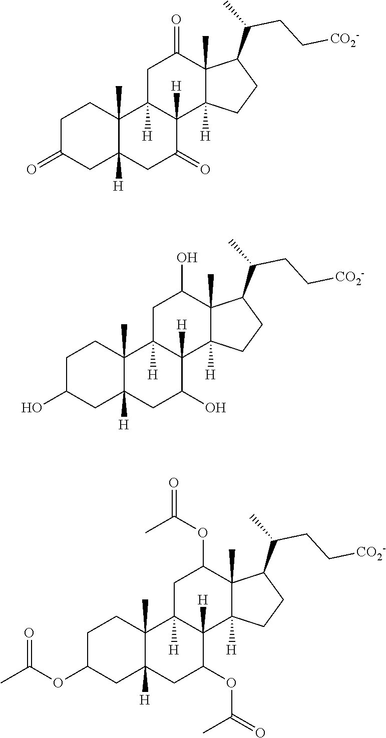

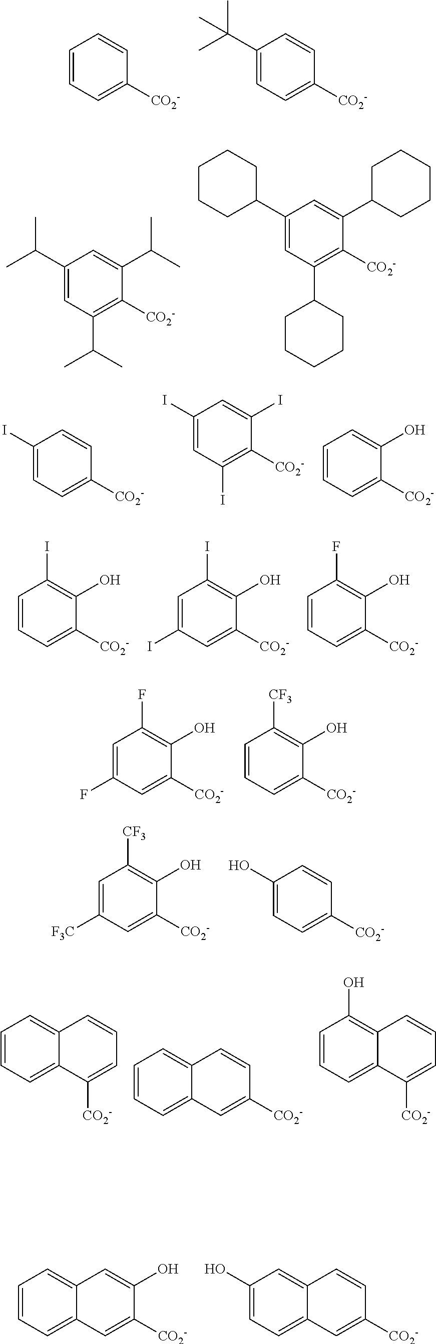

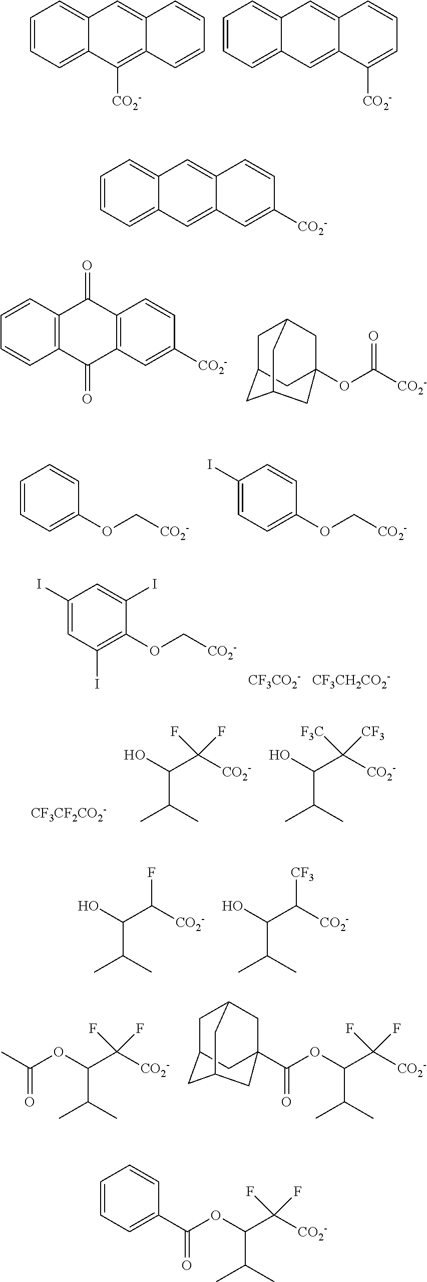

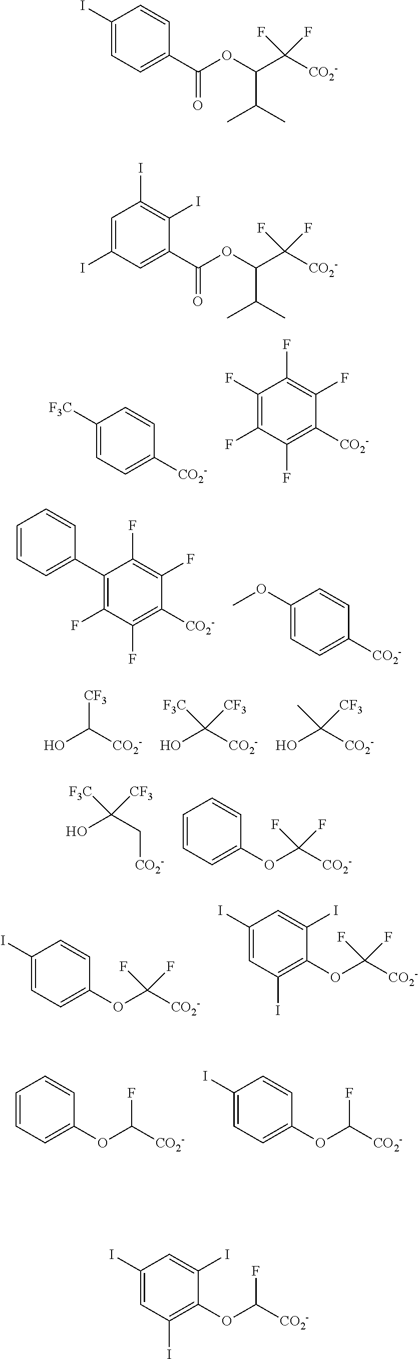

In the general formulae (2) and (3), Mq⁺ represents an onium cation. As the onium cation, preferable are the sulfonium cation represented by the general formula (cation-1), the iodonium cation represented by the general formula (cation-2), or the ammonium cation represented by the following general formula (cation-3).

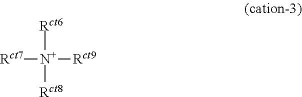

In the general formula (cation-3), R^{ct6} to R^{ct9} each independently represent a hydrocarbyl group having 1 to 40 carbon atoms and optionally containing a heteroatom. Furthermore, R^{ct6} and R^{ct7} may be bonded to each other to form a ring together with the nitrogen atom bonded thereto. Examples of the hydrocarbyl group include those given as examples of the hydrocarbyl groups represented by R^{ct1} to R^{ct5} in the description of the general formulae (cation-1) and (cation-2).

Specific examples of the ammonium cation represented by the general formula (cation-3) include the following, but are not limited thereto.

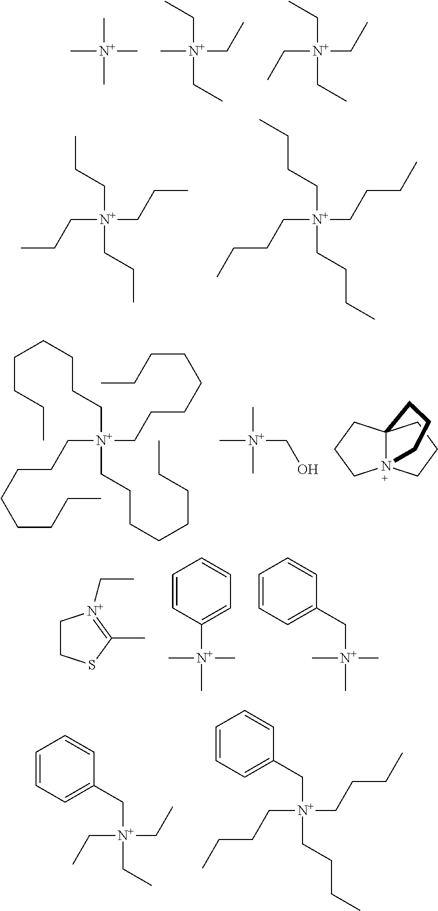

Specific examples of the onium salt represented by the general formula (2) or (3) include any combination of the above-described anions and cations. Incidentally, these onium salts can be easily prepared by an ion-exchange reaction using a known organic chemistry process. Regarding the ion-exchange reaction, JP 2007-145797 A may be consulted, for example.

The onium salt represented by the general formula (2) or (3) acts as a quencher in the inventive chemically-amplified resist composition. This results from the fact that each counter anion of the onium salts is a conjugate base of a weak acid. Here, weak acid means an acid that exhibits an acidity at which the acid-labile group of the unit, containing an acid-labile group, used in the base resin (A) cannot be deprotected. The onium salt represented by the general formula (2) or (3) functions as a quencher when used in combination with an onium-salt-type photo-acid generator having, as a counter anion, a conjugate base of a strong acid, such as a sulfonic acid fluorinated at the a position. That is, when an onium salt that generates a strong acid, such as a sulfonic acid fluorinated at the a position, and an onium salt that generates a weak acid, such as a sulfonic acid not fluorinated or a carboxylic acid, are used in mixture, collision between a strong acid generated from the photo-acid generator by irradiation with a high-energy beam and an onium salt having an unreacted weak acid anion causes a weak acid to be released by salt exchange, and an onium salt having a strong acid anion is generated. In this process, the strong acid is exchanged with a weak acid having a lower catalytic ability. Therefore, the acid appears to be deactivated, and acid diffusion can be controlled.

Furthermore, as the quencher (C), it is also possible to use: an onium salt disclosed in JP 6848776 B2, having a sulfonium cation and a phenoxide anion moiety within a single molecule; furthermore, an onium salt disclosed in JP 6583136 B2 or JP 2020-200311 A, having a sulfonium cation and a carboxylate anion moiety within a single molecule; or an onium salt disclosed in JP 6274755 B2, having an iodonium cation and a carboxylate anion moiety within a single molecule.

Here, in a case where the photo-acid generator that generates a strong acid is an onium salt, as described above, it is possible to exchange, with a weak acid, a strong acid generated by irradiation with a high-energy beam. However, on the other hand, it is assumed that it is difficult for a weak acid generated by irradiation with a high-energy beam to collide with an unreacted onium salt that generates a strong acid and undergo salt exchange. This results from the phenomenon that an onium cation forms an ion pair more easily with an anion of a stronger acid.

When the inventive chemically-amplified resist composition contains, as the quencher (C), an onium salt represented by the general formula (2) or (3), the contained amount is preferably 0.1 to 20 parts by mass, more preferably 0.1 to 10 parts by mass based on 80 parts by mass of the base resin (A). When the contained amount of the onium-salt-type quencher of the component (C) is in such ranges, resolution is favorable, and there is no remarkable degradation of sensitivity. Therefore, such ranges are preferable. One kind of the onium salt represented by the general formula (2) or (3) can be used, or two or more kinds thereof can be used in combination.

The inventive chemically-amplified resist composition may contain a nitrogen-containing compound as the quencher (C). Examples of the nitrogen-containing compound of the component (C) include primary, secondary, and tertiary amine compounds disclosed in paragraphs [0146] to [0164] of JP 2008-111103 A; in particular, amine compounds having a hydroxy group, an ether bond, an ester bond, a lactone ring, a cyano group, or a sulfonic acid ester bond. Furthermore, examples also include compounds in which a primary or secondary amine is protected with a carbamate group, such as the compounds disclosed in JP 3790649 B2.

It is also possible to use, as a nitrogen-containing compound, a sulfonic acid sulfonium salt having a nitrogen-containing substituent. Such a compound functions as a so-called photo-degradable base, which functions as a quencher in unexposed portions and loses the quencher function in exposed portions due to neutralization with the acid generated by itself. Using a photo-degradable base, the contrast between exposed and unexposed portions can be further enhanced. Regarding the photo-degradable base, JP 2009-109595 A, JP 2012-046501 A, etc. may be consulted, for example.

When the inventive chemically-amplified resist composition contains a nitrogen-containing compound as the quencher (C), the contained amount is preferably 0.001 to 12 parts by mass, more preferably 0.01 to 8 parts by mass based on 80 parts by mass of the base resin (A). One kind of the nitrogen-containing compound may be used, or two or more kinds thereof may be used in combination.

### [(D) Photo-Acid Generator]

The inventive chemically-amplified resist composition may contain, as a component (D), photo-acid generators (hereinafter, also referred to as other photo-acid generators) other than the photo-acid generators bonded to the polymer main chain in the component (A). The other photo-acid generators are not particularly limited as long as they are compounds that generate an acid due to irradiation with a high-energy beam. Suitable examples of the other photo-acid generators include ones represented by the following general formula (4) or (5).

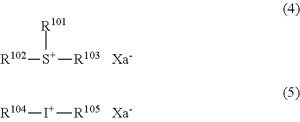

In the general formulae (4) and (5), R¹⁰¹ to R¹⁰⁵ each independently represent a hydrocarbyl group having 1 to 20 carbon atoms and optionally containing a heteroatom. Furthermore, any two of R¹⁰¹, R¹⁰², and R¹⁰³ may be bonded to each other to form a ring together with the sulfur atom bonded thereto. Examples of the hydrocarbyl groups include those given as examples of the hydrocarbyl groups represented by R^{ct1} to R^{ct5} in the description of the general formulae (cation-1) and (cation-2).

Specific examples of the cation of the sulfonium salt represented by the general formula (4) include those given as examples of the sulfonium cation represented by the general formula (cation-1). Specific examples of the cation of the iodonium salt represented by the general formula (5) include those given as examples of the iodonium cation represented by the general formula (cation-2).

In the general formulae (4) and (5), Xa⁻ represents an anion of a strong acid. Examples of the anion of the strong acid include ones represented by any of the general formulae (c1-1) to (c1-5).

Furthermore, as the other photo-acid generators of the component (D), ones represented by the following general formula (6) are also favorable.

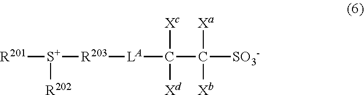

In the general formula (6), R²⁰¹ and R²⁰² each independently represent a hydrocarbyl group having 1 to 30 carbon atoms and optionally containing a heteroatom. R²⁰³ represents a hydrocarbylene group having 1 to 30 carbon atoms and optionally containing a heteroatom. In addition, any two of R²⁰¹, R²⁰², and R²⁰³ may be bonded to each other to form a ring together with a sulfur atom bonded thereto.

The hydrocarbyl groups represented by R²⁰¹ and R²⁰² having 1 to 30 carbon atoms may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, a tert-butyl group, a tert-pentyl group, an n-pentyl group, an n-hexyl group, an n-octyl group, a 2-ethylhexyl group, an n-nonyl group, and an n-decyl group; cyclic saturated hydrocarbyl groups having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups which are combinations of the groups. Furthermore, part or all of the hydrogen atoms of the hydrocarbyl groups may be substituted with a group containing a heteroatom, such as an oxygen atom, a sulfur atom, a nitrogen atom, or a halogen atom, and part of the —CH₂— of the hydrocarbyl groups may be substituted with a group containing a heteroatom such as an oxygen atom, a sulfur atom, or a nitrogen atom. The resulting hydrocarbyl groups may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

The hydrocarbylene group represented by R²⁰³ having 1 to 30 carbon atoms may be saturated or unsaturated, and may be linear, branched, or cyclic. Specific examples thereof include alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentanediyl group, a cyclohexanediyl group, a norbornanediyl group, and an adamantanediyl group; arylene groups, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group; etc. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— of the hydrocarbylene group may be substituted with a group containing a heteroatom such as an oxygen atom, a sulfur atom, or a nitrogen atom. The resulting hydrocarbylene group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. As the heteroatom, an oxygen atom is preferable.

In the general formula (6), L^{A} represents a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include ones having 1 to 20 carbon atoms among those given as examples of the hydrocarbylene group represented by R²⁰³.

In the general formula (6), X^{a}, X^{b}, X^{c}, and X^{d} each independently represent a hydrogen atom, a fluorine atom, or a trifluoromethyl group, provided that at least one of X^{a}, X^{b}, X^{c}, and X^{d} is a fluorine atom or a trifluoromethyl group.

As the photo-acid generator represented by the general formula (6), ones represented by the following general formula (6′) are preferable.

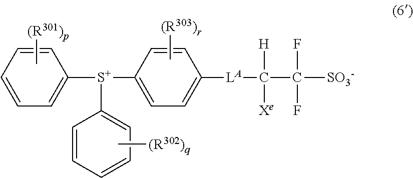

In the general formula (6′), L is as defined above. Xe represents a hydrogen atom or a trifluoromethyl group, preferably a trifluoromethyl group. R³⁰¹, R³⁰², and R³⁰³ each independently represent a hydrogen atom or a hydrocarbyl group having 1 to 20 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the general formula (c1-1-1). “p” and “q” each independently represent an integer of 0 to 5, and “r” represents an integer of 0 to 4.

Examples of the photo-acid generator represented by the general formula (6) include those given as examples of a photo-acid generator represented by a formula (2) in JP 2017-026980 A.

Among the other photo-acid generators, those containing the anion represented by the general formula (c1-1-1) or (c1-4) are particularly preferable because of low acid diffusion and excellent solubility in a solvent. A photo-acid generator represented by the general formula (6′) is also particularly preferable because the acid diffusion is very low.

When the inventive chemically-amplified resist composition contains a photo-acid generator (D), the contained amount is preferably 0.1 to 40 parts by mass, more preferably 0.5 to 20 parts by mass based on 80 parts by mass of the base resin (A). When the contained amount of the component (D) photo-acid generator is in the above-described ranges, resolution is favorable, and there is no risk of a problem of foreign substances occurring after the development of or when delaminating the resist film. Therefore, such ranges are preferable. One kind of the photo-acid generator (D) may be used, or two or more kinds thereof may be used in combination.

### [(E) Surfactant]

The inventive chemically-amplified resist composition may further contain a surfactant as a component (E). The surfactant (E) is preferably a surfactant that is insoluble or hardly soluble in water and soluble in alkaline developers, or a surfactant that is insoluble or hardly soluble in water and alkaline developers. As such a surfactant, those disclosed in JP 2010-215608 A and JP 2011-016746 A can be referred to.

As the surfactant insoluble or hardly soluble in water and alkaline developers, among the surfactants disclosed in the above-mentioned publications, preferable are FC-4430 (manufactured by 3M Company), Surflon (registered trademark) S-381 (manufactured by AGC Seimi Chemical Co., Ltd.), OLFINE (registered trademark) E1004 (manufactured by Nissin Chemical Industry Co., Ltd.), KH-20 and KH-30 (manufactured by AGC Seimi Chemical Co., Ltd.), an oxetane ring-opened polymer represented by the following general formula (surf-1), etc.

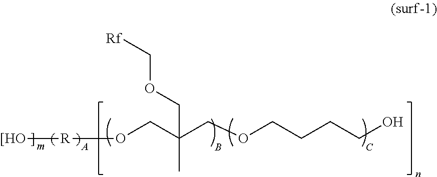

Here, R, Rf, A, B, C, “m”, and “n” apply only to the general formula (surf-1), regardless of the definitions given above. R represents an aliphatic group having a valency of 2 to 4 and having 2 to 5 carbon atoms. As the aliphatic group, examples of divalent groups include an ethylene group, a 1,4-butylene group, a 1,2-propylene group, a 2,2-dimethyl-1,3-propylene group, and a 1,5-pentylene group, and examples of trivalent or tetravalent groups include the following.

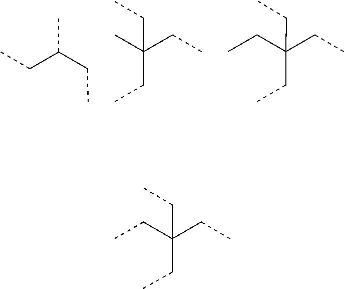

In the formulae, a broken line represents an attachment point, and each formula respectively represents a partial structure derived from glycerol, trimethylolethane, trimethylolpropane, and pentaerythritol.

Among these, a 1,4-butylene group, a 2,2-dimethyl-1,3-propylene group, etc. are preferable.

Rf represents a trifluoromethyl group or a pentafluoroethyl group, preferably a trifluoromethyl group. “m” represents an integer of 0 to 3, “n” represents an integer of 1 to 4, and the sum of “n” and “m” is the valence of R and is an integer of 2 to 4. A represents 1. B represents an integer of 2 to 25, preferably an integer of 4 to 20. C represents an integer of 0 to 10, preferably 0 or 1. Furthermore, each constitutional unit in the general formula (surf-1) does not define the arrangement, and the units may be bonded in blocks or at random. The production of the partially fluorinated oxetane ring-opened polymer-based surfactant is described in detail in U.S. Pat. No. 5,650,483 A etc.

A surfactant insoluble or hardly soluble in water and soluble in alkaline developers has a function of reducing penetration and leaching of water by being oriented on the resist film surface when a resist top coat is not used in ArF immersion lithography. Therefore, such a surfactant is useful for reducing damage to the exposure apparatus by suppressing elution of water-soluble components from the resist film, and is also useful since the surfactant is solubilized during development with an aqueous alkaline solution after exposure or after post-exposure baking (PEB) and hardly becomes a foreign substance that causes defects. Such a surfactant has a property that it is insoluble or hardly soluble in water and soluble in alkaline developers, and preferable is a polymer surfactant, in particular, one also called a hydrophobic resin and having a high water-repellency and capable of improving water-sliding property.

Specific examples of such a polymer surfactant include those including at least one kind selected from repeating units represented by any of the following general formulae (7A) to (7E).

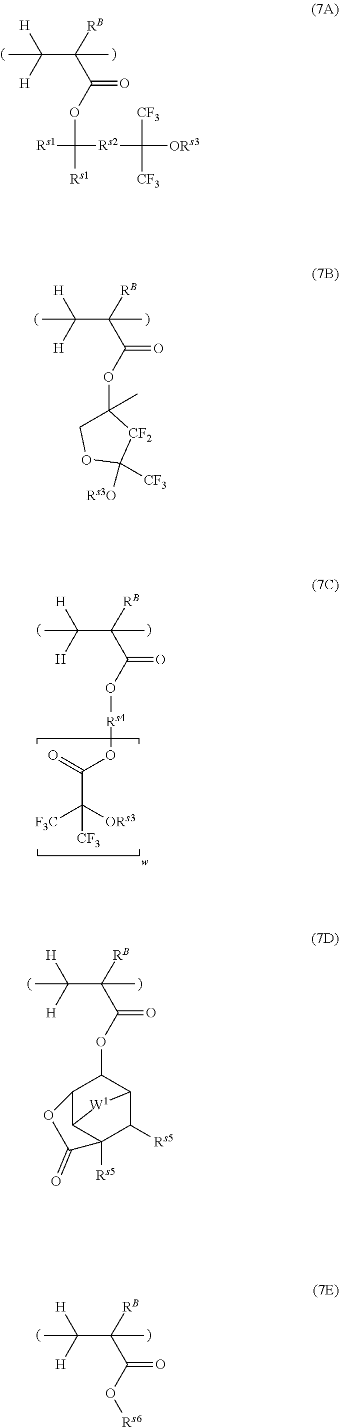

In the general formulae (7A) to (7E), R^{B} represents a hydrogen atom, a fluorine atom, a methyl group, or a trifluoromethyl group. W¹ represents —CH₂—, —CH₂CH₂—, —O—, or two —H groups that are separate from each other. Each R^{s1} independently represents a hydrogen atom or a hydrocarbyl group having 1 to 10 carbon atoms. R^{s2} represents a single bond or a linear or branched hydrocarbylene group having 1 to 5 carbon atoms. Each R^{sa} independently represents a hydrogen atom, a hydrocarbyl group or fluorinated hydrocarbyl group having 1 to 15 carbon atoms, or an acid-labile group. When R^{sa} is a hydrocarbyl group or a fluorinated hydrocarbyl group, the group may have an intervening ether bond or carbonyl group in a carbon-carbon bond. R^{s4} represents a (u+1)-valent hydrocarbon group or fluorinated hydrocarbon group having 1 to 20 carbon atoms. “u” represents an integer of 1 to 3. Each R^{s5} independently represents a hydrogen atom or a group represented by —C(═O)—O—R^{sa}. R^{sa} represents a fluorinated hydrocarbyl group having 1 to 20 carbon atoms. R^{s6} represents a hydrocarbyl group or fluorinated hydrocarbyl group having 1 to 15 carbon atoms, and optionally has an intervening ether bond or carbonyl group in a carbon-carbon bond thereof.

The hydrocarbyl group represented by R^{s1} having 1 to 10 carbon atoms is preferably a saturated hydrocarbyl group, and may be linear, branched, or cyclic. Specific examples thereof include: alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, an n-nonyl group, and an n-decyl group; and cyclic saturated hydrocarbyl groups having 3 to 10 carbon atoms, such as a cyclopropyl group, a cyclobutyl group, a cyclopentyl group, a cyclohexyl group, an adamantyl group, and a norbornyl group. Among these, groups having 1 to 6 carbon atoms are preferable.

The hydrocarbylene group represented by R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group represented by R^{s3} or R^{s6} may be saturated or unsaturated, and may be linear, branched, or cyclic. Specific examples thereof include saturated hydrocarbyl groups and aliphatic unsaturated hydrocarbyl groups, such as alkenyl groups and alkynyl groups, and saturated hydrocarbyl groups are preferable. Examples of the saturated hydrocarbyl groups include, besides those given as examples of the hydrocarbyl group represented by R^{s1}, an undecyl group, a dodecyl group, a tridecyl group, a tetradecyl group, and a pentadecyl group. Examples of the fluorinated hydrocarbyl group represented by R^{s3} or R^{s6} include groups which are the above-described hydrocarbyl groups in which part or all of the hydrogen atoms bonded to the carbon atoms of the hydrocarbyl groups are substituted with fluorine atoms. As described above, the hydrocarbyl group and the fluorinated hydrocarbyl group may have an intervening ether bond or carbonyl group in a carbon-carbon bond thereof.

Specific examples of the acid-labile group represented by R^{s3} include groups represented by the general formulae (AL-1) to (AL-3), trialkylsilyl groups in which each alkyl group has 1 to 6 carbon atoms, and oxo-group-containing alkyl groups having 4 to 20 carbon atoms.

The (u+1)-valent hydrocarbon group or fluorinated hydrocarbon group represented by R^{s4} may be linear, branched, or cyclic, and specific examples thereof include groups obtained by further “u” hydrogen atoms being removed from the above-described hydrocarbyl groups, fluorinated hydrocarbyl groups, etc.

The fluorinated hydrocarbyl group represented by R^{sa} is preferably a saturated group, and may be linear, branched, or cyclic. Specific examples thereof include groups in which part or all of the hydrogen atoms of the above hydrocarbyl group are substituted with fluorine atoms. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeating units represented by any of the general formulae (7A) to (7E) include the following, but are not limited thereto. Note that, in the following formulae, R^{B} is as defined above.

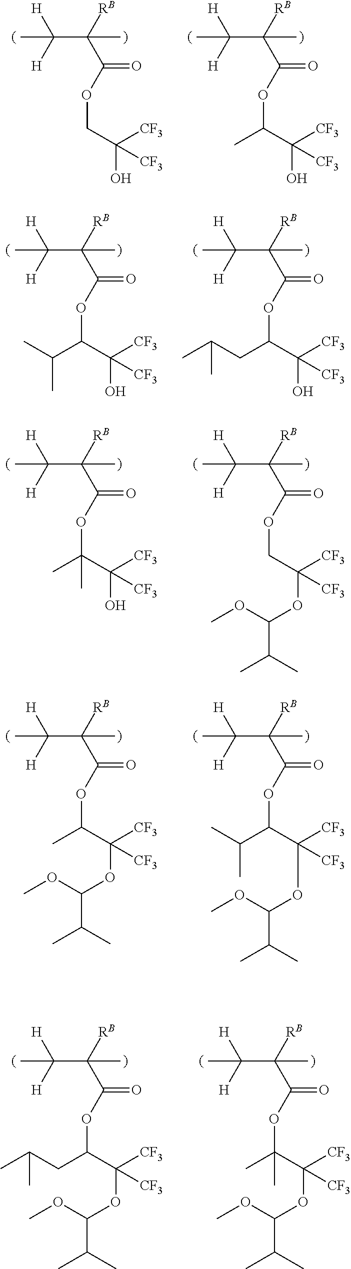

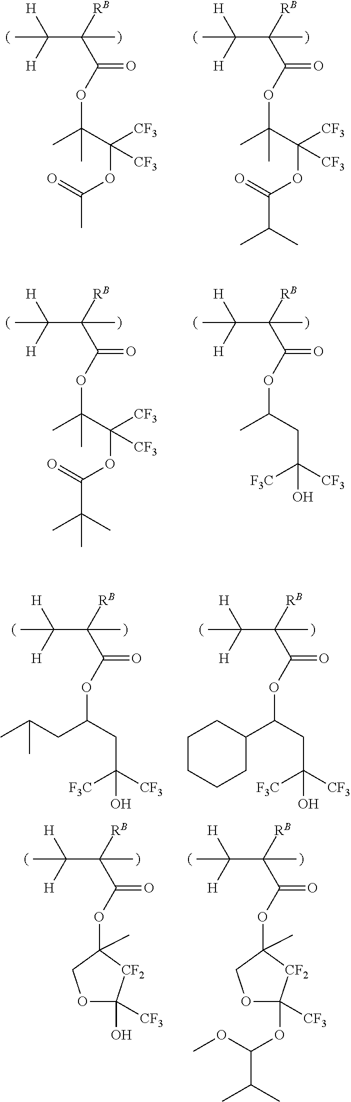

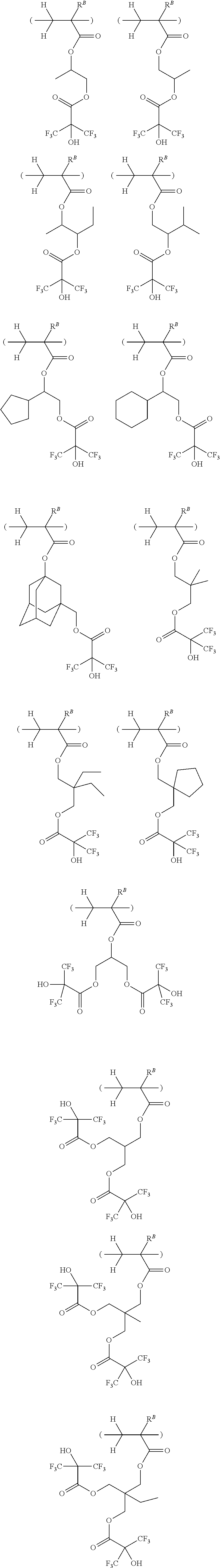

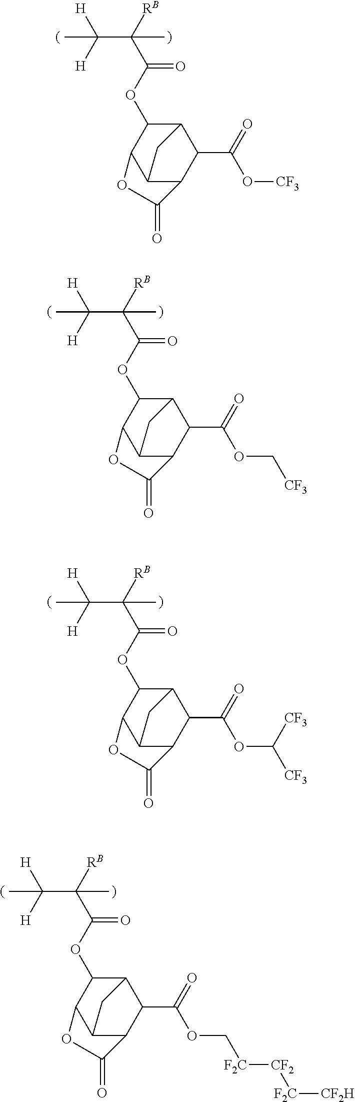

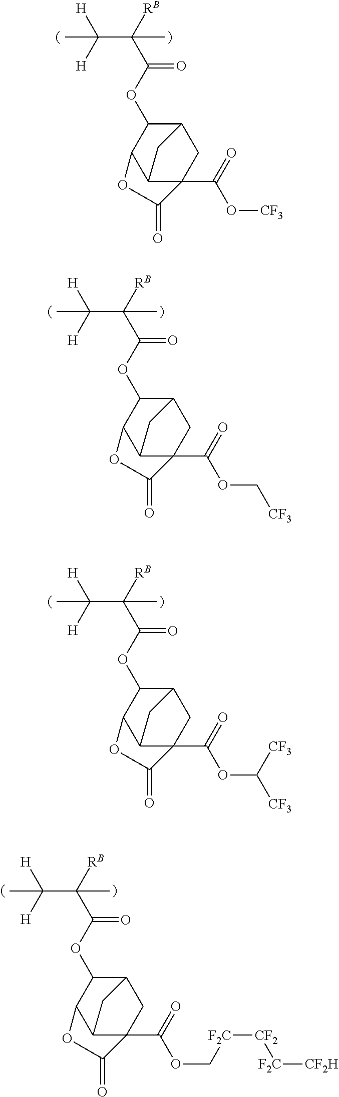

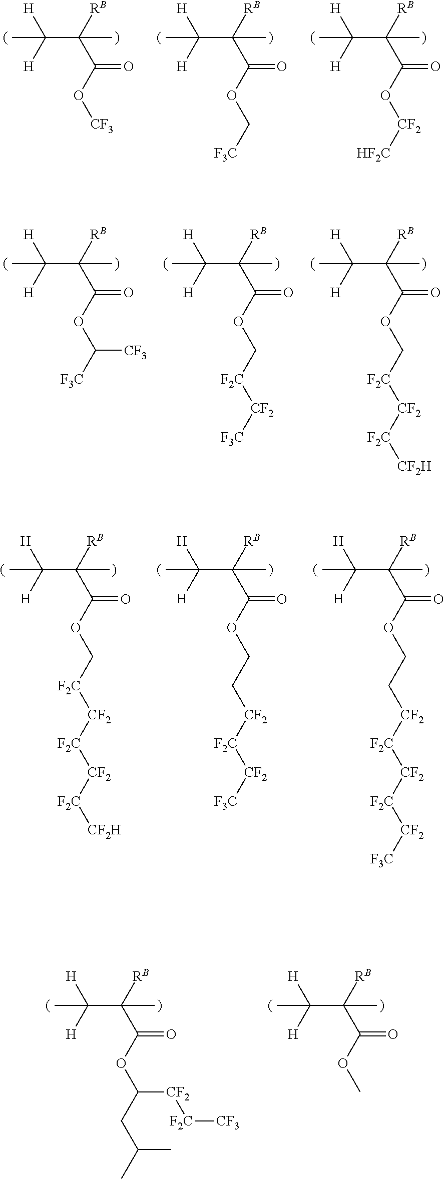

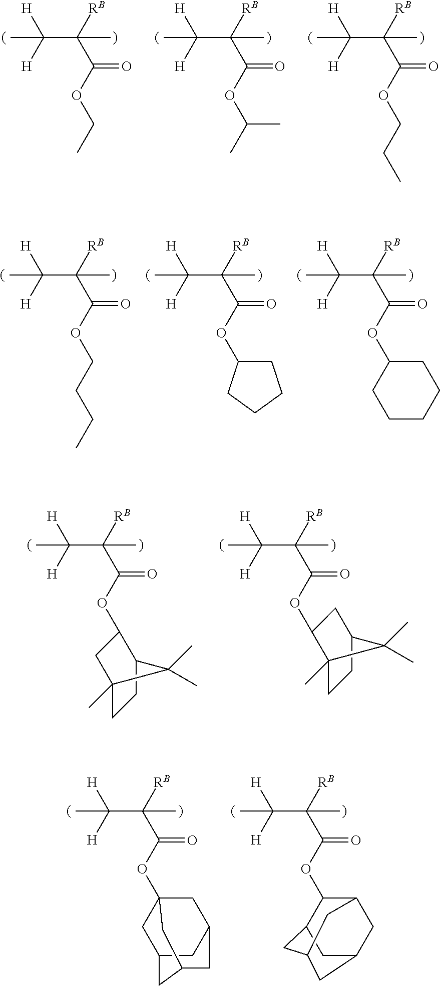

The polymer surfactant may further include a repeating unit other than the repeating unit represented by the general formulae (7A) to (7E). Examples of the other repeating unit include repeating units obtained from methacrylic acid, an α-trifluoromethylacrylic acid derivative, etc. In the polymer surfactant, the contained amount of the repeating units represented by the general formulae (7A) to (7E) is preferably 20 mol % or more, more preferably 60 mol % or more, and further preferably 100 mol % of all the repeating units.

The Mw of the polymer surfactant is preferably 1,000 to 500,000, more preferably 3,000 to 100,000. Mw/Mn is preferably 1.0 to 2.0, more preferably 1.0 to 1.6.

Examples of a method for synthesizing the polymer surfactant include a method in which, in an organic solvent, monomers having an unsaturated bond to yield the repeating unit represented by the general formulae (7A) to (7E) and, as necessary, other repeating units are added with a polymerization initiator and heated to be polymerized. Examples of the organic solvent used in the polymerization include toluene, benzene, THF, diethyl ether, and dioxane. Examples of the polymerization initiator include AIBN, 2,2′-azobis(2,4-dimethylvaleronitrile), dimethyl 2,2′-azobis(2-methylpropionate), benzoyl peroxide, and lauroyl peroxide. The reaction temperature is preferably 50 to 100° C. The reaction time is preferably 4 to 24 hours. The acid-labile group may be introduced into the monomer to be used as it is, or may be protected or partially protected after the polymerization.

When the polymer surfactant is synthesized, known chain transfer agents, such as dodecyl mercaptan and 2-mercaptoethanol may be used to regulate the molecular weight. In this case, the addition amount of these chain transfer agents is preferably 0.01 to 10 mol % relative to the total number of moles of the monomers to be polymerized.

When the inventive chemically-amplified resist composition contains the surfactant (E), the contained amount is preferably 0.1 to 50 parts by mass, more preferably 0.5 to 10 parts by mass based on 80 parts by mass of the base resin (A). When the contained amount of the surfactant (E) is 0.1 parts by mass or more, a sweepback contact angle between the resist film surface and water is sufficiently improved. When the contained amount is 50 parts by mass or less, the resist film surface has a low dissolution rate in the developer to sufficiently maintain the height of the formed fine pattern. One kind of the surfactant (E) may be used, or two or more kinds thereof may be used in combination.

### [(F) Other Components]

The inventive chemically-amplified resist composition may contain, as other components (F): a compound to be decomposed by an acid to generate an acid (acid amplifying compound); an organic acid derivative; a fluorine-substituted alcohol; a compound, having a Mw of 3,000 or less, whose solubility in a developer is changed by the action of an acid (dissolution inhibitor); etc.

As the acid amplifying compound, organic acid derivatives, fluorine-substituted alcohols, and dissolution inhibitors, compounds described in JP 2009-269953 A or JP 2010-215608 A can be referred to.

When the acid amplifying compound is contained, the contained amount is preferably 0 to 5 parts by mass, more preferably 0 to 3 parts by mass based on 80 parts by mass of the base resin (A). When the contained amount is in the above ranges, the acid diffusion can be controlled, and degradation of resolution and degradation of the pattern profile do not occur.

When the organic acid derivative is contained, the contained amount is preferably 0 to 5 parts by mass, more preferably 0 to 3 parts by mass based on 80 parts by mass of the base resin (A). When the contained amount is in the above ranges, the acid diffusion can be controlled. Therefore, such ranges are preferable.

When the fluorine-substituted alcohol is contained, the contained amount is preferably 0 to 5 parts by mass, more preferably 0 to 3 parts by mass based on 80 parts by mass of the base resin (A). When the contained amount is in the above ranges, development defects can be controlled. Therefore, such ranges are preferable.

When the dissolution inhibitor is contained, the contained amount is preferably 0 to 5 parts by mass, more preferably 0 to 3 parts by mass based on 80 parts by mass of the base resin (A). When the contained amount is in the above ranges, film loss after development can be controlled. Therefore, such ranges are preferable.

### [Patterning Process]

The inventive patterning process includes the steps of:
forming a resist film on a substrate by using the above-described chemically-amplified resist composition;
exposing the resist film by using a high-energy beam; and
developing the exposed resist film by using a developer.

As the substrate, it is possible to use, for example, a substrate (such as Si, SiO₂, SiN, SiON, TiN, WSi, BPSG, SOG, or organic antireflective film) for manufacturing an integrated circuit or a substrate (such as Cr, CrO, CrON, MoSi₂, or SiO₂) for manufacturing a mask circuit.

The resist film can be formed, for example, by applying the chemically-amplified resist composition onto a substrate by a method such as spin-coating so as to achieve a film thickness of preferably 0.05 to 2 μm, and prebaking the resultant on a hot plate preferably at 60 to 150° C. for 1 to 10 minutes, more preferably at 80 to 140° C. for 1 to 5 minutes.

Examples of the high-energy beam used for the exposure of the resist film include a KrF excimer laser beam, an ArF excimer laser beam, an electron beam (EB), and an extreme ultraviolet ray (EUV) having a wavelength of 3 to 15 nm. Regarding exposure, when a KrF excimer laser beam, an ArF excimer laser beam, or EUV is employed, the irradiation is performed while using a mask for forming a target pattern at an exposure dose of preferably 1 to 200 mJ/cm², more preferably 10 to 150 mJ/cm². When EB is employed, the irradiation is performed while using a mask for forming a target pattern or directly at an exposure dose of preferably 1 to 5000 μC/cm², more preferably 10 to 2000 μC/cm².

Incidentally, the exposure can be performed by a common exposure method, or can be performed by using an immersion method in which a liquid having a refractive index of 1.0 or more is interposed between a resist film and a projection lens. In this case, a protective film insoluble in water can also be used.

The protective film insoluble in water is used for preventing an eluted material from the resist film and for improving water-slipping property on the film surface, and roughly includes two types. One is an organic-solvent removal type, in which the protective film is required to be removed with an organic solvent not dissolving the resist film before development with an aqueous alkaline solution, and the other is an aqueous alkaline solution-soluble type, in which the protective film is soluble in an alkaline developer and is removed together with a soluble portion in the resist film. The latter is particularly preferably a material based on a polymer having a 1,1,1,3,3,3-hexafluoro-2-propanol residue, which is insoluble in water and soluble in the alkaline developer, and dissolved in an alcoholic solvent having 4 or more carbon atoms, an ether solvent having 8 to 12 carbon atoms, and a mixed solvent thereof. The protective film can also be a material in which the aforementioned surfactant insoluble or hardly soluble in water and soluble in an alkaline developer is dissolved in the alcoholic solvent having 4 or more carbon atoms, the ether solvent having 8 to 12 carbon atoms, or the mixed solvent thereof.

After the exposure, baking (PEB) may be performed as necessary. PEB can be performed, for example, by heating on a hot plate preferably at 60 to 200° C. for 1 to 5 minutes, more preferably at 80 to 180° C. for 1 to 3 minutes.

Regarding development, for example, using a developer of preferably 0.1 to 5 mass %, more preferably 2 to 3 mass %, aqueous alkaline solution, such as tetramethylammonium hydroxide (TMAH), development can be performed for preferably 0.1 to 3 minutes, more preferably 0.5 to 2 minutes, by a conventional technique, such as a dip, puddle, or spray method. Thus, exposed portions are dissolved, and the target pattern is formed on the substrate.

Furthermore, after forming the resist film, extraction of an acid generator or the like from the film surface or washing off of particles may be performed by rinsing with pure water, and rinsing for removing water remaining on the film after the exposure may be performed.

Furthermore, patterning may be performed by a double patterning method. Examples of the double patterning method include: a trench method including processing an underlay to a 1:3 trench pattern by a first exposure and etching, shifting the position, and forming a 1:3 trench pattern by a second exposure, thereby forming a 1:1 pattern; and a line method including processing a first underlay by a first exposure and etching to a 1:3 isolated pattern to be left, shifting the position, and processing a second underlay formed under the first underlay by a second exposure to a 1:3 isolated pattern to be left, thereby forming a half-pitch 1:1 pattern.

In the inventive patterning process, an organic solvent may be used instead of the aqueous alkaline solution as a developer to use a negative tone development method, where unexposed portions are dissolved.

The organic solvent used in the negative tone development is not particularly limited as long as the organic solvent is usable as a developer for patterning, and, for example, it is possible to use 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, phenylethyl acetate, benzyl formate, phenylethyl formate, methyl 3-phenylpropionate, benzyl propionate, 2-phenylethyl acetate, etc. One of these organic solvents can be used, or two or more thereof can be used in mixture.

### EXAMPLES

Hereinafter, the present invention will be specifically described with reference to Examples and Comparative Examples, but the present invention is not limited thereto.

### [1] Synthesis of Polymerizable Monomers

### [Example 1-1] Synthesis of Monomer A1

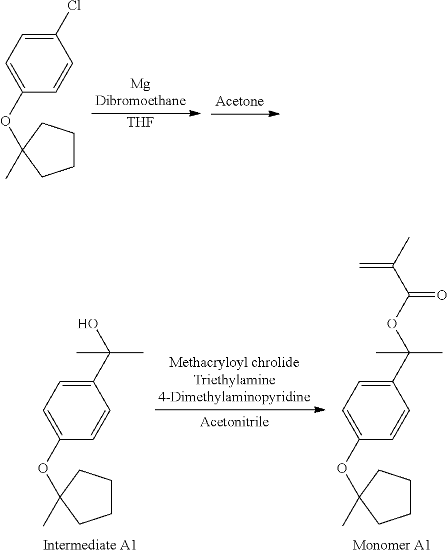 [SMILES: CC1(Oc2ccc(Cl)cc2)CCCC1 ; CC1(Oc2ccc(C(C)(C)O)cc2)CCCC1 ; C=C(C)C(=O)OC(C)(C)c1ccc(OC2(C)CCCC2)cc1 ; "THF"]

To a container containing 12.8 g of magnesium, a mixed solution of 105.4 g of 1-chloro-4-[(1-methylcyclopentyl)oxy]benzene, 0.9 g of dibromoethane, and 250 g of tetrahydrofuran (THF) was added dropwise under a nitrogen atmosphere, and then, while observing the generation of heat caused by the reaction, the temperature was adjusted so as to maintain an internal temperature of 60° C. or higher. Thus, a Grignard reagent was prepared. After that, acetone was added dropwise thereto under ice-cooling, and then the mixture was aged at 50° C. for 2 hours. The reaction solution was quenched with dilute hydrochloric acid under ice-cooling, and 250 g of n-hexane was added to extract the organic layer.

The obtained organic layer was washed with ultrapure water, and then concentrated under reduced pressure. The obtained residue was purified by distillation under reduced pressure to obtain 88.0 g of intermediate A1 (distillation temperature 109° C./15 Pa, 75% yield).

Subsequently, to the obtained intermediate A1, 68.3 g of triethylamine, 4.6 g of 4-dimethylaminopyridine, and 200 g of acetonitrile were added, and to this mixed solution, 54.9 g of methacryloyl chloride was added dropwise under room temperature. The reaction solution was aged for 12 hours, then the reaction solution was quenched with dilute hydrochloric acid, and then 200 g of toluene was added to extract the organic layer. The obtained organic layer was washed with ultrapure water, and then concentrated under reduced pressure to obtain 104.5 g of the target product, monomer A1, as an oil (92.1% yield).

### [Examples 1-2 to 1-7] Synthesis of Monomer A2 to Monomer A7

Monomer A2 to monomer A7, being polymerizable monomers, were synthesized using corresponding raw materials and by using various organic synthesis reactions. The structures of monomer A2 to monomer A7 are shown below.

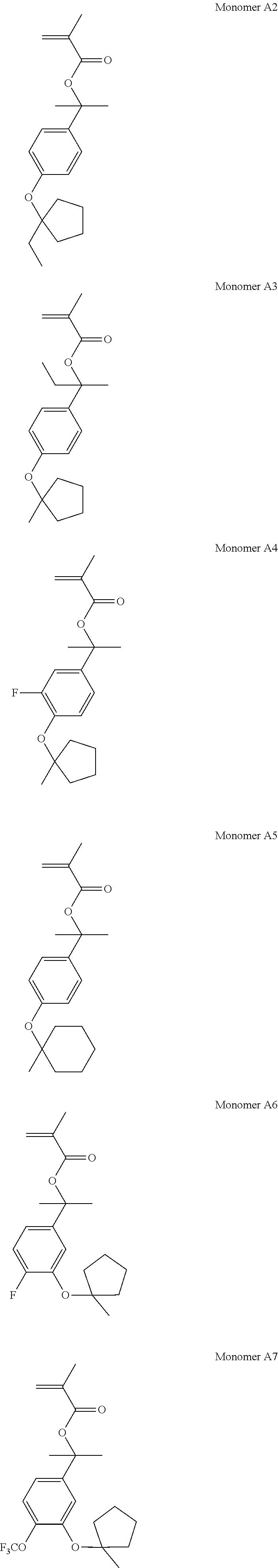 [SMILES: C=C(C)C(=O)OC(C)(C)c1ccc(OC2(CC)CCCC2)cc1 ; C=C(C)C(=O)OC(C)(CC)c1ccc(OC2(C)CCCC2)cc1 ; C=C(C)C(=O)OC(C)(C)c1ccc(OC2(C)CCCC2)c(F)c1 ; C=C(C)C(=O)OC(C)(C)c1ccc(OC2(C)CCCCC2)cc1 ; C=C(C)C(=O)OC(C)(C)c1ccc(F)c(OC2(C)CCCC2)c1 ; C=C(C)C(=O)OC(C)(C)c1ccc(OC(F)(F)F)c(OC2(C)CCCC2)c1]

### [2] Synthesis of Polymers

Among the monomers used for synthesizing polymers, monomers other than monomer A1 to monomer A7 are as follows.

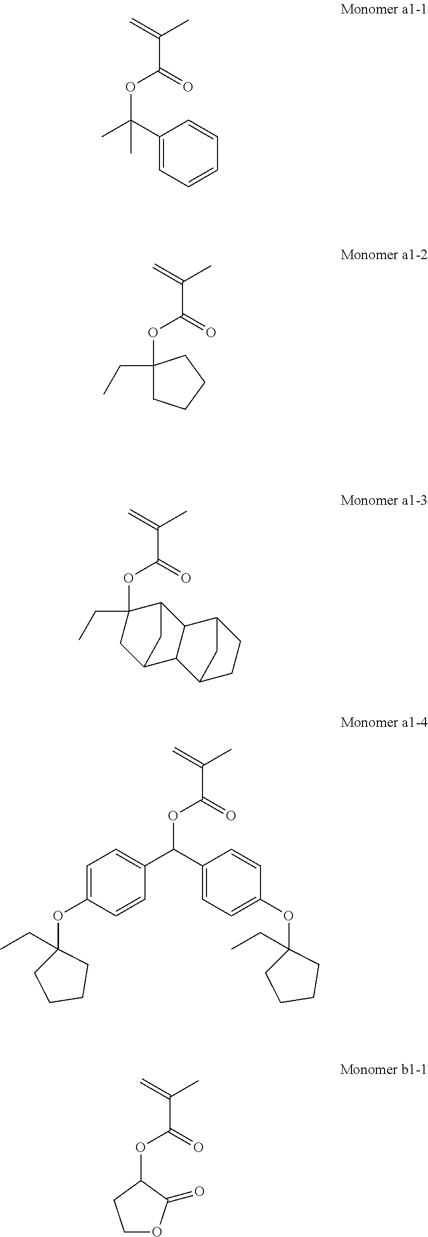 [SMILES: C=C(C)C(=O)OC(C)(C)c1ccccc1 ; C=C(C)C(=O)OC1(CC)CCCC1 ; C=C(C)C(=O)OC1(CC)CC2CC1C1C3CCC(C3)C21 ; C=C(C)C(=O)OC(c1ccc(OC2(CC)CCCC2)cc1)c1ccc(OC2(CC)CCCC2)cc1 ; C=C(C)C(=O)OC1CCOC1=O]

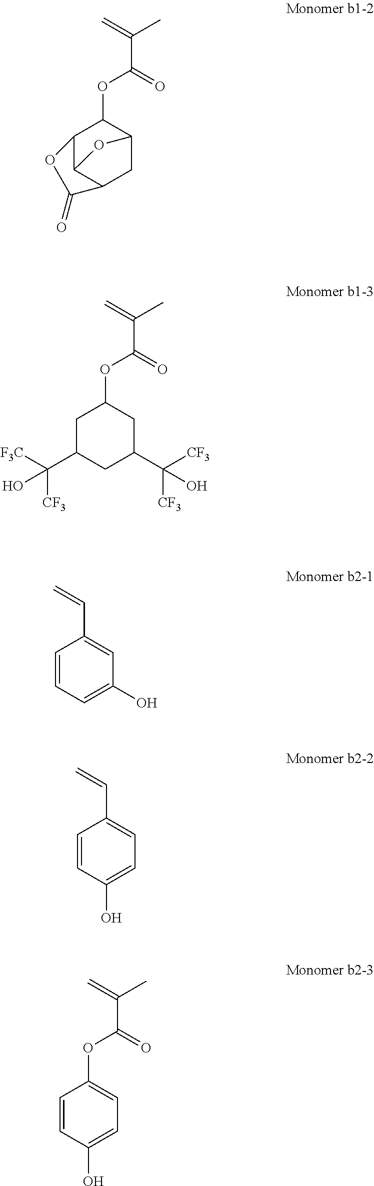

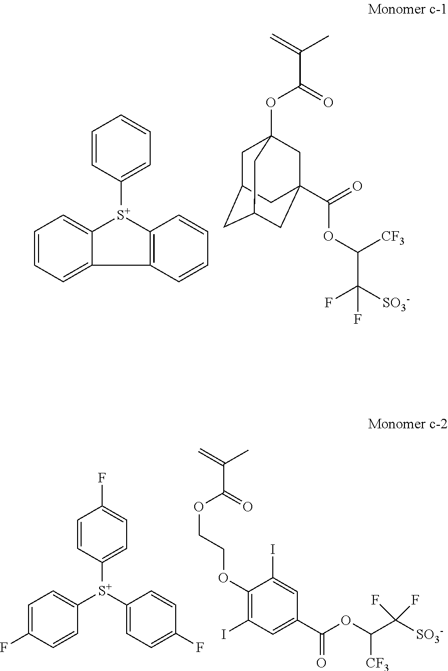

### [Example 2-1] Synthesis of Polymer P-1

Under a nitrogen atmosphere, monomer A1 (89.2 g), monomer b2-1 (24.8 g), monomer c-1 (38.0 g), 3.96 g of V-601 (manufactured by Wako Pure Chemical Industries, Ltd.), and 127 g of MEK were added into a flask to prepare a monomer-polymerization initiator solution. Into another flask with a nitrogen atmosphere, 46 g of MEK was added and heated to 80° C. with stirring, and then the monomer-polymerization initiator solution was added dropwise over 4 hours. After the dropwise addition, the polymerization liquid was further stirred for 2 hours with maintaining the temperature at 80° C., and then cooled to room temperature. The obtained polymerization liquid was added dropwise to 2,000 g of vigorously stirred hexane, and a precipitated polymer was filtered. Furthermore, the obtained polymer was washed twice with 600 g of hexane, and then dried in vacuo at 50° C. for 20 hours to obtain a white powder polymer P-1 (146 g, 96% yield). Polymer P-1 had Mw of 9,300 and Mw/Mn of 1.79. The Mw is a polystyrene-converted measurement value obtained by GPC using DMF as an eluent.

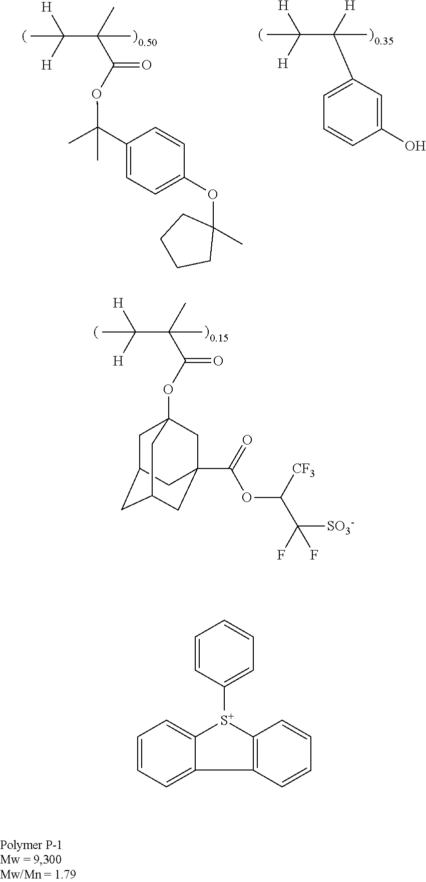

### [Examples 2-2 to 2-35 and Comparative Examples 1-1 to 1-17] Synthesis of Polymers P-2 to P-35 and Comparative Polymers CP-1 to CP-17

The polymers shown in Tables 1 and 2 were synthesized in the same manner as in Example 2-1 except that the kinds and blending ratios of the monomers were changed.

TABLE 1

| | | | Introduction | | Introduction | | Introduction | | Introduction | | |
|---|---|---|---|---|---|---|---|---|---|---|---|
| | | Unit | rate | Unit | rate | Unit | rate | Unit | rate | | |
| | Polymer | A | (mol %) | a | (mol %) | b | (mol %) | c | (mol %) | Mw | Mw/Mn |
| Example 2-1 | P-1 | A1 | 50 | — | — | b2-1 | 35 | c-1 | 15 | 9300 | 1.79 |
| Example 2-2 | P-2 | A1 | 50 | — | — | b2-2 | 35 | c-1 | 15 | 9600 | 1.83 |
| Example 2-3 | P-3 | A1 | 55 | — | — | b2-1 | 35 | c-1 | 10 | 9700 | 1.80 |
| Example 2-4 | P-4 | A1 | 50 | — | — | b2-1 | 35 | c-2 | 15 | 9800 | 1.77 |
| Example 2-5 | P-5 | A2 | 50 | — | — | b2-1 | 35 | c-1 | 15 | 10700 | 1.81 |
| Example 2-6 | P-6 | A2 | 50 | — | — | b2-2 | 35 | c-1 | 15 | 10700 | 1.81 |
| Example 2-7 | P-7 | A2 | 55 | — | — | b2-1 | 35 | c-1 | 10 | 9900 | 1.77 |
| Example 2-8 | P-8 | A2 | 50 | — | — | b2-1 | 35 | c-2 | 15 | 9700 | 1.84 |
| Example 2-9 | P-9 | A3 | 50 | — | — | b2-1 | 35 | c-1 | 15 | 10200 | 1.78 |
| Example 2-10 | P-10 | A3 | 50 | — | — | b2-2 | 35 | c-1 | 15 | 10000 | 1.82 |
| Example 2-11 | P-11 | A3 | 55 | — | — | b2-1 | 35 | c-1 | 10 | 9800 | 1.81 |
| Example 2-12 | P-12 | A3 | 50 | — | — | b2-1 | 35 | c-2 | 15 | 9600 | 1.79 |
| Example 2-13 | P-13 | A4 | 50 | — | — | b2-1 | 35 | c-1 | 15 | 9900 | 1.81 |
| Example 2-14 | P-14 | A4 | 50 | — | — | b2-2 | 35 | c-1 | 15 | 7300 | 1.76 |
| Example 2-15 | P-15 | A4 | 55 | — | — | b2-1 | 35 | c-1 | 10 | 7400 | 1.74 |
| Example 2-16 | P-16 | A4 | 50 | — | — | b2-1 | 35 | c-2 | 15 | 7700 | 1.78 |
| Example 2-17 | P-17 | A5 | 50 | — | — | b2-1 | 35 | c-1 | 15 | 10700 | 1.81 |
| Example 2-18 | P-18 | A5 | 50 | — | — | b2-2 | 35 | c-1 | 15 | 10700 | 1.81 |
| Example 2-19 | P-19 | A5 | 50 | — | — | b2-3 | 30 | c-1 | 10 | 10700 | 1.81 |
| Example 2-20 | P-20 | A5 | 55 | — | — | b2-1 | 35 | c-1 | 10 | 9900 | 1.77 |
| Example 2-21 | P-21 | A5 | 50 | — | — | b2-1 | 35 | c-2 | 15 | 9700 | 1.84 |
| Example 2-22 | P-22 | A6 | 50 | — | — | b2-1 | 35 | c-1 | 15 | 10200 | 1.78 |
| Example 2-23 | P-23 | A6 | 50 | — | — | b2-2 | 35 | c-1 | 15 | 10000 | 1.82 |
| Example 2-24 | P-24 | A6 | 55 | — | — | b2-1 | 35 | c-1 | 10 | 9800 | 1.81 |
| Example 2-25 | P-25 | A6 | 50 | — | — | b2-1 | 35 | c-2 | 15 | 9600 | 1.79 |
| Example 2-26 | P-26 | A7 | 50 | — | — | b2-1 | 35 | c-1 | 15 | 9900 | 1.81 |
| Example 2-27 | P-27 | A7 | 50 | — | — | b2-2 | 35 | c-1 | 15 | 8800 | 1.76 |
| Example 2-28 | P-28 | A7 | 55 | — | — | b2-1 | 35 | c-1 | 10 | 9000 | 1.74 |
| Example 2-29 | P-29 | A7 | 50 | — | — | b2-1 | 35 | c-2 | 15 | 8700 | 1.78 |
| Example 2-30 | P-30 | A1 | 30 | a1-1 | 20 | b2-1 | 35 | c-1 | 15 | 9300 | 1.79 |
| Example 2-31 | P-31 | A1 | 30 | a1-2 | 20 | b2-1 | 35 | c-1 | 15 | 9600 | 1.73 |
| Example 2-32 | P-32 | A1 | 50 | — | — | b1-1 | 10 | c-1 | 10 | 8900 | 1.76 |
| | | | | | | b2-1 | 30 | | | | |
| Example 2-33 | P-33 | A1 | 50 | — | — | b1-2 | 10 | c-1 | 10 | 9100 | 1.79 |
| | | | | | | b2-1 | 30 | | | | |
| Example 2-34 | P-34 | A1 | 50 | — | — | b1-3 | 10 | c-1 | 10 | 8800 | 1.75 |
| | | | | | | b2-1 | 30 | | | | |
| Example 2-35 | P-35 | A1 | 60 | — | — | b2-1 | 40 | — | — | 8600 | 1.73 |

TABLE 2

| | | | Introduction | | Introduction | | Introduction | | Introduction | | |
|---|---|---|---|---|---|---|---|---|---|---|---|
| | | Unit | rate | Unit | rate | Unit | rate | Unit | rate | | |
| | Polymer | A | (mol %) | a | (mol %) | b | (mol %) | c | (mol %) | Mw | Mw/Mn |
| Comparative | CP-1 | — | — | a1-1 | 50 | b2-1 | 35 | c-1 | 15 | 9500 | 1.81 |
| Example 1-1 | | | | | | | | | | | |
| Comparative | CP-2 | — | — | a1-1 | 50 | b2-2 | 35 | c-1 | 15 | 9800 | 1.84 |
| Example 1-2 | | | | | | | | | | | |
| Comparative | CP-3 | — | — | a1-1 | 55 | b2-1 | 35 | c-1 | 10 | 9499 | 1.83 |
| Example 1-3 | | | | | | | | | | | |
| Comparative | CP-4 | — | — | a1-1 | 50 | b2-1 | 35 | c-2 | 15 | 10000 | 1.77 |
| Example 1-4 | | | | | | | | | | | |
| Comparative | CP-5 | — | — | a1-2 | 50 | b2-1 | 35 | c-1 | 15 | 9900 | 1.79 |
| Example 1-5 | | | | | | | | | | | |
| Comparative | CP-6 | — | — | a1-2 | 50 | b2-2 | 35 | c-1 | 15 | 10500 | 1.80 |
| Example 1-6 | | | | | | | | | | | |
| Comparative | CP-7 | — | — | a1-2 | 55 | b2-1 | 35 | c-1 | 10 | 10100 | 1.79 |
| Example 1-7 | | | | | | | | | | | |
| Comparative | CP-8 | — | — | a1-2 | 50 | b2-1 | 35 | c-2 | 15 | 9400 | 1.84 |
| Example 1-8 | | | | | | | | | | | |
| Comparative | CP-9 | — | — | a1-3 | 50 | b2-1 | 35 | c-1 | 15 | 10100 | 1.79 |
| Example 1-9 | | | | | | | | | | | |
| Comparative | CP-10 | — | — | a1-3 | 50 | b2-2 | 35 | c-1 | 15 | 9700 | 1.80 |
| Example 1-10 | | | | | | | | | | | |
| Comparative | CP-11 | — | — | a1-3 | 55 | b2-1 | 35 | c-1 | 10 | 9300 | 1.83 |
| Example 1-11 | | | | | | | | | | | |
| Comparative | CP-12 | — | — | a1-3 | 50 | b2-1 | 35 | c-2 | 15 | 9599 | 1.79 |
| Example 1-12 | | | | | | | | | | | |
| Comparative | CP-13 | — | — | a1-4 | 50 | b2-3 | 30 | c-1 | 10 | 9600 | 1.81 |
| Example 1-13 | | | | | | | | | | | |
| Comparative | CP-14 | — | — | a1-3 | 50 | b1-1 | 10 | c-1 | 10 | 8900 | 1.77 |
| Example 1-14 | | | | | | b2-1 | 30 | | | | |
| Comparative | CP-15 | — | — | a1-3 | 50 | b1-2 | 10 | c-1 | 10 | 9000 | 1.81 |
| Example 1-15 | | | | | | b2-1 | 30 | | | | |
| Comparative | CP-16 | — | — | a1-3 | 50 | b1-3 | 10 | c-1 | 10 | 8900 | 1.78 |
| Example 1-16 | | | | | | b2-1 | 30 | | | | |
| Comparative | CP-17 | — | — | a1-3 | 55 | b2-1 | 45 | — | — | 8900 | 1.74 |
| Example 1-17 | | | | | | | | | | | |

### [3] Preparation of Chemically-Amplified Resist Compositions

### Examples 3-1 to 3-39 and Comparative Examples 2-1 to 2-21

Prepared was a solution of predetermined components selected from the polymers (P-1 to P-35) of the present invention, comparative polymers (CP-1 to CP-17), photo-acid generators (PAG-X and PAG-Y), and quenchers (SQ-1 to SQ-3 and AQ-1) at the constitution shown in the following Tables 3 to 5 by dissolving the components in a solvent containing 0.01 mass % of surfactant A (OM4NOVA Solutions Inc.). The solution was filtered with a 0.2-μm Teflon (registered trademark) type filter to prepare a chemically-amplified resist composition (R-1 to R-39 and CR-1 to CR-21).

TABLE 3

| | | | Photo-acid | | | |
|---|---|---|---|---|---|---|
| | | Polymer | generator | Quencher | Solvent 1 | Solvent 2 |
| | Resist | (parts by | (parts by | (parts by | (parts by | (parts by |
| | composition | mass) | mass) | mass) | mass) | mass) |
| Example 3-1 | R-1 | P-1 | — | SQ-1 | PGMEA | DAA |
| | | (80) | | (7.3) | (2200) | (900) |
| Example 3-2 | R-2 | P-2 | — | SQ-1 | PGMEA | DAA |
| | | (80) | | (7.3) | (2200) | (900) |
| Example 3-3 | R-3 | P-3 | — | SQ-1 | PGMEA | DAA |
| | | (80) | | (7.3) | (2200) | (900) |
| Example 3-4 | R-4 | P-4 | — | SQ-1 | PGMEA | DAA |
| | | (80) | | (7.3) | (2200) | (900) |
| Example 3-5 | R-5 | P-5 | — | SQ-1 | PGMEA | DAA |
| | | (80) | | (7.3) | (2200) | (900) |
| Example 3-6 | R-6 | P-6 | — | SQ-1 | PGMEA | DAA |
| | | (80) | | (7.3) | (2200) | (900) |
| Example 3-7 | R-7 | P-7 | — | SQ-1 | PGMEA | DAA |
| | | (80) | | (7.3) | (2200) | (900) |
| Example 3-8 | R-8 | P-8 | — | SQ-1 | PGMEA | DAA |
| | | (80) | | (7.3) | (2200) | (900) |
| Example 3-9 | R-9 | P-9 | — | SQ-1 | PGMEA | DAA |
| | | (80) | | (7.3) | (2200) | (900) |
| Example 3-10 | R-10 | P-10 | — | SQ-1 | PGMEA | DAA |
| | | (80) | | (7.3) | (2200) | (900) |
| Example 3-11 | R-11 | P-11 | — | SQ-1 | PGMEA | DAA |
| | | (80) | | (7.3) | (2200) | (900) |
| Example 3-12 | R-12 | P-12 | — | SQ-1 | PGMEA | DAA |
| | | (80) | | (7.3) | (2200) | (900) |
| Example 3-13 | R-13 | P-13 | — | SQ-1 | PGMEA | DAA |
| | | (80) | | (7.3) | (2200) | (900) |
| Example 3-14 | R-14 | P-14 | — | SQ-1 | PGMEA | DAA |
| | | (80) | | (7.3) | (2200) | (900) |
| Example 3-15 | R-15 | P-15 | — | SQ-1 | PGMEA | DAA |
| | | (80) | | (7.3) | (2200) | (900) |
| Example 3-16 | R-16 | P-16 | — | SQ-1 | PGMEA | DAA |
| | | (80) | | (7.3) | (2200) | (900) |
| Example 3-17 | R-17 | P-17 | — | SQ-1 | PGMEA | DAA |
| | | (80) | | (7.3) | (2200) | (900) |
| Example 3-18 | R-18 | P-18 | — | SQ-1 | PGMEA | DAA |
| | | (80) | | (7.3) | (2200) | (900) |
| Example 3-19 | R-19 | P-19 | — | SQ-1 | PGMEA | DAA |
| | | (80) | | (7.3) | (2200) | (900) |
| Example 3-20 | R-20 | P-20 | — | SQ-1 | PGMEA | DAA |
| | | (80) | | (7.3) | (2200) | (900) |

TABLE 4

| | | | Photo-acid | | | |
|---|---|---|---|---|---|---|
| | | Polymer | generator | Quencher | Solvent 1 | Solvent 2 |
| | Resist | (parts by | (parts by | (parts by | (parts by | (parts by |
| | composition | mass) | mass) | mass) | mass) | mass) |
| Example 3-21 | R-21 | P-21 | — | SQ-1 | PGMEA | DAA |
| | | (80) | | (7.3) | (2200) | (900) |
| Example 3-22 | R-22 | P-22 | — | SQ-1 | PGMEA | DAA |
| | | (80) | | (7.3) | (2200) | (900) |
| Example 3-23 | R-23 | P-23 | — | SQ-1 | PGMEA | DAA |
| | | (80) | | (7.3) | (2200) | (900) |
| Example 3-24 | R-24 | P-24 | — | SQ-1 | PGMEA | DAA |
| | | (80) | | (7.3) | (2200) | (900) |
| Example 3-25 | R-25 | P-25 | — | SQ-1 | PGMEA | DAA |
| | | (80) | | (7.3) | (2200) | (900) |
| Example 3-26 | R-26 | P-26 | — | SQ-1 | PGMEA | DAA |
| | | (80) | | (7.3) | (2200) | (900) |
| Example 3-27 | R-27 | P-27 | — | SQ-1 | PGMEA | DAA |
| | | (80) | | (7.3) | (2200) | (900) |
| Example 3-28 | R-28 | P-28 | — | SQ-1 | PGMEA | DAA |
| | | (80) | | (7.3) | (2200) | (900) |
| Example 3-29 | R-29 | P-29 | — | SQ-1 | PGMEA | DAA |
| | | (80) | | (7.3) | (2200) | (900) |
| Example 3-30 | R-30 | P-1 | — | SQ-2 | PGMEA | DAA |
| | | (80) | | (14.9) | (2200) | (900) |
| Example 3-31 | R-31 | P-1 | — | SQ-3 | PGMEA | DAA |
| | | (80) | | (6.2) | (2200) | (900) |
| Example 3-32 | R-32 | P-1 | — | AQ-1 | PGMEA | DAA |
| | | (80) | | (1.9) | (2200) | (900) |
| Example 3-33 | R-33 | P-30 | — | SQ-1 | PGMEA | DAA |
| | | (80) | | (7.3) | (2200) | (900) |
| Example 3-34 | R-34 | P-31 | — | SQ-1 | PGMEA | DAA |
| | | (80) | | (7.3) | (2200) | (900) |
| Example 3-35 | R-35 | P-32 | — | SQ-1 | PGMEA | DAA |
| | | (80) | | (7.3) | (2200) | (900) |
| Example 3-36 | R-36 | P-33 | — | SQ-1 | PGMEA | DAA |
| | | (80) | | (7.3) | (2200) | (900) |
| Example 3-37 | R-37 | P-34 | — | SQ-1 | PGMEA | DAA |
| | | (80) | | (7.3) | (2200) | (900) |
| Example 3-38 | R-38 | P-35 | PAG-X | SQ-1 | PGMEA | DAA |
| | | (80) | (28.0) | (7.3) | (2200) | (900) |
| Example 3-39 | R-39 | P-35 | PAG-Y | SQ-1 | PGMEA | DAA |
| | | (80) | (29.6) | (7.3) | (2200) | (900) |

TABLE 5

| | | | Photo-acid | | | |
|---|---|---|---|---|---|---|
| | | Polymer | generator | Quencher | Solvent 1 | Solvent 2 |
| | Resist | (parts by | (parts by | (parts by | (parts by | (parts by |
| | composition | mass) | mass) | mass) | mass) | mass) |
| Comparative | CR-1 | CP-1 | — | SQ-1 | PGMEA | DAA |
| Example 2-1 | | (80) | | (7.3) | (2200) | (900) |
| Comparative | CR-2 | CP-2 | — | SQ-1 | PGMEA | DAA |
| Example 2-2 | | (80) | | (7.3) | (2200) | (900) |
| Comparative | CR-3 | CP-3 | — | SQ-1 | PGMEA | DAA |
| Example 2-3 | | (80) | | (7.3) | (2200) | (900) |
| Comparative | CR-4 | CP-4 | — | SQ-1 | PGMEA | DAA |
| Example 2-4 | | (80) | | (7.3) | (2200) | (900) |
| Comparative | CR-5 | CP-5 | — | SQ-1 | PGMEA | DAA |
| Example 2-5 | | (80) | | (7.3) | (2200) | (900) |
| Comparative | CR-6 | CP-6 | — | SQ-1 | PGMEA | DAA |
| Example 2-6 | | (80) | | (7.3) | (2200) | (900) |
| Comparative | CR-7 | CP-7 | — | SQ-1 | PGMEA | DAA |
| Example 2-7 | | (80) | | (7.3) | (2200) | (900) |
| Comparative | CR-8 | CP-8 | — | SQ-1 | PGMEA | DAA |
| Example 2-8 | | (80) | | (7.3) | (2200) | (900) |
| Comparative | CR-9 | CP-9 | — | SQ-1 | PGMEA | DAA |
| Example 2-9 | | (80) | | (7.3) | (2200) | (900) |
| Comparative | CR-10 | CP-10 | — | SQ-1 | PGMEA | DAA |
| Example 2-10 | | (80) | | (7.3) | (2200) | (900) |
| Comparative | CR-11 | CP-11 | — | SQ-1 | PGMEA | DAA |
| Example 2-11 | | (80) | | (7.3) | (2200) | (900) |
| Comparative | CR-12 | CP-12 | — | SQ-1 | PGMEA | DAA |
| Example 2-12 | | (80) | | (7.3) | (2200) | (900) |
| Comparative | CR-13 | CP-13 | — | SQ-1 | PGMEA | DAA |
| Example 2-13 | | (80) | | (7.3) | (2200) | (900) |
| Comparative | CR-14 | CP-1 | — | SQ-2 | PGMEA | DAA |
| Example 2-14 | | (80) | | (14.9) | (2200) | (900) |
| Comparative | CR-15 | CP-1 | — | SQ-3 | PGMEA | DAA |
| Example 2-15 | | (80) | | (6.2) | (2200) | (900) |
| Comparative | CR-16 | CP-1 | — | AQ-1 | PGMEA | DAA |
| Example 2-16 | | (80) | | (1.9) | (2200) | (900) |
| Comparative | CR-17 | CP-14 | — | SQ-1 | PGMEA | DAA |
| Example 2-17 | | (80) | | (7.3) | (2200) | (900) |
| Comparative | CR-18 | CP-15 | — | SQ-1 | PGMEA | DAA |
| Example 2-18 | | (80) | | (7.3) | (2200) | (900) |
| Comparative | CR-19 | CP-16 | — | SQ-1 | PGMEA | DAA |
| Example 2-19 | | (80) | | (7.3) | (2200) | (900) |
| Comparative | CR-20 | CP-17 | PAG-X | SQ-1 | PGMEA | DAA |
| Example 2-20 | | (80) | (28.0) | (7.3) | (2200) | (900) |
| Comparative | CR-21 | CP-17 | PAG-Y | SQ-1 | PGMEA | DAA |
| Example 2-21 | | (80) | (29.6) | (7.3) | (2200) | (900) |

In Tables 3 to 5, the components are as follows.
Solvents:
   PGMEA (propylene glycol monomethyl ether acetate)
   DAA (diacetone alcohol)
Photo-acid generators: PAG-X and PAG-Y

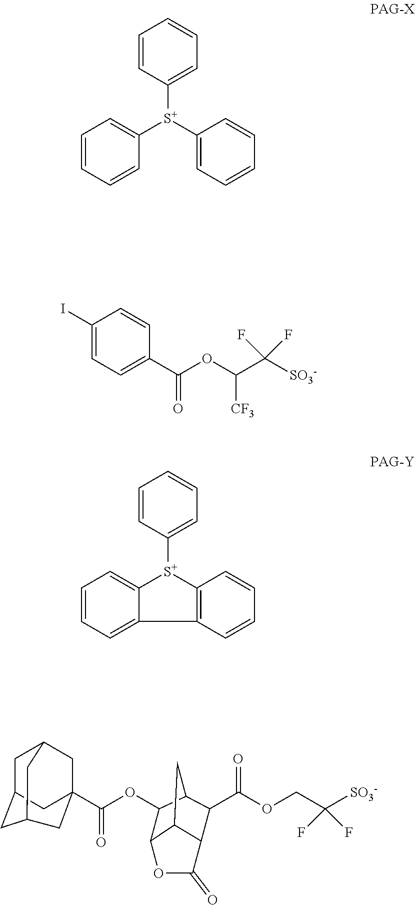

Quenchers: SQ-1 to SQ-3 and AQ-1

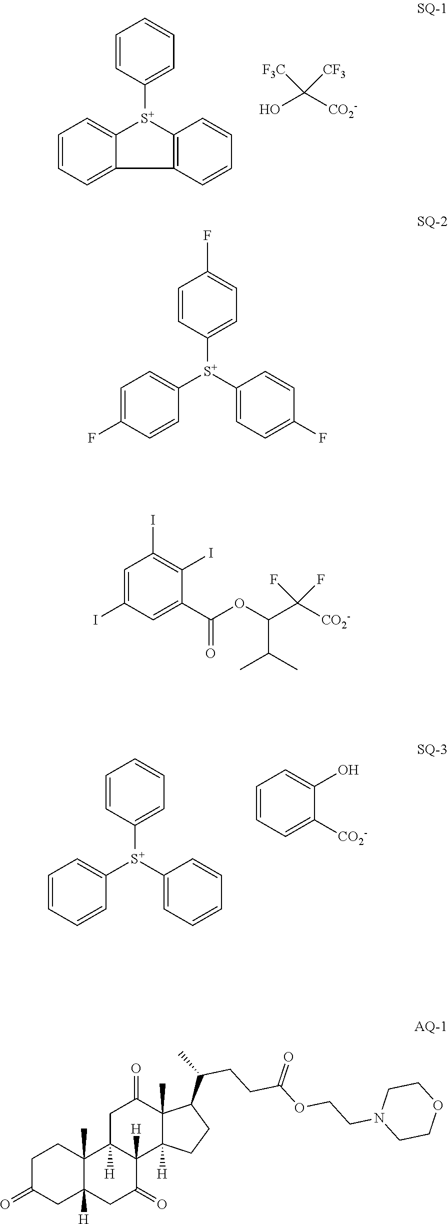

Surfactant A: copolymer of 3-methyl-3-(2,2,2-trifluoroethoxymethyl)oxetane, tetrahydrofuran, and 2,2-dimethyl-1,3-propanediol (manufactured by OMNOVA Solutions Inc.)

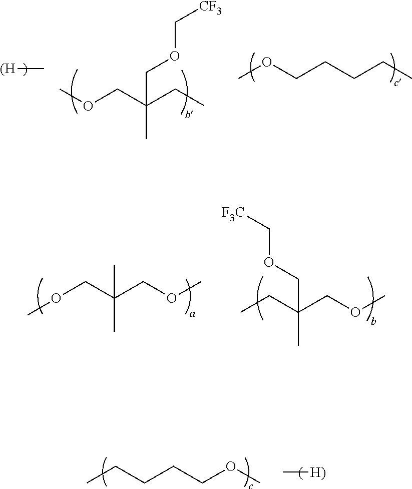

a:(b+b′):(c+c′)=1:4 to 7:0.01 to 1 (molar ratio)
Mw=1,500

### [4] EUV Lithography Evaluation

### Examples 4-1 to 4-39 and Comparative Examples 3-1 to 3-21

Each of the chemically-amplified resist compositions shown in Tables 3 to 5 was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 105° C. for 60 seconds using a hot plate to produce a resist film with 35 nm in film thickness. The resist film was exposed using an EUV scanner NXE3400 (NA 0.33, σ 0.9/0.6, 90° dipole illumination, 32 nm in pitch on wafer size, line-and-space 1:1 pattern mask) manufactured by ASML Holding N.V. Then, PEB was performed on a hot plate at a temperature shown in Tables 6 and 7 for 60 seconds. Thereafter, development was performed with a 2.38 mass % aqueous TMAH solution for 30 seconds to obtain a line-and-space pattern with a line size of 16 nm.

An exposure dose at which the line was formed with a line size of 16 nm was measured and determined as sensitivity. Furthermore, using a length-measurement SEM (CG6300), manufactured by Hitachi High-Technologies Corporation, LWR was measured. Furthermore, as the process window (PW), obtained was the size of the thickest line where no stringy bridges are formed between lines at an exposure dose smaller than the sensitivity of the resist film minus the thinnest size where no resist pattern collapse occurs and no film loss occurs at an exposure dose greater than the sensitivity of the resist film. The results are shown together in Tables 6 and 7.

TABLE 6

| | | | Optimum | | |
|---|---|---|---|---|---|
| | | PEB | exposure | | |
| | Resist | temperature | dose | LWR | PW |
| | composition | (° C.) | (mJ/cm²) | (nm) | (nm) |
| Example 4-1 | R-1 | 95 | 48 | 2.4 | 4.0 |
| Example 4-2 | R-2 | 95 | 49 | 2.3 | 4.1 |
| Example 4-3 | R-3 | 95 | 45 | 2.5 | 3.9 |
| Example 4-4 | R-4 | 95 | 44 | 2.6 | 4.1 |
| Example 4-5 | R-5 | 85 | 46 | 2.5 | 3.8 |
| Example 4-6 | R-6 | 85 | 45 | 2.6 | 3.7 |
| Example 4-7 | R-7 | 85 | 42 | 2.5 | 3.8 |
| Example 4-8 | R-8 | 85 | 40 | 2.5 | 3.9 |
| Example 4-9 | R-9 | 80 | 38 | 2.7 | 3.7 |
| Example 4-10 | R-10 | 80 | 35 | 2.8 | 3.8 |
| Example 4-11 | R-11 | 80 | 37 | 2.7 | 3.6 |
| Example 4-12 | R-12 | 80 | 37 | 2.8 | 3.8 |
| Example 4-13 | R-13 | 95 | 45 | 2.4 | 4.1 |
| Example 4-14 | R-14 | 95 | 46 | 2.5 | 4.0 |
| Example 4-15 | R-15 | 90 | 45 | 2.5 | 3.8 |
| Example 4-16 | R-16 | 90 | 44 | 2.6 | 3.9 |
| Example 4-17 | R-17 | 100 | 49 | 2.1 | 4.2 |
| Example 4-18 | R-18 | 100 | 46 | 2.2 | 4.1 |
| Example 4-19 | R-19 | 100 | 44 | 2.4 | 3.9 |
| Example 4-20 | R-20 | 100 | 45 | 2.1 | 4.2 |
| Example 4-21 | R-21 | 100 | 48 | 2.0 | 4.3 |
| Example 4-22 | R-22 | 100 | 47 | 2.2 | 4.1 |
| Example 4-23 | R-23 | 100 | 43 | 2.2 | 4.2 |
| Example 4-24 | R-24 | 100 | 43 | 2.1 | 4.2 |
| Example 4-25 | R-25 | 100 | 45 | 2.0 | 4.3 |
| Example 4-26 | R-26 | 105 | 44 | 2.1 | 4.1 |
| Example 4-27 | R-27 | 105 | 46 | 1.9 | 4.2 |
| Example 4-28 | R-28 | 105 | 42 | 2.2 | 4.1 |
| Example 4-29 | R-29 | 105 | 42 | 2.1 | 4.3 |
| Example 4-30 | R-30 | 90 | 47 | 2.4 | 3.9 |
| Example 4-31 | R-31 | 90 | 48 | 2.5 | 3.8 |
| Example 4-32 | R-32 | 90 | 49 | 2.4 | 3.9 |
| Example 4-33 | R-33 | 90 | 51 | 2.4 | 3.9 |
| Example 4-34 | R-34 | 90 | 52 | 2.5 | 3.7 |
| Example 4-35 | R-35 | 90 | 49 | 2.4 | 3.8 |
| Example 4-36 | R-36 | 95 | 54 | 2.6 | 3.6 |
| Example 4-37 | R-37 | 90 | 48 | 2.5 | 3.8 |
| Example 4-38 | R-38 | 90 | 45 | 2.6 | 3.8 |
| Example 4-39 | R-39 | 90 | 46 | 2.7 | 3.9 |

TABLE 7

| | | PEB | Optimum | | |
|---|---|---|---|---|---|
| | Resist | temper- | exposure | | |
| | compo- | ature | dose | LWR | PW |
| | sition | (° C.) | (mJ/cm²) | (nm) | (nm) |
| Comparative Example 3-1 | CR-1 | 95 | 68 | 3.0 | 0.9 |
| Comparative Example 3-2 | CR-2 | 95 | 63 | 2.9 | 0.7 |
| Comparative Example 3-3 | CR-3 | 95 | 66 | 3.1 | 0.8 |
| Comparative Example 3-4 | CR-4 | 95 | 67 | 3.2 | 0.9 |
| Comparative Example 3-5 | CR-5 | 105 | 70 | 3.2 | 0.6 |
| Comparative Example 3-6 | CR-6 | 105 | 68 | 3.1 | 0.6 |
| Comparative Example 3-7 | CR-7 | 105 | 69 | 3.0 | 0.4 |
| Comparative Example 3-8 | CR-8 | 105 | 70 | 3.1 | 0.5 |
| Comparative Example 3-9 | CR-9 | 90 | 64 | 3.3 | 0.3 |
| Comparative Example 3-10 | CR-10 | 85 | 61 | 3.2 | 0.2 |
| Comparative Example 3-11 | CR-11 | 90 | 64 | 3.4 | 0.3 |
| Comparative Example 3-12 | CR-12 | 90 | 64 | 3.4 | 0.4 |
| Comparative Example 3-13 | CR-13 | 85 | 60 | 3.6 | 0.2 |
| Comparative Example 3-14 | CR-14 | 95 | 68 | 2.9 | 0.7 |
| Comparative Example 3-15 | CR-15 | 95 | 65 | 3.0 | 0.8 |
| Comparative Example 3-16 | CR-16 | 95 | 65 | 3.1 | 0.8 |
| Comparative Example 3-17 | CR-17 | 85 | 61 | 3.5 | 0.2 |
| Comparative Example 3-18 | CR-18 | 90 | 66 | 3.4 | 0.2 |
| Comparative Example 3-19 | CR-19 | 85 | 62 | 3.2 | 0.4 |
| Comparative Example 3-20 | CR-20 | 85 | 63 | 3.6 | 0.4 |
| Comparative Example 3-21 | CR-21 | 85 | 64 | 3.5 | 0.3 |

From the results shown in Tables 6 and 7, in Examples 4-1 to 4-39, where used were R-1 to R-39, being chemically-amplified resist compositions each containing a polymer including a repeating unit obtained from the inventive polymerizable monomer, sensitivity was high, LWR was low, and the process window was wide.

On the other hand, in Comparative Examples 4-1 to 4-21, where used were CR-1 to CR-21, being chemically-amplified resist compositions each containing a polymer not including the inventive polymerizable monomer and including a repeating unit obtained from a monomer having an acid-labile group of only one kind of structure, sensitivity was low, LWR was high, and the process window was narrow.

The present description includes the following embodiments.
[1]: A polymerizable monomer having one or more of each of acid-labile groups having different structures.
[2]: The polymerizable monomer according to [1], represented by the following general formula (A1),

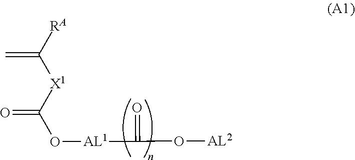

wherein R^{A} represents a hydrogen atom, a fluorine atom, a methyl group, or a trifluoromethyl group; X¹ represents a single bond or an arylene group; “n” represents 0 or 1; and AL¹ and AL² each represent an acid-labile group, provided that AL¹ and AL² each have a different structure.
[3]: The polymerizable monomer according to [2], wherein the polymerizable monomer of the general formula (A1) is represented by the following general formula (A1-1) or (A1-2),

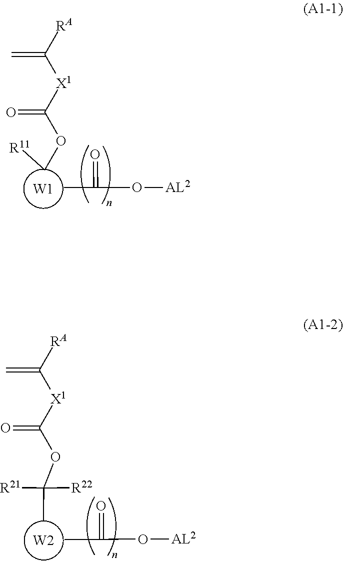

wherein R^{A}, X¹, “n”, and AL² are as defined above; R¹¹, R²¹, and R²² each independently represent a halogen atom, a hydroxy group, a nitro group, or a hydrocarbyl group having 1 to 40 carbon atoms and optionally containing a heteroatom; W1 represents an alicyclic hydrocarbon group; and W2 represents an alicyclic hydrocarbon group or an aromatic hydrocarbon group.
[4]: The polymerizable monomer according to [3], wherein the polymerizable monomer of the general formula (A1-1) is represented by either of the following general formulae (A2-1) and (A2-2), and the polymerizable monomer of the general formula (A1-2) is represented by either of the following general formulae (A2-3) and (A2-4),

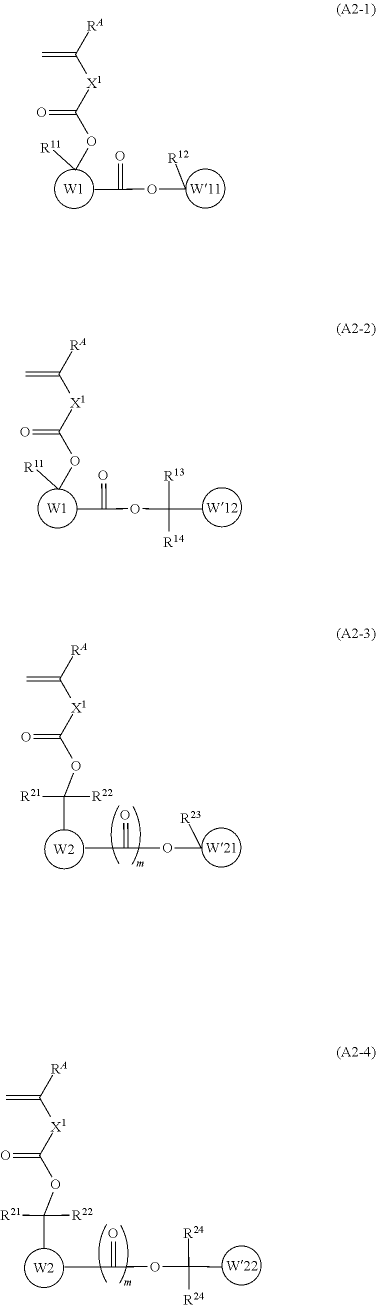

wherein R^{A}, X¹, R¹¹, R²¹, R²², W1, and W2 are as defined above; R¹², R¹³, R¹⁴, R²³, R²⁴, and R²⁵ each independently represent a halogen atom, a hydroxy group, a nitro group, or a hydrocarbyl group having 1 to 40 carbon atoms and optionally containing a heteroatom; W′11 and W′21 each independently represent an alicyclic hydrocarbon group; W′12 and W′22 each independently represent an alicyclic hydrocarbon group or an aromatic hydrocarbon group; and “m” represents 0 or 1, provided that when the W2 is an alicyclic hydrocarbon group, “m” is 1, and when the W2 is an aromatic hydrocarbon group, “m” is 0 or 1. [5]: A polymer comprising a repeating unit obtained from the polymerizable monomer according to any one of
[1] to [4]. [6]: The polymer according to [5], further comprising a repeating unit represented by the following general formula (b1),

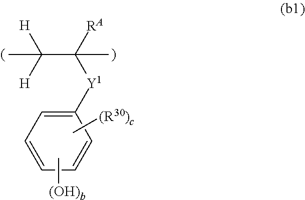

wherein R^{A} represents a hydrogen atom, a fluorine atom, a methyl group, or a trifluoromethyl group; Y¹ represents a single bond or *—C(═O)—O—; “*” represents an attachment point to the carbon atom of the main chain; R³⁰ represents a halogen atom, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and “b” represents an integer of 1 to 4 and “c” represents an integer of 0 to 4, provided that 1≤b+c≤5. [7]: The polymer according to [5] or [6], further comprising at least one selected from a repeating unit represented by the following general formula (c1), a repeating unit represented by the following general formula (c2), a repeating unit represented by the following general formula (c3), and a repeating unit represented by the following general formula (c4),

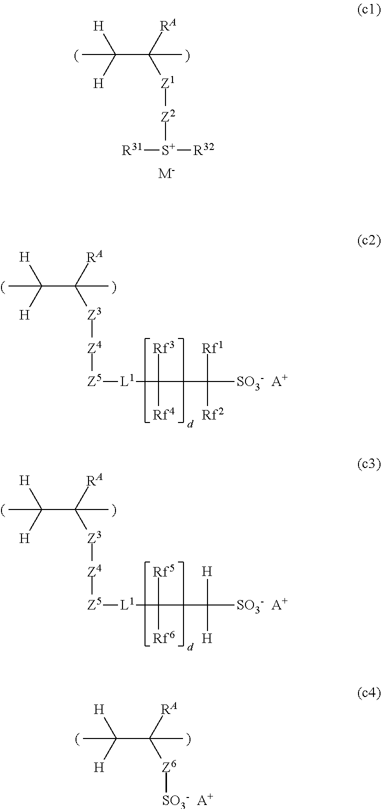

wherein R^{A} represents a hydrogen atom, a fluorine atom, a methyl group, or a trifluoromethyl group; Z¹ represents a single bond or a phenylene group; Z² represents *—C(═O)—O—Z²¹—, *—C(═O)—NH—Z²¹—, or *—O—Z²¹; Z²¹ represents an aliphatic hydrocarbylene group having 1 to 6 carbon atoms, a phenylene group, or a divalent group derived from a combination of these groups, and optionally contains a carbonyl group, an ester bond, an ether bond, or a hydroxy group; each Z³ independently represents a single bond, a phenylene group, a naphthylene group, or *—C(═O)—O—Z³¹—; Z³¹ represents an aliphatic hydrocarbylene group having 1 to 10 carbon atoms, a phenylene group, or a naphthylene group, the aliphatic hydrocarbylene group optionally containing a hydroxy group, an ether bond, an ester bond, or a lactone ring; each Z⁴ independently represents a single bond, **—Z⁴—C(═O)—O—, **—C(═O)—NH—Z⁴¹—, or **—O—Z⁴¹—; Z⁴¹ represents a hydrocarbylene group having 1 to 20 carbon atoms and optionally containing a heteroatom; each Z⁵ independently represents a single bond, ***—Z⁵¹—C(═O)—O—, ***—C(═O)—NH—Z⁵¹—, or ***—O—Z⁵¹—; Z⁵¹ represents a hydrocarbylene group having 1 to 20 carbon atoms and optionally containing a heteroatom; Z⁶ represents a single bond, a methylene group, an ethylene group, a phenylene group, a fluorinated phenylene group, a phenylene group having a substituent which is a trifluoromethyl group, *—C(═O)—O—Z⁶¹—, *—C(═O)—NH—Z⁶¹—, or *—O—Z⁶¹—; Z⁶¹ represents an aliphatic hydrocarbylene group having 1 to 6 carbon atoms, a phenylene group, a fluorinated phenylene group, or a phenylene group having a substituent which is a trifluoromethyl group, and optionally contains a carbonyl group, an ester bond, an ether bond, or a hydroxy group; “*” represents an attachment point to the carbon atom of the main chain; “**” represents an attachment point to Z³; “***” represents an attachment point to Z⁴; R³¹ and R³² each independently represent a hydrocarbyl group having 1 to 20 carbon atoms and optionally containing a heteroatom, the R³¹ and the R³² optionally being bonded to each other to form a ring together with the sulfur atom bonded thereto; L¹ represents a single bond, an ether bond, an ester bond, a carbonyl group, a sulfonic acid ester bond, a carbonate bond, or a carbamate bond; Rf¹ and Rf² each independently represent a fluorine atom or a fluorinated saturated hydrocarbyl group having 1 to 6 carbon atoms; Rf³ and Rf⁴ each independently represent a hydrogen atom, a fluorine atom, or a fluorinated saturated hydrocarbyl group having 1 to 6 carbon atoms; Rf⁵ and Rf⁶ each independently represent a hydrogen atom, a fluorine atom, or a fluorinated saturated hydrocarbyl group having 1 to 6 carbon atoms, provided that not all of the Rf⁵ and the Rf⁶ are hydrogen atoms simultaneously; M⁻ represents a non-nucleophilic counter ion; A⁺ represents an onium cation; and “d” represents an integer of 0 to 3.
[8]: A chemically-amplified resist composition comprising: a base resin including the polymer according to any one of [5] to [7]; and an organic solvent.
[9]: The chemically-amplified resist composition according to [8], further comprising one or more selected from a quencher, a photo-acid generator, and a surfactant.
[10]: A patterning process comprising the steps of:
   forming a resist film on a substrate by using the chemically-amplified resist composition according to [8] or [9];
   exposing the resist film by using a high-energy beam; and
   developing the exposed resist film by using a developer.
[11]: The patterning process according to [10], wherein the high-energy beam is a KrF excimer laser beam, an ArF excimer laser beam, an electron beam, or an extreme ultraviolet ray having a wavelength of 3 to 15 nm.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A polymerizable monomer having one or more of each of acid-labile groups having different structures.

2. The polymerizable monomer according to claim 1, represented by the following general formula (A1), 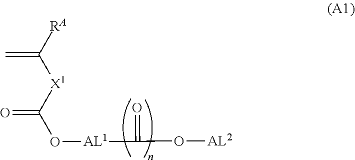
wherein R^{A} represents a hydrogen atom, a fluorine atom, a methyl group, or a trifluoromethyl group; X¹ represents a single bond or an arylene group; “n” represents 0 or 1; and AL¹ and AL² each represent an acid-labile group, provided that AL¹ and AL² each have a different structure.

3. The polymerizable monomer according to claim 2, wherein the polymerizable monomer of the general formula (A1) is represented by the following general formula (A1-1) or (A1-2), 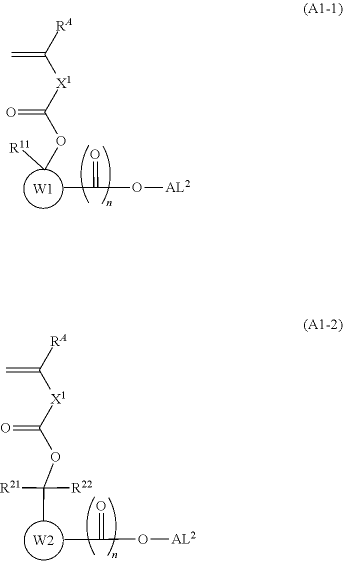
wherein R^{A}, X¹, “n”, and AL² are as defined above; R¹¹, R²¹, and R²² each independently represent a halogen atom, a hydroxy group, a nitro group, or a hydrocarbyl group having 1 to 40 carbon atoms and optionally containing a heteroatom; W1 represents an alicyclic hydrocarbon group; and W2 represents an alicyclic hydrocarbon group or an aromatic hydrocarbon group.

4. The polymerizable monomer according to claim 3, wherein the polymerizable monomer of the general formula (A1-1) is represented by either of the following general formulae (A2-1) and (A2-2), and the polymerizable monomer of the general formula (A1-2) is represented by either of the following general formulae (A2-3) and (A2-4), 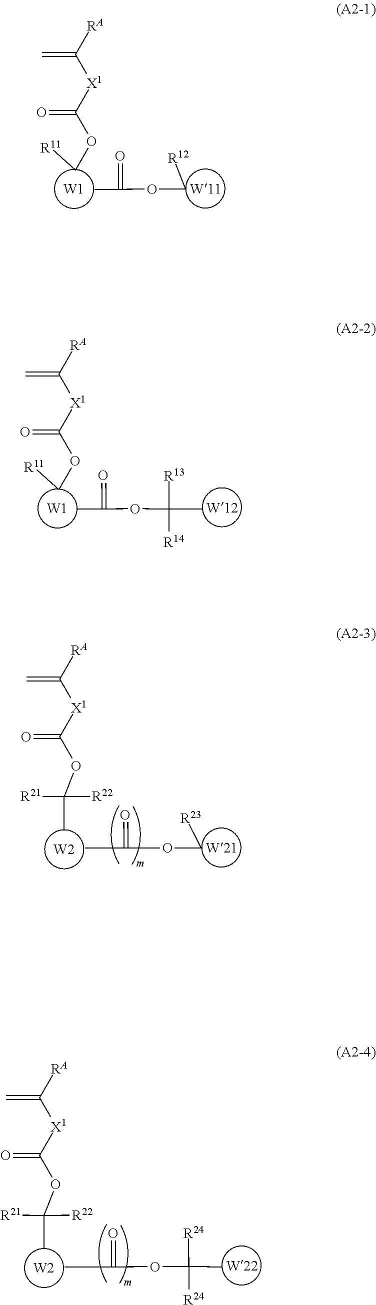
wherein R^{A}, X¹, R¹¹, R²¹, R²², W1, and W2 are as defined above; R¹², R¹³, R¹⁴, R²³, R²⁴, and R²⁵ each independently represent a halogen atom, a hydroxy group, a nitro group, or a hydrocarbyl group having 1 to 40 carbon atoms and optionally containing a heteroatom; W′11 and W′21 each independently represent an alicyclic hydrocarbon group; W′12 and W′22 each independently represent an alicyclic hydrocarbon group or an aromatic hydrocarbon group; and “m” represents 0 or 1, provided that when the W2 is an alicyclic hydrocarbon group, “m” is 1, and when the W2 is an aromatic hydrocarbon group, “m” is 0 or 1.

5. A polymer comprising a repeating unit obtained from the polymerizable monomer according to claim 1.

6. The polymer according to claim 5, further comprising a repeating unit represented by the following general formula (b1), 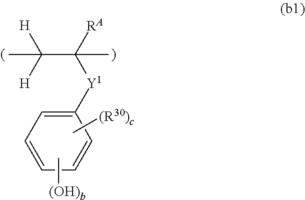
wherein R^{A} represents a hydrogen atom, a fluorine atom, a methyl group, or a trifluoromethyl group; Y¹ represents a single bond or *—C(═O)—O—; “*” represents an attachment point to the carbon atom of the main chain; R³⁰ represents a halogen atom, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and “b” represents an integer of 1 to 4 and “c” represents an integer of 0 to 4, provided that 1≤b+c≤5.

7. The polymer according to claim 5, further comprising at least one selected from a repeating unit represented by the following general formula (c1), a repeating unit represented by the following general formula (c2), a repeating unit represented by the following general formula (c3), and a repeating unit represented by the following general formula (c4), 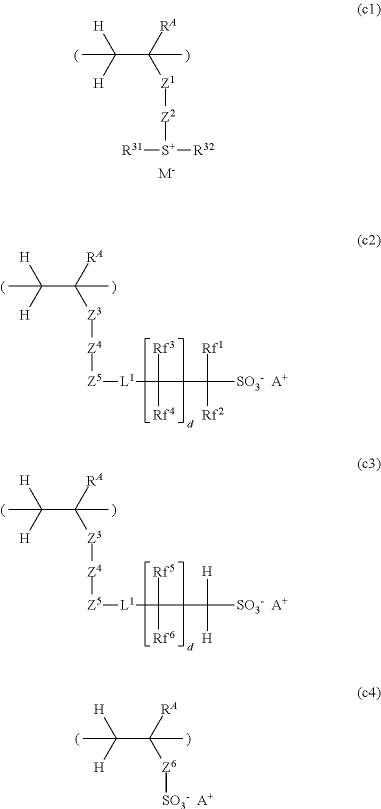
wherein R^{A} represents a hydrogen atom, a fluorine atom, a methyl group, or a trifluoromethyl group; Z¹ represents a single bond or a phenylene group; Z² represents *—C(═O)—O—Z²¹—, *—C(═O)—NH—Z²¹—, or *—O—Z²¹; Z²¹ represents an aliphatic hydrocarbylene group having 1 to 6 carbon atoms, a phenylene group, or a divalent group derived from a combination of these groups, and optionally contains a carbonyl group, an ester bond, an ether bond, or a hydroxy group; each Z³ independently represents a single bond, a phenylene group, a naphthylene group, or *—C(═O)—O—Z³¹—; Z³¹ represents an aliphatic hydrocarbylene group having 1 to 10 carbon atoms, a phenylene group, or a naphthylene group, the aliphatic hydrocarbylene group optionally containing a hydroxy group, an ether bond, an ester bond, or a lactone ring; each Z⁴ independently represents a single bond, **—Z⁴—C(═O)—O—, **—C(═O)—NH—Z⁴¹—, or **—O—Z⁴¹—; Z⁴¹ represents a hydrocarbylene group having 1 to 20 carbon atoms and optionally containing a heteroatom; each Z⁵ independently represents a single bond, ***—Z⁵¹—C(═O)—O—, ***—C(═O)—NH—Z⁵¹—, or ***—O—Z⁵¹—; Z⁵¹ represents a hydrocarbylene group having 1 to 20 carbon atoms and optionally containing a heteroatom; Z⁶ represents a single bond, a methylene group, an ethylene group, a phenylene group, a fluorinated phenylene group, a phenylene group having a substituent which is a trifluoromethyl group, *—C(═O)—O—Z⁶¹—, *—C(═O)—NH—Z⁶¹—, or *—O—Z⁶¹—; Z⁶¹ represents an aliphatic hydrocarbylene group having 1 to 6 carbon atoms, a phenylene group, a fluorinated phenylene group, or a phenylene group having a substituent which is a trifluoromethyl group, and optionally contains a carbonyl group, an ester bond, an ether bond, or a hydroxy group; “*” represents an attachment point to the carbon atom of the main chain; “**” represents an attachment point to Z³; “***” represents an attachment point to Z⁴; R³¹ and R³² each independently represent a hydrocarbyl group having 1 to 20 carbon atoms and optionally containing a heteroatom, the R³¹ and the R³² optionally being bonded to each other to form a ring together with the sulfur atom bonded thereto; L¹ represents a single bond, an ether bond, an ester bond, a carbonyl group, a sulfonic acid ester bond, a carbonate bond, or a carbamate bond; Rf¹ and Rf² each independently represent a fluorine atom or a fluorinated saturated hydrocarbyl group having 1 to 6 carbon atoms; Rf³ and Rf⁴ each independently represent a hydrogen atom, a fluorine atom, or a fluorinated saturated hydrocarbyl group having 1 to 6 carbon atoms; Rf⁵ and Rf⁶ each independently represent a hydrogen atom, a fluorine atom, or a fluorinated saturated hydrocarbyl group having 1 to 6 carbon atoms, provided that not all of the Rf⁵ and the Rf⁶ are hydrogen atoms simultaneously; M⁻ represents a non-nucleophilic counter ion; A⁺ represents an onium cation; and “d” represents an integer of 0 to 3.

8. A chemically-amplified resist composition comprising: a base resin including the polymer according to claim 5; and an organic solvent.

9. The chemically-amplified resist composition according to claim 8, further comprising one or more selected from a quencher, a photo-acid generator, and a surfactant.

10. A patterning process comprising the steps of:
forming a resist film on a substrate by using the chemically-amplified resist composition according to claim 8;
exposing the resist film by using a high-energy beam; and
developing the exposed resist film by using a developer.

11. The patterning process according to claim 10, wherein the high-energy beam is a KrF excimer laser beam, an ArF excimer laser beam, an electron beam, or an extreme ultraviolet ray having a wavelength of 3 to 15 nm.
